(19) United States
(12) Patent Application Publication
(10) Pub. No.: US 2003/0073850 A1
(43) Pub. Date: Apr. 17, 2003

(54) 4-Imidazole derivatives of benzyl and restricted benzyl sulfonamides, sulfamides, ureas, carbamates, and amides and their use

(76) Inventors: Robert J. Altenbach, Chicago, IL (US); Michael D. Meyer, Lake Villa, IL (US); James F. Kerwin JR., Grayslake, IL (US); Albert Khilevich, Buffalo Grove, IL (US); Teodozyj Kolasa, Lake Villa, IL (US); Jeffrey Rohde, Evanston, IL (US); William A. Carroll, Evanston, IL (US); Xenia Searle, Grayslake, IL (US); Fan Yang, Highwood, IL (US)

Correspondence Address: STEVEN F. WEINSTOCK; ABBOTT LABORATORIES, 100 ABBOTT PARK ROAD, DEPT. 377/AP6A, ABBOTT PARK, IL, 60064-6008, US

(21) Appl. No.: 09/506,750

(22) Filed: Feb. 17, 2000

Related U.S. Application Data
(63) Continuation-in-part of application No. 09/364,901, filed on Jul. 29, 1999.
Application No. 09/364,901 is a continuation-in-part of application No. 09/130,799, filed on Aug. 7, 1998, now abandoned.

(51) Int. Cl. C07D 233/54; C07D 233/84

(52) U.S. Cl. 548/320.1; 548/326.5

(57) ABSTRACT

Compounds of formula I

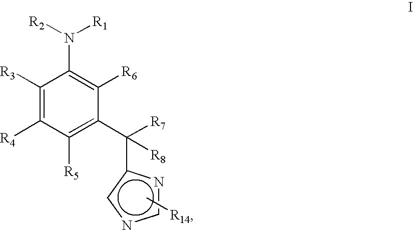

are useful in treating diseases prevented by or ameliorated with α_{1A} agonists. Also disclosed are α_{1A} agonist compositions and a method of activating α₁ adrenoceptors in a mammal.

This application is a continuation-in-part of U.S. application Ser. No. 09/364,901, filed Jul. 29, 1999, which is a continuation-in-art of U.S. Provisional application serial No. 60/095,659 filed Aug. 7, 1998, incorporated herein by reference.

### TECHNICAL FIELD

This invention relates to compounds, which are α_{1A} agonists, pharmaceutical compositions containing these compounds, and methods of treatment using these compounds.

### BACKGROUND OF THE INVENTION

Urinary stress incontinence is the involuntary loss of urine due to a stress such as coughing, sneezing, bending or lifting heavy objects. This condition may occur as a result of an unstable urethra, a loss of pelvic floor support and urethral wall defects from trauma, surgery, childbirth and neurological diseases. An agent which increases urethral pressure may be useful for the treatment of stress incontinence.

The α₁ adrenoceptor plays a part in the sympathetic maintenance of smooth muscle tone and α₁ adrenergic agonists are known to increase muscle tone in the lower urinary tract (Testa, R. Eur. J. Pharmacol. (11993), 249, 307-315). Urethral tone in the human is largely maintained by activation of postsynaptic α adrenoceptors (Anders son, K-E. Pharmacol. Rev. (1993), 45, 253). Phenylpropanolamine (Cummings, J. M. Drugs of Today (11996), 32, 609-614) and midodrine are α₁ agonists which have been used for the treatment of urinary incontinence. These agents are reported to work by increasing the tone of the smooth muscle of the bladder base and urethra (Nasu, K. Br. J. Pharmacol. (1998), 123, 1289-1293). However, these agents suffer from cardiovascular related side effects (Taniguchi, N. Eur. J. Pharmacol. (1996), 318, 117-122). Thus an agent that is effective in the treatment of urinary incontinence without cardiovascular side effects is needed.

At least 3 subtypes of the a, adrenoceptor (α_{1A}, α_{1B}, and α_{1D}) have been classified via pharmacological techniques and their corresponding molecular clones (α₁ₐ, α_{1b}, and α_{1d}) have been identified (Ford, A. P. D. W. Trends. Pharmacol. Sci. (1994), 15, 167-170; Hieble J. P. Pharmacol. Rev. (1995), 47, 267-270; Hancock, A. H. Drug Development Research (1996), 39, 54-107). Another subtype, the α_{1L}, has been proposed on the basis of pharmacological and functional studies but has not been cloned (Muramatsu, I. Pharmacol. Commun. (1995), 6,23-28; Bylund, D. B. Pharmacol. Rev. (1994), 46, 121; Graham, R. M. Circ. Res. (1996), 78, 737). It has been proposed that the α_{1L} subtype represents a particular conformational state of the α_{1A} adrenoceptor (Ford, A. P. D. W. Br. J. Pharmacol. (1997), 121, 1127).

Studies have shown that the α_{1A} adrenoceptor is present in the lower urinary tract (Testa, R. Eur. J. Pharmacol. (1993), 249, 307-315). Binding and molecular biological studies indicate that the α_{1A} subtype is the predominant cc, subtype in the lower urinary tract (Chapple, C. R. Br. J. Urol. (1994), 74, 585-589; Kawabe, K. Int. J. Urol. (1994), 1, 203-211; Moriyama, N. Jistochem. J. (1996), 28, 283-288; Nasu, K., Br. J. Pharmacol. (1996), 119, 797-803; Takahashi, H. Neurourol. Urodyn. (1996), 15, 342-343). It has been proposed that, of the three cloned α₁ subtypes, the α_{1A} subtype is most likely to be responsible for the contraction of the human urethra (Nasu, K., Br. J. Pharm. (1998), 123, 1289-1293). Other research suggests that the human urethral contractions are mediated mainly through α_{1L} adrenoceptors (Ford, A. P. D. W. Mol. Pharmacol. (1996), 49, 209-215; Nishimatsu, H. BJU International (1999), 84, 515-520). Therefore an agent which stimulates either the α_{1A} adrenoceptor or the proposed α_{1L} adrenoceptor (or both the α_{1A} and α_{1L} adrenoceptors) will lead to constriction of the lower urinary tract.

Selective stimulation of the α_{1A} adrenoceptor may result in the contraction of the bladder neck and urethra leading to an increase in intraurethral pressure without cardiovascular side effects. It is known that some α_{1A} adrenoceptor agonists may be useful for the treatment of urinary incontinence (Craig, et al., WO 96/38143). The compounds of the present invention are α_{1A} agonists that may be useful in the treatment of urinary incontinence.

The bladder neck, also know as the bladder base or trigone, can be stimulated by α agonists such as noradrenaline (Taki, N. J. of Urol. (1999), 162, 1829-1832). Agents which contract trigonal smooth muscle may have utility for treatment of ejaculation disorders (FR 2768054-A1;, WO 99/12535; FR 2768055-A1; WO 99/12536). The compounds of the present invention are α_{1A} agonists which stimulate the bladder neck and may be useful in the treatment of ejaculatory dysfunction.

The compounds of the present invention may also be useful in the treatment of nasal congestion (Proctor Pharmac. Ther. B. (1976) 2, 493-509) and septic shock (Cole, L. Blood Purif (1997) 15, 309-318).

EP 0887346 A2 discloses a group of 4-imidazole derivatives of phenyl-alkylsulfonamides as alpha_{1A/1L} adrenoceptor agonists for the treatment of urinary incontinence and nasal congestion.

WO 99/05115 discloses a group of substituted imidazole derivatives that are proposed as H₃ (histamine-3) receptor ligands potentially useful as sedatives, as sleep regulators, as anticonvulsants, as regulators of hypothalamo-hypophyseal secretion, as antidepressants, as modulators of cerebral circulation, in the treatment of asthma, in the treatment of irritable bowel syndrome and as tools in the study of the role of histamine.

WO 97/40017 discloses a group of compounds which modulate protein-tyrosine phosphatases or other molecules with tyrosine phosphonate recognition units for the treatment of type I diabetis, type II diabetis, impaired glucose tolerance, insulin resistance, obesity, immune dysfunction including autoimmunity diseases and AIDS, diseases with dysfunctions of the coagulation system, allergic diseases, osteoporosis, proliferative disorders including cancer and psoriasis, diseases with decreased or increased synthesis or effects of growth hormone, diseases with decreased or increased synthesis of hormones or cytokines that regulate the releases of/or response to growth hormone, diseases of the brain including Alzheimer's disease and schizophrenia, and infectious disease.

WO 95/14007 and U.S. Pat. No. 5,578,616 disclose a group of 4-imidazoles proposed as antagonists of the histamine H₃ receptor useful for the treatment of various allergic, inflammatory, GI-tract or cardiovascular diseases. In addition, these compounds are proposed to posses CNS activity and may be useful as sleep regulators, anticonvulsants, cognition enhancers, antidepressants, regulators of hypothalamo-hypophyseal secretions, and the like.

WO 97/36876 discloses a group of compounds which inhibit farnesyl-protein transferase and are proposed for treating or preventing cancer, neurofibromin benign proliferative disorder, retinal vascularization, infections from hepatitis delta and related viruses, polycystic kidney disease and restenosis.

WO 95/01967 discloses a group of heterocycles proposed for use as an agent in the treatment of acute and chronic neuropsychiatric disorders characterised by progressive processes that sooner or later lead to neuronal cell death and dysfunction. The compounds of the invention are proposed for the treatment of stroke, cerebral ischaemia, dysfunctions resulting from brain and/or spinal trauma, hypoxia and anoxia, multi-infarct dementia; AIDS dementia, neurodegenerative diseases, brain dysfunction in connection with surgery, and CNS dysfunctions as a result of exposure to neurotoxins or radiation.

U.S. Pat. No. 4,443,466 discloses a group of imidazoles as hypertensive agents.

U.S. Pat. No. 5,073,566, U.S. Pat. No. 5,312,936 and U.S. Pat. No. 5,571,925 discloses a group of 4-imidazole derivatives that antagonize angiotensin II for the treatment of hypertension and congestive heart failure.

U.S. Pat. No. 5,756,528 discloses a group of compounds which inhibit farnesyl-protein transferase and are proposed for the treatment of cancer. The compounds are also proposed for the treatment or prevention of a benign proliferative disorder component of NF-1, infections from hepatitis delta and related viruses, restenosis, polycystic kidney disease and fungal infections.

EP 717 037 A1 and U.S. Pat. No. 5,658,938 disclose a group of substituted 1-H-imidazoles.

Imidazole containing compounds that are α₂ adrenergic ligands are disclosed in Zhang, et. al., J. Med. Chem (1997), 40, 3014-4024.

U.S. Pat. No. 4,634,705 discloses a group of amidines as antihypertensive agents.

U.S. Pat. No. 5,610,174 discloses a method for treating urinary incontinence with a group of amidines.

WO 98/42679 discloses a group of benzenesulfonamide derivatives as smooth muscle agents and more particularly for treating stress incontinence.

WO 96/38143 discloses a method of treating urinary incontinence in a subject which comprises administering to the subject a therapeutically effective amount of an α_{1A} (previously α_{1C}) selective agonist which activates a human α_{1A} (previously α_{1C}) adrenoceptor at least ten-fold more than it activates a human α_{1D} (previously α_{1A}) and α_{1B} adrenoceptor.

FR 2768054-A1 and WO 99/12535 discloses certain sulfonamide benzene derivatives and FR 2768055-A1 and WO 99/12536 disclose certain sulfonanilide derivatives that contract trigonal smooth muscle and may have utility for treatment of ejaculation disorders.

The compounds of the present invention are structurally and pharmacologically distinct from the previously reported compounds.

### SUMMARY OF THE INVENTION

In its principle embodiment, the present invention discloses compounds having formula I:

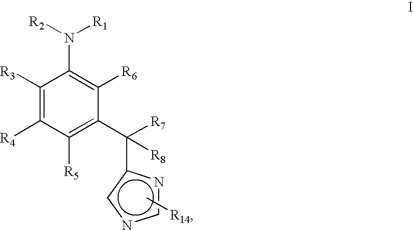

or a pharmaceutically acceptable salt thereof, wherein

R₁ is selected from —S(O)₂R₉ and —C(O)R₁₀;

R₉ is selected from alkenyl, alkyl, alkynyl, aryl, arylalkenyl, arylalkyl, cycloalkyl, cycloalkylalkyl, haloalkyl, heterocycle, and —NZ₁Z₂ wherein Z₁ and Z₂ are independently selected from hydrogen, alkyl, aryl, and arylalkyl;

R₁₀ is selected from alkenyl, alkoxy, alkyl, aryl, arylalkyl, aryloxy, cycloalkyl, cycloalkylalkyl, cycloalkyloxy, haloalkoxy, haloalkyl, and —NZ₃Z₄ wherein Z₃ and Z₄ are independently selected from hydrogen, alkoxyalkyl, alkyl, aryl, arylalkyl, and cycloalkyl, or Z₃ and Z₄ taken together with the nitrogen atom to which they are attached form a heterocycle selected from azetidin-1-yl, piperazin-1-yl, piperidin-1-yl, pyrrolidin 1-yl, and morpholin-4-yl wherein azetidin-1-yl, piperazin-1-yl, piperidin-1-yl, pyrrolidin-1-yl, and morpholin-4-yl are unsubstituted or substituted with 1 or 2 substituents independently selected from alkoxy, lower alkyl, and hydroxy;

R₂ is selected from hydrogen, lower alkyl, aryl, arylalkyl, cycloalkyl, cycloalkylalkyl, and haloalkyl;

R₃, R₄, R₅, and R₆ are independently selected from hydrogen, lower alkoxy, lower alkenyl, lower alkyl, lower haloalkyl, cycloalkyl, halo, and hydroxy; or

R₆ and R₇ together with the carbon atoms to which they are attached form a 5, 6, or 7 membered carbocyclic ring; or

R₆ and R₇ together with the carbon atoms to which they are attached form a 5 or 6 membered ring containing 1 heteroatom selected from O, NR₁₁, and S(O), wherein n is 0-2;

R₁₁ is selected from the group consisting of hydrogen, alkoxycarbonyl, alkyl, alkylcarbonyl, arylalkyl, formyl, —C(O)NZ₃Z₄, and —SO₂NZ₁Z₂;

R₈ is absent or hydrogen; or

R₇ and R₈ together form

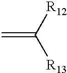

wherein R₁₂ and R₁₃ are independently selected from hydrogen, lower alkoxy, lower alkyl, aryl, arylalkyl, cycloalkyl, and cycloalkylalkyl provided that R₁ is S(O)₂R₉; or

R₁₂ and R₁₃ together with the carbon atom to which they are attached form a 3, 4, 5, 6, or 7 membered carbocyclic ring; or

R₁₂ and R₆ together with the carbon atoms to which they are attached form a 5, 6, or 7 membered carbocyclic ring provided that R₁₃ is hydrogen; or R₁₂ and R₆ together with the carbon atoms to which they are attached form a 5 or 6 membered ring containing 1 heteroatom selected from O, NR₁₁, and S(O), provided that R₁₃ is hydrogen; and

R₁₄ is selected from hydrogen and lower alkyl.

In another embodiment of the present invention, compounds have formula I wherein,

R₁ is selected from —S(O)₂R₉ and —C(O)R₁₀;

R₉ is selected from alkyl, aryl, arylalkenyl, arylalkyl, cycloalkyl, haloalkyl, heterocycle, and —NZ₁Z₂ wherein Z, and Z₂ are independently selected from hydrogen and alkyl;

R₁₀ is selected from alkoxy, alkyl, aryloxy, cycloalkyl, cycloalkyloxy, haloalkoxy, haloalkyl, and —NZ₃Z₄ wherein Z₃ and Z₄ are independently selected from hydrogen, alkoxyalkyl, alkyl, and cycloalkyl, or Z₃ and Z₄ taken together with the nitrogen atom to which they are attached form a heterocycle selected from piperidin-1-yl and morpholin-4-yl wherein piperidin-1-yl, may be unsubstituted or substituted with 1 or 2 substituents selected from lower alkyl;

R₂ is selected from hydrogen and lower alkyl;

R₃ is selected from hydrogen, lower alkoxy, lower alkyl, lower haloalkyl, halo, and hydroxy;

R₄ is selected from hydrogen, lower alkoxy, lower alkyl, lower haloalkyl, cycloalkyl, halo, and hydroxy;

R₅ is selected from hydrogen, lower alkoxy, lower alkyl, lower haloalkyl, halo, and hydroxy;

R₆ is selected from hydrogen, lower alkoxy, lower alkenyl, lower alkyl, lower haloalkyl, halo, and hydroxy; or

R₆ and R₇ together with the carbon atoms to which they are attached form a 5, 6, or 7 membered carbocyclic ring; or

R₆ and R₇ together with the carbon atoms to which they are attached form a 5 or 6 membered ring containing 1 heteroatom selected from the group consisting of O, NR₁₁, and S(O)ₙ wherein n is 0-2;

R₁₁ is selected from hydrogen, alkoxycarbonyl, alkyl, alkylcarbonyl, arylalkyl, formyl, —C(O)NZ₃Z₄ wherein Z₃ and Z₄ are as defined in formula I, and —SO₂NZ₁Z₂ wherein Z, and Z₂ are as defined in formula I;

R₈ is absent or hydrogen; or

R₇ and R₈ together form

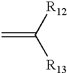

wherein R₁₂ and R₁₃ are independently selected from hydrogen, lower alkoxy, lower alkyl, aryl, arylalkyl, cycloalkyl, and cycloalkylalkyl provided that R₁ is S(O)₂R₉; or

R₁₂ and R₁₃ together with the carbon atom to which they are attached form a 3, 4, 5, 6, or 7 membered carbocyclic ring; or

R₁₂ and R₆ together with the carbon atoms to which they are attached form a 5, 6, or 7 membered carbocyclic ring provided that R₁₃ is hydrogen; or

R₁₂ and R₆ together with the carbon atoms to which they are attached form a 5 or 6 membered ring containing 1 heteroatom selected from the group consisting of O, NR₁₁, and S(O)ₙ provided that R₁₃ is hydrogen; and

R₁₄ is selected from hydrogen and lower alkyl.

In another embodiment of the present invention, compounds have formula I wherein,

R₁ is selected from —S(O)₂R₉ and —C(O)R₁₀;

R₉ is selected from alkyl, aryl wherein aryl is selected from 2-methylphenyl, 4-methylphenyl, 4-methoxyphenyl, arylalkenyl wherein arylalkenyl is 2-phenylethenyl, arylalkyl wherein arylalkyl is benzyl, cycloalkyl wherein cycloalkyl is cyclopropyl, haloalkyl, heterocycle wherein heterocycle is selected from 3,5-dimethylisoxazol-4-yl, 1-methyl-1H-imidazol-4-yl, 5-chlorothien-2-yl, 5-chloro-1,3-dimethyl-1H-pyrazol-4-yl, quinolin-8-yl, 2-(methoxycarbonyl)thien-3-yl, 4-methyl-2-(acetylamino)thiazol-5-yl, and 5-chloro-3-methyl-1-benzothien-2-yl, and —NZ₁Z₂ wherein Z, and Z₂ are independently selected from hydrogen and alkyl;

R₁₀ is selected from alkoxy, alkyl, aryloxy wherein aryloxy is 4-methylphenoxy, cycloalkyloxy wherein cycloalkyloxy is ((1R,2S,5R)-2-isopropyl-5-methylcyclohexyl)oxy, haloalkoxy, haloalkyl, and —NZ₃Z₄ wherein Z₃ and Z₄ are independently selected from hydrogen, alkoxyalkyl, alkyl, and cycloalkyl wherein cycloalkyl is cyclohexyl, or Z₃ and Z₄ taken together with the nitrogen atom to which they are attached form a heterocycle selected from piperidin-1-yl and morpholin-4-yl wherein piperidin-1-yl may be unsubstituted or substituted with 1 or 2 substituents independently selected from lower alkyl;

R₂ is selected from hydrogen and lower alkyl;

R₃ is selected from hydrogen, lower alkoxy, lower alkyl, and hydroxy;

R₄ is selected from hydrogen, cycloalkyl wherein cycloalkyl is cyclohexyl, and halo;

R₅ is selected from hydrogen, lower alkoxy, lower alkyl, halo, and hydroxy;

R₆ is hydrogen; or

R₆ and R₇ together with the carbon atoms to which they are attached form a 5, 6, or 7 membered carbocyclic ring; or

R₆ and R₇ together with the carbon atoms to which they are attached form a 5 or 6 membered ring containing 1 heteroatom selected from O and S(O)ₙ wherein n is 0-2;

R₈ is absent or hydrogen; or

R₇ and R₈ together form

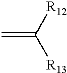

wherein R₁₂ and R₁₃ are independently selected from the group consisting of hydrogen, lower alkoxy, and lower alkyl provided that R₁ is S(O)₂R₉; or

R₁₂ and R₁₃ together with the carbon atom to which they are attached form a 6 membered carbocyclic ring; or

R₁₂ and R₆ together with the carbon atoms to which they are attached form a 6 membered carbocyclic ring provided that R₁₃ is hydrogen; and

R₁₄ is selected from hydrogen and lower alkyl.

In another embodiment of the present invention compounds have formula II

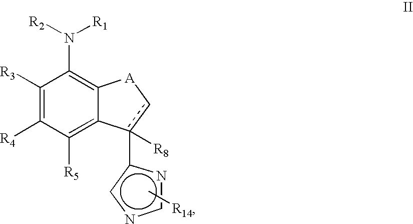

or a pharmaceutically acceptable salt thereof, wherein A is selected from —CH₂—, —CH₂CH₂, and —CH₂CH₂CH₂—; [symbol] represents a single bond or a double bond; and R₁, R₂, R₃, R₄, R₅, R₈ and R₁₄ are as defined in formula I.

In another embodiment of the present invention compounds have formula II wherein A is —CH₂—; [symbol] is a single bond; R₁ is C(O)R₁₀; R₈ is hydrogen; and R₂, R₃, R₄, R₅, R₁₀, and R₁₄ are as defined in formula I.

In another embodiment of the present invention compounds have formula II wherein A is —CH₂—; [symbol] is a single bond; R₁ is S(O)₂R₉; R₈ is hydrogen; and R₂, R₃, R₄, R₅, R₉, and R₁₄ are as defined in formula I.

In another embodiment of the present invention compounds have formula II wherein A is —CH₂CH₂—; [symbol] is a double bond; R₁ is C(O)R₁₀; R₈ is absent; and R₂, R₃, R₄, R₅, R₁₀, and R₁₄ are as defined in formula I.

In another embodiment of the present invention compounds have formula II 9 wherein A is —CH₂CH₂—; [symbol] is a double bond; R₁ is S(O)₂R₉; R₈ is absent; and R₂, R₃, R₄, R₅, R₉, and R₁₄ are as defined in formula I.

In another embodiment of the present invention compounds have formula II wherein A is —CH₂CH₂—; [symbol] is a single bond; R₁ is C(O)R₁₀; R₈ is hydrogen; and R₂, R₃, R₄, R₅, R₁₀, and R₁₄ are as defined in formula I.

In another embodiment of the present invention compounds have formula II wherein A is —CH₂CH₂—; [symbol] is a single bond; R₁ is S(O)₂R₉; R₈ is hydrogen; and R₂, R₃, R₄, R₅, R₉, and R₁₄ are as defined in formula I.

In another embodiment of the present invention compounds have formula II wherein A is —CH₂CH₂CH₂—; [symbol] is a single bond; R₁ is C(O)R₁₀; R₈ is hydrogen; and R₂, R₃, R₄, R₅, R₁₀, and R₁₄ are as defined in formula I.

In another embodiment of the present invention compounds have formula II wherein A is —CH₂CH₂CH₂—; [symbol] is a single bond; R₁ is S(O)₂R₉; R₈ is hydrogen; and R₂, R₃, R₄, R₅, R₉, and R₁₄ are as defined in formula I.

In another embodiment of the present invention compounds have formula III

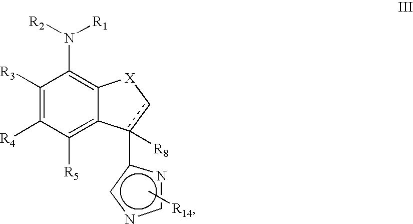

or a pharmaceutically acceptable salt thereof, wherein X is selected from O, NR₁₁, and S(O)ₙ; [symbol] represents a single bond or a double bond; and R₁, R₂, R₃, R₄, R₅, R₈, R₁₁, R₁₄, and n are as defined in formula I.

In another embodiment of the present invention compounds have formula IV

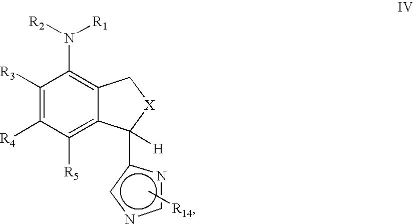

or a pharmaceutically acceptable salt thereof, wherein X is selected from O, NR₁₁, and S(O)ₙ; and R₁, R₂, R₃, R₄, R₅, R₁₁, R₁₄, and n are as defined in formula I.

In another embodiment of the present invention compounds have formula IV wherein X is O; R₁ is C(O)R₁₀; and R₂, R₃, R₄, R₅, R₁₀, and R₁₄ are as defined in formula I.

In another embodiment of the present invention compounds have formula IV wherein X is O; R₁ is S(O)₂R₉; and R₂, R₃, R₄, R₅, R₉, and R₁₄ are as defined in formula I.

In another embodiment of the present invention compounds have formula V

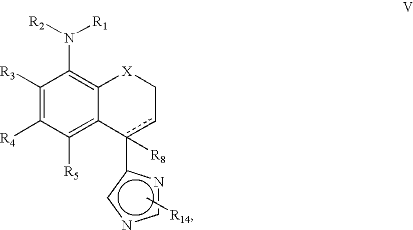

or a pharmaceutically acceptable salt thereof, wherein X is selected O, NR₁₁, and S(O)ₙ; [symbol] represents a single bond or a double bond; and R₁, R₂, R₃, R₄, R₅, R₈, R₁₁, R₁₄ and n are as defined in formula I.

In another embodiment of the present invention compounds have formula V wherein [symbol] is a single bond; X is selected from O, NR₁₁, and S(O)ₙ; R₁ is C(O)R₁₀; R₈ is hydrogen; and R₂, R₃, R₄, R₅, R₁₀, R₁₁, R₁₄ and n are as defined in formula I.

In another embodiment of the present invention compounds have formula V wherein [symbol] is a single bond; X is selected from O and S; R₁ is S(O)₂R₉; R₈ is hydrogen; and R₂, R₃, R₄, R₅, R₉, and R₁₄ are as defined in formula I.

In another embodiment of the present invention compounds have formula VI

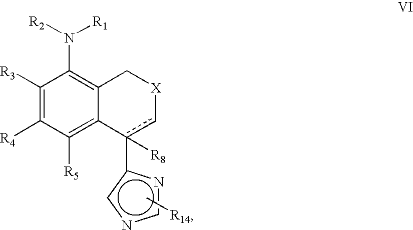

or a pharmaceutically acceptable salt thereof, wherein X is selected from O, NR₁₁, and S(O)ₙ; [symbol] represents a single bond or a double bond; and R₁, R₂, R₃, R₄, R₅, R₈, R₁₁, R₁₄ and n are as defined in formula I.

In another embodiment of the present invention compounds have formula VII

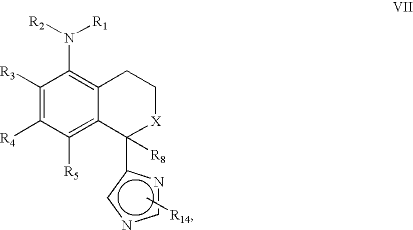

or a pharmaceutically acceptable salt thereof, wherein X is selected from O, NR₁₁, and S(O)ₙ; and R₁, R₂, R₃, R₄, R₅, R₈, R₁₁, R₁₄ and n are as defined in formula I.

In another embodiment of the present invention compounds have formula VIII

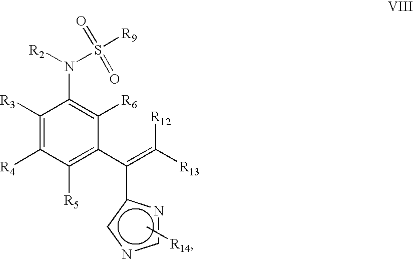

or a pharmaceutically acceptable salt thereof, wherein R₆ is selected from hydrogen, lower alkoxy, lower alkenyl, lower alkyl, lower haloalkyl, halo, and hydroxy; and R₂, R₃, R₄, R₅, R₁₂, R₁₃, and R₁₄ are as defined in formula I.

In another embodiment of the present invention compounds have formula VIII wherein R₆ is hydrogen; R₁₂ and R₁₃ are independently selected from hydrogen, lower alkoxy, and lower alkyl; and R₂, R₃, R₄, R₅, R₉, and R₁₄ are as defined in formula I.

In another embodiment of the present invention compounds have formula VIII wherein R₆ is hydrogen; R₁₂ and R₁₃ together with the carbon atom to which they are attached form a 3, 4, 5, 6, or 7 membered carbocyclic ring; and R₂, R₃, R₄, R₅, R₉, and R₁₄ are as defined in formula I.

Another embodiment of the the present invention includes a pharmaceutical composition comprising a therapeutically effective amount of a compound of formula I-VIII in combination with a pharmaceutically acceptable carrier.

Another embodiment of the the present invention includes a method of activating α₁ adrenoceptors in a host mammal in need of such treatment comprising administering a therapeutically effective amount of a compound of formula I-VIII.

Another embodiment of the the present invention includes a method of treating urinary incontinence in a host mammal in need of such treatment comprising administering a therapeutically effective amount of a compound of formula I-VIII.

Another embodiment of the the present invention includes a method of treating retrograde ejaculation in a host mammal in need of such treatment comprising administering a therapeutically effective amount of a compound of formula I-VIII.

### Definition of Terms

The term “alkenyl,” as used herein, refers to a straight or branched chain hydrocarbon containing from 2 to 10 carbons and containing at least one carbon-carbon double bond formed by the removal of two hydrogens. Representative examples of “akenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like.

The term “alkenyloxy,” as used herein, refers to a alkenyl group, as defined herein, appended to the parent molecular moiety through an oxygen atom. Repesentative examples of alkenyloxy include, but are not limited to 4-pentenyloxy, 3 butenyloxy, ethenyloxy, and the like

The term “alkxyl,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like.

The term “alkoxyalkyl,” as used herein, refers to an alkoxy group, as defined herein, appended to the parent molecular moiety through an alkyl group, as defined herein. Representative examples of alkoxyalkyl include, but are not limited to, methoxymethyl, 2-(methoxy)ethyl, and the like.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, tert-butoxycarbonyl, and the like.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, 1-oxopentyl, and the like.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, 3-oxopentyl, and the like.

The term “alkylcarbonyloxy,” as used herein, refers to an alkylcarbonyl group, as defined herein, appended to the parent molecular moiety through an oxy group, as defined herein. Representative examples of alkylcarbonyloxy include, but are not limited to, acetyloxy, ethylcarbonyloxy, tert-butylcarbonyloxy, and the like.

The term “alkylthio,” as used herein, refers to an alkyl group, as defined herein, appended to the parent molecular moiety through a thio group, as defined herein. Representative examples of alkylthio include, but are not limited, methylsulfanyl, ethylsulfanyl, tert-butylsulfanyl, hexylsulfanyl, and the like.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like.

The term “alkynyloxy,” as used herein, refers to a alkynyl group, as defined herein, appended to the parent molecular moiety through an oxygen atom. Repesentative examples of alkynyloxy include, but are not limite to 4-pentynyloxy, 3 butynyloxy, ethynyloxy, and the like.

The term “amino,” as used herein, refers to a —NH₂ group.

The term “aryl,” as used herein, refers to a monocyclic-ring system or a bicyclic-fused ring system wherein one or more of the fused rings are aromatic. Representative examples of aryl include, but are not limited to, azulenyl, indanyl, indenyl, naphthyl, phenyl, tetrahydronaphthyl, and the like.

The aryl groups of this invention can be substituted with 1, 2, 3, 4, or 5 substituents independently selected from alkenyl, alkoxy, alkoxycarbonyl, alkyl, alkylcarbonyl, alkylcarbonyloxy, alkylthio, alkynyl, arylalkoxycarbonyl, carboxy, cyano, cycloalkyl, cycloalkylalkyl, formyl, halo, haloalkyl, hydroxy, hydroxyalkyl, mercapto, nitro, —NZ₁₀Z₁₁, (NZ₁₀Z₁₁)alkyl, —C(O)Z₁₀Z₁₁, and —S(O)₂Z₁₀Z₁₁,

The term “arylalkenyl,” as used herein, refers to an aryl group, as defined herein, appended to the parent molecular moiety through an alkenyl group, as defined herein. Representative examples of arylalkenyl include, but are not limited to, 2-phenylethenyl, 3-phenylpropen-1-yl, 2-naphth-2-ylethenyl, and the like.

The term “arylalkoxy,” as used herein, refers to an aryl group, as defined herein, appended to the parent molecular moiety through an alkoxy group, as defined herein.

Representative examples of arylalkoxy include, but are not limited to, 2-phenylethoxy, 3-naphth-2-ylpropoxy, 5-phenylpentyloxy, and the like.

The term “arylalkoxycarbonyl,” as used herein, refers to an arylalkoxy group, as defined herein, appended to the parent molecular moiety through a carbonyl group, as defined herein. Representative examples of arylalkoxycarbonyl include, but are not limited to, benzyloxycarbonyl, naphth-2-ylmethoxycarbonyl, and the like.

The term “arylalkyl,” as used herein, refers to an aryl group, as defined herein, appended to the parent molecular moiety through an alkyl group, as defined herein.

Representative examples of arylalkyl include, but are not limited to, benzyl, 2-phenylethyl, 3-phenylpropyl, 2-naphth-2-ylethyl, and the like.

The term “aryloxy,” as used herein, refers to an aryl group, as defined herein, appended to the parent molecular moiety through an oxy group, as defined herein. Representative examples of aryloxy include, but are not limited to, phenoxy, 4-methylphenoxy, and the like.

The term “carbonyl,” as used herein, refers to a —C(O)— group.

The term “carboxy,” as used herein, refers to a —CO₂H group.

The term “cyano,” as used herein, refers to a —CN group.

The term “cycloalkyl,” as used herein, refers to a saturated cyclic hydrocarbon group containing from 3 to 8 carbons. Representative examples of cycloalkyl include, but are not limited to, cyclopropyl, cyclobutyl, cyclopentyl, cyclohexyl, cycloheptyl, cyclooctyl and the like.

The cycloalkyl groups of this invention can be substituted with 1, 2, or 3 substituents independently selected from alkoxy, alkoxycarbonyl, alkyl, alkylthio, carboxy, formyl, halo, haloalkyl, hydroxy, lower alkyl, mercapto, —N Z₁₀Z₁₁, and —C(O)N Z₁₀Z₁₁.

The term “cycloalkylalkyl,” as used herein, refers to cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, 4-cycloheptylbutyl, and the like.

The term “cycloalkyloxy,” as used herein, refers to cycloalkyl group, as defined herein, appended to the parent molecular moiety through an oxy group, as defined herein. Representative examples of cycloalkyloxy include, but are not limited to, cyclohexyloxy, 2-isopropyl-5-methylcyclohexyloxy, and the like.

The term “formyl,” as used herein, refers to a —C(O)H group.

The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein. Representative examples of haloalkoxy include, but are not limited to, 2-chloroethoxy, 2,2,2-trichloroethoxy, 2,2,2-trichloro-2,2-dimethylethoxy trifluoromethoxy, and the like.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

The term “heterocycle” or “heterocyclic,” as used herein, refers to a monocyclic or bicyclic ring system. The monocyclic ring system is exemplified by any 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepinyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothiophenyl, tetrazinyl, tetrazolyl, thiadiazolyl, thiadiazolinyl, thiadiazolidinyl, thiazolyl, thiazolinyl, thiazolidinyl, thienyl, thiomorpholinyl, thiomorpholine 1,1-dioxide, thiopyranyl, triazinyl, triazolyl, trithianyl, and the like. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzothiazolyl, benzothienyl, benzoxazolyl, benzofuranyl, benzopyranyl, benzothiopyranyl, benzodioxinyl, 1,3-benzodioxolyl, cinnolinyl, indazolyl, indolyl, indolinyl, indolizinyl, naphthyridinyl, isobenzofuranyl, isobenzothienyl, isoindolyl, isoindolinyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, thiopyranopyridinyl, and the like.

The heterocycles of this invention can be substituted with 1, 2, or 3 substituents independently selected from alkenyl, alkoxy, alkoxycarbonyl, alkyl, alkylcarbonyl, alkylcarbonyloxy, alkylthio, alkynyl, arylalkoxycarbonyl, carboxy, cyano, cycloalkyl, cycloalkylalkyl, formyl, halo, haloalkyl, hydroxy, hydroxyalkyl, mercapto, nitro, —NZ₁₀Z₁₁, (NZ₁₀Z₁₁)alkyl, —C(O)NZ₁₀Z₁₁, and —SO₂NZ₁₀Z₁₁.

The term “hydroxy,” as used herein, refers to an —OH group.

The term “hydroxyalkyl,” as used herein, refers to a hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2-ethyl-4-hydroxyheptyl, and the like.

The term “lower alkenyl,” as used herein, is a subset of alkenyl as defined herein and refers to a straight or branched chain hydrocarbon group containing from 2 to 4 carbon atoms and containing at least one carbon-carbon double bond formed by the removal of two hydrogens. Representative examples of “lower alkenyl” include, but are not limited to, ethenyl, 1-propenyl, 2-propenyl, 2-butenyl, and the like.

The term “lower alkoxy,” as used herein, refers to a lower alkyl group, as defined herein, appended to the parent molecular moiety through an oxy group, as defined herein. Representative examples of lower alkoxy include, but are not limited to, methoxy, ethoxy, propoxy, 2-propoxy, butoxy, tert-butoxy, and the like.

The term “lower alkyl,” as used herein, refers to a straight or branched chain hydrocarbon group containing from 1-to-4 carbon atoms. Representative examples of lower alkyl include, but are not limited to, methyl, ethyl, n-propyl, iso-propyl, n-butyl, iso-butyl, tert-butyl, and the like.

The term “lower haloalkyl,” as used herein, refers to at least one halogen, as defined herein, appended to the parent molecular moiety through a lower alkyl group, as defined herein. Representative examples of lower haloalkyl include, but are not limited to, fluoromethyl, difluoromethyl, trifluoromethyl, pentafluoroethyl, chloromethyl, 3-chloropropyl, 2-fluoroethyl, 2,2,2-trifluoroethyl, 2,2-difluoroethyl, and the like.

The term “mercapto,” as used herein, refers to a —SH group. The term “nitro,” as used herein, refers to a —NO₂ group.

The term “—NZ₁₀Z₁₁,” as used herein, refers to two groups, Z₁₀ and Z₁₁, which are appended to the parent molecular moiety through a nitrogen atom. Z₁₀ and Z₁₁ are independently selected from hydrogen, alkyl, alkylcarbonyl, aryl, arylalkyl, and formyl. Representative examples of —NZ₁₀Z₁₁ include, but are not limited to, amino, benzylamino, methylamino, acetylamino, acetylmethylamino, and the like.

The term “(NZ₁₀Z₁₁)alkyl,” as used herein, refers to a —NZ₁₀Z₁₁ group, as defined herein, appended to the parent molecular moiety through an alkyl group, as defined herein. Representative examples of (NZ₁₀Z₁₁)alkyl include, but are not limited to, aminomethyl, benzylaminomethyl, methylaminomethyl, acetylaminomethyl, acetylmethylaminomethyl, and the like.

The term “oxy,” as used herein, refers to (—O—).

The term “sulfonyl,” as used herein, refers to a —S(O)₂— group.

The term “thio,” as used herein, refers to (—S—).

Compounds of the present invention may exist as stereoisomers where asymmetric or chiral centers are present. The present invention contemplates various stereoisomers and mixtures thereof. Stereoisomers include enantiomers and diastereomers. Individual stereoisomers of compounds of the present invention can be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Geometric isomers can also exist in the compounds of the present invention. The present invention contemplates the various geometric isomers and mixtures thereof resulting from the arrangement of substituents around a carbon-carbon double bond. Substituents around a carbon-carbon double bond are designated as being in the (Z) or (E) configuration where the term (Z) represents substituents on the same side of the carbon-carbon double bond and the term (E) represents substituents on opposite sides of the carbon-carbon double bond. Geometric isomers of the present invention can be separated into individual (E) and (Z) isomers by chromatography such as flash chromatography, medium pressure liquid chromatography, or high pressure liquid chromatography. Geometric isomers can also exist in the compounds of the present invention resulting from the arrangement of substituents around a ring. The arrangement of substituents around a ring are designated as cis or trans where the term “cis” represents substituents on the same side of the plane of the ring and the term “trans” represents substituents on opposite sides of the plane of the ring. Mixtures of compounds where the substitutients are disposed on both the same and opposite sides of plane of the ring are designated “cis/trans.”

Preferred compounds of formula I include,

N-[5,6,7,8-tetrahydro-5-(5-methyl-1H-imidazol-4-yl)-1-naphthalenyl]ethanesulfonamide;

N-[1-(1H-imidazol-4-yl)-1,3-dihydro-2-benzothien-4-yl]ethanesulfonamide;

5 N-[3-(1H-imidazol-4-yl)-2,3-dihydro-1-benzothien-7-yl]ethanesulfonamide;

N-[5-(1H-imidazol-4-yl)-5,6,7,8-tetrahydro-1-naphthalenyl]-1-piperidinesulfonamide;

benzyl 5-(1H-imidazol-4-yl)-5,6,7,8-tetrahydro-1-naphthalenylcarbamate;

N-[5-(H-imidazol-4-yl)-5,6,7,8-tetrahydro-1-naphthalenyl]urea;

N-[5-(1H-imidazol-4-yl)-5,6,7,8-tetrahydro-1-naphthalenyl]-N′-phenylurea;

N-[5-(1H-imidazol-4-yl)-5,6,7,8-tetrahydro-1-naphthalenyl]-N′-isopropylurea;

N-[4-(1H-imidazol-4-yl)-2-methyl-1,2,3,4-tetrahydro-8-isoquinolinyl]ethanesulfonamide;

N-[4-(2-ethyl-1H-imidazol-4-yl)-1,2,3,4-tetrahydro-8-isoquinolinyl]ethanesulfonamide;

N-[2-ethyl-4-(1H-imidazol-4-yl)-1,2,3,4-tetrahydro-8-isoquinolinyl]ethanesulfonamide;

N-[1-(2-ethyl-1H-imidazol-4-yl)-1,2,3,4-tetrahydro-5-isoquinolinyl]ethanesulfonamide;

N-[2-ethyl-1-(1H-imidazol-4-yl)-1,2,3,4-tetrahydro-5-isoquinolinyl]ethanesulfonamide;

N-[4-(1H-imidazol-4-yl)-1,2,3,4-tetrahydro-8-quinolinyl]ethanesulfonamide;

N-[1-(1H-imidazol-4-yl)-3,4-dihydro-1H-isothiochromen-5-yl]ethanesulfonamide;

N-[4-(1H-imidazol-4-yl)-3,4-dihydro-1H-isothiochromen-8-yl]ethanesulfonamide;

N-{3-[cyclopentylidene(1H-imidazol-4-yl)methyl]phenyl}ethanesulfonamide;

N-[5-(1H-imidazol-4-yl)-2-methoxy-5,6,7,8-tetrahydro-1-naphthalenyl]methanesulfonamide;

N-[2-hydroxy-5-(1H-imidazol-4-yl)-5,6,7,8-tetrahydro-1-naphthalenyl]methanesulfonamide;

N-[2-hydroxy-5-(2-methyl-1H-imidazol-4-yl)-5,6,7,8-tetrahydro-1-naphthalenyl]methanesulfonamide;

N-[2-hydroxy-5-(1-methyl-1H-imidazol-5-yl)-5,6,7,8-tetrahydro-1-naphthalenyl]methanesulfonamide;

N-[2-hydroxy-5-(1-methyl-1H-imidazol-4-yl)-5,6,7,8-tetrahydro-1-naphthalenyl]methanesulfonamide;

N-[5-(1-ethyl-1H-imidazol-4-yl)-2-hydroxy-5,6,7,8-tetrahydro-1-naphthalenyl]methanesulfonamide;

N-[2-hydroxy-5-(1-propyl-1H-imidazol-4-yl)-5,6,7,8-tetrahydro-1-naphthalenyl]methanesulfonamide;

N-[5-(1H-imidazol-4-yl)-5,6,7,8-tetrahydro-1-naphthalenyl]methanesulfonamide;

(R)-N-[5-(1H-imidazol-4-yl)-5,6,7,8-tetrahydro-1-naphthalenyl]methanesulfonamide;

(S)-N-[5-(1H-imidazol-4-yl)-5,6,7,8-tetrahydro-1-naphthalenyl]methanesulfonamide;

N-[5-(1H-imidazol-4-yl)-5,6,7,8-tetrahydro-1-naphthalenyl]ethanesulfonamide;

N-[5,6,7,8-tetrahydro-5-(1-methyl-1H-imidazol-4-yl)-1-naphthalenyl]methanesulfonamide;

N-[5,6,7,8-tetrahydro-5-(1H-imidazol-4-yl)-1-naphthalenyl]-N-methylmethanesulfonmamide;

N-[5,6,7,8-tetrahydro-5-(1H-imidazol-4-yl)-1-naphthalenyl]acetamide;

2,2,2-trifluoro-N-[5-(1H-imidazol-4-yl)-5,6,7,8-tetrahydro-1-naphthalenyl]acetamide;

N-[5,6,7,8-tetrahydro-5-(1H-imidazol-4-yl)-1-naphthalenyl]-2-methylethanesulfonamide;

N-[4-(1H-imidazol-4-yl)-3,4-dihydro-2H-chromen-8-yl]methanesulfonamide;

N-[5,6,7,8-tetrahydro-5-(1H-imidazol-4-yl)-1-naphthalenyl]-2,2,2-trifluoroethanesulfonamide;

N-[1-(1H-imidazol-4-yl)-2,3-dihydro-1H-inden-4-yl]methanesulfonamide;

N-[5,6,7,8-tetrahydro-5-(1H-imidazol-4-yl)-4-methyl-1-naphthalenyl]methanesulfonamide;

N-[5,6,7,8-tetrahydro-4-hydroxy-5-(1H-imidazol-4-yl)-1-naphthalenyl]methanesulfonamide;

N-[5,6,7,8-tetrahydro-(1H-imidazol-4-yl)-4-methoxy-1-naphthalenyl]ethanesulfonamide;

N-[5,6,7,8-tetrahydro-(1H-imidazol-4-yl)-4-methoxy-1-naphthalenyl]methanesulfonamide;

N-[5,6,7,8-tetrahydro-(1H-imidazol-4-yl)-1-naphthalenyl]cyclopropanesulfonamide;

(+)-N-[5-(1H-imidazol-4-yl)-5,6,7,8-tetrahydro-1-napthalenyl]ethanesulfonamide;

is (−)-N-[5-(1H-imidazol-4-yl)-5,6,7,8-tetrahydro-1-naphthalenyl]ethanesulfonamide;

(−)-N-[5,6,7,8-tetrahydro-5-(1H-imidazol-4-yl)-1-naphthalenyl]-2,2,2-trifluoroethanesulfonamide;

(+)-N-[5,6,7,8-tetrahydro-5-(1H-imidazol-4-yl)-1-naphthalenyl]-2,2,2-trifluoroethanesulfonamide;

N-[5-(1H-imidazol-4-yl)-6,7,8,9-tetrahydro-5H-benzo[a]cyclohepten-1-yl]methanesulfonamide;

N-[1-(1H-imidazol-4-yl)-2,3-dihydro-1H-inden-4-yl]ethanesulfonamide;

N-[5-(1H-imidazol-4-yl)-6,7,8,9-tetrahydro-5H-benzo[a]cyclohepten-1-yl]ethanesulfonamide;

N-[4-chloro-5-(1H-imidazol-4-yl)-5,6,7,8-tetrahydro-1-naphthalenyl]ethanesulfonamide;

N-[4-chloro-5-(1H-imidazol-4-yl)-5,6,7,8-tetrahydro-1-naphthalenyl]methanesulfonamide;

N-[4-fluoro-5-(1H-imidazol-4-yl)-5,6,7,8-tetrahydro-1-naphthalenyl]methanesulfonamide;

N-[3-(1-(1H-imidazol-4-yl)vinyl)phenyl]ethanesulfonamide;

N-{3-[1-(1H-imidazol-4-yl)-2-methoxyethenyl]phenyl}ethanesulfonamide;

N-[5-(1H-imidazol-4-yl)-7,8-dihydro-1-naphthalenyl]methanesulfonamide;

N-[3-(cyclohexylidene-(1H-imidazol-4-ylmethyl)phenyl]ethanesulfonamide;

N-[5-(1H-imidazol-4-yl)-5,6,7,8-tetrahydro-1-naphthalenyl]-3,5-dimethyl-4-isoxazolesulfonamide;

N-[5-(1H-imidazol-4-yl)-5,6,7,8-tetrahydro-1-naphthalenyl]-1-propanesulfonamide;

N-[5-(1H-imidazol-4-yl)-5,6,7,8-tetrahydro-1-naphthalenyl]-1-butanesulfonamide;

3-chloro-N-[5-(1H-imidazol-4-yl)-5,6,7,8-tetrahydro-1-naphthalenyl]-1-propanesulfonamide;

N-[5-(1H-imidazol-4-yl)-5,6,7,8-tetrahydro-1-naphthalenyl]-1-methyl-1H-imidazole-4-sulfonamide;

N-[5-(1H-imidazol-4-yl)-5,6,7,8-tetrahydro-1-naphthalenyl](phenyl)methanesulfonamide;

N-[5-(1H-imidazol-4-yl)-5,6,7,8-tetrahydro-1-naphthalenyl]-4-methylbenzenesulfonamide;

N-[5-(1H-imidazol-4-yl)-5,6,7,8-tetrahydro-1-naphthalenyl]-2-methylbenzenesulfonamide;

N-[5-(1H-imidazol-4-yl)-5,6,7,8-tetrahydro-1-naphthalenyl]-2-phenyl-1-ethenesulfonamide;

N-[5-(1H-imidazol-4-yl)-5,6,7,8-tetrahydro-1-naphthalenyl]-4-methoxybenzenesulfonamide;

5-chloro-N-[5-(1H-imidazol-4-yl)-5,6,7,8-tetrahydro-1-naphthalenyl]-2-thiophenesulfonamide;

N-[5-(1H-imidazol-4-yl)-5,6,7,8-tetrahydro-1-naphthalenyl]-8-quinolinesulfonamide;

5-chloro-N-[5-(1H-imidazol-4-yl)-5,6,7,8-tetrahydro-1-naphthalenyl]-1,3-dimethyl-1H-pyrazole-4-sulfonamide;

methyl 2-{[(5-(1H-imidazol-4-yl)-5,6,7,8-tetrahydro-1-naphthalenyl)amino] sulfonyl}-3-thiophenecarboxylate;

N-(5-{[(5-(1H-imidazol-4-yl)-5,6,7,8-tetrahydro-1-naphthalenyl)amino]sulfonyl}-4-methyl-1,3-thiazol-2-yl)acetamide;

5-chloro-N-[5-(1H-imidazol-4-yl)-5,6,7,8-tetrahydro-1-naphthalenyl]-3-methyl-2,3-dihydro-1-benzothiophene-2-sulfonamide;

N-[4-(1H-imidazol-4-yl)-3,4-dihydro-2H-chromen-8-yl]ethanesulfonamide;

N-[6-fluoro-4-(1H-imidazol-4-yl)-3,4-dihydro-2H-chromen-8-yl]ethanesulfonamide;

N-[5-(2-methyl-1H-imidazol-4-yl)-5,6,7,8-tetrahydro-1-naphthalenyl]ethanesulfonamide;

N-[1-(1H-imidazol-4-yl)-1,3-dihydro-2-benzofuran-4-yl]ethanesulfonamide;

2,2,2-trifluoro-N-[4-(1H-imidazol-4-yl)-3,4-dihydro-2H-chromen-8-yl]ethanesulfonamide;

N-[4-(1H-imidazol-4-yl)-3,4-dihydro-2H-thiochromen-8-yl]ethanesulfonamide;

N-[6-fluoro-4-(1H-imidazol-4-yl)-3,4-dihydro-2H-chromen-8-yl]methanesulfonamide;

2,2,2-trifluoro-N-{3-[1-(1H-imidazol-4-yl)vinyl]phenyl}ethanesulfonamide;

N-{3-[1-(1H-imidazol-4-yl)vinyl]phenyl}methanesulfonamide;

(+) N-[4-(1H-imidazol-4-yl)-3,4-dihydro-2H-chromen-8-yl]methanesulfonamide;

N-{3-[1-(1H-imidazol-4-yl)-2-methyl-1-propenyl]phenyl}ethanesulfonamide;

(+) N-[4-(1H-imidazol-4-yl)-3,4-dihydro-2H-chromen-8-yl]ethanesulfonamide;

N-[3-cyclohexyl-5-(1H-imidazol-4-yl)-5,6,7,8-tetrahydro-1-naphthalenyl]ethanesulfonamide;

N-[5-(1H-imidazol-4-yl)-2-methyl-5,6,7,8-tetrahydro-1-naphthalenyl]ethanesulfonamide;

N′-[5-(1H-imidazol-4-yl)-5,6,7,8-tetrahydro-1-naphthalenyl]-N,N-dimethylsulfamide;

N′-[5-(1H-imidazol-4-yl)-5,6,7,8-tetrahydro-1-naphthalenyl]-N,N-dipropylurea;

N-cyclohexyl-N-ethyl-N′-[5-(1H-imidazol-4-yl)-5,6,7,8-tetrahydro-1-naphthalenyl]urea;

N-[5-(1H-imidazol-4-yl)-5,6,7,8-tetrahydro-1-naphthalenyl]-1-piperidinecarboxamide;

N-[5-(1H-imidazol-4-yl)-5,6,7,8-tetrahydro-1-naphthalenyl]-3,5-dimethyl-1-piperidinecarboxamide;

N′-[5-(1H-imidazol-4-yl)-5,6,7,8-tetrahydro-1-naphthalenyl]-N,N-bis(2-methoxyethyl)urea;

N-[5-(1H-imidazol-4-yl)-5,6,7,8-tetrahydro-1-naphthalenyl]-4-morpholinecarboxamide;

N-ethyl-N′-[5-(1H-imidazol-4-yl)-5,6,7,8-tetrahydro-1-naphthalenyl]-N-isopropylurea;

methyl 5-(1H-imidazol-4-yl)-5,6,7,8-tetrahydro-1-naphthalenylcarbamate;

ethyl 5-(1H-imidazol-4-yl)-5,6,7,8-tetrahydro-1-naphthalenylcarbamate;

2,2,2-trichloroethyl 5-(1H-imidazol-4-yl)-5,6,7,8-tetrahydro-1-naphthalenylcarbamate;

2,2,2-trichloro-1,1-dimethylethyl-5-(1H-imidazol-4-yl)-5,6,7,8-tetrahydro-1-naphthalenylcarbamate;

(1S,2R,5S)-2-isopropyl-5-methylcyclohexyl 5-(1H-imidazol-4-yl)-5,6,7,8-tetrahydro-1-naphthalenylcarbamate;

4-methylphenyl 5-(1H-imidazol-4-yl)-5,6,7,8-tetrahydro-1-naphthalenylcarbamate;

N-[3-fluoro-5-(1H-imidazol-4-yl)-5,6,7,8-tetrahydro-1-naphthalenyl]ethanesulfonamide; and

N-[3-chloro-5-(1H-imidazol-4-yl)-5,6,7,8-tetrahydro-1-naphthalenyl]ethanesulfonamide and pharmaceutically acceptable salts, thereof.

### Abbreviations

Abbreviations which have been used in the descriptions of the schemes and the examples that follow are: DMF for N,N-dimethylformamide; DMSO for dimethylsulfoxide, NBS for N-bromosuccinimide, NCS for N-chlorosuccinimide, PPA for polyphosphoric acid, pyr for pyridine, and THF for tetrahydrofuran.

Preparation of Compounds of the Invention

The compounds and processes of the present invention will be better understood in connection with the following synthetic schemes and methods which illustrate a means by which the compounds of the invention can be prepared. All references cited in the following schemes and examples are herein incorporated by reference.

The compounds of this invention can be prepared by a variety of synthetic routes. Representative procedures are shown in Schemes 1-26.

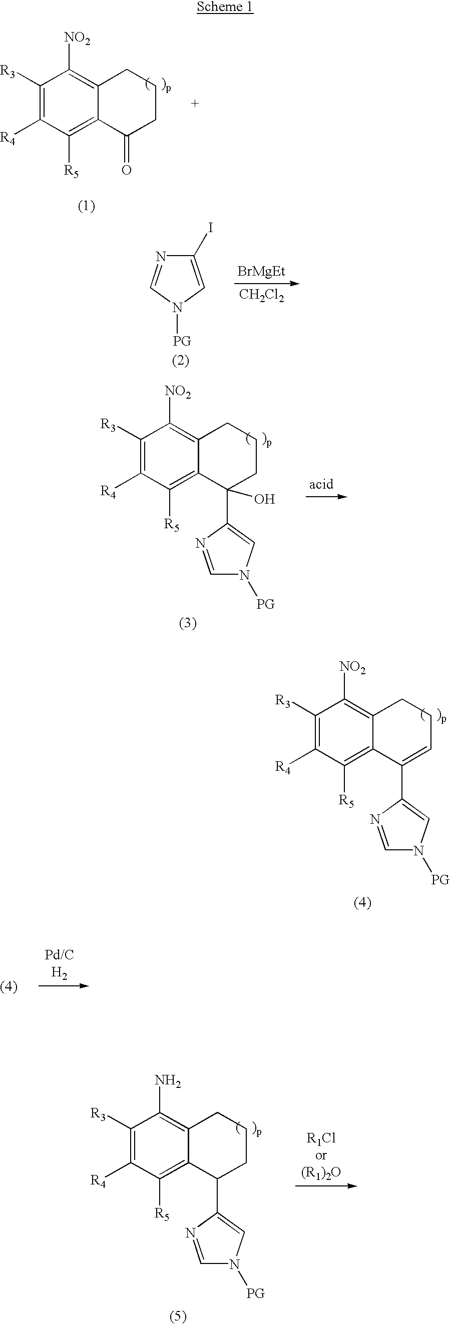

Indanes, tetrahydronaphthalenes, or tetrahydrobenzo[a]cycloheptenes of general formula (8), wherein p is 0, 1, or 2 and R₁, R₂, R₃, R₄, and R₅ are as defined in formula I, can be prepared as described in Scheme 1. Nitrocompounds of general formula (1), from Schemes 3 and 4, can be treated with 4-iodoimidazole of general formula (2), wherein PG may be N,N-dimethylsulfamoyl prepared according to (R. M. Turner, J. Org. Chem. (1991), 56, 5739-5740) or PG may be trityl prepared according to (K. Kirk, J. Het. Chem. (1985), 22, 57-59), in the presence of ethyl magnesium bromide to provide alcohols of general formula (3). Alcohols of general formula (3) can be dehydrated under acidic conditions (such as aqueous HCl, para-toluenesulfonic acid, trifluoroacetic acid or the like) to provide dihydro-compounds of general formula (4). The acidic conditions may cause removal of the protecting group (PG) necessitating reprotection with a nitrogen protecting reagent such as di-tert-butyl-dicarbonate. Dihydro-compounds of general formula (4) can be treated with a catalyst (such as palladium on carbon or the like) in a solvent (such as methanol, ethyl acetate or the like) under a hydrogen atmosphere to provide anilines of general formula (5). Anilines of general formula (5) can be treated with sulfonylating agents (such as sulfonyl chlorides) or acylating agents (such as anhydrides, acid chlorides, isocyanates, chloroformates, and carbamyl chlorides ) using a mild base (such as pyridine) in a solvent (such as dichloromethane) to provide compounds of general formula (6). Compounds of general formula (6) wherein R₁ is phenoxycarbonyl can be treated with a primary or secondary amines to provide compounds of general formula (6) wherein R₁ is C(O)NZ₃Z₄, wherein Z₃ and Z₄ are as defined in formula I. Compounds of general formula (6) can be treated with a strong non nucleophilic base (such as sodium hydride or the like) in a solvent (such as DMF or the like) and electrophiles such as alkyl halides, arylalkyl halides, cycloalkyl halides, or cycloalkylalkyl halides to provide compounds of general formula (7). The imidazole protecting group, N,N-dimethylsulfamoyl or tert-butoxycarbonyl, can be cleaved under acidic conditions such as trifluoroacetic acid or refluxing aqueous HCl to provide indanes, tetrahydronaphthalenes, or tetrahydrobenzo[a]cycloheptenes of general formula (8).

Indenes, dihydronaphthalenes, or dihydrobenzo[a]cycloheptenes of general formula (8A), wherein p is 0, 1, or 2 and R₁, R₂, R₃, R₄, and R, are as defined in formula I, can be prepared as described in Scheme 1. Dihydro comnpounds of general formula (4) can be treated with a metal such as zinc in a solvent such as acetic acid to provide anilines of general formula (5A). Anilines of general formula (5A) can be processed as described for the conversion of compounds of general formula (5) to compounds of general formula (8) to provide indenes, dihydronaphthalenes, or dihydrobenzo[a]cycloheptenes of general formula (8A).

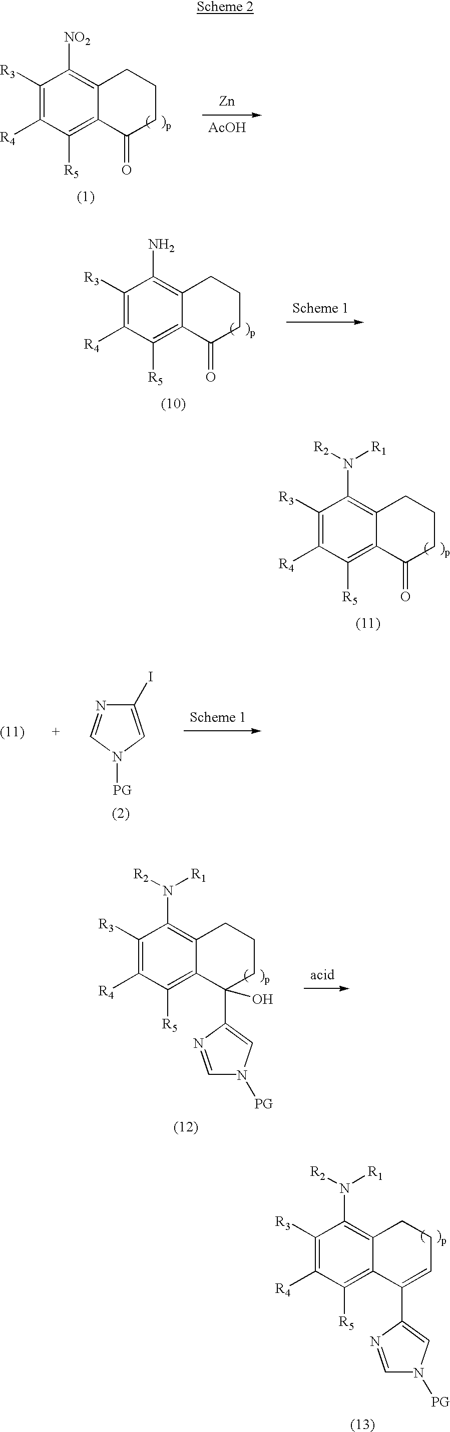

An alternate method of preparing indanes, tetrahydronaphthalenes, or tetrahydrobenzo[a]cycloheptenes of general formula (8), wherein p is 0, 1, or 2 and R₁, R₂, R₃, R₄, and R₅ are as defined in formula I, can be used as described in Scheme 2. Nitrocompounds of general formula (1), from Schemes 3 and 4, can be treated with a metal such as zinc in acetic acid to provide anilines of general formula (10). Anilines of general formula (10) can be treated as described in Scheme 1 to provide compounds of general formula (11). Compounds of general formula (11), wherein R₂ is other than hydrogen, can be treated with imidazoles of general formula (2), from Scheme 1, as described in Scheme 1 to provide alcohols of general formula (12). Alcohols of general formula (12) can be treated in a stepwise fashion with acid, hydrogenation conditions, and then acid as described in Scheme 1 to provide indanes, tetrahydronaphthalenes, or tetrahydrobenzo[a]cycloheptenes of general formula (8).

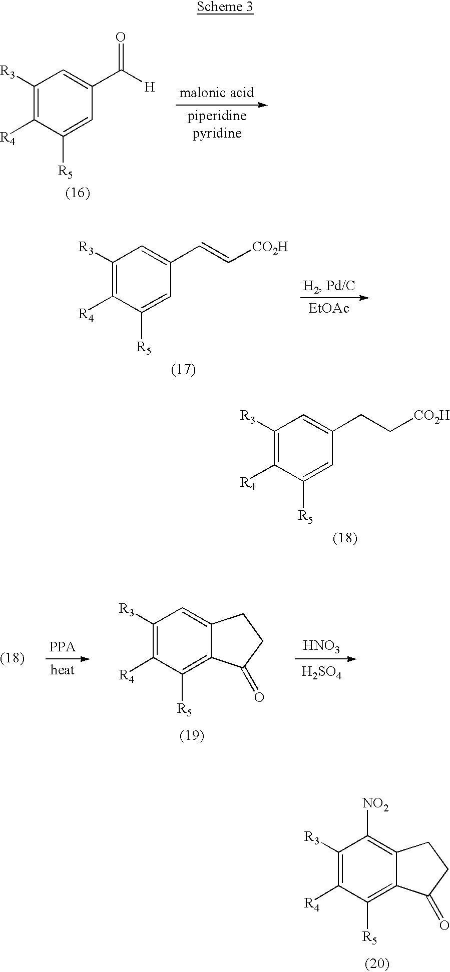

Nitroindanones of general formula (20) wherein R₃, R₄, and R₅ are as defined in formula I, can be prepared as described in Scheme 3. Benzaldehydes of general formula (16) can be treated with malonic acid in the presence of a base such as piperidine in a solvent such as pyridine to provide unsaturated propionic acids of general formula (17). Unsaturated propionic acids of general formula (17) can be hydrogenated using a catalyst such as palladium on carbon in a solvent such as ethyl acetate to provide saturated acids of general formula (18). Acids of general formula (18) can be heated in the presence of acid such as polyphosphoric acid (PPA) to provide indanones of general formula (19). Indanones of general formula (19) can be treated with fuming nitric acid and concentrated sulfirric acid in a solvent such as sulfuric acid or acetic acid to provide nitroindanones of general formula (20).

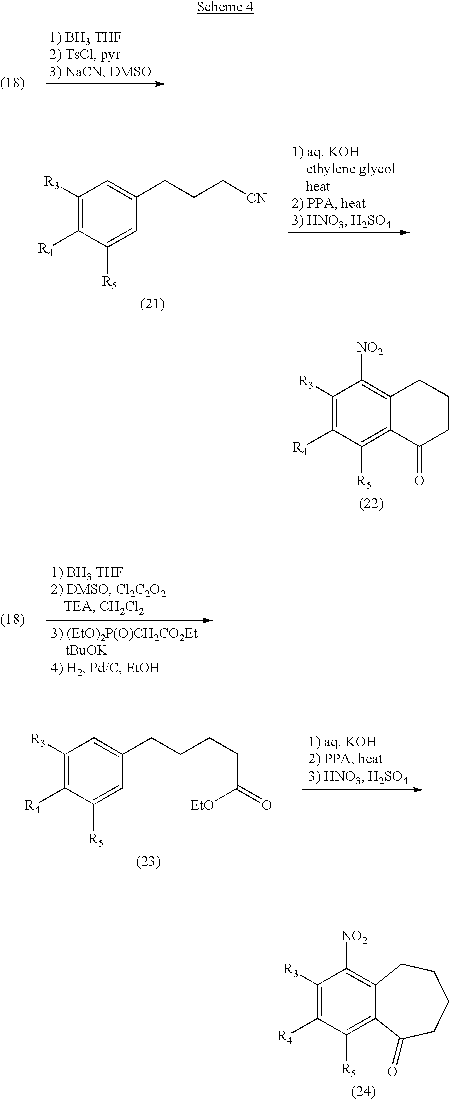

Nitrodihydronaphthalenones of general formula (22) and nitrotetrahydrobenzo[a]cycloheptenones of general formula (24), wherein R₃, R₄, and R₅ are as defined in formula I, can be prepared as described in Scheme 4. Acids of general formula (18), from Scheme 3, can be reduced to the alcohol, tosylated or mesylated, and then treated with sodium cyanide in a stepwise fashion to provide nitrites of general formula (21). Nitriles of general formula (21) can be treated with aqueous base, cyclized under acidic or Friedel-Crafts acylation conditions, and nitrated in a stepwise fashion to provide nitrodihydronaphthalenones of general formula (22).

Acids of general formula (18), from Scheme 3, can be reduced to the alcohol, oxidized to the aldehyde, treated with triethyl phosphonoacetate, and hydrogenated in a stepwise fashion to provide esters of general formula (23). Esters of general formula (23) can be treated with aqueous base, cyclized under acidic or Friedel-Crafts acylation conditions, and nitrated in a stepwise fashion to provide nitrotetrahydrobenzo[a]cycloheptenones of general formula (24).

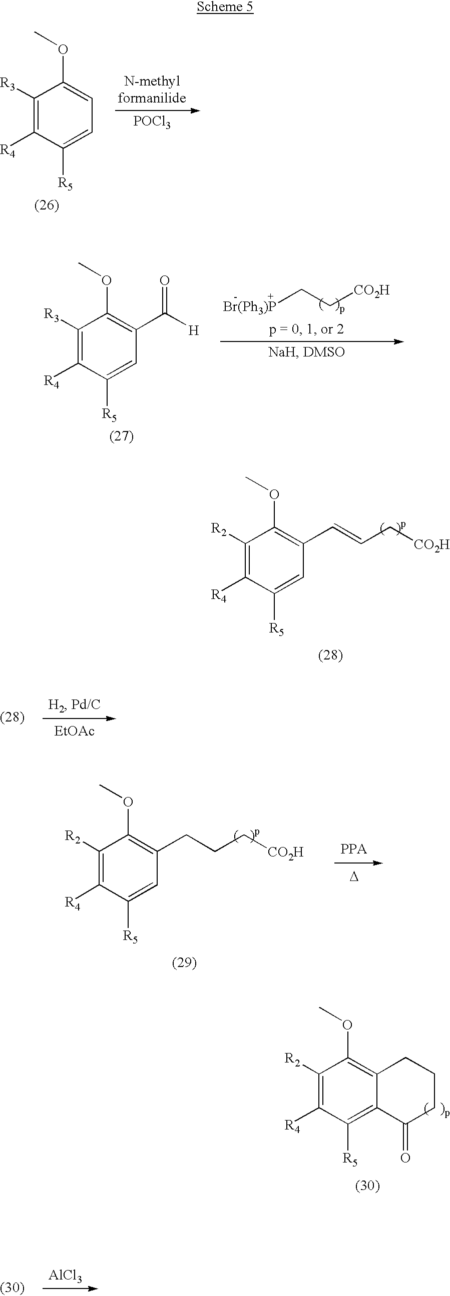

Another method of preparing indanes, tetrahydronaphthalenes, or tetrahydrobenzo[a]cycloheptenes of general formula (8), wherein p is 0, 1, or 2, and R₁, R₂, R₃, R₄, and R₅ are as defined in formula I, can be used as described in Scheme 5. Anisoles of general formula (26) can be treated with N-methylformanilide in phosphorous oxychloride as described in (Hunsberger, J.Amer.Chem.Soc.(1955), 77, 2466,2474) to provide aldehydes of general formula (27). Alternatively, anisoles of general formula (26) can be deprotonated with butyllithium in a solvent such as ether and the resulting anion quenched with a formamide such as N,N-dimethylformamide as described in (Murray, P. J. Bioorg.Med.Chem.Lett (1996), 6, 403-408) to provide aldehydes of general formula (27). Aldehydes of general formula (27) can be treated with phosphonates or phophonium reagents such as (2-carboxyethyl)triphenylphosphonium bromide, prepared as described in (Abdukakharov, V. S. Chem.Nat.Compd.(Engl.Transl.) (1990), 4, 486-487), in the presence of sodium hydride in a solvent such as dimethylsulfoxide to provide acids of general formula (28), wherein p is 0, 1, or 2. Acids of general formula (28) can be hydrogenated using a catalyst such as palladium on carbon in a solvent such as ethyl acetate to provide acids of general formula (29). Acids of general formula (29) can be cyclizated to provide methoxy compounds of general formula (30) under acidic conditions (such as heating in polyphosphoric acid for example) or Friedel-Crafts acylation conditions. Methoxy compounds of general formula (30) can be treated with a Lewis acid (AlCl₃ or the like) and a solvent (dichloromethane or the like) to provide phenols of general formula (31). Phenols of general formula (31) can be treated with 4-chloro-2-phenylquinazoline as described in (Newman, A. H. J. Med. Chem. (1992), 35, 4135-4142) to provide anilines of general formula (10). Anilines of general formula (10) can be processed as described in Schemes 1 and 2 to provide indanes, tetrahydronaphthalenes, or tetrahydrobenzo[a]cycloheptenes of general formula (8).

Alternatively, phenols of general formula (31) can be treated with trifluoromethane sulfonic anhydride in the presence of a non nucleophilic base (such as 2,6-di-tert-butyl-4-methylpyridine or the like) in a solvent (such as dichloromethane) to provide trifluoromethanesulfonates of general formula (32). Treatment of sulfonates (32) with primary amines such as benzyl amine or optionally substituted anilines in the presence of a palladium catalyst such as palladium (II) acetate under conditions described by (Buchwald, J. Org. Chem. (1997), 62, 1264-1267) can provide compounds of general formula (33). Compounds of general formula (33) can be processed as described in Schemes 1 or 2 to provide tetrahydronaphthalenes of general formula (8).

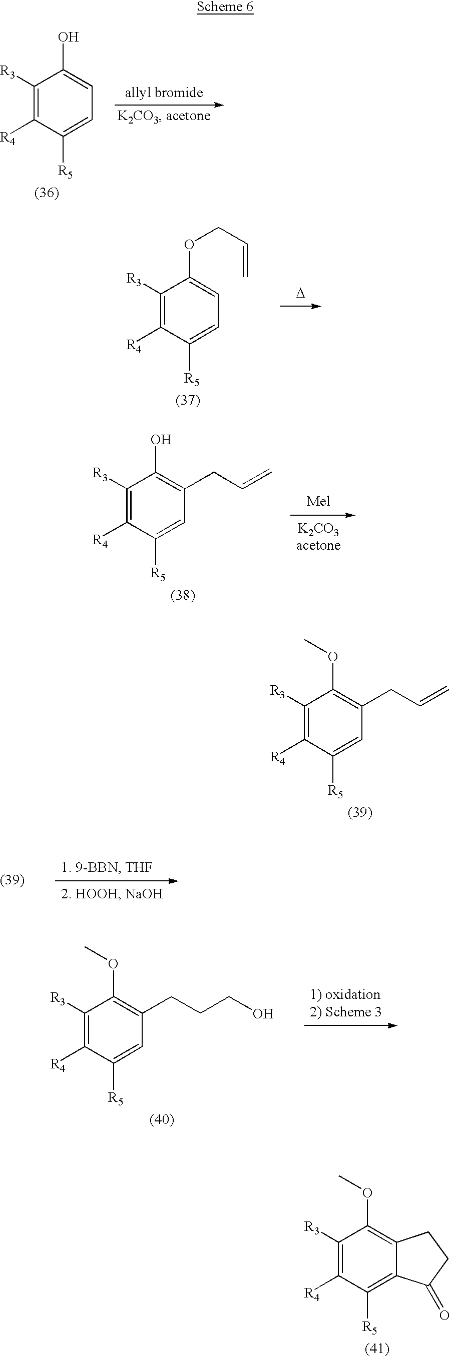

An alternate method for preparing methoxyindanones (41), methoxytetrahydronaphthalenones (42), and methoxytetrahydrobenzo[a]cycloheptenones (43), wherein R₃, R₄, and R₅ are as defined in formula I, can be used as described in Scheme 6. Phenols of general formula (36) can be treated with allyl bromide in the presence of a base such as potassium carbonate in a solvent such as acetone to provide allylic ethers of general formula (37). Claisen rearrangement of ethers of general formula (37) via heating with or without a solvent such as N,N-diethylaniline provides phenols of general formula (38). Phenols of general formula (38) can be methylated with methyl iodide or the like using a base such as potassium carbonate in a solvent such as acetone to provide anisoles of general formula (39). Anisoles of general formula (39) can be treated with a hydroborating agent such as 9-borabicyclo[3.3.1]nonane or the like in a solvent such as THF followed by oxidation with hydrogen peroxide in aqueous sodium hydroxide or the like to provide alcohols of general formula (40). Alcohols of general formula (40) can be treated with an oxidizing agent such as nitric acid or chromic acid to provide the corresponding carboxylic acid which can then be processed as described in Scheme 3 to provide methoxyindanones of general formula (41). Alcohols of general formula (40) can be processed as described in Scheme 4 to provide methoxytetrahydronaphthalenones of general formula (42) and methoxytetrahydrobenzo[a]cycloheptenones of general formula (43).

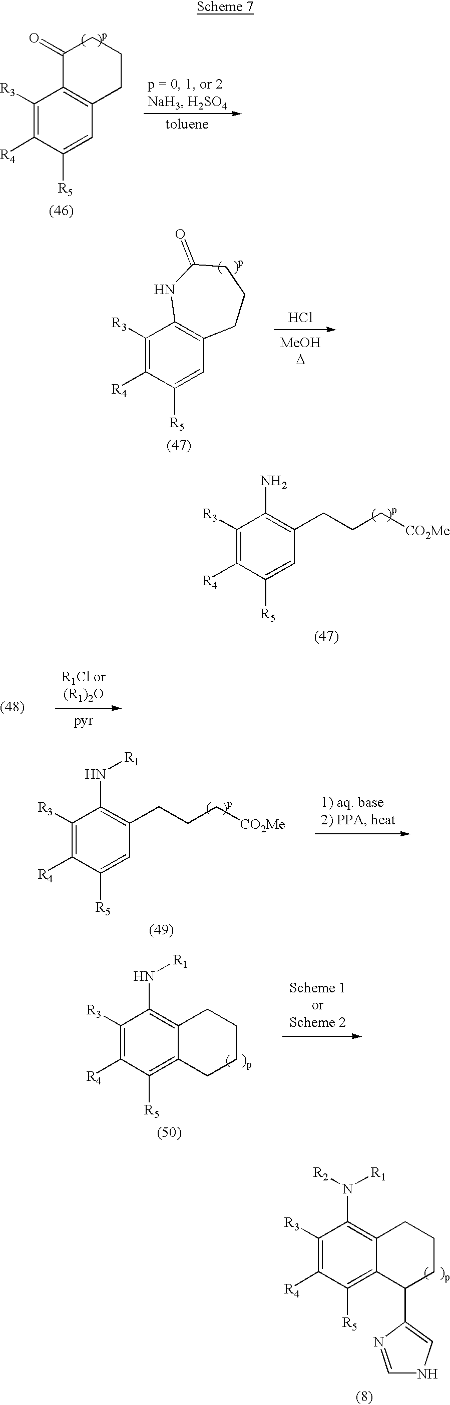

Another method of preparing indanes, tetrahydronaphthalenes, or tetrahydrobenzo[a]cycloheptenes of general formula (8), wherein p is 0, 1, or 2, and R₁, R₂, R₃, R₄, and R₅ are as defined in formula I, can be used as described in Scheme 7. Indanones, tetrahydronaphthalenones, or tetrahydrobenzo[a]cycloheptenones of general formula (46), can be treated with sodium azide in the presence of sulfuric acid in a solvent such as toluene to provide lactams of general formula (47). Lactams of general formula (47) can be treated with hydrochloric acid in methanol with heat to provide anilines of general formula (48). Anilines of general formula (48) can be treated with acylating or sulfonating agents in a solvent such pyridine to provide esters of general formula (49). Esters of general formula (49) can be cyclized to provide indanones, tetrahydronaphthalenones, or tetrahydrobenzo[a]cycloheptenones of general formula (50) by heating in an acid such as polyphosphoric acid for example. Indanones, tetrahydronaphthalenones, or tetrahydrobenzo[a]cycloheptenones of general formula (50) can be processed as described in Schemes 1 and 2 to provide indanes, tetrahydronaphthalenes, or tetrahydrobenzo[a]cycloheptenes of general formula (8).

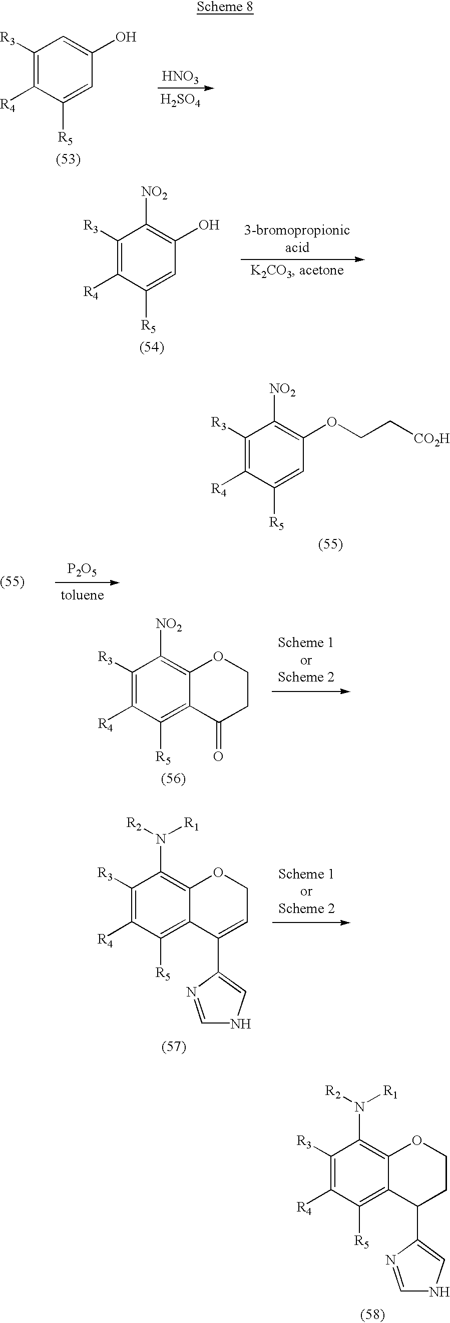

Chromanes of general formula (58), wherein R₁, R₂, R₃, R₄, and R₅ are as defined in formula I, can be prepared as described in Scheme 8. Phenols of general formula (53) can be nitrated (54) and then treated with 3-bromopropionic acid to provide acids of general formula (55). Acids of general formula (55) can be cyclized with phosphorous pentoxide to provide chromanones of general formula (56). Chromanones of general formula (56) can be processed as described in Schemes 1 and 2 to provide chromenes of general formula (57) and chromanes of general formula (58).

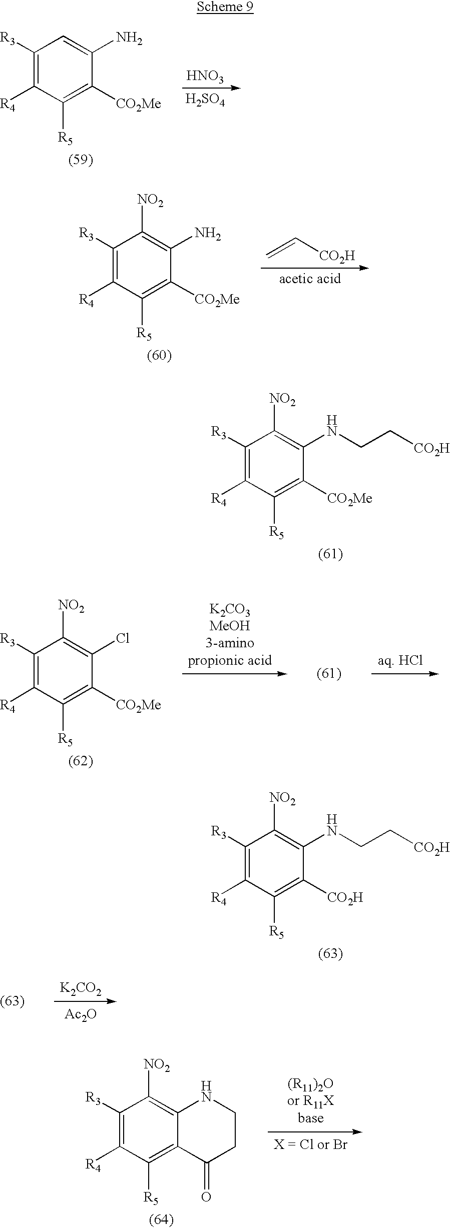

Tetrahydroquinolines of general formula (69), wherein R₁, R₂, R₃, R₄, R₅, and R₁₁ are as defined in formula I, can be prepared as described in Scheme 9. Anilines of general formula (59) can be treated with a nitrating agent such as fuming nitric acid to provide nitroanilines of general formula (60). Nitroanilines of general formula (60) can be treated with acrylic acid in a solvent such as acetic acid to provide propionic acids of general formula (61). Propionic acids of general formula (61) can also be prepared from substituted nitrohalides of general formula (62). Nitrohalides of general formula (62) can be treated with 3-aminopropionic acid in the presence of a base such as potassium carbonate to provide propoionic acids of general formula (61). Propionic acids of general formula (61) can be saponified under aqueous acidic conditions to provide diacids of general formula (63). Diacids of general formula (63) can be cyclized using potassium acetate and acetic anhydride as described in (Bolotina, L. A Chem.Het.Compd.(Engl.Transl.), (1982), 18, 671-673) to provide nitroquinolinones of general formula (64). Nitroquinolinones of general formula (64) can be treated with acylating or sulfonylating agents (such as sulfonyl chlorides, anhydrides, acid chlorides, or the like) using a mild base (such as pyridine) in a solvent (such as dichloromethane) to provide N-acylated nitroquinolinones of general formula (65) or N-sulfonated nitroquinolinones of general formula (65). Alternatively, nitroquinolinones of general formula (64) also can be alkylated with alkyl halides such as methyl iodide, ethyl iodide, benzyl bromide, or the like in the presence of a base such as potassium carbonate to provide or N-alkylated nitroquinolinones of general formula (65). Nitroquinolinones of general formula (65) can be processed as described in previous Schemes 1 and 2 to provide compounds of general formula (66). Compounds of general formula (66) can be treated with acid to provide dihydroquinolines of general formula (68). Compounds of general formula (66) can also be exposed to hydrogenation conditions followed by treatment with acid to provide tetrahydroquinolines of general formula (69).

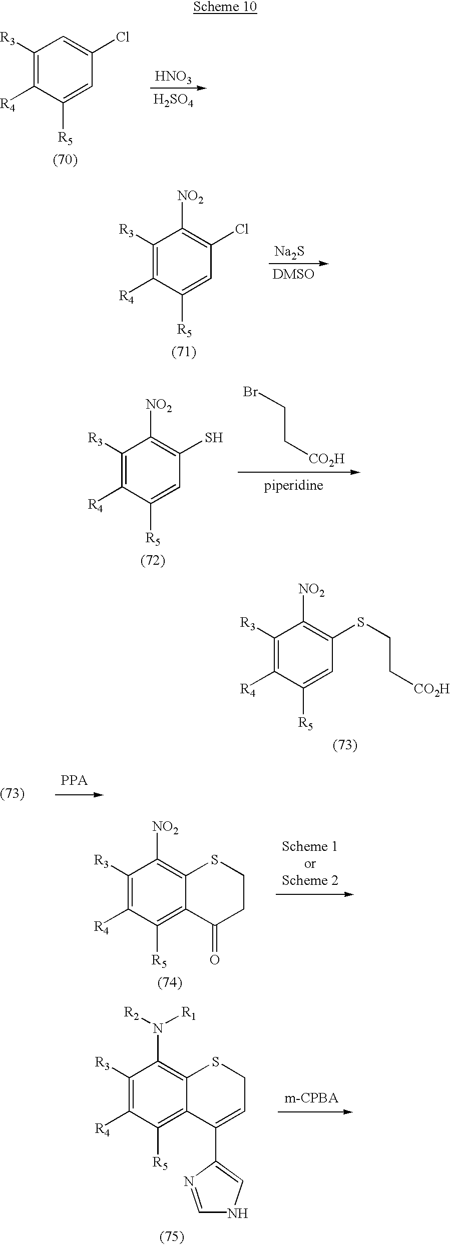

Thiochromanes of general formula (77) and (78), wherein R₁, R₂, R₃, R₄, and R₅ are as defined in formula I and n is 1 or 2, can be prepared as described in Scheme 10. Chlorobenzenes of general formula (70) can be nitrated at the ortho position to provide ortho-chloronitrobenzenes of general formula (71). Ortho-chloronitrobenzenes of general formula (71) can be treated with sodium sulfide in dimethylsulfoxide to provide nitrothiophenols of general formula (72). Nitrothiophenols of general formula (72) can be treated with 3-bromopropionic acid in the presence of piperidine to provide acids of general formula (73). Acids of general formula (73) can be cyclized as described in (Schaefer, T. Can.J.Chem. (1987), 65, 908-914) to provide thiochromenones of general formula (74). Thiochromenones of general formula (74) can be processed as described in Schemes 1 and 2 to provide thiochromenes of general formula (75) which can be selectively oxidized to the sulfoxides or sulfones of general fomula (76) using one or two equivalents respectively of an oxidant such as 3-chloroperoxybenzoic acid (m-CPBA) or the like. Thiochromenes of general formula (75) can be treated with a reducing agent such as hydrazine in a solvent such as methanol or catalytic hydrogenation using palladium in the presence of barium sulfate to provide thiochromanes of general formula (77) which can be selectively oxidized to the sufoxides or sulfones of general formula (78) using one or two equivalents respectively of an oxidant such as 3-chloroperoxybenzoic acid (m-CPBA) or the like.

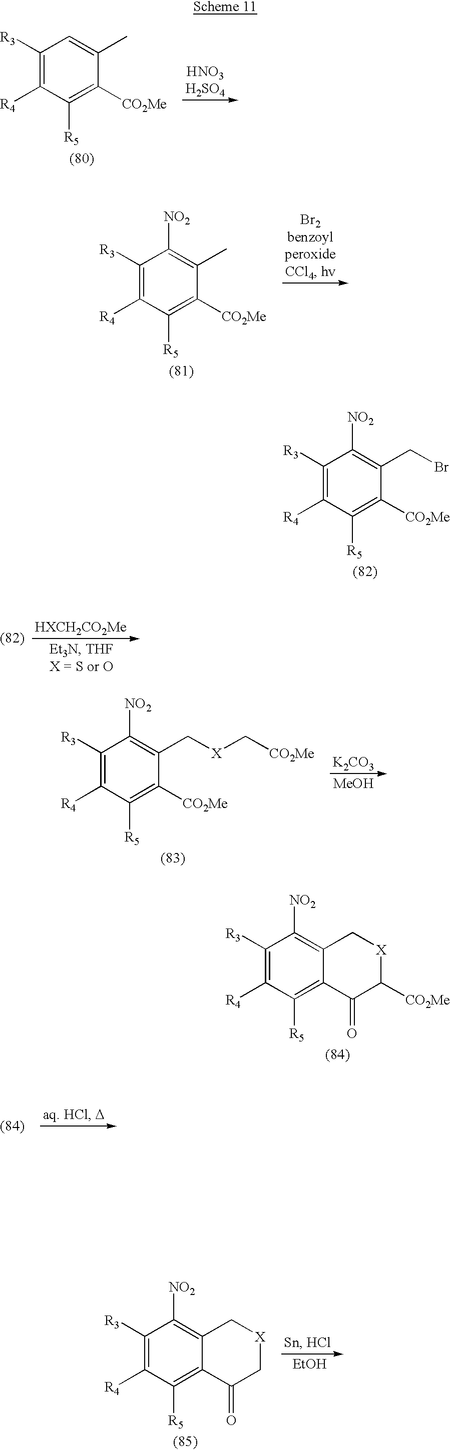

Isochromenes and isothiochromenes of general formula (88), wherein R₁, R₂, R₃, R₄, and R₅ are as defined in formula I and X is O or S, can be prepared as described in Scheme 11. 2-Methylbenzoates of general formula (80) can be nitrated to provide nitro compounds of general formula (81). Nitro compounds of general formula (81) can be treated with bromine in the presence of benzoyl peroxide and light as described in (Soederberg, B. J.Org.Chem. (1997), 62, 5838-5845) to provide benzyl bromides of general formula (82). Benzyl bromides of general formula (82) can be treated with methyl thioglycolate or methyl hydroxyglycolate in the presence of triethyl amine, with silver oxide when X is O, in THF to provide diesters of general formula (83). Diesters of general formula (83) can be cyclized under basic conditions (potassium carbonate in methanol) to provide ketoesters of general formula (84). Ketoesters of general formula (84) can be decarboxylated by heating in aqueous acid to provide nitroisothiochromenones or nitroisochromenones of general formula (85). An alternate method of preparing nitroisochromenones of general formula (85) can be used as described in (Anzalone, L. J.Org.Chem. (1985) 50, 2128-2133). Nitroisothiochromenones or nitroisochromenones of general formula (85) can be reduced using a metal such as tin to provide anilines of general formula (86). Anilines of general formula (86) can be processed as described in Schemes 1 and 2 to provide compounds of general formula (87). Compounds of general formula (87) can be reduced using zinc in hydrochloric acid to provide isochromenes and isothiochromenes of general formula (88). Isothiochromenes of general formula (88) can be selectively oxidized to the sufoxides or sulfones of general formula (89) using one or two equivalents respectively of an oxidant such as 3-chloroperoxybenzoic acid (m-CPBA) or the like.

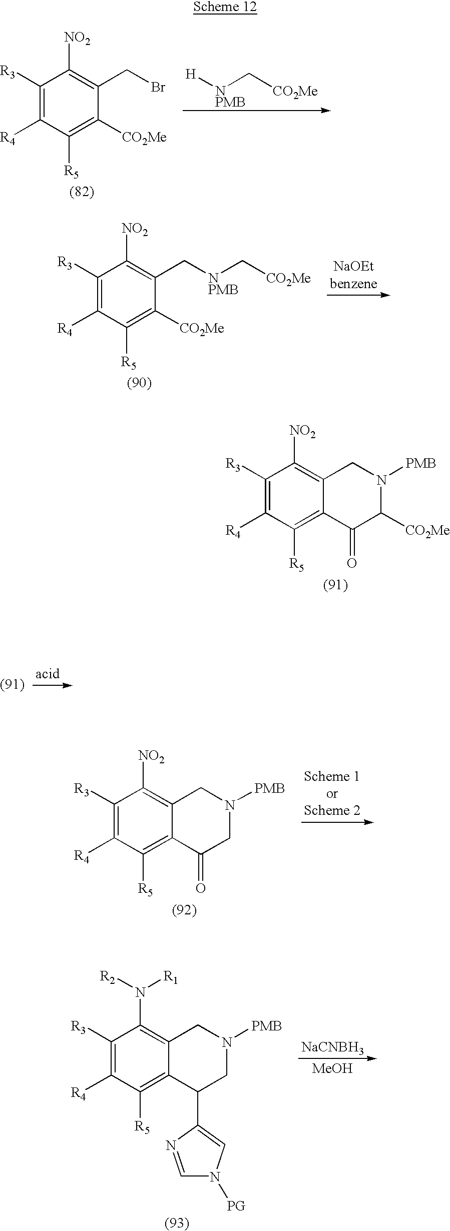

Tetrahydroisoquinolines of general formula (97), wherein R₁, R₂, R₃, R₄, R₅, and R₁₁ are as defined in formula I, can be prepared as described in Scheme 12. Benzyl bromides of general formula (82), from Scheme 11, can be treated with methyl [(4-methoxybenzyl)amino]acetate as described in (Weygand,F. Chem.Ber. (1968) 101, 3623-3641) in the presence of a base such as triethylamine to provide diesters of general formula (90). Diesters of general formula (90) can be treated with a base such as sodium ethoxide in a solvent such as benzene to provide ketoesters of general formula (91). Ketoesters of general formula (91) can be decarboxylated under acidic conditions to provide isoquinolinones of general formula (92). Isoquinolinones of general formula (92) can be processed as described in Schemes 1 and 2 to provide dihydroisoquinolines of general formula (93). Dihydroisoquinolines of general formula (93) can be treated with reducing agents such as sodium cyanoborohydride in methanol to provide tetrahydroisoquinolines of general formula (94). The protecting group (PMB) can be removed with ceric ammonium nitrate to provide secondary amines of general formula (95). Secondary amines of general formula (95) can be treated with electrophiles in the presence of a base such as pyridine or potassium carbonate to provide N-substituted tetrahydroisoquinolines of general formula (96). N-Substituted tetrahydroisoquinolines of general formula (96) can be deprotected with acid as described in previous schemes to provide tetrahydroisoquinolines of general formula (97).

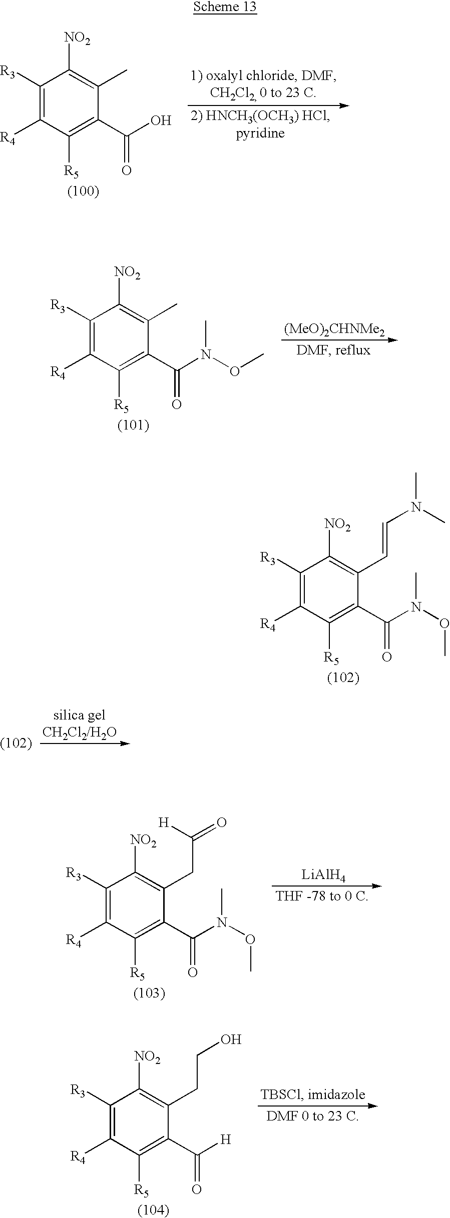

Tetrahydroisoquinolines of general formula (113), wherein R₁, R₂, R₃, R₄, R₅, and R₆, are as defined in formula I, can be prepared as described in Scheme 13. 2-Methyl-3-nitrobenzoic acids of general formula (100) can be treated with oxalyl chloride and DMF in methylene chloride starting at 0° C. and warming to 23° C. to form acid chlorides which are immediately treated with N,O-dimethylhydroxylamine hydrochloride and pyridine to form amides of general formula (101). Amides of general formula (101) can be treated with dimethylformamide dimethyl acetal in dimethylformamide at reflux to provide enamines of general formula (102). Enamines of general formula (102) can be treated with silica gel in a mixture of methylene chloride and water to provide aldehydes of general formula (103). Aldehydes of general formula (103) can be treated with lithium aluminum hydride in tetrahydrofuran to provide alcohols of general formula (104) on warming from −78° C. to 0° C. Alcohols of general formula (104) can be treated with tert-butyldimethylsilyl chloride and imidazole in DMF at 0° C. and warmed to 23° C. to form silylethers of general formula (105). Silylethers of general formula (105) can be treated with iron and NH₄Cl in a solution of refluxing ethanol and water to provide anilines of general formula (106). Anilines of general formula (106) can be processed as described in previous Schemes 1 and 2 to provide substituted anilines of general formula (107). Substituted anilines of general formula (107) can be treated with di-tert-butyl dicarbonate and N,N-dimethylaminopyridine in acetonitrile at 23° C. to provide N-protected anilines of general formula (108). N-Protected anilines of general formula (108) can be treated at 23° C. with a pre-mixed solution of 4-iodo-N,N-dimethyl-1H-imidazole-1-sulfonamide and ethyl magnesium bromide in methylene chloride to provide alcohols of general formula (109). Alcohols of general formula (109) can be treated with tetrabutylammonium fluoride in tetrahydrofuran between 0° C. and 23° C. to provide diols of general formula (110). Diols of general formula (110) can be treated with 2 equivalents of methanesulfonyl chloride and triethylamine in methylene chloride to provide bis methanesulfonates of general formula (111). Bis methanesulfonates of general formula (111) can be treated with primary amines in methylene chloride at ambient temperature to provide isoquinolines of general formula (112). Isoquinolines of general formula (112) can be treated with trifluoroacetic acid in dichloromethane and electrophiles in a two step procedure to provide isoquinolines of general formula (114). Isoquinolines of general formula (114) can be treated with 2N HCl and dioxane at reflux to remove the sulfamoyl protecting group providing isoquinolines of general formula (115).

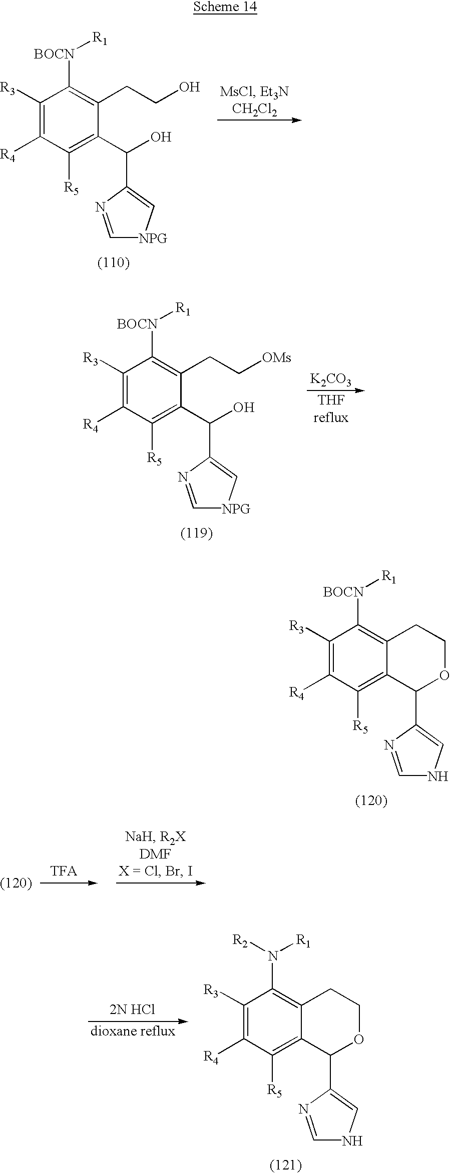

Isochromenes of general formula (121), wherein R₁, R₂, R₃, R₄, and R, are as defined in formula I, can be prepared as described in scheme 14. Diols of general formula (110), from Scheme 13, can be treated with one equivalent of methanesulfonyl chloride and a base such as triethylamine to provide methanesulfonates of general formula (119). Methanesulfonates of general formula (119) can be treated with K₂CO₃ in tetrahydrofuran at reflux to provide isochromenes of general formula (120). Isochromenes of general formula (120) can be treated with trifluoroacetic acid, a strong non nucleophilic base (such as sodium hydride or the like) in a solvent (such as DMF or the like) and electrophiles such as alkyl halides, arylalkyl halides, cycloalkyl halides, or cycloalkylalkyl halides, and 2N HCl in dioxane at reflux in a stepwise fashion to provide isochromenes of general formula (121).

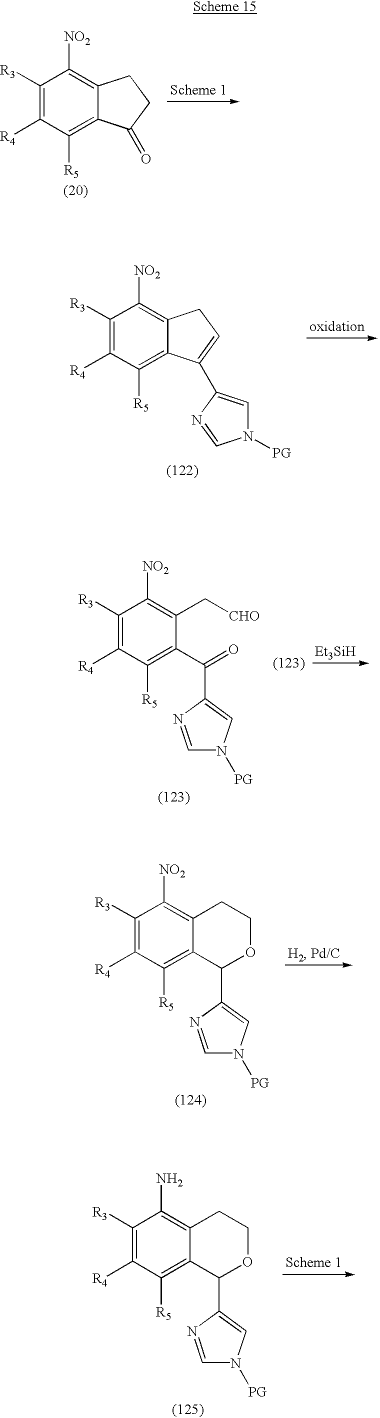

An alternate route to ischromenes of general formula (121), wherein R₁, R₂, R₃, R₄, and R, are as defined in formula I, can be used as described in Scheme 15. Nitroindanones of general structure (20), from Scheme 3, can be processed as described in Scheme 1 to provide indenes of general formula (122). Indenes of general formula (122) can be exposed to oxidative conditions as described in (Jiancheng, Zhang, Tetrahedron Lett, 27, 51, (1986) 6153-6156; Wuensch, Thomas J.Org.Chem. 55, 14, (1990) 4233-4235; Kometani, Tadashi, J. Chem. Soc. Perkin Trans.1, (1981) 1191-1196) to provide ketoaldehydes of general formula (123). Ketoaldehydes of general formula (123) can be cyclized to isochromenes of general formula (124) using triethylsilane as described in (McCullough, K., J.Chem.Soc.Perkin Trans.1, 15, (1998) 2353-2362). Isochromenes of general formula (124) can be treated with a palladium catalyst such as palladium on carbon in a solvent such as methanol, ethanol or ethyl acetate under a hydrogen atmosphere to provide anilines of general formula (125). Anilines of general formula (125) can be processed as described in Scheme 1 to provide ischromenes of general formula (121).

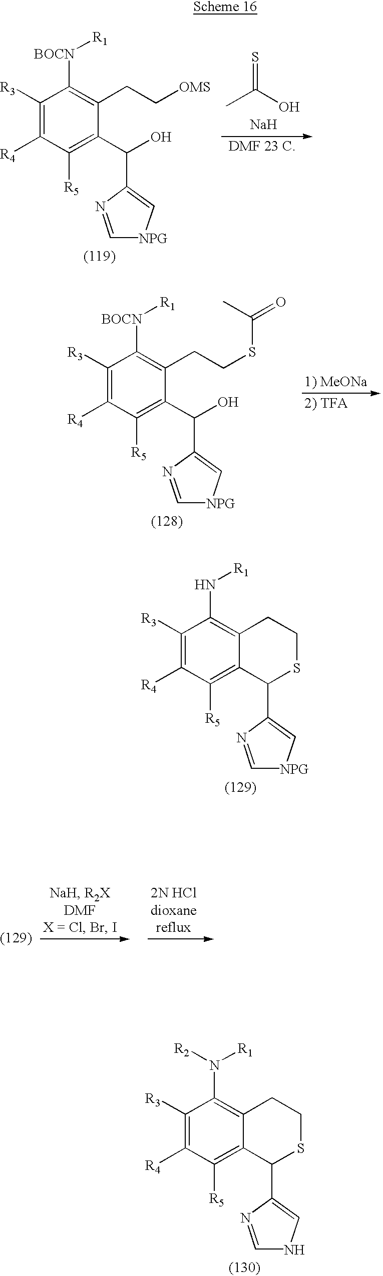

Isothiochromenes of general formula (130), wherein R₁, R₂, R₃, R₄, and R₅ are as defined in formula I, can be prepared as described in Scheme 16. Methanesulfonates of general formula (119), from Scheme 14, can be treated with thioacetic acid and sodium hydride to provide thioates of general formula (128). Thioates of general formula (128) can be treated with sodium methoxide and then trifluoroacetic acid to provide isothiochromenes of general formula (129). Isothiochromenes of general formula (129) can be processed as described in Scheme 1 to provide isothiochromenes of general formula (130).

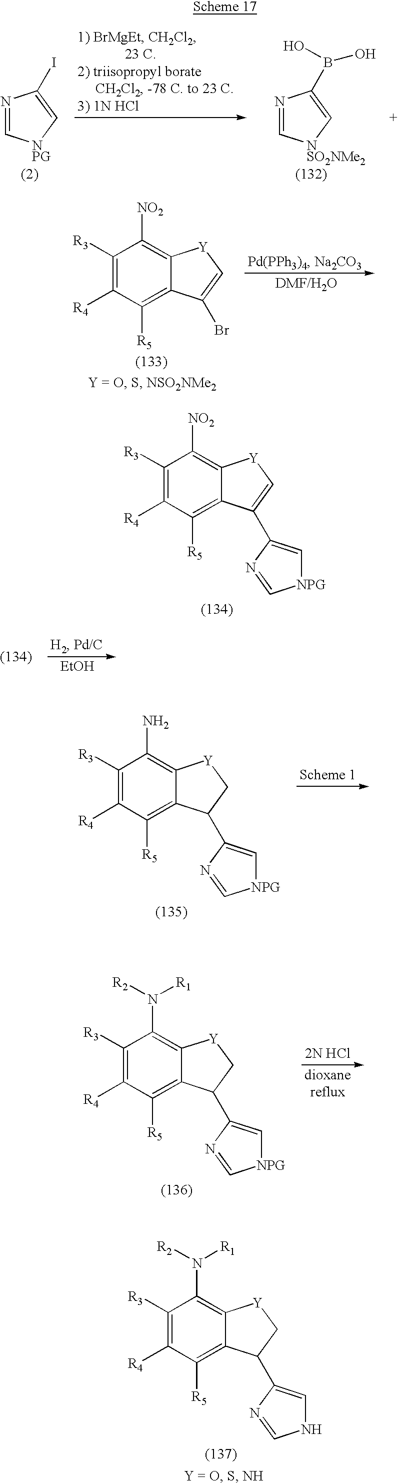

Indolines, dihydrobenzofurans, and dihydrobenzothiophenes of general formula (137), wherein R₁, R₂, R₃, R₄, and R₅ are as defined in formula I, can be prepared as described in Scheme 17. 4-Iodo-N,N-dimethyl-1H-imidazole-1-sulfonamide (2), from Scheme 1 wherein PG is N,N-dimethylsulfamoyl, can be treated with ethyl magnesium bromide in methylene chloride at 23° C.; triisopropyl borate in methylene chloride between −78° C. and 23° C.; and 1N HCl in water to provide 1-[(dimethylamino)sulfonyl]-1H-imidazol-4-ylboronic acid (132). 3-Bromobenzofurans, 3-bromobenzothiophenes, and 3-bromoindoles, from Schemes 18 and 19, can be treated with boronic acid (132), palladium tetrakistriphenylphosphine, and sodium carbonate in water and DMF to provide nitroimidazoles of general formula (134). Nitroimidazoles of general formula (134) can be treated with hydrogen and Pd/C in ethanol to provide anilines of general formula (135). Anilines of general formula (135) can be processed as described in Scheme 1 to provide compounds of general formula (136). Compounds of general formula (136) can be treated with 2N HCl and dioxane at reflux to provide compounds of general formula (137), wherein Y is selected from O, S, and NH. Indoles of general formula (137), wherein Y is NH, can be treated with one equivalent of di-tert-butyl dicarbonate and then processed as described in Scheme 12 to provide indoles of general formula (137) wherein Y is other than NH.

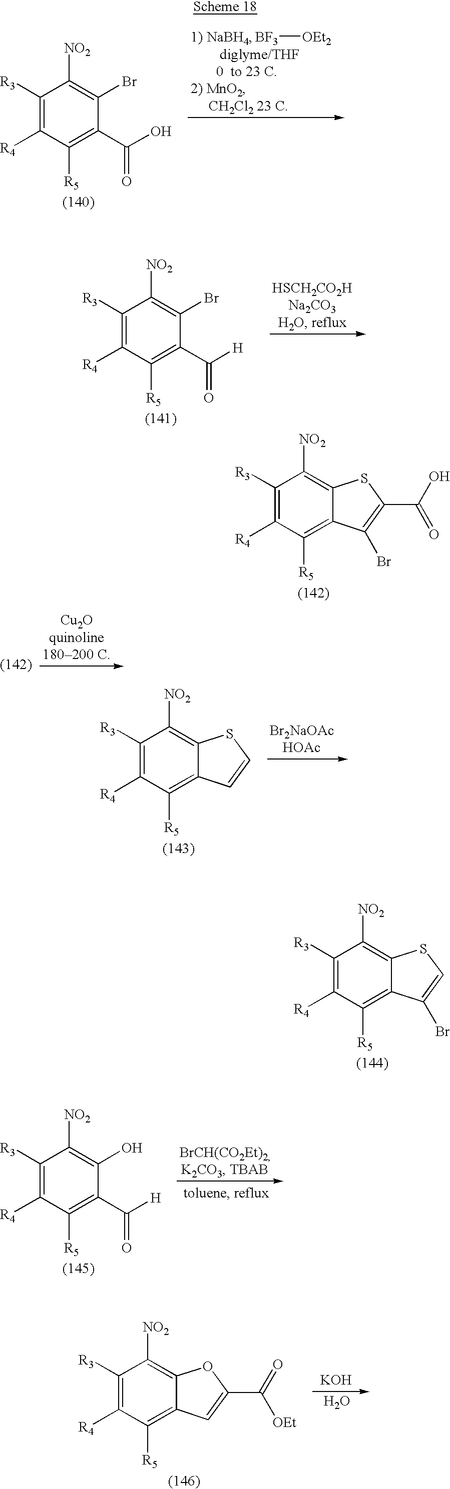

3-Bromobenzothiophenes of general formula (144), wherein R₃, R₄, and R₅ are as defined in formula I, can be prepared as describeded in Scheme 18. Nitrobenzoic acids of general formula (140) can be treated with sodium borohydride and boron trifluoride etherate in diglyme and THF between 0° C. and 23° C. and then treated with manganese dioxide in chloroform at 23° C. to provide aldehydes of general formula (141). Aldehydes of general formula (141) can be treated with mercaptoacetic acid in aqueous sodium carbonate at reflux to provide 7-nitrobenzothiophene-2-carboxylic acids of general formula (142) which can be decarboxylated with cuprous oxide in quinoline between 180° C. and 200° C. to provide 7-nitrobenzothiophenes of general formula (143). 7-Nitrobenzothiophenes of general formula (143) can be treated with bromine and anhydrous sodium acetate in acetic acid to form 3-bromobenzothiophenes of general formula (144).

3-Bromobenzofurans of general formula (150), wherein R₃, R₄, and R₅ are as defined in formula I, can be prepared as describeded in Scheme 18. Nitrobenzaldehydes of general formula (145) can be treated with diethyl bromomalonate, potassium carbonate, and tetrabutylammonium bromide in toluene at reflux to provide nitrobenzofurans of general formula (146). Nitrobenzofurans of general formula (146) can be hydrozyled with potassium hydroxide in water to provide acids of general formula (147). Acids of general formula (147) can be decarboxylated with cuprous oxide in quinoline between 180° C. and 200° C. to form the 7-nitrobenzofurans of general formula (148). 7-Nitrobenzofurans of general formula (148) can be dibrominated by treatment with bromine in acetic acid to provide dibromobenzoflrans of general formula (149). Dibromobenzofurans of general formula (149) can be treated with potassium ethoxide in ethanol to provide 3-bromonitrobenzofurans of general formula (150).

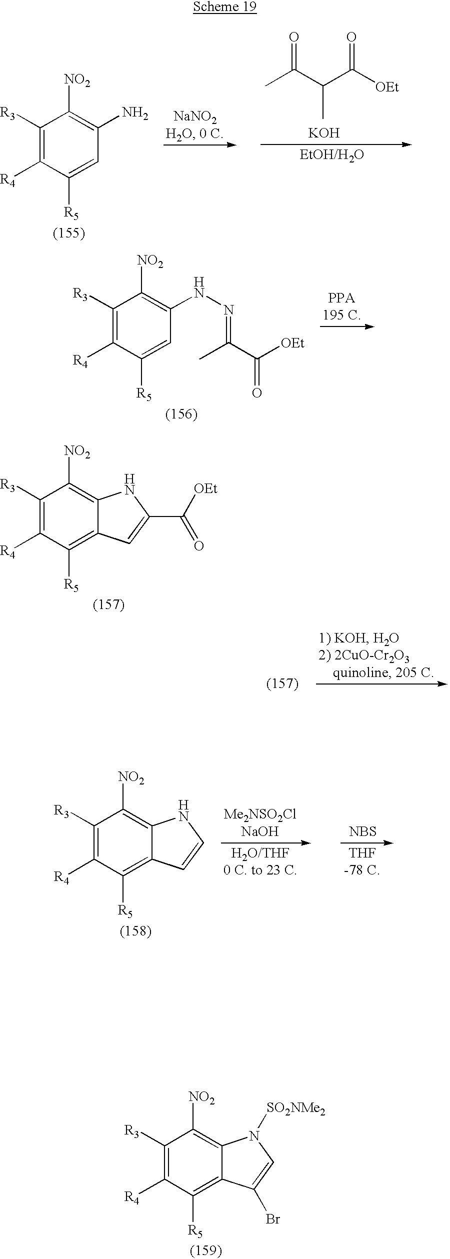

3-Bromoindoles of general fomula (159), wherein R₃, R₄, and R₅ are as defined in formula I, can be prepared as describeded in Scheme 19. 2-Nitroanilines of general formula (155) can be treated with sodium nitrate in water at 0° C. to provide diazonium compounds which can then be treated with ethyl 2-methyl-3-oxobutanoate and potassium hydroxide in ethanol and water to provide hydrazones of general formula (156). Hydrazones of general formula (156) can be heated in polyphosphoric acid at 195° C. to facilitate ring closure to provide indoles of general formula (157). Indoles of general formula (157) can be saponified by treatment with potassium hydroxide and water (may require heating) and then decarboxylated with copper chromite in quinoline at 205° C. to provide 7-nitroindoles of general formula (158). 7-Nitroindoles of general formula (158) can be N-protected by treatment with N,N-dimethylsulfamoyl chloride and sodium hydroxide in THF and water between 0° C. and 23° C. and then treated with N-bromosuccinimide in THF at −78° C. to provide 3-bromoindoles of general fomula (159).

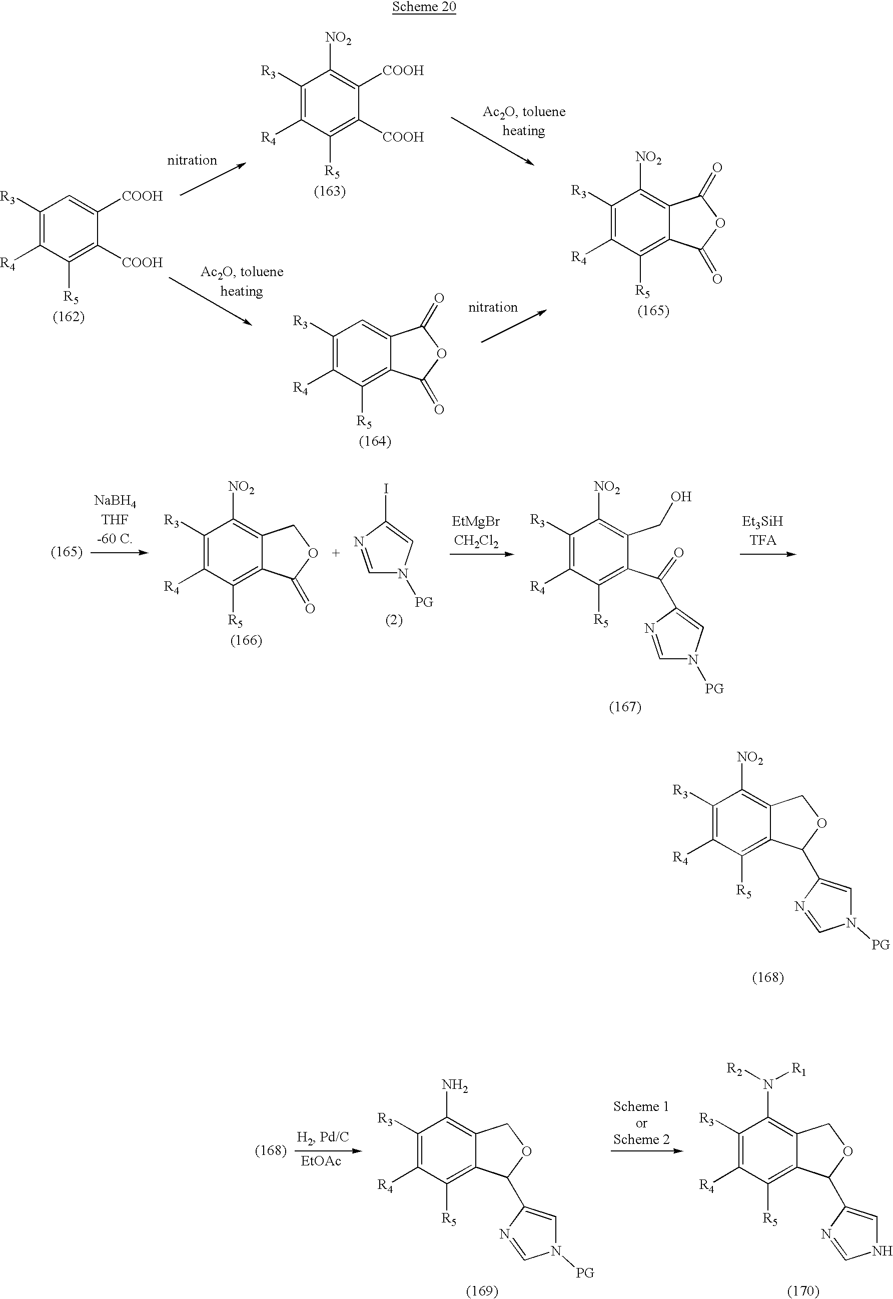

Isobenzofurans of general formula (170), wherein R₁, R₂, R₃, R₄, and R₅ are as defined in formula I, can be prepared as described in scheme 20. Phthalic acids of general formula (162) can be nitrated under standard conditions to provide the nitro phthalic acids of general formula (163) which can be treated with acetic anhydride in toluene to provide nitro phthalic anhydrides of general formula (165). Alternatively, phthalic acids of general formula (162) can be converted to anhydrides of general formula (164) and then nitrated to provide nitro phthalic anhydrides of general formula (165). Phthalic anhydrides of general formula (165) can be reduced as described in (Stanetty, Peter J.Prakt.Chem./Chem.-Ztg. 335; 1; (1993) 17-22) to provide benzofuranones of general formula (166). Benzofuranones of general formula (166) can be treated with 4-iodo-N,N-dimethyl-1H-imidazole-1-sulfonamide (2), from Scheme 1 wherein PG is N,N-dimethylsulfamoyl, and ethylmagnesium bromide to provide ketoalcohols of general formula (167). Ketoalcohols of general formula (167) can be treated with triethylsilane in trifluoroacetic acid to provide isobenzofurans of general formula (168), which can then be processed as described in previous schemes to isobenzofurans of general formula (170).

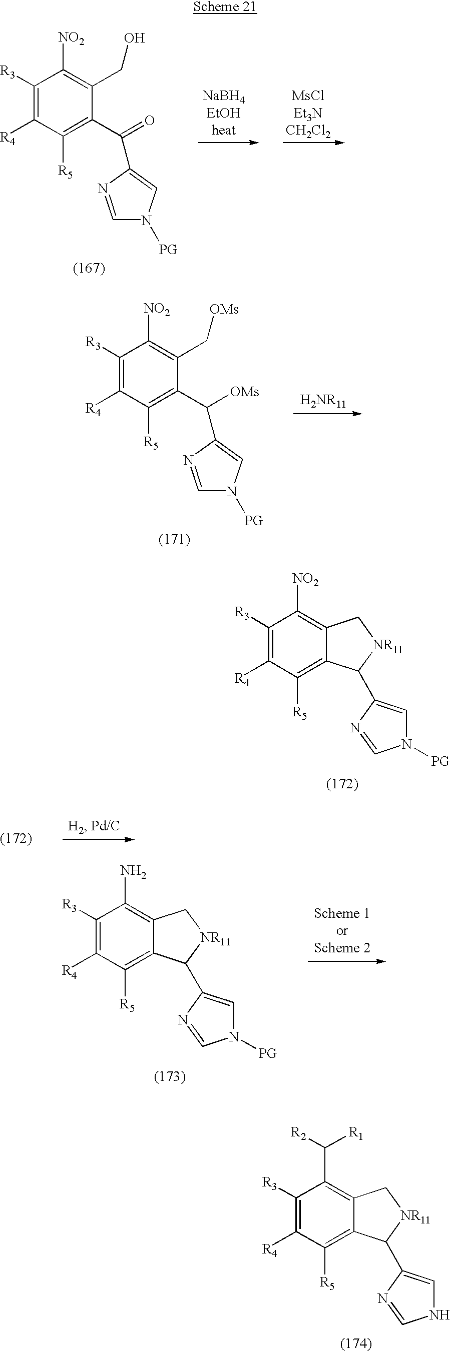

Isoindolines of general formula (174), wherein R₁, R₂, R₃, R₄, R₅, and R₁₁ are as defined in formula I, can be prepared as described in Scheme 21. Ketoalcohols of general formula (167), from Scheme 20, can be treated with sodium borohydride and then 2.0 equivalents of methanesulfonyl chloride to provide bismethanesulfonates of general formula (171). Bismethanesulfonates of general formula (171) can be treated with primary amines to provide nitroisoindolines of general formula (172). Nitroisoindolines of general formula (172) can be treated with a palladium catalyst such as palladium on carbon under a hydrogen atmosphere or a metal reducing agent such as zinc or iron to provide anilines of general formula (173). Anilines of general formula (173) can be processed as described in Schemes 1 or 2 to provide isoindolines of general formula (174).

Isoindoles of general formula (174) wherein R₁₁ is benzyl can be treated with di-tert-butyl dicarbonate and then reduced using a palladium catalyst under a hydrogen atmosphere to provide isoindoles of general formula (175). Isoindoles of general formula (175) wherein R₁₁ is hydrogen can be processed as described in Scheme 12 to provide isoindoles of general formula (174) wherein R₁₁ is other than benzyl or hydrogen.

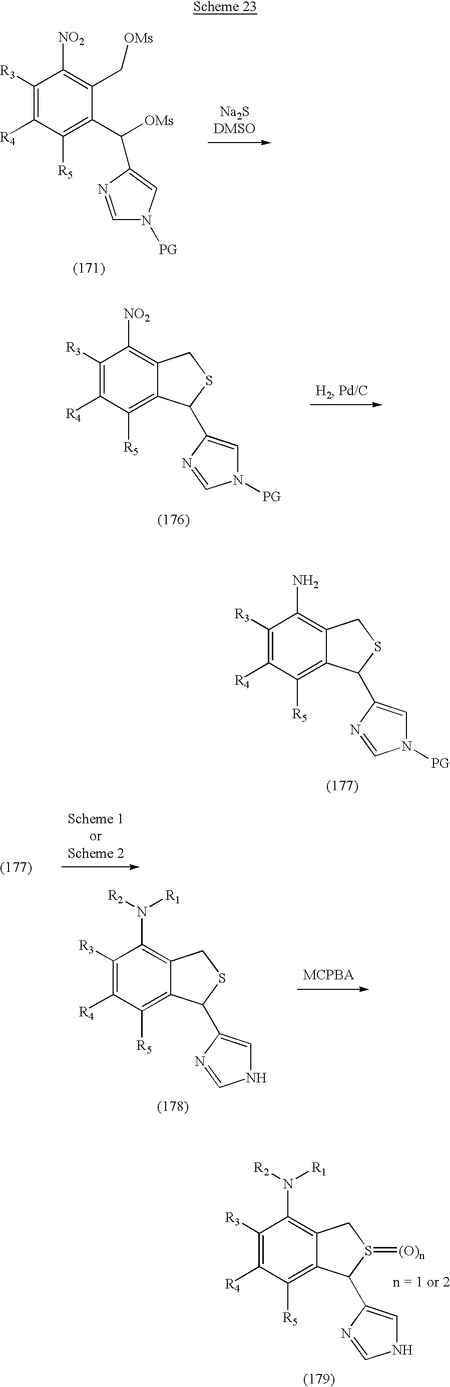

1,3-Dihydro-2-benzothiophenes of general formula (178) and (179), wherein R₁, R₂, R₃, R₄, and R₅ are as defined in formula I, can be prepared as described in Scheme 23. Bismethanesulfonates of general formula (171), from Scheme 21, can be treated with sodium sulfide in a solvent such as dimethylsulfoxide as described in (Mann, John, J.Chem.Soc.Perkin Trans.1, (1984) 2081-2088) to provide 4-nitro-1,3-dihydro-2-benzothiophenes of general formula (176). 4-Nitro-1,3-dihydro-2-benzothiophenes of general formula (176) can be treated with zinc in acetic acid to provide anilines of structure (177) which can be processed as described in Schemes 1 or 2 to provide 1,3-dihydro-2-benzothiophenes of general formula (178). 1,3-Dihydro-2-benzothiophenes of general formula (178) can be treated with 1 or 2 equivalents of meta-chloroperoxybenzoic acid to provide sulfoxides or sulfones of general formula (179).

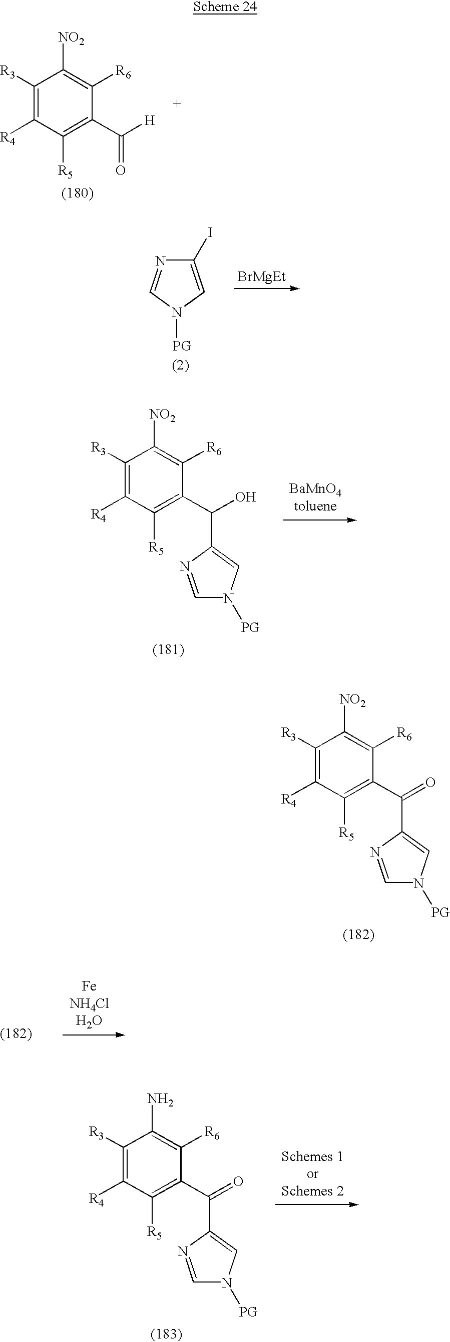

Olefins of general formula (185), wherein R₁, R₂, R₃, R₄, R₅, R₆, R₁₂ and R₁₃ are as defined in formula I, can be prepared as described in Scheme 24. Nitrobenzaldehydes of general formula (180) can be treated with 4-iodo-N,N-dimethyl-1H-imidazole-1 sulfonamide (2), from Scheme 1 wherein PG is N,N-dimethylsulfamoyl, and ethylmagnesium bromide to provide alcohols of general formula (181). Alcohols of general formula (181) can be treated with barium manganate or manganese dioxide to provide ketones of general formula (182). Compounds of general formula (182) can be treated with iron to provide anilines of general formula (183) which can be processed as described in Schemes 1 or 2 to provide compounds of general formula (184). Compounds of general formula (184) can be treated with phosphonium or phosphonate compounds in the presence of an appropriate base to provide olefins of general formula (185). An alternate method of preparing olefins of general formula (185) can be used. Ketones of general formula (184) can be treated with alkyl, cycloalkyl, cycloalkylalkyl, or arylalkyl Grignard or lithium reagents to provide alcohols of general formula (186). Alcohols of general formula (186) can be dehydrated and deprotected under acidic conditions (such as aqueous HCl, para-toluenesulfonic acid, trifluoroacetic acid or the like) to provide olefins of general formula (185).

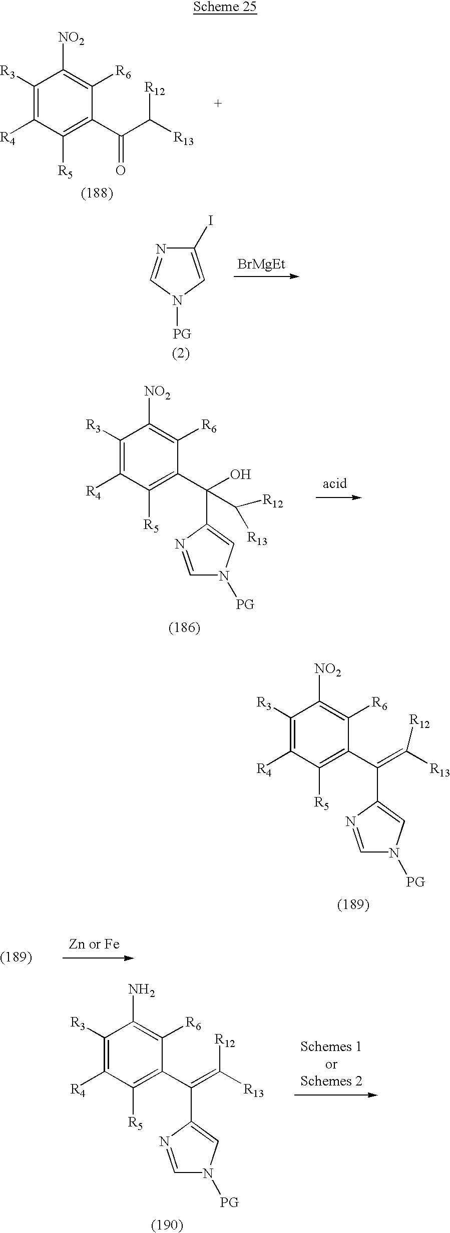

Olefins of general formula (185), wherein R₁, R₂, R₃, R₄, R₅, R₆, R₁₂ and R₁₃ are as defined in formula I, can be prepared as described in Scheme 25. Nitroketones of general formula (188) can be treated with 4-iodo-N,N-dimethyl-1H-imidazole-1-sulfonamide (2), from Scheme 1, wherein PG is N,N-dimethylsulfamoyl, and ethylmagnesium bromide to provide alcohols of general formula (186). Alcohols of general formula (186) can be dehydrated under acidic conditions (such as aqueous HCl, para-toluenesulfonic acid, trifluoroacetic acid or the like) to provide olefins of general formula (189). Olefins of general formula (189) can be treated with zinc or iron to provide anilines of general formula (190). Anilines of general formula (190) can be processed as describd in Scheme 1 or 2 to provide olefins of general formula (185).

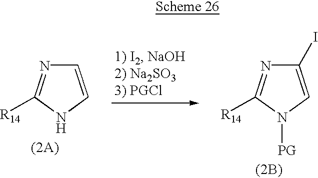

2-Alkyl-4-iodoimidazoles of general formula (2B), wherein R₁₄ is as defined in formula I, can be prepared as described in Scheme 26. 2-Alkylimidazoles of general formula (2A) can be treated with iodine in the presence of aquous sodium hydroxide, treated with sodium sulfite, and protected (PG) with trityl or N,N-dimethylsulfamoyl to provide 2-alkylimidazoles of general formula (2B) (Pyne, S. G., Synthesis (1994) 7, 681-682). 2-Alkyl-4-iodoimidazoles of general formula (2B) can be used as described in previous Schemes.

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography, or a combination of these procedures. Specific illustrations of suitable separation and isolation procedures can be had by reference to the Examples herein below. However, other equivalent separation or isolation procedures could, of course, also be used.

### EXAMPLE 1

### N-[5-(1H-imidazol-4-yl)-2-methoxy-5,6,7,8-tetrahydro-1-nahthalenyl]methanesulfonamide Hydrochloride

### Example 1A

### 4-(1-hydroxy-6-methoxy-5-nitro-1,2,3,4-tetrahydro-1-naphthalenyl)-N,N-dimethyl-1H-imidazole-1-sulfonamide

A solution of 4-iodo-N,N-dimethyl-1H-imidazole-1-sulfonamide (3.0 g, 10 mmol) (R. M. Turner, J. Org. Chem. (1991), 56, 5739-5740) in dichloromethane (40 mL) was treated with ethyl magnesium bromide (3.0M in diethyl ether, 3.3 mL) over 5 minutes, stirred for 30 minutes, treated with 6-methoxy-5-nitro-1-tetralone (2.6 g, 11.8 mmol), stirred for 16 hours, treated with ammonium chloride solution and extracted with dichloromethane. The extract was dried (MgSO₄), filtered and concentrated to provide the desired compound.

MS (DCI/NH₃) m/z 397 (M+H)⁺.

### Example 1B

### 4-(6-methoxy-5-nitro-3,4-dihydro-1-naphthalenyl)-1H-imidazole

A suspension of Example 1A (1.1 g, 2.2 mmol) in 1M HCl (30 mL) was heated to 90° C. for 16 hours, cooled to ambient temperature, treated with Na2CO₃ solution and extracted with 5:1 dichloromethane/ethanol. The extract was dried (MgSO₄), filtered, and concentrated. Purification of the residue on silica gel with 2% ethanol/ammonia-saturated dichloromethane provided the desired compound.

MS (DCI/NH₃) m/z 272 (M+H)⁺.

### Example 1C

### 5-(1H-imidazol-4-yl)-2-methoxy-5,6,7,8-tetrahydro-1-naphthalenamine

A mixture of Example 1B and 10% palladium on carbon (60 mg) in methanol (40 mL) was stirred under a hydrogen atmosphere for 16 hours, filtered through Celite,® and concentrated. Purification of the residue on silica gel with 2% ethanol/ammonia-saturated dichloromethane provided the desired compound.

MS (DCI/NH₃) m/z 244 (M+H)⁺.

### Example 1D

### tert-butyl 4-(5-amino-6-methoxy-1,2,3,4-tetrahydro-1-naphthalenyl)-1H-imidazole-1-carboxylate

A suspension of Example 1C (370 mg, 1.5 mmol) in acetonitrile (25 mL) was treated with di-tert-butyl dicarbonate (370 mg, 1.7 mmol), stirred at ambient temperature for 5 hours, stored at 0° C. for 16 hours, and concentrated. Purification of the residue on silica gel with 3:2 hexanes:ethyl acetate provided the desired compound.

MS (DCI/NH₃) m/z 344 (M+H)⁺.

### Example 1E

### N-[5-(1H-imidazol-4-yl)-2-methoxy-5,6,7,8-tetrahydro-1-naphthalenyl]methanesulfonamide hydrochloride

A solution of Example 1D (460 mg, 1.34 mmol) in dichloromethane (20 mL) was treated sequentially with pyridine (0.16 mL, 2.0 mmol) and methanesulfonyl chloride (0.12 mL, 1.6 mmol), stirred for 60 hours allowing the solvent to evaporate. Purification of the residue on silica gel with 2% ethanol/ammonia-saturated dichloromethane provided an oil which was converted to the hydrochloride salt to provide the title compound.

mp 209-211° C.;

¹H NMR (300 MHz, DMSO-d₆) δ 1.65-1.72 (m, 2H), 1.88-2.01 (m, 2H), 1.88 (t, 2H), 3.00 (s, 3H), 3.79 (s, 3H), 4.27 (t, 1H), 6.88 (q, 2H), 7.20 (s, 1H), 8.66 (s, 1H), 9.03 (s, 1H), 14.33 (bs, 2H);

MS (DCI/NH₃) m/z 322 (M+H)⁺;

Anal. calcd for C₁₅H₂₀N₃O₃S C, 50.35; H, 5.63; N, 11.74. Found: C, 50.12; H, 5.80; N, 11.65.

### EXAMPLE 2

### N-[2-hydroxy-5-(1H-imidazol-4-yl)-5,6,7,8-tetrahydro-1-naphthalenyl]methanesulfonamide, Hydrochloride

A suspension of Example 1E (320 mg, 1.0 mmol) in dichloromethane (100 mL) at 0° C. was treated with BBr₃ (1.0M in dichloromethane, 4.0 mL) over 5 minutes, stirred at 0° C. for 2 hours, cooled to −78° C., treated with methanol (10 mL), warmed to ambient temperature, and concentrated. Purification of the residue on silica gel with 20% ethanol/ammonia-saturated dichloromethane provided an oil which was converted to the hydrochloride salt to provide the title compound.

mp 135-137° C. (foam);

¹H NMR (300 MHz, DMSO-d₆) δ 1.61-1.74 (m, 2H), 1.88-2.00 (m, 2H), 2.86 (t, 2H), 3.03 (s, 3H), 4.21 (t, 1H), 6.69 (d, 1H), 6.75 (d, 1H), 7.18 (d, 1H), 8.58 (s, 1H), 9.05 (d, 1H), 9.85 (s, 1H), 14.38 (bs, 2H);

MS (DCI/NH₃) m/z 308 (M+H)⁺;

Anal. calcd for C₁₄H₁₈ClN₃O₃SCH₃CH₂OH: C, 49.29; H, 6.20; N, 10.78. Found: C, 48.98; H, 5.73;N, 10.70.

### EXAMPLE 3

### N-[2-hydroxy-5-(2-methyl-1H-imidazol-4-yl)-5,6,7,8-tetrahydro-1-naphthalenyl]methanesulfonamide, hydrochloride

### Example 3A

### 4-(6-methoxy-5-nitro-3,4-dihydro-1-naphthalenyl)-N,N-dimethyl- 1H-imidazole-1-sulfonamide

A solution of 4-iodo-N,N-dimethyl-1H-imidazole-1-sulfonamide (4.8 g, 16 mmol) in dichloromethane (65 mL) was treated with ethyl magnesium bromide (3.0M in diethyl ether, 5.4 mL) over 5 minutes, stirred for 30 minutes, treated with 6-methoxy-5-nitro-1-tetralone (3.9 g, 18 mmol), stirred for 16 hours, and concentrated. The residue was treated with 1M HCl (100 mL), heated to 100° C. for 1 hour, cooled to ambient temperature and filtered. The filtrate was neutralized with Na₂CO₃ and extracted with 5:1 dichloromethane/ethanol. The extract was dried (MgSO₄), filtered, and concentrated. The residue was combined with the filtered solid and purified on silica gel with a gradient of 20%-33% ethyl acetate/dichloromethane to provide the desired compound. Further elution with 10% ethanol/dichloromethane provided Example 1B.

MS (DCI/NH₃) m/z 379 (M+H)⁺.

### Example 3B

### 4-(6-methoxy-5-nitro-3,4-dihydro-1-nanhthalenyl)-2-methyl-1H-imidazole

A solution of diisopropylamine (0.60 mL, 4.3 mmol) in THF (10 mL) at −78° C. was treated with n-butyllithium (2.5M in hexane, 1.4 mL), stirred at −78° C. for 30 minutes, treated with Example 3A in THF (20 mL) over 5 minutes, stirred at −78° C. for 2 hours, treated with methyl iodide (1 mL), stirred at ambient temperature for 1 hour, treated with saturated ammonium chloride solution, and extracted with ethyl acetate. The extract was dried (MgSO₄), filtered, and concentrated. The residue was treated with 1M HCl, heated to 100° C. for 12 hours, cooled to ambient temperature, neutralized with NaHCO₃, and extracted with dichloromethane. The extract was dried (MgSO₄), filtered, and concentrated. Purification of the residue on silica gel with 2% ethanol/ammonia-saturated dichloromethane provided the desired compound.

MS (DCI/NH₃) m/z 286 (M+H)⁺.

### Example 3C

### tert-butyl 4-(6-methoxy-5-nitro-3,4-dihydro-1-naphthalenyl)-2-methyl-1H-imidazole-1-carboxulate

A solution of Example 3B (400 mg, 1.4 mmol) in DMF (20 mL) was treated with di-tert-butyl dicarbonate (1 g, 4.6 mmol), stirred for 30 minutes, heated to 75° C. for 15 minutes and concentrated. Purification of the residue on silica gel with 3:2 hexanes:ethyl acetate provided the desired compound.

MS (DCI/NH₃) m/z 386 (M+H).

### Example 3D

### tert-butyl 4-(5-amino-6-methoxy-1,2,3,4-tetrahydro-1-naphthalenyl)-2-methyl-1H-imidazole-1-carboxylate

### Example 3C was processed as in Example 1C to provide the desired compound. MS (DCI/NH₃) m/z 358 (M+H)⁺.

### Example 3E

### tert-butyl 4-{6-methoxy-5-[(methylsulfonyl)amino]-1,2,3,4-tetrahydro-1-naphthalenyl}-2-methyl-1H-imidazole-1-carboxylate

A solution of Example 3D (440 mg, 1.2 mmol) in dichloromethane (15 mL) was treated sequentially with pyridine (0.30 mL, 3.7 mmol), and methanesulfonyl chloride (0.14 mL, 1.8 mmol) and stirred for 16 hours, treated with NaHCO₃ solution and extracted with dichloromethane. The extract was dried (MgSO₄), filtered, and concentrated. Purification of the residue on silica gel with 2:3 hexanes:ethyl acetate provided the desired compound.

MS (DCI/NH₃) m/z 436 (M+H)⁺.

### Example 3F

### N-[2-hydroxy-5-(2-methyl-1H-imidazol-4-yl)-5,6,7,8-tetrahydro-1-naphthalenvylmethanesulfonamide, Hydrochloride

Example 3E was processed as in Example 2 to provide the desired compound.

mp 233-235° C.;

¹H NMR (300 MHz, DMSO-d₆) δ 1.61-1.78 (m, 2H), 1.82-1.97 (m, 2H), 2.52 (s, 3H), 2.86 (t, 2H), 3.03 (s, 3H), 4.13 (t, 1H), 6.73 (q, 2H), 7.04 (s, 1H), 8.58 (s, 1H), 9.83 (s, 1H), 13.98 (bs, 2H);

MS (DCI/NH₃) m/z 322 (M+H)⁺;

Anal. calcd for C₁₅H₂₀N₃O₃SCl C, 50.35; H, 5.63; N, 11.74. Found: C, 50.07; H, 5.67; N, 11.55.

### EXAMPLE 4

### N-[2-hydroxy-5-(1-methyl-1H-imidazol-5-yl)-5,6,7,8-tetrahydro-1-naphthalenyl]methanesulfonamide, Hydrochloride

### Example 4A and 4B

### 4A (Minor) 5-(3,4-dihydro-6-methoxy-5-nitro-1-naphthalenyl)-1-methyl-1H-imidazole 4B (Major) 4-(3,4-dihydro-6-methoxy-5-nitro-1-naphthalenyl)-1-methyl-1H-imidazole

A solution of Example 1B (1. 14 g, 4.2 mmol) in DMF (5 mL) was treated with sodium hydride (60% dispersion, 200 mg, 5.0 mmol), stirred for 30 minutes, treated with methyl iodide (0.32 mL, 5.0 mmol), stirred for 1.5 hours, treated with water (300 mL) and extracted with diethyl ether. The extract was washed sequentially with water and brine, dried (MgSO₄), filtered, and concentrated. Purification of the residue on silica gel with 12:1:1 ethyl acetate/water/formic acid provided (after conversion of each to the free base by partitioning between dichloromethane and sodium bicarbonate solution and then drying (MgSO₄), filtering and concentrating each of the dichloromethane layers) the less polar isomer (designated 4A) and the more polar isomer (designated 4B). MS (DCI/NH₃) m/z 286 (M+H)⁺ for each product.

### Example 4C

### 2-methoxy-5-(1-methyl-1H-imidazol-5-yl)-5,6,7,8-tetrahydro-1-naphthalenamine

Example 4A was processed as in Example 1C to provide the desired compound. MS (DCI/NH₃) m/z 258 (M+H)⁺.

### Example 4D

### N-[2-methoxy-5-(1-methyl-1H-imidazol-5-yl)-5,6,7,8-tetrahydro-1-naphthalenyl]methanesulfonamide

Example 4C was processed as in Example 1E to provide the desired compound. MS (DCI/NH₃) m/z 336 (M+H)⁺.

### Example 4E

### N-[2-hydroxy-5-(1-methyl-1H-imidazol-5-yl)-5,6,7,8-tetrahydro-1-naphthalenyl]methanesulfonamide, Hydrochloride

A solution of Example 4D (0.27 g, 0.80 mmol) in dichloromethane (50 mL) at-78° C. was treated with BBr₃ (1M) in dichloromethane (3.2 mL), stirred at 0° C. for 1.5 hours, cooled to-78° C., treated with methanol (5 mL), warmed to ambient temperature and concentrated. Purification of the residue on silica gel with 10% ethanol in ammonia-saturated dichloromethane provided an oil which was converted to the hydrochloride salt to provide the desired compound.

mp 260° C.;

¹H NMR (300 MHz, DMSO-d₆) δ 1.62-1.73 (m, 2H), 1.71-1.85 (m, 1H), 1.88-2.01 (m, 1H), 2.77-2.94 (m, 2H), 3.03 (s, 3H), 3.80 (s, 3H), 4.33 (t, 1H), 6.73 (q, 2H), 6.97 (d, 1H), 8.59 (s, 1H), 9.03 (s, 1H), 9.86 (s, 1H), 14.25 (bs, 1H);

MS (DCI/NH₃) m/e 322 (M+H)⁺;

Anal. calcd for C₁₅H₁₉N₃O₃SCl: C, 50.35; H, 5.63; N, 11.74. Found: C, 50.34; H, 5.60; N, 11.53.

### EXAMPLE 5

### N-[2-hydroxy-5-(i-methyl-1H-imidazol-4-yl)-5,6,7,8-tetrahydro-1-naphthalenyl]methanesulfonamide, Hydrochloride

### Example 5A

### 2-methoxy-5-(1-methyl-1H-imidazol-4-yl)-5,6,7,8-tetrahydro-1-naphthalenamine

Example 4B was processed as in Example 1C to provide the desired compound. MS (DCI/NH₃) m/z 258 (M+H)⁺.

### Example 5B

### N-[2-methoxy-5-(1-methyl-1H-imidazol-4-yl)-5,6,7,8-tetrahydro-1-naphthalenyl]methanesulfonamide

Example 5A was processed as in Example 1E to provide the desired compound. MS (DCI/NH₃) m/z 336 (M+H)⁺.

### Example 5C

### N-[2-hydroxy-5-(1-methyl-1H-imidazol-4-yl)-5,6,7,8-tetrahydro-1-naphthalenyl]methanesulfonamide Hydrochloride

Example 5B was processed as in Example 2 to provide the desired compound.

mp 256-258° C.;

¹H NMR (300 MHz, DMSO-d₆) δ 1.61-1.72 (m, 2H), .1.87-1.98 (m, 2H), 2.85 (t, 2H), 3.03 (s, 3H), 3.78 (s, 3H), 4.20 (t, 1H), 6.75 (q, 2H), 7.17 (s, 1H), 8.59 (s, 1H), 9.00 (s, 1H);

MS (DCI/NH₃) m/z 322 (M+H)⁺;

Anal. calcd. for C₁₅H₂₀N₃O₃SC1: C, 50.35; H, 5.63; N, 11.74. Found: C, 50.15; H, 5.57; N, 11.45.

### EXAMPLE 6

### N-[5-(1-ethyl-1H-imidazol-4-yl)-2-hydroxy-5,6,7,8-tetrahydro-1-naphthalenyl]methanesulfonamide, Hydrochloride

### Example 6A

### 1-ethyl-4-(6-methoxy-5-nitro-3,4-dihydro-1-naphthalenyl)-1H-imidazole

A solution of Example 1B (1.5 g, 5.5 mmol) in DMF (25 mL) was treated with sodium hydride (60% dispersion, 270 mg, 6.6 mmol), stirred for 30 minutes, treated with ethyl iodide (0.53 mL, 6.6 mmol), stirred for 1 hour, treated with water (300 mL) and extracted with diethyl ether (200 mL). The extract was washed sequentially with water, and brine, dried (MgSO₄), filtered and concentrated. Purification of the residue on silica gel with ammonia-saturated ethyl acetate provided, as the less polar isomer, 0.95 g (57%) of the desired compound.

MS (DCI/NH₃) M/Z 300 (M+H)⁺.

### Example 6B

### 5-(1-ethyl-1H-imidazol-4-yl)-2-methoxy-5,6,7,8-tetrahydro-1-naphthalenamine

Example 6A (0.91 g, 3.0 mmol) was processed as in Example 1C to provide the desired compound.

MS (DCI/NH₃) m/z 272 (M+H)⁺.

### Example 6C

### N-[5-(1-ethyl-1H-imidazol-4-yl)-2-methoxy-5,6,7,8-tetrahydro-1-naphthalenyl]methanesulfonamide

Example 6B was processed as in Example 1E to provide the desired compound.

MS (DCI/NH₃) m/z 350 (M+H)⁺.

### Example 6D

### N-[5-(1-ethyl-1H-imidazol-4-yl)-2-hydroxy-5,6,7,8-tetrahydro-1-naphthalenyl]methanesulfonamide, Hydrochloride

### Example 6C was processed as in Example 2 to provide the desired compound.

mp 230-234° C. (decomp.);

¹H NMR (300 MHz, DMSO-d₆) δ 1.40 (t, 3H), 1.62-1.73 (m, 2H), 1.88-2.01 (m, 2H), 2.85 (t, 2H), 3.03 (s, 3H), 4.13 (q, 2H), 4.20 (t, 1H), 6.77 (q, 2H), 7.29 (d, 1H), 8.61 (s, 1H), 9.12 (d, 1H), 9.91 (s, 1H), 14.64 (bs, 1H); MS (DCI/NH₃) m/z 336 (M+H)⁺;

Anal. calcd for C₁₆H₂₂ClN₃O₃S: C, 51.68; H, 5.96; N, 11.30. Found: C, 51.64; H, 5.91; N, 11.10.

### EXAMPLE 7

### N-[2-hydroxy-5-(1-propyl-1H-imidazol-4-yl)-5,6,7,8-tetrahydro-1-naphthalenyl]methanesulfonamide, Hydrochloride

### Example 7A

### 4-(3,4-dihydro-6-methoxy-5-nitro-1-nahthalenyl)-1-propyl-1H-imidazole

Example 7B was processed as in Example 6A but substituting propyl iodide for ethyl iodide to provide the less polar isomer as the desired compound.

MS (DCI/NH₃) m/z 314 (M+H)⁺.

### Example 7B

### 2-methoxy-5-(1-propyl-1H-imidazol-4-yl)-5,6,7,8-tetrahydro-1-naphthalenamine

Example 7A was processed as in Example 1C to provide the desired compound. MS (DCI/NH₃) m/z 286 (M+H)⁺.

### Example 7C

### N-[2-methoxy-5-(1-propyl-1H-imidazol-4-yl)-5,6,7,8-tetrahydro-1-naphthalenyl]methanesulfonamide

Example 7B was processed as in Example 1E to provide the desired compound.

MS (DCI/NH₃) m/z 364 (M+H)⁺.

### Example 7D

N-[2-hydroxy-5-(1-propyl-1H-imidazol-4-yl)-5,6,7,8-tetrahydro-1-naphthalenyl]methanesulfonamide, Hydrochloride

Example 7C was processed as in Example 2 to provide the desired compound.

mp 128-133° C. (foam);

¹H NMR (300 MHz, DMSO-d₆) δ 0.83 (t, 3H), 1.61-1.72 (m, 2H), 1.72-1.85 (m, 2H), 1.86-2.02 (m, 2H), 2.85 (t, 2H), 3.03 (s, 3H), 4.07 (t, 2H), 4.20 (t, 1H), 6.75 (q, 2H), 7.28 (s, 1H), 8.59 (s, 1H), 9.10 (d, 1H), 9.83 (s, 1H), 14.59 (bs, 1H); MS (DCI/NH₃) m/z 350 (M+H);

Anal. calcd for C₁₇H₂₄ClN₃O₃S 0.75 CH₃OH :C, 52.01; H, 6.64; N, 10.25. Found: C, 52.15; H, 6.24; N, 9.84

### EXAMPLE 8

### N-[5-(1H-imidazol-4-yl)-5,6,7,8-tetrahydro-1-nanhthalenyl]methanesulfonamide, Hydrochloride

### Example 8A

### N-benzyl-N-(5-oxo-5,6,7,8-tetrahydro-1-nanhthalenyl)methanesulfonamide

5-Amino-1-tetralone was processed as in Meyer, M. D, J. Med. Chem. (1997), 40, 1049-1062 to provide the desired compound.

### Example 8B

### N-benzyl-N-[5-(1H-imidazol-4-yl)-7,8-dihydro-1-naphthalenyl]methanesulfonamide

A solution of 4-iodo-N,N-dimethyl-1H-imidazole-1-sulfonamide (1.3 g, 4.2 mmol) in dichloromethane (17 mL) was treated with ethylmagnesium bromide (3.0 M in diethyl ether, 1.4 mL) over 2 minutes, stirred for 30 minutes, treated with Example 8A (1.1 g, 3.5 mmol), stirred for 16 hours and concentrated. The residue was treated with 2 M HCl (30 mL), heated for 2 hours at 100° C., cooled to ambient temperature, neutralized with NaHCO₃ and extracted with dichloromethane. The extract was dried (MgSO₄), filtered, and concentrated. Purification of the residue on silica gel with 2% ethanol/ammonia-saturated dichloromethane provided the desired compound.

### Example 8C

### N-[5-(1H-imidazol-4-yl)-5,6,7,8-tetrahydro-1-naphthalenyl]methanesulfonamide, Hydrochloride

Example 8B was processed as in Example 1C to provide the desired compound.

mp 113-114° C. (foam);

¹H NMR (300 MHz, DMSO-d₆) δ 1.70-1.82 (m, 2H), 1.92-2.04 (m, 2H), 2.83 (t, 2H), 3.03 (s, 3H), 4.34 (t, 1H), 6.82 (d, 1H), 7.14 (t, 1H), 7.23 (d, 1H), 7.26 (s, 1H), 9.03 (s, 1H), 9.07 (s, 1H), 14.36 (bs, 2H);

MS (DCI/NH₃) m/z 292 (M+H)⁺;

Anal. calcd for C₁₄H₁₈ClN₃O₂S0.25H₂O: C, 50.60; H, 5.61; N, 12.64. Found: 50.75; H, 5.74; N, 12.31.

### EXAMPLE 9

### (+)-N-[(5R)-5-(1H-imidazol-4-yl)-5,6,7,8-tetrahydro-1-naphthalenyl]methanesulfonamide

### Example 9A

### tert-butyl 4-{5-[(methylsulfonyl)amino]-1,2,3,4-tetrahydro-1-naphthalenyl}-1H-imidazole-1-carboxylate

A solution of the free base of Example 8C (3.6 g, 12 mmol) in DMF (50 mL) was treated with di-tert-butyl dicarbonate (3.0 g, 14 mmol), stirred for 8 hours, treated with diethyl ether (500 mL), washed sequentially with water, and brine, dried (MgSO₄), filtered, and concentrated. Purification of the residue on silica gel with 2:1 hexanes:ethyl acetate provided 3.6 g (74%) of the desired compound.

MS (DCI/NH₃) m/z 392 (M+H)⁺.

### Example 9B

### (+)-tert-butyl 4-{5-[(methylsulfonyl)amino]-1,2,3,4-tetrahydro-1-naphthalenyl}-1H-imidazole-1-carboxylate

The enantiomers of Example 9A were separated by chiral chromatography on a Chiralcel OJ column (5.0 cm inner diameter, 50 cm length, 20 micron packing) using 90:10 hexanes:ethanol at a flow rate of 200 mL/minute as the mobile phase. Four separate injections of 150 mg each in 95:5 ethanol:dichloromethane (6 mL) provided 320 mg of the faster moving enantiomer.

[α]²³_{D}+71.5° (c 1.0, MeOH);

MS (DCI/NH₃) m/z 392 (M+H)⁺.

### Example 9C

### (+)-N-[(5R)-5-(1H-imidazol-4-yl)-5,6,7,8-tetrahydro-1-naphthalenyl]methanesulfonamide

A solution of Example 9B (130 mg, 0.33 mmol) in methanol (10 mL) was treated with 1N HCl (5 mL), stirred for 1.5 hours, concentrated at 45° C., and dried under vacuum for 30 minutes. The residue was dissolved in methanol, filtered through cotton, concentrated and dried under vacuum for 3 hours to provide the desired compound

mp 118-123° C. (foam);

[α]²³_{D}+41.80 (c 1.0, MeOH);

MS (DCI/NH₃) m/z 292 (M+H)⁺;

¹H NMR (300 MHz, DMSO-d₆) δ 1.70-1.82 (m, 2H), 1.92-2.04 (m, 2H), 2.83 (t, 2H), 3.03 (s, 3H), 4.34 (t, 1H), 6.82 (d, 1H), 7.14 (t, 1H), 7.23 (d, 1H), 7.26 (s, 1H), 9.03 (s, 1H), 9.07 (s, 1H), 14.36 (bs, 2H);

Anal. calcd for C₁₄H₁₈ClN₃O₂S.0.5H₂O.0.5 MeOH: C, 49.36; H, 6.00; N, 11.91. Found: C, 49.36; H, 6.00; N, 11.91.

### EXAMPLE 10

### (−)-N-[(5S)-5-(1H-imidazol-4-yl)-5,6,7,8-tetrahydro-1-naphthalenyl]methanesulfonamide

### Example 10A

### (−)-tert-butyl 4-{5-[(methylsulfonylvamino-1,2,3,4-tetrahydro-1-naphthalenyl}-1H-imidazole-1-carboxylate

The title compound (340 mg) was provided as the slower moving enantiomer from the procedure described in Example 9B.

[α]₂₃^{D}−69.40 (c 1.0, MeOH);

MS (DCI/NH₃) m/z 392 (M+H)⁺.

### Example 10B

### (−)-N-[(5S)-5-(1H-imidazol-4-yl)-5,6,7,8-tetrahydro-1-naphthalenyl]methanesulfonamide

A solution of the Example 10A (95 mg, 0.24 mmol) in methanol (10 mL) was treated with 1N HCl (5 mL) then processed as in Example 9C to provide the desired compound.

mp 118-123° C. (foam);

[α]₂₃^{D}−40.80 (c 1.0, MeOH);

¹H NMR (300 MHz, DMSO-d₆) δ 1.70-1.82 (m, 2H), 1.92-2.04 (m, 2H), 2.83 (t, 2H), 3.03 (s, 3H), 4.34 (t, 1H), 6.82 (d, 1H), 7.14 (t, 1H), 7.23 (d, 1H), 7.26 (s, 1H), 9.03 (s, 1H), 9.07 (s, 1H), 14.36 (bs, 2H);

MS (DCI/NH₃) m/z 292 (M+H)⁺;

Anal. calcd for C₁₄H₁₈ClN₃O₂.S0.5 CH₃OH.0.5H₂O: C, 49.36; H, 6.00; N, 11.91. Found: C, 49.63; H, 6.04; N, 11.65.

### EXAMPLE 11

### N-[2-hydroxy-5-(1H-imidazol-4-ylmethyl)phenyl]methanesulfonamide, Hydrochloride

### Example 11A

### 1H-imidazol-4-yl(4-methoxy-3-nitrophenyl)methanol

A solution of 4-iodo-N,N-dimethyl-1H-imidazole-1-sulfonamide (3.0 g, 10 mmol) in dichloromethane (40 mL) under nitrogen was treated with ethylmagnesium bromide (3.0M in diethyl ether, 3.3 mL) over 2 minutes, stirred for 30 minutes, treated with 4-methoxy-5-nitrobenzaldehyde (2.0 g, 11 mmol), stirred for 1 hour, stored at 0° C. for 16 hours, concentrated to dryness, treated with 1 M HCl(100 mL), heated to 100° C. for 16 hours, cooled to ambient temperature, neutralized with NaHCO₃ and extracted with 3:1 dichloromethane:ethanol (5×). The combined extractions were dried (MgSO₄), filtered and concentrated. Purification on silica gel with 10% and then 20% ethanol/ammonia-saturated dichloromethane provided the desired compound.

MS (DCI/NH₃) m/z 250 (M+H)⁺.

### Example 11B

### (3-amino-4-methoxyphenyl)(1H-imidazol-4-yl)methanol

Example 11A (3.2 g, 13 mmol) was processed as in Example 1C to provide the desired compound.

MS (DCI/NH₃) m/z 220 (M+H)⁺.

### Example 11C

### N-{5-[hydroxy(1H-imidazol-4-yl)methyl]-2-methoxyphenyl}methanesulfonamide Fumarate

A solution of Example 11B (1.5 g, 6.8 mmol) in 8:1 pyridine:dichloromethane (45 mL) was treated with methanesulfonyl chloride (0.56 mL, 7.2 mmol) over 10 minutes and the mixture was concentrated. Purification of the residue on silica gel using 8:1:1 ethyl acetate:H₂O:HCOOH provided the formic acid salt of the desired compound which was converted to the free base with silica gel 20% ethanol/ammonia-saturated dichloromethane provided the desired compound which was converted to the fumaric acid salt.

mp 90-93° C. (foam);

¹H NMR (300 MHz, DMSO-d₆) δ 2.93 (s, 3H), 3.80 (s, 3H), 5.57 (s, 1H), 6.61 (s, 1H), 6.72 (s, 1H), 6.99 (d, 1H), 7.18 (dd, 1H), 7.30 (d, 1H), 7.55 (d, 1H), 8.72 (bs, 1H): MS (DCI/NH₃) m/z 298 (M+H)⁺.

Anal. calcd for C₁₂H₁₅N₃O₄S C₄H₄O₄.0.75 (C₂H₆O): C, 47.75; H, .5.56; N, 10.78. Found: C, 47.40; H, 5.32; N, 10.52.

### Example 11D

### N-[5-(1H-imidazol-4-ylmethyl)-2-methoxnphenyl]methanesulfonamide, Hydrochloride

A solution of the free base of Example 11C (0.59 g, 2.0 mmol) in trifluoroacetic acid was treated with triethylsilane (3 mL, 20 mmol), stirred for 30 minutes and concentrated to dryness. Purification of the residue on silica gel using 10% ethanol/ammonia-saturated dichloromethane provided the desired compound, which was converted to the hydrochloric acid salt.

mp 206-208° C.;

¹H NMR (300 MHz, DMSO-d₆) δ 2.91 (s, 3H), 3.76 (s, 3H), 3.93 (s, 2H), 6.99 (d, 1H), 7.07 (dd, 1H), 7.10 (d, 1H), 7.37 (d, 1H), 8.84 (s, 1H), 8.97 (d, 1H), 14.33 (bs, 2H);

MS (DCI/NH₃) m/z 282 (M+H)⁺;

Anal. calcd for C₁₂H₁₆ClN₃O₃S: C, 45.35; H, 5.07; N, 13.22. Found: C, 45.45; H, 5.27; N, 13.05.

### Example 11E

### N-[2-hydroxy-5-(1H-imidazol-4-ylmethyl)phenyl]methanesulfonamide, Hydrochloride

Example 11D was processed as in Example 2 to provide the desired compound.

mp 167-169° C.;

¹H NMR (300 MHz, DMSO-d₆) δ 2.94 (s, 3H), 3.92 (s, 2H), 6.87 (d, 1H), 6.96 (dd, 1H), 77.10 (d, 1H), 7.40 (s, 1H), 8.77 (s, 1H), 9.00 (s, 1H), 9.93 (s, 1H), 14.31 (bs, 2H); MS (DCI/NH₃) m/z 268 (M+H)⁺;

Anal. calcd for C₁₁H₁₄ClN₃O₃S: C, 43.49; H, 4.65; N. 13.83. Found: C, 43.58; H, 4.76; N, 13.80.

### EXAMPLE 12

### N-[5-(1H-imidazol-4-yl)-5,6,7,8-tetrahydro-1-naphthalenyl]ethanesulfonamide Maleate

### Example 12A

### 4-(5-nitro-3,4-dihydro-1-naphthalenyl)-1H-imidazole

A solution of 4-iodo-1-trityl-1H-imidazole (5.5 g, 13 mmol) (prepared as described by Kirk, K. J. Heterocyclic Chem. (1985), 22, 57-59) in dichloromethane (50 mL) was treated with ethylmagnesium bromide (3.0 M in diethyl ether, 4.2 mL) over 4 minutes, stirred for 30 minutes, treated with 5-nitrotetralone (prepared as described by Zhang, M J. Amer. Chem. SoC, (1994), 116, 4852-4857), stirred for 6 hours, treated with ammonium chloride solution (50 mL) and extracted with a mixture of diethyl ether (300 mL) and ethyl acetate (50 mL). The organic layer was isolated, treated with dichloromethane (500 mL) to dissolve the product which started to crystallize, dried (MgSO₄), filtered, concentrated, treated with trifluoroacetic acid (80 mL), stirred for 48 hours, concentrated to an oil, neutralized with sodium bicarbonate solution and extracted twice with dichloromethane. The combined dichloromethane layers were dried (MgSO₄), filtered and concentrated. The residue was purified on silica gel with a gradient of 5%-10% methanol/dichloromethane to provide the desired compound.

MS (DCIINH₃) m/z 242 (M+H)⁺.

### Example 12B

### tert-butyl 4-(5-nitro-3,4-dihydro-1-naphthalenyl)-1H-imidazole-1-carboxylate

A solution of Example 12A (1.9 g, 7.9 mmol) in N,N-dimethylformamide (25 mL) was treated with di-tert-butyl bicarbonate (3.4 g, 16 mmol), stirred at ambient temperature for 2 hours, heated to 50° C. for 15 minutes, cooled, diluted with diethyl ether (250 mL), washed with water (2×, 100 mL), washed with brine, dried (MgSO₄), filtered and concentrated. Purification of the residue on silica gel with 3:1 hexanes:ethyl acetate provided the desired compound.

MS (DCI/NH₃) m/z 342 (M+H)⁺.

### Example 12C

### tert-butyl 4-(5-amino-1,23,4-tetrahydro-1-naphthalenyl)-1H-imidazole-1-carboxylate

Example 12B was processed as in Example 1C substituting ethyl acetate for methanol as the solvent. Purification of the residue on silica gel with 1:1 hexanes:ethyl acetate provided the desired compound.

MS (DCIINH₃) m/z 314 (M+H)⁺.

### Example 12D

### N-[5-(1H-imidazol-4-yl)-5,6,7,8-tetrahydro-1-naphthalenyl]ethanesulfonamide Maleate

A solution of Example 12C (260 mg, 0.83 mmol) in dichloromethane (5 mL) was treated sequentially with pyridine (0.20 mL, 2.5 mmol) and ethanesulfonyl chloride (0.087 mL, 0.91 mmol), stirred for 16 hours, treated with trifluoroacetic acid (3 mL), stirred for 30 minutes and concentrated. Purification of the residue on silica gel with a gradient of 5%-10% ethanol in ammonia-saturated dichloromethane provided a solid, which was converted to the maleic acid salt to provide the desired compound.

mp 129-132° C.;

¹H NMR (DMSO-d₆) δ 1.28 (t, 3H), 1.67-1.85 (m, 2H), 1.87-2.06 (m, 2H), 2.83 (t, 2H), 3.13 (q, 2H), 4.30 (t, 1H), 6.05 (s, 2H), 6.80 (d, 1H), 7.12 (t, 1H), 7.16-7.23 (m, 2H); MS (DCI/NH₃) m/z 306 (M+H)⁺;

Anal. calcd for C₁₅H₁₉N₃O₂SC₄H₄O₄: C, 54.15; H, 5.50; N, 9.97. Found: C, 54.24; H, 5.53; N, 9.87.

### EXAMPLE 14

### N-[5,6,7,8-tetrahydro-5-(1-methyl-1H-imidazol-4-yl) -1-naphthalenyl]methanesulfonamide hydrochloride

### Example 14A

### N-benzyl-N-[5-(1-methyl-1H-imidazol-4-yl)-7,8-dihydro-1-naphthalenyl]methanesulfonamide

Example 8B was processed as in Example 4A and 4B to provide the desired product as the more polar isomer.

MS (DCI/NH₃) m/z 394 (M+H)⁺.

### Example 14B

### N-[5,6,7,8-tetrahydro-5-(1-methyl-1H-imidazol-4-yl)-1-naphthalenyl]methanesulfonamide, Hydrochloride

Example 14A was processed as in Example 1C to provide the desired product which was converted to the hydrochloride salt.

mp 130-135° C.;

¹H NMR (DMSO-d₆) δ 1.68-1.79 (m, 2H), 1.93-2.03 (m, 2H), 2.88 (t, 2H), 3.03 (s, 3H), 3.79 (s, 3H), 4.33 (t, 1H), 6.87 (d, 1H), 7.15 (t, 1H), 7.20-7.26 (m, 2H), 9.01 (s, 1H), 9.06 (s, 1H), 14.57 (bs, 1H); MS (DCI/NH₃) m/z 306 (M+H)⁺; Anal. calcd for C₁₅H₁₉N₃O₂SHCl0.5H₂O: C, 51.35; H, 6.03; N, 11.98. Found: C, 51.10; H, 5.98; N, 11.82.

### EXAMPLE 15

### N-[5,6,7,8-tetrahydro-5-(1H-imidazol-4-yl)-1-naphthalenyl]-N-methylmethanesulfonmamide, Maleate

### Example 15A

### N-(5-oxo-5,6,7,8-tetrahydro-1-naphthalenyl)methanesulfonamide

5-Amino-1tetralone (Itoh, K. Chem. Pharm. Bull. (1984), 32, 130-151) was processed as in Meyer, M.d. J. Med. Chem. (1997), 40, 1049-1062 to provide the desired product.

### Example 15B

### N-(methoxymethyl)-N-(5-oxo-5,6,7,8-tetrahydro-1-naphthalenyl)methanesulfonamide

A solution of Example 15A (4.0 g, 17 mmol) in anhydrous DMF (40 mL) under a nitrogen atmosphere was treated with a 60% dispersion of sodium hydride (0.74 g, 18 mmol) in portions over 5 minutes, stirred for 45 minutes, cooled to 3° C., treated dropwise with chloromethyl methyl ether (1.3 mL, 18 mmol), stirred at ambient temperature for 2 hours, treated with cold water (250 mL) and extracted with diethyl ether (3×). The combined diethyl ether extracts were washed with water, washed with brine, dried (MgSO₄), filtered and concentrated. Purification of the residu e on silica gel with 1:1 hexanes:ethyl acetate provided the desired product.

MS (DCI/NH₃) m/z 265 (M+NH₄)⁺.

### Example 15C

### N,N-dimethyl-4-{5-[(methylsulfonyl)amino]- 3,4-dihydro-1-naphthalenyl}-1H-imidazole-1-sulfonamide

Example 15B was processed as in Example 3A to provide the desired product. MS (DCI/NH₃) m/z 397 (M+H)⁺.

### Example 15D

### N,N-dimethyl-4-{5-[(methylsulfonyl)amino]-1,2,3,4-tetrahydro-1-naphthalenyl}-1H-imidazole-1-sulfonamide

Example 15C was processed as in Example 1C to provide the desired product. MS (DCI/NH₃) m/z 399 (M+H)⁺.

### Example 15E

### N,N-dimethyl-4-{5-methyl(methylsulfonyl)amino]-1,2,3,4-tetrahydro-1-naphthalenyl}-1H-imidazole-1-sulfonamide

A solution of Example 15D (0.30 g, 0.75 mmol) in anhydrous DMF (3 mL) under nitrogen was treated with 60% sodium hydride (0.033 g, 0.83 mmol), stirred for 15 minutes, treated with iodomethane (0.056 mL, 0.90 mmol), stirred for 16 hours, diluted with diethyl ether (100 mL), washed with water, washed with brine, dried (MgSO₄), filtered and concentrated. Purification of the residue on silica gel with ethyl acetate provided the desired product.

MS (DCI/NH₃) m/z 413 (M+H)⁺.

### Example 15F

### N-[5-(1H-imidazol-4-yl)-5,6,7,8-tetrahydro-1-naphthalenyl]-N-methylmethanesulfonamide, Maleate

A solution of Example 15E (0.28 mg, 0.68 mmol) in 1M HCl (10 mL) and THF (10 mL) was refluxed for 48 hours, cooled to ambient temperature, treated with dichloromethane, washed with sodium bicarbonate solution, dried (MgSO₄), filtered and concentrated. Purification of the residue on silica gel with 4% ethanol/ammonia-saturated dichloromethane provided a solid, which was converted to the maleic acid salt to provide the desired product.

mp 146-147° C.;

¹H NMR (DMSO-d₆) δ 1.67-2.07 (m, 4H), 2.70-2.86 (m, 1H), 2.87-3.01 (m, 1H), 3.08 and 3.09 (s and s, 3H), 3.12 and 3.13 (s and s, 3H), 4.24-4.35 (m, 1H), 6.05 (s, 2H), 6.94 (t, 1H), 7.13-7.24 (m, 2H), 7.37 (d, 1H), 8.85 (s, 1H);

MS (DCI/NH₃) m/z 306 (M+H)⁺;

Anal. calc'd for C₁₅H₁₉N₃O₂S.C₄H₄O₄: C, 54.15; H, 5.50; N, 9.97. Found: C, 54.15; H, 5.67; N, 9.77.

### EXAMPLE 16

### N-[5,6,7,8-tetrahydro-5-(1H-imidazol-4-yl)-1-naphthalenyl]acetamide, Maleate

Example 12C was processed as in Example 12D but substituting acetic anhydride for ethanesulfonyl chloride to provide the desired product which was converted to the maleic acid salt.

mp 159-160° C.;

¹H NMR (DMSO-d₆) 61.67-1.86 (m, 2H), 1.88-2.04 (m, 2H), 2.06 (s, 3H), 2.68 (t, 2H), 4.30 (t, 1H), 6.05 (s, 2H), 6.73 (d, 1H), 7.19 (t, 1H), 7.21 (s, 1H), 7.30 (d, 1H), 8.86 (s, 1H), 9.22 (s, 1H);

MS (DCI/NH₃) m/z 256 (M+H)⁺;

Anal. calcd for C₁₅H₁₁N₃OC₄H₄O₄: C, 61.45; H, 5.70; N, 11.31. Found: C, 61.47; H, 5.87; N, 11.33.

### EXAMPLE 17

### 2,2,2-trifluoro-N-[5-(1H-imidazol-4-yl)-5,6,7,8-tetrahydro-1-naphthalenyl]acetamide, maleate

Example 12C was processed as in Example 12D but substituting trifluoroacetic anhydride for ethanesulfonyl chloride to provide the desired product which was converted to the maleic acid salt.

mp 181-182° C.;

¹H NMR (DMSO-d₆) δ 1.67-1.85 (m, 2H), 1.92-2.06 (m, 2H), 2.65 (t, 2H), 4.33 (t, 1H), 6.05 (s, 2H), 6.93 (dd, 1H), 7.16-7.23 (m, 3H), 8.83 (s, 1H), 10.92 (s, 1H);

MS (DCI/NH₃) m/z 310 (M+H)⁺;

Anal. calcd for C₁₅H₁₄N₃OF₃ C₄H₄O₄: C, 53.65; H, 4.27; N, 9.88. Found: C, 53.53; H, 4.17;N,9.87.

### EXAMPLE 18

### N-[5,6,7,8-tetrahydro-5-(1H-imidazol-4-yl)-1-naphthalenyl]-2-methylethanesulfonamide, Maleate

Example 12C was processed as in Example 12D but substituting isopropylsulfonyl chloride for ethanesulfonyl chloride to provide the desired product which was converted to the maleic acid salt.

mp 124-125° C.;

¹H NMR (DMSO-d₆) δ 1.30 (d, 6H), 1.69-1.83 (m, 2H), 1.89-2.02 (m, 2H), 2.83 (t, 2H), 3.25-3.36 (m, 1H), 4.28 (t, 1H), 6.04 (s, 2H), 6.79 (d, 1H), 7.10 (t, 1H), 7.16-7.23 (m, 2H), 8.82 (bs, 1H), 8.94 (s, 1H);

MS (DCI/NH₃) m/z 320 (M+H)⁺;

Anal. calcd for C₁₆H₂₁N₃O₂S.C₄H₄O₄: C, 55.16; H, 5.79; N, 9.65. Found: C, 55.12; H, 5.82; N, 9.56.

### EXAMPLE 19

### N-[4-(1H-imidazol-4-yl)-3,4-dihydro-2H-chromen-8-yl]methanesulfonamide, Maleate

### Example 19A

### 4-(8-nitro-2H-chromen-4-l)-1H-imidazole

8-Nitrochroman-4-one (Chakravarti, D. J.Indian Chem.Soc. (1939), 16, 639-644) was processed as in Example 12A to provide the desired product.

MS (DCI/NH₃) m/z 244 (M+H)⁺.

### Example 19B

### tert-butyl 4-(8-nitro-2H-chromen-4-yl)-1H-imidazole-1-carboxylate

Example 19A was processed as described in Example 12B to provide the desired product.

MS (DCI/NH₃) m/z 344 (M+H)⁺.

### Example 19C

### tert-butyl 4-(8-amino-3,4-dihydro-2H-chromen-4-yl)-1H-imidazole-1-carboxylate

Example 19B was processed as in Example 1C but substituting ethyl acetate for methanol as the solvent to provide the desired product.

MS (DCI/NH₃) m/z 299 (M+H)⁺.

### Example 19D

### N-[4-(1H-imidazol-4-yl)-3,4-dihydro-2H-chromen-8-yl]methanesulfonamide Maleate

Example 19C was processed as in Example 12D but substituting methanesulfonyl chloride for ethanesulfonyl chloride to provide the desired product which was converted to the maleic acid salt.

mp 172-174° C.;

¹H NMR (DMSO-d₆) δ 2.22 (m, 2H), 2.99 (s, 3H), 4.25 (m, 2H), 4.40 (t, 1H), 6.06 (s, 2H), 6.78 (dd, 1H), 6.83 (t, 1H), 7.16 (dd, 1H), 7.29 (s, 1H), 8.80 (s, 1H), 8.88 (s, 1H);

MS (APCI+) ml/z 294 (M+H)⁺;

Anal. calcd for C₁₃H₁₅N₃O₃S C₄H₄O₄: C, 49.87; H, 4.68; N, 10.26. Found: C, 50.03; H, 4.88; N, 10.24.

### EXAMPLE 20

### N-[5,6,7,8-tetrahydro-5-(1H-imidazol-4-yl)-1-naphthalenyl]-2,2,2trifluoroethanesulfonamide, Maleate

### Example 20A

### tert-butyl 4-(5-{[(2,2,2-trifluoroethyl)sulfonyl]amino}-1,2,3,4-tetrahydro-1-naphthalyl)-1H-imidazole-1-carboxylate

Example 12C was processed as in Example 33A but substituting 2,2,2-trifluoroethanesulfonyl chloride for ethanesulfonyl chloride to provide the desired product.

MS (DCI/NH₃)mnlz 460 (M+H)⁺.

### Example 20B

### N-[5,6,7,8-tetrahydro-5-(1H-imidazol-4-yl)-1-naphthalenyl]-2,2,2-trifluoroethanesulfonamide, Maleate

A solution of Example 20A in trifluoroacetic acid (10 mL) was mixed for 15 minutes, concentrated, dissolved in 5:1 methanol:water (6 mL) and applied to an ion exchange resin (25 g of Dowex® 50 x 8-200 ion-exchange resin). The resin was washed with water until neutral, washed with methanol and the desired product was then flushed from the resin using 5% ammonium hydroxide solution in 1:1 methanol:dichloromethane. Concentration of the product containing fraction provided a solid which was converted to the maleic acid salt providing the desired product.

mp 138-140° C.;

¹H NMR (DMSO-d₆) δ 1.68-1.82 (m, 2H), 1.90-2.05 (m, 2H), 2.81 (t, 2H), 4.30 (t, 1H), 4.52 (q, 2H), 6.05 (s, 2H), 6.88 (d, 1H), 7.10-7.20 (m, 2H), 7.21 (d, 1H), 8.83 (s, 1H);

MS (DCI/NH₃) m/z 360 (M+H)⁺;

Anal. calcd for C₁₅H₁₆N₃O₂SF₃ C₄H₄O₄: C, 48.00; H, 4.24; N, 8.84. Found: C, 47.99; H, 4.35; N, 9.09.

### EXAMPLE 21

### N-[3-(1H-imidazol-4-ylmethyl)phenyl]methanesulfonamide, Maleate

### Example 21A

### 4-[hydroxy(3-nitrophenyl)methyl]-N,N-dimethyl-1H-imidazole-1-sulfonamide

3-Nitrobenzaldehyde was substituted for 6-methoxy-5-nitro-1-tetralone and processed as in Example 1A to provide the desired product.

### Example 21B

### 4-[(3-aminophenyl)(hydroxy)methyl]-N,N-dimethyl-1H-imidazole-1-sulfonamide

Example 21A was processed as in Example 1C but substituting ethyl acetate for methanol to provide the desired product.

MS (DCI/NH₃) m/z 297 (M+H)⁺.

### Example 21B

### 4-(3-aminobenzl)-N,N-dimethyl-1H-imidazole-1-sulfonamide

A solution of Example 21B (0.72 g, 2.4 mmol) in trifluoroacetic acid (20 mL) was treated with triethylsilane (3.5 mL), refluxed for 3 hours and concentrated. Purification of the residue on silica gel using 2% ethanol/ammonia-saturated dichloromethane provided a product which was purified on silica gel using ethyl acetate to provide the desired product. MS (DCI/NH₃) m/z 281 (M+H)⁺.

### Example 21C

### N-[3-(1H-imidazol-4-ylmethyl)phenyl]methanesulfonamide, Maleate

A solution of Example 21C (0.22 g, 0.78 mmol) in dichloromethane (3 mL) was treated with pyridine (0.19 mL, 2.4 mmol), treated with methanesulfonyl chloride (0.067 mL, 0.86 mmol), stirred for 1 hour, concentrated to dryness, treated with 1M HCl (5 mL) and tetrahydrofuran (2 mL), refluxed for 2 hours and concentrated. Purification of the residue on silica gel with 10% and then 20% ethanol/ammonia-saturated dichloromethane provided a product, which was converted to the maleic acid salt to provide the desired product.

mp 142-144° C.;

¹H NMR (DMSO-d₆) δ 2.99 (s, 3H), 3.99 (s, 2H), 6.05 (s, 2H), 6.98 (d, 1H), 7.08 (m, 2H), 7.30 (t, 1H), 7.39 (s, 1H), 8.83 (s, 1H), 9.75 (s, 1H);

MS (DCI/NH₃) m/z 352 (M+H)⁺;

Anal. calcd for C₁₁H₁₃N₃O₂S.C₄H₄O₄: C, 49.04; H, 4.66; N, 11.44. Found: C, 49.02; H, 4.67; N, 11.24.

### EXAMPLE 22

### N-[1-(1H-imidazol-4-yl)-2,3-dihydro-1H-inden-4-yl]methanesulfonamide, Maleate

### Example 22A

### 4-(7-nitro-1H-inden-3-yl)-1H-imidazole

4-Nitroindanone (Hasbun, J. A. J. Med. Chem. (1973), 16, 847-847) was processed as in Example 26B to provide the desired product.

MS (DCI/NH₃) m/z 228 (M+H)⁺.

### Example 22B

### tert-butyl 4-(7-nitro-1H-inden-3-yl)-1H-imidazole-1-carboxylate

Example 22A was processed as in Example 38C to provide the desired product.

### Example 22C

### tert-butyl 4-(4-amino-2,3-dihydro-1H-inden-1-yl)-1H-imidazole-1-carboxylate

Example 22B was processed as in Example 1C but substituting ethyl acetate for methanol as the solvent to provide the desired product.

MS (DCI/NH₃) m/z 300 (M+H)⁺.

### Example 22D

### N-[1-(1H-imidazol-4-yl)-2,3-dihydro-1H-inden-4-yl]methanesulfonamide, Maleate

Example 22C was processed as in Example 12D but substituting methanesulfonyl chloride for ethanesulfonyl chloride and substituting triethyl amine for pyridine to provide the desired product which was converted to the maleic acid salt.

mp 168-169° C.;

¹H NMR (CD₃OD) δ 2.17 (m, 1H), 2.64 (m, 1H), 2.97-3.09 (m, 1H), 3.01 (s, 3H), 3.19 (m, 1H), 4.62 (t, 1H), 6.25 (s, 2H), 6.95 (d, 1H), 7.23 (t, 1H), 7.29 (d, 1H), 7.31 (d, 1H), 8.75 (d, 1H);

MS (DCI/NH₃) m/z 278 (M+H)⁺;

Anal. calcd for C₁₃H₁₅N₃O₂S.C₄H₄O₄: C, 51.90; H, 4.87; N, 10.68. Found: C, 52.12; H, 4.72; N, 10.57.

### EXAMPLE 23

### N-[5,6,7,8-tetrahydro-5-(1H-imidazol-4-yl)-4-methyl-1-naphthalenyl]methanesulfonamide, Maleate

### Example 23A

### N-(4-methyl-5-oxo-5,6,7,8-tetrahydro-1-naphthalenyl)methanesulfonamide

A solution of 5-amino-8-methyltetralone (De, B. Synth. Commun. (1988), 18, 481-486) (0.25 g, 1.4 mmol) in dichloromethane (7 mL) was treated with pyridine (0.35 mL, 4.3 mmol), treated with methanesulfonyl chloride (0.12 mL, 1.5 mmol), stirred at ambient temperature for 1.5 hours, treated with aqueous ammonium chloride solution (20 mL) and extracted with dichloromethane (4×25 mL). The combined dichloromethane extracts were washed with brine, dried (Na₂SO₄) and concentrated. Purification of the residue on silica gel with ethyl acetate:hexanes 1:1 provided the desired product.

MS (APCI+) m/z 244 (M+H)⁺.

### Example 23B

### N-(methoxymethyl)-N-(4-methyl-5-oxo-5,6,7,8-tetrahydro-1-naphthalenyl)methanesulfonamide

Example 23A was processed as in Example 15B to provide the desired product.

MS (APCI+) m/z 298 (M+H)⁺.

### Example 23C

### N-[5-(1H-imidazol-4-yl)-4-methyl-7,8-dihydro-1-naphthalenyl]methanesulfonamide

A solution of 4-iodo-1-trityl-1H-imidazole (0.44 g, 1.0 mmol) (prepared as described by Kirk, K. J. J. Heterocyclic Chem. (1985), 22, 57-59) in dichloromethane (5 mL) under nitrogen was treated with ethylmagnesium bromide (0.33 mL, 1.0 mmol) over 4 minutes, stirred for 1 hour, cooled to 0° C., treated with Example 23B, stirred at ambient temperature for 2 hours, treated with water and extracted with ethyl acetate (3×50 mL). The combined ethyl acetate extracts were washed with brine, dried (Na₂SO₄), concentrated, treated with trifluoroacetic acid (20 mL), stirred for 1.5 hours, treated with water (7 mL), stirred over night and concentrated. Purification of the residue on silica gel with 7% ethanol/ammonia-saturated dichloromethane provided the desired product.

MS (APCI+) m/z 304 (M+H)⁺.

### Example 23D

### N-[5,6,7,8-tetrahydro-5-(1H-imidazol-4-yl)-4-methyl-1-naphthalenyl]methanesulfonamide, Maleate

Example 23C was processed as in Example 1C to provide the desired product, which was converted to the maleic acid salt.

mp 192-195° C.;

¹H NMR (DMSO-d₆) δ 1.38 (m, 1H), 1.69-2.07 (m, 3H), 2.01 (s, 3H), 2.66 (m, 1H), 2.94 (m, 1H), 3.00 (s, 3H), 4.31 (m, 1H), 6.06 (s, 2H), 6.75 (s, 1H), 7.05 (d, 1H), 7.19 (d, 1H), 8.92 (s, 2H);

MS (APCI+) m/z 306 (M+H)⁺;

MS (APCI−) m/z 304 (M−H)⁻, 340 (M+C1)⁻;

Anal. calcd for C₁₅H₁₉N₃O₂S.C₄H₄O₄.0.5H₂O.0.25 C₄C₈O₂: C, 53.09; H, 5.79; N, 9.29.

Found: C, 52.87; H, 5.58; N, 9.20.

### EXAMPLE 24

### N-[5,6,7,8-tetrahydro-4-hydroxy-5-(1H-imidazol-4-yl)-1-naphthalenyl]methanesulfonamide, Maleate

Example 26F was processed as in Example 2 to provide the desired product which was converted to the maleic acid salt.

mp 127-131° C.;

¹H NMR (DMSO-d₆) δ 1.44 (m, 1H), 1.74 (m, 1H), 1.85 (m, 1H), 1.96 (m, 1H), 2.62 (m, 1H), 2.91 (m, 1H), 2.95 (s, 3H), 4.29 (d, 1H), 6.04 (s, 2H), 6.66 (d, 1H), 6.85 (s, 1H), 7.07 (d, 1H), 8.75 (s, 1H), 8.85 (s, 1H);

MS (DCI/NH₃) m/z 308 (M+H)⁺;

Anal. calcd for C₁₄H₁₇N₃O₂S.C₄H₄O₄: C, 49.99; H, 5.13; N, 9.72. Found: C, 49.96; H, 5.21; N, 9.60.

### EXAMPLE 25

### N-[5,6,7,8-tetrahydro-(1H-imidazol-4-yl)-4-methoxy-1-naphthalenyl]ethanesulfonamide, Maleate

Example 26D was processed as in Example 12D to provide the desired product which was converted to the maleic acid salt.

mp 149-151° C.;

¹H NMR (DMSO-d₆) δ 1.28 (t, 3H), 1.42 (m, 1H), 1.74 (m, 1H), 1.84 (m, 1H), 1.98 (m, is 1H), 2.66 (m, 1H), 2.94 (m, 1H), 3.08 (q, 2H), 3.63 (s, 3H), 4.33 (d, 1H), 6.04 (s, 2H), 6.78 (s, 1H), 6.84 (d, 1H), 7.20 (d, 1H), 8.83 (s, 2H);

MS (DCI/NH₃) m/z 336 (M+H)⁺;

Anal. calcd for C₁₆H₂₁N₃O₃SC₄H₄O₄: C, 53.21; H, 5.58; N, 9.31. Found: C, 53.11; H, 5.72; N, 9.14.

### EXAMPLE 26

### N-[5,6,7,8-tetrahydro-(1H-imidazol-4-yl)-4-methoxy-1-naphthalenyl]methanesulfonamide, Maleate

### Example 26A

### 8-methoxy-5-nitro-3,4-dihydro-1 (2H)-naphthalenone

A solution of 8-methoxy-1-tetralone (2.26 g, 13 mmol) (prepared as described in Chatteee, A. Tetrahedron, (1980), 36, 2513-2520) in acetic anhydride (11.5 mL) was cooled to 0° C., treated with a mixture of fuming nitric acid (0.90 mL) in acetic acid (0.70 mL) dropwise over 1 hour, stirred at 0° C. for 1.5 hours, treated with water (150 mL) and extracted with diethyl ether (300 mL). The diether ether layer was washed with water (150 mL), washed with sodium bicarbonate solution (3×), washed with brine, dried (MgSO₄), filtered and concentrated. Purification of the residue on silica gel using a gradient of 2:1 and then 3:2 and finally 1 :1 hexanes:ethyl acetate provided the desired product as the more polar isomer.

mp 65-71° C.;

¹H NMR (CDCl₃) δ 2.09 (m, 2H), 2.68 (7, 2H), 3.21 (t, 2H), 4.00 (s, 3H), 6.96 (d, 1H), 8.13 (d, 1H);

MS (DCI/NH₃) m/z 222 (M+H)⁺.

### Example 26B

### 4-(8-methoxy-5-nitro-3,4-dihydro-1-naphthalenyl)-1H-imidazole

A solution of 4-iodo-1-trityl-1H-imidazole (prepared as described by Kirk, K. J. J. Heterocyclic Chem. (1985), 22, 57-59) (2.2 g, 5.1 mmol), in dichloromethane (20 mL) under nitrogen was treated with ethylmagnesium bromide (1.7 mL, 5.1 mmol) over 2 minutes, stirred for 30 minutes, treated with Example 26A (0.94 g, 4.2 mmol) in dichloromethane (5 mL), stirred for 2 hours, treated with ammonium chloride solution and extracted with dichloromethane (×2). The combined dichloromethane layers were dried (MgSO₄), filtered, concentrated, treated with ethyl acetate and hexane at which time the product was allowed to crystallize for 15 minutes. The crystals were collected by filtration, washed with 5:1 hexanes:ethyl acetate, dried under vacuum, treated with trifluoroacetic acid (25 mL), heated to reflux for 30 minutes, concentrated, treated with sodium bicarbonate solution and extracted with dichloromethane (×2). The combined dichloromethane extracts were dried (MgSO₄), filtered and concentrated to provide the desired product.

### Example 26C

### tert-butyl 4-(8-methoxy-5-nitro-3,4-dihydro-1-naphthalenyl)-1H-imidazole-1-carboxylate

A suspension of the product from Example 26B in acetonitrile (20 mL) was treated with di-tert-butyl dicarbonate (1 g, 4.6 mmol), heated on a steam bath for 20 minutes and concentrated. Purification of the residue on silica gel with 1:1 hexanes:ethyl acetate provided the desired product.

MS (DCI/NH₃) m/z 372 (M+H).

### Example 26D

### tert-butyl 4-(5-amino-8-methoxy-12,3,4-tetrahydro-1-naphthalenyl)-1H-imidazole-1-carboxylate

Example 26C was processed as in Example 1C substituting ethyl acetate for methanol as the solvent to provide the desired crude product.

MS (DCI/NH₃) m/z 344 (M+H)⁺.

### Example 26E

### tert-butyl 4-{8-methoxy-5-[(methylsulfonyl)amino]-1,2,3,4-tetrahydro-1-naphthalenyl}-1H-imidazole-1-carboxylate

A solution of Example 26D (0.50 g, 1.5 mmol) in dichloromethane (5 mL) was treated with pyridine (0.34 mL, 4.4 mmol), treated with methanesulfonyl chloride (0.17 mL, 2.2 mmol) and stirred for 1.5 hours. Purification of the mixture on silica gel eluting with ammonia-saturated dichloromethane and then with 10% ethyl acetate/ammonia-saturated dichloromethane provided the desired product which was dried under vacuum.

MS (DCI/NH₃) m/z 422 (M+H)⁺.

### Example 26F

### N-[5,6,7,8-tetrahydro-(1H-imidazol-4-yl)-4-methox-1-naphthalenyl]methanesulfonamide, Maleate

Example 26E was processed as in Example 33C to provide the desired product which was converted to the maleic acid salt.

mp 181-184° C.;

¹H NMR (DMSO-d₆) δ 1.43 (m, 1H), 1.75 (m, 1H), 1.85 (m, 1H), 1.97 (m, 1H), 2.66 (m, 1H), 2.93 (m, 1H), 2.98 (s, 3H), 3.64 (s, 3H), 4.34 (d, 1H), 6.04 (s, 2H), 6.82 (s, 1H), 6.86 (d, 1H), 7.24 (d, 1H), 8.85 (s, 1H), 8.87 (s, 1H);

MS (DCI/NH₃) m/z 322 (M+H)⁺;

Anal. calcd for C₁₅H₁₉N₃O₃SC₄H₄O₄: C, 52.17; H, 5.30; N, 9.61. Found: C, 51.95; H, 5.34; N, 9.31.

### EXAMPLE 27

### N-[5,6,7,8-tetrahydro-(1H-imidazol-4-yl)-1-naphthalenyl]cyclopropanesulfonamide, Maleate

Example 12C was processed as in Example 12D but substituting cyclopropylsulfonyl chloride (prepared as described in King, J. F. J. Org. Chem., (1993), 58, 1128-1135) for ethanesulfonyl chloride to provide the desired product which was converted to the maleic acid salt.

mp 156-157° C.;

¹H NMR (DMSO-d₆) δ 0.88 (m, 2H), 0.97 (m, 2H), 1.76 (m, 2H), 1.97 (m, 2H), 2.65 (m, 1H), 2.87 (t, 2H), 4.30 (t, 1H), 6.04 (s, 2H), 6.82 (d, 1H), 7.12 (t, 1H), 7.17 (s, 1H), 7.24 (d, 1H), 8.85 (s, 1H), 9.07 (s, 1H);

MS (DCI/NH₃) m/z 318 (M+H)⁺;

Anal. calcd for C₁₆H₁₉N₃O₂S.C₄H₄O₄: C, 55.42; H, 5.35; N, 9.69. Found: C, 55.40; H, 5.35; N, 9.67.

### EXAMPLE 28

### N-[3-(1H-imidazol-4-ylmethyl)-2-methylphenyl]methanesulfonamide, Maleate

### Example 28A

### 2-methyl-3-nitrobenzaldehyde

o-Tolualdehyde was nitrated and the majority of the undesired 2-methyl-5-nitrobenzaldehyde was removed as described in (Pitzele, B. S. J. Med. Chem., (1988), 31, 138-144) to provide a 2.7:1 ratio of 2-methyl-3-nitrobenzaldehyde: 2-methyl-5-nitrobenzaldehyde.

### Example 28B

### 4-[hydroxy(2-methyl-3-nitrophenyl)methyl]-N,N-dimethyl-1H-imidazole-1-sulfonamide

Example 28A (0.66 g) was processed as in Example 1A but was purified by recrystallization from ethyl acetate instead of by chromatography to provide the desired products as a mixture enriched in the 3-nitro isomer.

MS (DCI/NH₃) m/z 341 (M+H)⁺.

### Example 28C

### N,N-dimethyl-4-(2-methyl-3-nitrobenzyl)-1H-imidazole-1-sulfonamide

A solution of Example 28B in trifluoroacetic acid (15 mL) was treated with triethyl silane (1.5 mL), heated to reflux for 16 hours, cooled, concentrated, tritrated with hexanes, treated with sodium bicarbonate solution and extracted with dichloromethane (×2). The combined dichloromethane layers were dried (MgSO₄), filtered and concentrated. Purification of the residue on silica gel with ether provided the desired product enriched in the 3-nitro isomer.

MS (DCI/NH₃) m/z 325 (M+H)⁺.

### Example 28D

### 4-(3-amino-2-methylben:yl)-N.N-dimethyl-1H-imidazole-1-sulfonamide

Example 28C was processed as in Example 1C substituting ethyl acetate for methanol as the solvent. Purification of the residue on silica gel with 2% ethyl acetate/ammonia-saturated dichloromethane provided the desired product as the less polar isomer.

MS (DCI/NH₃) m/z 295 (M+H)⁺.

### Example 28E

### N-3-(1H-imidazol-4-ylmethyl)-2-methylphenyl]methanesulfonamide, Maleate

Example 28D was processed as in Example 31D to provide the desired product which was converted to the maleic acid salt.

mp 143-144° C.;

¹H NMR (DMSO-d₆) δ 2.25 (s, 3H), 2.96 (s, 3H), 4.02 (s, 2H), 6.05 (s, 2H), 7.05 (dd, 1H), 7.18 (t, 1H), 7.22 (dd, 1H), 7.26 (s, 1H), 8.83 (d, 1H), 9.12 (s, 1H);

MS (DCI/NH₃) m/z 266 (M+H)⁺;

Anal. calcd for C₁₂H₁₅N₃O₂SC₄H₄O₄: C, 50.39; H, 5.02; N, 11.02. Found: C, 50.32; H, 4.86; N, 10.90.

### EXAMPLE 29

### N-[3-(1H-imidazol-4-ylmethyl)-2-methalphenyl]ethanesulfonamide, Maleate

Example 28D was processed as in Example 3 ID but substituting ethanesulfonyl chloride for methanesulfonyl chloride to provide the desired product which was converted to the maleic acid salt.

mp 146-147° C.;

¹H NMR (DMSO-d₆) δ 1.26 (t, 3H), 3.25 (s, 3H), 3.06 (q, 2H), 4.01 (s, 2H), 6.05 (s, 2H), 7.02 (dd, 1H), 7.17 (m, 2H), 7.24 (d, 1H), 8.80 (d, 1H), 9.07 (s, 1H);

MS (DCI/NH₃) m/z 280 (M+H)⁺;

Anal. calcd for C₁₃H₁₇N₃O₂S.C₄H₄O₄: C, 51.64; H, 5.35; N, 10.63. Found: C, 51.64; H, 5.08; N, 10.45.

### EXAMPLE 30

### N-[3-(1H-imidazol-4-ylmethyl) phenyl]ethanesulfonamide, Maleate

Example 21C was processed as in Example 21D but substituting ethanesulfonyl chloride for methanesulfonyl chloride to provide the desired product which was converted to the maleic acid salt.

mp 107-109° C.;

¹H NMR (DMSO-d₆) δ 1.18 (t, 3H), 3.08 (q, 2H), 3.99 (s, 2H), 6.05 (s, 2H), 6.96 (d, 1H), 7.08 (m, 2H), 7.28 (m, 1H), 7.37 (d, 1H), 8.80 (d, 1H), 9.77 (s, 1H);

MS (DCI/NH₃) m/z 266 (M+H)⁺;

Anal. calcd for C₁₂H₁₅N₃O₂SC₄H₄O₄: C, 50.39; H, 5.02; N, 11.02. Found: C, 50.44; H, 4.91; N, 10.89.

### EXAMPLE 31

### N-[3-[1-(1H-imidazol-4-yl)ethyl]phenyl]methanesulfonamide, Maleate

### Example 31A

### 4-[1-hydroxy-1-(3-nitrophenyl)ethyl]-N,N-dimethyl-1H-imidazole-1-sulfonamide

3-nitroacetophenone was processed as in Example 1A to provide the desired product.

MS (DCI/NH₃) m/z 341 (M+H)⁺.

### Example 31B

### N,N-dimethyl-4-[1-(3-nitrophenyl)vinyl]-1H-imidazole-1-sulfonamide

Example 31A was treated with trifluoroacetic acid (30 mL), heated briefly on a steam bath, stirred at ambient temperature for 16 hours, heated to reflux for 1 hour, concentrated, treated with sodium bicarbonate solution and extracted with dichloromethane (2×). The combined dichloromethane extracts were dried (MgSO₄), filtered and concentrated. Purification of the residue on silica gel with 4:1 ethyl acetate:hexanes and then ethyl acetate provided the desired product.

MS (DCI/NH₃) ni/z 323 (M+H)⁺.

### Example 31C

### 4-[1-(3-aminophenyl)ethyl]-N,N-dimethyl-1H-imidazole-1-sulfonamide

Example 31B was processed as in Example 1C but substituting ethyl acetate for methanol as the solvent to provide the desired product.

MS (DCI/NH₃) m/z 295 (M+H)⁺.

### Example 31D

### N-[3-[1-(1H-imidazol-4-yl)ethyl]phenyl]methanesulfonamide, Maleate

A solution of Example 31C (0.19 g, 0.55 mmol) in dichloromethane (7 mL) was treated with pyridine (0.14 mL, 1.7 mmol), treated with methanesulfonyl chloride (0.65 mL, 0.83 mmol), stirred for 16 hours at room temperature, concentrated to dryness, treated with 2M HCl (7 mL), refluxed for 16 hours and concentrated. Purification of the residue on silica gel with 10% ethanol/ammonia-saturated dichloromethane provided the desired product which was converted to the maleic acid salt.

mp 135-136° C.;

¹H NMR (DMSO-d₆) δ 1.55 (d, 3H), 2.98 (s, 3H), 4.20 (q, 1H), 6.05 (s, 2H), 6.98 (d, 1H), 7.05 (s, 1H), 7.08 (d, 1H), 7.30 (t, 1H), 7.47 (s, 1H), 8.84 (s, 1H), 9.75 (s, 1H);

MS (DCI/NH₃) m/z 266 (M+H)⁺;

Anal. calcd for C₁₂H₁₅N₃O₂S.C₄H₄O₄: C, 50.39; H, 5.02; N, 11.02. Found: C, 50.27; H, 4.99; N, 10.90.

### EXAMPLE 33

### (+)-N-[5-(1H-imidazol-4-yl)-5,6,7,8-tetrahydro-1-naphthalenyl]ethanesulfonamide, Maleate

### Example 33A

### tert-butyl 4-{5-[(ethylsulfonyl)amino]-1,2,3,4-tetrahydro-1-naphthalenyl}-1H-imidazole-1-carboxylate

A solution of Example 12C (2.0 g, 6.4 mmol) in dichloromethane (30 mL) was treated with pyridine (1.6 mL, 19 mmol), treated with ethanesulfonyl chloride (0.91 mL, 9.6 mmol), stirred for 16 hours, diluted with dichloromethane and washed with 1M HCl. The aqueous layer was extracted with dichloromethane (2×) and the combined dichloromethane layers were dried (MgSO₄), filtered and concentrated. Purification of the residue on silica gel with 2:1:1 ethyl acetate:dichloromethane:hexane provided the desired product.

MS (DCI/NH₃) m/z 406 (M+H)⁺.

### Example 33B

### (+)-tert-butyl 4-(1R)-5-[(ethylsulfonyl)amino-1,2,3,4-tetrahydro-1-naphthalenyl]-1H-imidazole-1-carboxylate

The enantiomers of Example 33A were separated by chiral chromatography on a Chiracel OJ column (5.0 cm inner diameter, 50 cm length, 20 micron packing) using 95:5 hexanes:ethanol at a flow rate of 117 mL/minute as the mobile phase.

[α]²³_{D}+59.9 (c 1.1, CHCl₃).

### Example 33C

### (+)-N-[5-(1H-imidazol-4-yl)-5,6,7,8-tetrahydro-1-naphthalenyl]ethanesulfonamide, Maleate

A solution of the faster moving enantiomer from Example 33B (0.26 g, 0.64 mmol) in dichloromethane (4 mL) was treated with trifluoroacetic acid (5 mL), heated on a steam bath for 1 minute and concentrated. Purification of the residue on silica gel using 5% and then 10% methanol/ammonia-saturated dichloromethane provided a solid, which was converted to the maleic acid salt.

mp 129-130° C.;

[α]²³_{D} (free base)+55.2 (c 1.1, 1:1 methanol:chloroform);

¹H NMR (DMSO-d₆) δ 1.28 (t, 3H), 1.67-1.85 (m, 2H), 1.87-2.06 (m, 2H), 2.83 (t, 2H), 3.13 (q, 2H), 4.30 (t, 1H), 6.05 (s, 2H), 6.80 (d, 1H), 7.12 (t, 1H), 7.16-7.23 (m, 2H);

MS (DCI/NH₃) m/z 306 (M+H)⁺;

Anal. calcd for C₁₅H₁₉N₃O₂S.C₄H₄O₄: C, 54.15; H, 5.50; N, 9.97. Found: C, 54.03; H, 5.40;N,9.87.

### EXAMPLE 34

### (−)-N-[5-(1H-imidazol-4-yl)-5,6,7,8-tetrahydro-1-naphthalenyl]ethanesulfonamide, Maleate

### Example 34A

### (−)-tert-butyl 4-{(1R)-5-[(ethylsulfonyl)amino]-1,2,3,4-tetrahydro-1-naphthalenyl}-1H-imidazole-1-carboxylate

The title compound was provided by Example 33B as the slower moving enantiomer.

[α]²³_{D}−60.4 (c 1.1, CHCl₃).

### Example 34B

### (−)-N-[5-(1H-imidazol-4-yl)-5,6,7,8-tetrahydro-1-naphthalenyl]ethanesulfonamide, Maleate

Example 34A was processed as described in 33C to provide the desired product which was converted to the maleic acid salt.

mp 129-130° C.;

[α]²³_{D} (free base) −56.1° (c 1.0, 1:1 methanol:chloroform);

¹H NMR (DMSO-d₆) δ 1.28 (t, 3H), 1.67-1.85 (m, 2H), 1.87-2.06 (m, 2H), 2.83 (t, 2H), 3.13 (q, 2H), 4.30 (t, 1H), 6.05 (s, 2H), 6.80 (d, 1H), 7.12 (t, 1H), 7.16-7.23 (m, 2H);

MS (DCI/NH₃) m/z 306 (M+H)⁺;

Anal. calcd for C₁₅H₁₉N₃O₂SC₄H₄O₄: C, 54.15; H, 5.50; N, 9.97. Found: C, 54.44; H, 5.70; N, 9.97.

### EXAMPLE 35

### (−)-N-[5,6,7,8-tetrahydro-5-(1H-imidazol-4-yl)-1-naphthalenlyl]-2,2,2-trifluoroethanesulfonamide

### Example 35A

### (−)-tert-butyl 4-(-5-{[(2,2,2-trifluoroethyl)sulfonyl]amino}-1,2,3,4-tetrahydro-1-naphthalenyl)-1H-imidazole-1-carboxylate

The enantiomers of Example 20A were separated by chiral chromatography on a Chiralpak AD column (5.0 cm inner diameter, 26 cm length, 20 uDp) using 96:4 hexanes:ethanol at a flow rate of 117 mL/minute as the mobile phase to provide the title compound as the faster moving enantiomer.

[α]²³_{D}−48.9° (c 0.95, CHCl₃).

### Example 35B

### (−)-N-[5,6,7,8-tetrahydro-5-(1H-imidazol-4-yl)-1-naphthalenyl]-2,2.2-trifluoroethanesulfonamide

A solution of Example 35A (0.20 g, 0.44 mmol) in dichloromethane (4 mL) was treated with trifluoroacetic acid (5 mL), heated on a steam bath for 1 minute and concentrated. Purification of the residue on silica gel using 10% and then 20% methanol/ammonia-saturated dichloromethane provided the desired product.

mp>260° C.;

¹H NMR (DMSO-d₆) δ 1.61-1.83 (m, 2H), 1.83-2.06 (m, 2H), 2.67-2.87 (m, 2H), 4.06 (t, 1H), 4.48 (q, 2H), 6.64 (s, 1H), 6.95 (d, 1H), 7.08 (t, 1H), 7.17 (d, 1H), 7.54 (s, 1H), 9.8 (bs, 1H), 11.5 (bs, 1H);

[α]²³_{D}−30.20 (c 0.95, acetic acid);

MS (DCI/NH₃) m/z 360 (M+H)⁺;

Anal. calcd for C₁₅H₁₆N₃O₂SF₃: C, 50.13; H, 4.49; N, 11.69. Found: C, 50.30; H, 4.52; N, 11.51.

### EXAMPLE 36

### (+)-N-[5,6,7,8-tetrahydro-5-(1H-imidazol-4-yl)-1-napbthalenyl]-2,2,2-trifluoroethanesulfonamide

The slower moving enantiomer from Example 35A was processed as in Example 35B to provide the title compound.

mp>260° C.;

[α]²³_{D}+30.40 (c 0.97, acetic acid);

¹H NMR (DMSO-d₆) δ 1.61-1.83 (m, 2H), 1.83-2.06 (m, 2H), 2.67-2.87 (m, 2H), 4.06 (t, 1H), 4.48 (q, 2H), 6.64 (s, 1H), 6.95 (d, 1H), 7.08 (t, 1H), 7.17 (d, 1H), 7.54 (s, 1H), 9.8 (bs, 1H), 11.5 (bs, 1H);

MS (DCI/NH₃) m/z 360 (M+H)⁺;

Anal. calcd for C₁₅H₁₆N₃O₂SF₃: C, 50.13; H, 4.49; N, 11.69. Found: C, 50.26; H, 4.47; N, 11.49.

### EXAMPLE 37

### N-{3-[1-(1H-imidazol-4-yl)ethyl]phenyl}ethanesulfonamide, Maleate

Example 31C was processed as in Example 21D but substituting ethanesulfonyl chloride for methanesulfonyl chloride to provide the desired product, which was converted to the maleic acid salt.

mp 114-119° C.;

¹H NMR (DMSO-d₆) δ 1.17 (t, 3H), 1.55 (d, 3H), 3.07 (q, 2H), 4.20 (q, 1H), 6.05 (s, 2H), 6.96 (d, 1H), 7.04-7.12 (m, 2H), 7.28 (t, 1H), 7.45 (s, 1H), 8.82 (d, 1H), 9.76 (s, 1H), 14.00 (bs, 1H);

MS (DCI/NH₃) m/z 280 (M+H)⁺;

Anal. calc'd for C₁₃H₁₇N₃O₂S.C₄H₄O₄0.25H₂O: C, 51.05; H, 5.52; N, 10.51. Found: C, 51.20; H, 5.53; N, 10.31.

### EXAMPLE 38

### N-[5-(1H-imidazol-4-yl)-6,7,8,9-tetrahydro-5H-benzo[a]cyclohepten-1-yl]methanesulfonamide, Maleate

### Example 38A

### 1-nitro-6,7,8,9-tetrahydro-5H-benzo[a]cyclohepten-5-one

6,7,8,9-Tetrahydro-5H-benzo[a]cyclohepten-5-one (18.5 g, 11.5 mmol) was mechanically stirred at −15° C. and treated with concentrated sulfuric acid (41 mL) over 5 minutes, stirred 10 minutes, treated dropwise over 10 minutes with a mixture of fuming nitric acid (9 mL) and concentrated sulfuric acid (14 mL), stirred at −15° C. for 15 minutes and poured carefully onto a mixture of ice (200 g) and water (200 mL). The resulting solid was collected by filtration, washed with water (100 mL, 2×), dried and recrystallized from ethanol (200 mL). The resulting solid was removed by filtration and the filtrate was suspended on silica gel and purified on silica gel eluting with ethyl acetate:hexanes 12:88 to provide the desired product.

¹H NMR (CDCl₃) δ 1.78-1.87 (m, 2H), 1.97-2.06 (m, 2H), 2.74 (7, 2H), 2.98 (t, 2H), 7.44 (t, 1H), 7.82 (dd, 1H), 7.91 (dd, 1H).

### Example 38B

### 4-(4-nitro-6,7-dihydro-5H-benzo[a]cyclohepten-9-yl)-1H-imidazole

Example 38A was processed as in Example 26B to provide the desired product, which was carried onto the next step without purification.

### Example 38C

### tert-butyl 4-(4-nitro-6,7-dihydro-5H-benzo[a]cyclohepten-9-yl)-1H-imidazole-1-carboxylate

Example 38B was processed as in Example 3C but instead of concentrating the dimethylformamide, the mixture was partitioned between ether and water. The ether layer was isolated, washed with water, brine, dried (MgSO₄), filtered and concentrated.

MS (DCI/NH₃) m/z 356 (M+H)⁺.

### Example 38D

### tert-butyl 4-(1-amino-6,7,8,9-tetrahydro-5H-benzo[a]cyclohepten-5-yl)-1H-imidazole-1-carboxylate

Example 38C was processed as in Example 1C but substituting ethyl acetate for methanol as the solvent to provide the desired product.

MS (DCI/NH₃) m/z 328 (M+H)⁺.

### Example 38E

### N-[5-(1H-imidazol-4-yl)-6,7,8,9-tetrahydro-5H-benzo[a]cyclohepten-1-yl]methanesulfonamide, Maleate

Example 38D was processed as in Example 12D but substituting methanesulfonyl chloride for ethanesulfonyl chloride to provide the desired product, which was converted to the maleic acid salt.

mp 162-164° C.;

¹H NMR (CD₃OD) δ 1.58 (m, 1H), 1.83 (m, 3H), 2.06 (m, 1H), 2.17 (m, 1H), 2.97 (s, 3H), 3.00 (m, 1H), 3.18 (m, 1H), 4.54 (dd, 1H), 6.25 (s, 2H), 7.69 (d, 1H), 7.14 (t, 1H), 7.26 (dd, 1H), 7.29 (s, 1H), 8.81 (d, 1H);

MS (DCI/NH₃) m/z 306 (M+H)⁺;

Anal. calcd for C₁₅H₁₉N₃O₂S C₄H₄O₄0.5 C₄H₈O₂: C, 54.18; H, 5.85; N, 9.03. Found: C, 53.97; H, 5.82; N, 8.86.

### EXAMPLE 39

### N-[1-(1H-imidazol-4-yl)-2,3-dihydro-1H-inden-4-yl]ethanesulfonamide, Maleate

Example 22C was processed as in Example 12D but substituting triethylamine for pyridine to provide the desired product, which was converted to the maleic acid salt.

mp 148-149° C.;

¹H NMR (CD₃OD) δ 1.36 (t, 3H), 2.16 (m, 1H), 2.64 (m, 1H), 2.96-3.24 (m, 2H), 3.14 (q, 2H), 4.62 (t, 1H), 6.25 (s, 2H), 6.92 (d, 1H), 7.21 (t, 1H), 7.29 (m, 2H), 8.76 (d, 1H); MS (DCI/NH₃) m/z 292 (M+H)⁺;

Anal. calcd for C₁₄H₁₇N₃O₂S C₄H₄O₄: C, 53.06; H, 5.20; N, 10.31. Found: C, 53.06; H, 5.17; N, 10.30.

### EXAMPLE 40

### N-[5-(1H-imidazol-4-yl)-6,7,8,9-tetrahydro-5H-benzo[a]cyclohepten-1-yl]ethanesulfonamide, Maleate

Example 38D was processed as in Example 12D to provide the desired product, which was converted to the maleic acid salt.

mp 155-156° C.;

¹H NMR (CD₃OD) δ 1.39 (t, 3H), 1.58 (m, 1H), 1.73-1.92 (m, 3H), 2.05 (m, 1H), 2.18 (m, 1H), 2.99 (m, 1H), 3.10 (q, 2H), 3.19 (m, 1H), 4.54 (dd, 1H), 6.25 (s, 2H), 6.67 (d, 1H), 7.13 (t, 1H), 7.24 (dd, 1H), 7.29 (s, 1H), 8.81 (d, 1H);

MS (CDI/NH₃) m/z 320 (M+H)⁺;

Anal. calcd for C₁₆H₂₁N₃O₆S.C₄H₄O₄: C, 55.16; H, 5.79; N, 9.65. Found: C, 54.96; H, 5.67; N, 9.47.

### EXAMPLE 41

### N-[4-fluoro-3-(1H-imidazol-4-ylmethyl)phenyl]methanesulfonamide, Maleate

### Example 41A

### 4-[(2-fluoro-5-nitrophenyl)(hydroxy)methyl]-N,N-dimethyl-1H-imidazole-1-sulfonamide

2-Fluoro-5-nitrobenzaldehyde was substituted for 6-methoxy-5-nitro-1-tetralone and processed as described in Example 1A to provide the desired product.

MS (DCI/NH₃) m/z 345 (M+H)⁺.

### Example 41B

### 4-(2-fluoro-5-nitrobenzyl)-N,N-dimethyl-1H-imidazole-1-sulfonamide

A mixture of Example 41A (0.45 g, 1.3 mmol) and triethylsilane (0.5 g, 4.3 mmol) in trifluoroacetic acid (5 mL) was refluxed for 6 hours, cooled to ambient temperature, concentrated, neutralized with aqueous sodium bicarbonate and extracted (2×) with dichloromethane. The combined dichloromethane extracts were dried (MgSO₄), filtered and concentrated. Purification of the residue on silica gel eluting with ethyl acetate:hexanes 1:1 provided the desired product.

MS (DCI/NH₃) m/z 329 (M+H)⁺.

### Example 41C

### 4-(5-amino-2-fluorobenzyl)-N,N-dimethyl-1H-imidazole-1-sulfonamide

Example 41B was processed as in Example 1C but substituting ethyl acetate for methanol as the solvent to provide the desired product.

MS (DCI/NH₃) m/z 299 (M+H)⁺.

### Example 41D

### N-[4-fluoro-3-(1H-imidazol-4-ylmethyl)phenyl]methanesulfonamide, Maleate

Example 41C was processed as described in Example 31D to provide the desired product, which was converted to the maleic acid salt.

mp 146-147° C.;

¹H NMR (DMSO-d₆) δ 2.95 (s, 3H), 4.01 (s, 2H), 6.06 (s, 2H), 7.12 (m, 2H), 7.21 (t, 1H), 7.32 (s, 1H), 8.75 (s, 1H), 9.65 (s, 1H); MS (DCI/NH₃) m/z 270 (M+H)⁺;

Anal. calcd for C₁₁H₁₂N₃O₂SFC₄H₄O₄: C, 46.75; H, 4.18; N, 10.90. Found: C, 46.63; H, 4.32; N, 10.85.

### EXAMPLE 42

### N-[4-chloro-5-(1H-imidazol-4-yl)-5,6,7,8-tetrahydro-1-naphthalenyl]ethanesulfonamide, Maleate

### Example 42A

### 5-amino-8-chloro-3,4-dihydro-1 (2H)-naphthalenone

A solution of 5-amino-1-tetralone (Itoh, K. Chem. Pharm. Bull. (1984), 32, 130-151) (0.50 g, 3.1 mmol) in dimethylformamide (15 mL) was treated with N-chlorosuccinimide (0.49 g, 3.7 mmol), stirred for 60 hours, treated with water and extracted with ether (4×30 mL). The combined ether extracts were washed with brine, dried (Na₂SO₄) and concentrated. Purification of the residue on silica gel with ethyl acetate:hexanes 1:1 provided the desired product.

¹H NMR (CDCl₃) δ 2.16 (m, 2H), 2.67 (m, 4H), 3.72 (s, 2H), 6.75 (d, 1H), 7.14 (d, 1H); MS (APCI+) m/z 196 (M+H)⁺.

### Example 42B

### N-(4-chloro-5-oxo-5,6,7,8-tetrahydro-1-naphthalenyl)ethanesulfonamide

A solution Example 42A (0.14 g, 0.72 mmol) in dichloromethane (5 mL) was treated with pyridine (0.18 mL, 2.2 mL), treated with ethanesulfonyl chloride (0.11 mL, 1.1 mmol), stirred for 16 hours, treated with pyridine (1 mL), treated with ethanesulfonyl chloride (0.5 mL), stirred for 3 hours and concentrated. Purification of the residue on silica gel with 5% ethanol/ammonia-saturated dichloromethane provided the desired product.

MS (ESI−) m/z 286 (M−H)⁻.

### Example 42C

### N-(4-chloro-5-oxo-5,6,7,8-tetrahydro-1-nanhthalenyl)-N-(methoxymethyl)ethanesulfonamide

Example 42B was processed as described in Example 15B to provide the desired product.

MS (ESI+) m/z 332 (M+H)⁺, 349 (M+NH₄)+.

### Example 42D

### N-[4-chloro-5-(1H-imidazol-4-yl)-7,8-dihydro-1-naphthalenyl]ethanesulfonamide

Example 42C was processed as described in Example 8B except that the 2M HCl mixture was heated to reflux for 16 hours and the mixture was then concentrated to dryness and used without further purification.

### Example 42E

### N-[4-chloro-5-(1H-imidazol-4-yl)-5,6,7,8-tetrahydro-1-naphthalenyl]ethanesulfonamide, Maleate

Example 42D was processed as described in Example 43D to provide the desired product, which was converted to the maleic acid salt.

mp 151-155° C.;

¹H NMR (DMSO-d₆) δ 1.28 (t, 3H), 1.36-1.49 (m, 1H), 1.72-2.06 (m, 3H), 2.57-2.74 (m, 1H), 2.96 (dd, 1H), 3.16 (q, 2H), 4.43 (d, 1H), 6.05 (s, 2H), 6.80 (s, 1H), 7.31 (s, 2H), 8.81 (s, 1H), 9.12 (s, 1H);

MS (DCI/NH₃) m/z 340 (M+H)⁺;

Anal. calcd for C₁₅H₁₈N₃O₂SClC₄H₄O₄ 0.25 C₄H₈O₂: C, 50.26; H, 5.06; N, 8.79. Found: C, 50.44; H, 5.11; N, 8.70.

### EXAMPLE 43

### N-[4-chloro-5-(1H-imidazol-4-yl)-5,6,7,8-tetrahydro-1-naphthalenyl]methanesulfonamide, Maleate

### Example 43A

### N-(4-chloro-5-oxo-5,6,7,8-tetrahydro-1-naphthalenyl)methanesulfonamide

Example 42A was processed as in Example 42B but substituting methanesulfonyl chloride for ethanesulfonyl chloride to provide the desired product.

MS (APCI−) m/z 272 (M−H)⁻.

### Example 43B

### N-(4-chloro-5-oxo-5,6,7,8-tetrahydro-1-naphthalenyl)-N-(methoxymethyl)methanesulfonamide

Example 43A was processed as in Example 15B to provide the desired product.

MS (APCI+) m/z 318 (M+H)⁺, 335 (M+NH₄)+.

### Example 43C

### N-[4-chloro-5-(1H-imidazol-4-yl)-7,8-dihydro-1-naphthalenyl]methanesulfonamide

Example 43B was processed as described in Example 8B except that the 2M HCl mixture was heated to reflux for 16 hours and the mixture was then concentrated to dryness and used without further purification.

### Example 43D

### N-[4-chloro-5-(1H-imidazol-4-yl)-5,6,7,8-tetrahydro-1-naphthalenyl]methanesulfonamide, Maleate

A mixture of Example 43C (0.16 g, 0.50 mmol) and 10% Pd/C in 5:1 tetrahydrofuran:5 M HCl (6 mL) was stirred under a hydrogen (1 atmosphere) for 1 hour, filtered and concentrated. Purification of the residue on silica gel with 10% methanol/ammonia-saturated dichloromethane provided the desired product, which was converted to the maleic acid salt.

mp 175-178° C.;

¹H NMR (DMSO-d₆) δ 1.30-1.85 (m, 2H), 1.86-2.08 (m, 2H), 2.60-3.00 (m, 2H), 3.06 (s, 3H), 4.44 (m, 1H), 6.05 (s, 2H), 6.82 (s, 1H), 7.32 (s, 2H), 8.80 (s, 1H), 9.15 (s, 1H); MS (APCI+) m/z 326 (M+H)⁺;MS (APCI−) m/z 324 (M−H)⁻;

Anal. calcd for C₁₄H₁₆N₃O₂SCl.C₄H₄O₄: C, 48.91; H, 4.56; N, 9.51. Found: C, 48.62; H, 4.51; N, 9.26.

### EXAMPLE 44

### N-[4-fluoro-5-(1H-imidazol-4-yl)-5,6,7,8-tetrahydro-1-naphthalenyl]methanesulfonamide, Maleate

### Example 44A

### 8-fluoro-5-hydroxy-3,4-dihydro-1 (2H)-naphthalenone

A solution of 8-fluoro-5-methoxytetralone (Owton, W. M. J. Chem. Soc., Perkin Trans. 1 (1994), 2131-2135) (7.0 g, 36 mmol) in 1,2-dichlorethane (150 mL) was treated with aluminum chloride (21 g, 157 mmol), refluxed for 3.5 hours, cooled to ambient temperature, poured carefully into 4M HCl (500 mL), stirred for 16 hours, treated with dichloromethane (400 mL) and thoroughly shaken. A black solid was removed by filteration through Celite®. The dichloromethane layer was isolated, combined with the black solid and extracted with 5% sodium hydroxide solution (3×150 mL). The combined sodium hydroxide extracts were acidified with 4M hydrochloric acid and the resulting solid was collected by filtration to provide the desired product as a brown solid.

MS (APCI+) m/z 181 (M+H).

### Example 44B

### 4-fluoro-5-oxo-5,6,7,8-tetrahydro-1-naphthalenyl trifluoromethanesulfonate

A solution of Example 44A (1.0 g, 5.5 mmol) in pyridine (3 mL) under nitrogen was cooled to 0° C., treated dropwise with trifluoromethanesulfonic anhydride (1.0 mL, 6.2 mmol), stirred for 16 hours at ambient temperature, treated with 2M HCl (25 mL), stirred for 30 minutes and extracted with ethyl acetate (3×70 mL). The combined ethyl acetate extracts were washed with brine and concentrated. Purification of the residue on silica gel with 40% ethyl acetate/hexanes provided the desired product.

MS (APCI+) m/z 330 (M+NH₄)⁺.

### Example 44C

### 5-(benzylamino)-8-fluoro-3,4-dihydro-1(2H)-naphthalenone

A mixture of tris(dibenzylideneacetone)dipalladium(0) (0.36 g, 0.34 mmol) under nitrogen in toluene (136 mL) was treated with (R)-(+)-2,2′-bis(diphenylphosphino)-1,1′-binaphthyl (0.96 g, 1.5 mmol), treated with sodium tert-butoxide (0.98 g, 10 mmol), treated with benzyl amine (1.1 mL, 10 mmol), warmed to 85° C., treated dropwise over 45 minutes with a solution of Example 44B (2.1 g, 6.8 mmol) in toluene (30 mL), stirred at 85° C. for 1 hour and treated with water (50 mL). The organic layer was isolated and the aqueous layer was extracted with ethyl acetate. The combined organic layers were washed with brine, dried (Na₂SO₄) and concentrated. Purification of the residue on silica gel with 30% ethyl acetate/hexanes provided the desired product.

MS (APCI+) m/z 348 (M+H)⁺, 365 (M+NH₄)⁺.

### Example 44D

### N-benzyl-N-(4-fluoro-5-oxo-5,6,7,8-tetrahydro-1-naphthalenyl)methanesulfonamide

A solution of Example 44C (0.40 g, 1.5 mmol) in dichloromethane (9 mL) was treated with pyridine (0.36 mL, 4.4 mmol), treated with methanesulfonyl chloride (0.13 mL, 1.6 mmol), stirred for 4 hours, treated with pyridine (0.2 mL, 2.5 mmol), treated with methanesulfonyl chloride (0.10 mL, 1.3 mmol), stirred for 16 hours, refluxed for 9 hours, cooled to ambient temperature, treated with water (25 mL) and extracted with dichloromethane (3×20 mL). The combined dichloromethane extracts were washed with brine, dried (Na₂SO₄) and concentrated. Purification of the residue on silica gel with 1:1 ethyl acetatge:hexanes provided the desired product.

### Example 44E

### N-benzyl-N-[4-fluoro-5-(1H-imidazol-4-yl)-7,8-dihydro-1-naphthalenyl]methanesulfonamide

Example 44D was processed as in Example 8B to provide the desired product.

MS (APCI+) m/z 398 (M+H)⁺.

### Example 44F

### N-[4-fluoro-5-(1H-imidazol-4-yl)-5,6,7,8-tetrahydro-1-naphthalenyl]methanesulfonamide, Maleate

Example 44E was processed as in Example 1C to provide the desired product which was converted to the maleic acid salt.

mp 182-186° C.;

¹H NMR (DMSO-d₆) δ 1.50 (m, 1H), 1.76 (m, 1H), 1.95 (m, 2H), 2.70 (m, 1H), 2.92 (m, 1H), 3.02 (s, 3H), 4.42 (m, 1H), 6.07 (s, 2H), 6.99 (s, 1H), 7.05 (t, 1H), 7.30 (dd, 1H), 8.86 (s, 1H), 9.08 (s, 1H);

MS (APCI+) m/z 310 (M+H)⁺;

MS (APCI−) m/z 308 (M−H)⁻;

Anal. calcd for C₁₄H₁₆N₃O₂SF.C₄H₄O₄: C, 50.81; H, 4.74; N, 9.87. Found: C, 50.71; H, 4.87; N, 9.72.

### EXAMPLE 45

### N-{3-[1-(1H-imidazol-4-yl)vinyl]phenyl}ethanesulfonamide, Maleate

### Example 45A

### 4-[1-(3-aminophenyl)vinyl]-N,N-dimethyl-1H-imidazole-1-sulfonamide

Example 31B was processed as in Example 46B to provide the desired product. MS (APCI+) m/z 293 (M+H)⁺.

### Example 45B

### N-{3-[1-(1H-imidazol-4-yl)vinyl]phenyl}ethanesulfonamide, Maleate

Example 45A was processed as in Example 31D except ethanesulfonyl chloride was used instead of methanesulfonyl chloride to provide the desired product which was converted to the maleic acid salt.

mp 151-155° C.;

¹H NMR (DMSO-d₆) δ 1.20 (t, 3H), 3.11 (q, 2H), 5.44 (s, 1H), 5.81 (s, 1H), 6.11 (s, 2H), 7.15 (d, 1H), 7.25 (d, 1H), 7.27 (s, 1H), 7.34 (s, 1H), 7.38 (dd, 1H), 8.65 (s, 1H), 9.89 (s, 1H);

MS (APCI+) m/z 278 (M+H)⁺;

MS (APCI−) m/z 276 (M−H)⁻;

Anal. calcd for C₁₃H₁₅N₃O₂S C₄H₄O₄: C, 51.31; H, 4.94; N, 10.56. Found: C, 51.37; H, 5.07; N, 10.22.

### EXAMPLE 46

### N-{3-[(Z)-1-(1H-imidazol-4-yl)-2-methoxyethenyl]phenyl}ethanesulfonamide, Maleate

### Example 46A

### 4-[(Z)-2-methoxy-1-(3-nitrophenyl)ethenyl]-N,N-dimethyl-1H-imidazole-1-sulfonamide

A solution of (methoxymethyl)triphenylphosphonium chloride (0.67 g, 1.9 mmol) in tetrahydrofuran (6.4 mL) under a nitrogen atmosphere was treated with a solution of 2.5M n-butyllithium in hexanes (0.78 mL, 1.9 mmol), treated with a solution of Example 55B (0.67 g, 2.0 mmol) in THF (30 mL), stirred for 16 hours, treated with ammonium chloride solution and extracted with ethyl acetate (3×60 mL). The combined ethyl acetate extracts were washed with brine, dried (Na2SO4) and concentrated. Purification of the residue on silica gel with ethyl acetate provided the desired product as the less polar isomer.

¹H NMR (CDCl₃) δ 2.90 (s, 6H), 3.94 (s, 3H), 6.49 (s, 1H), 7.50 (dd, 1H), 7.66 (d, 1H), 7.74 (m, 1H), 7.83 (d, 1H), 8.12 (m, 1H), 8.24 (t, 1H);

MS (APCI+) m/z 353 (M+H)⁺.

### Example 46B

### 4-[(Z)-1-(3-aminophenyl)-2-methoxyethenyl]-N,N-dimethyl-1H-imidazole-1-sulfonamide

A solution of Example 46A (0.15 g, 0.43 mmol) in methanol (0.70 mL) was cooled to 0° C., treated with concentrated HCl (0.35 mL), treated with zinc (0.28 g, 4.3 mmol) in portions, stirred at ambient temperature for 20 minutes, neutralized with aqueous sodium bicarbonate solution (15 mL) and extracted with ethyl acetate (4×20 mL). The combined ethyl acetate extracts were dried (Na₂SO₄) and concentrated to provide the desired product.

MS (APCI+) m/z 323 (M+H)⁺.

### Example 46C

### 4-((Z)-1-{3-[(ethylsulfonyl)amino]phenyl}-2-methoxyethenyl)-N,N-dimethyl-1H-imidazole-1-sulfonamide

A solution of Example 46B (0.32 g, 0.99 mmol) in dichloromethane (5 mL) was treated with pyridine (0.24 mL, 3.0 mmol), treated with ethanesulfonyl chloride (0.10 mL, 1.1 mmol), stirred for 5 hours, treated with 1 M HCl and extracted with dichloromethane (3×). The combined dichloromethane extractions were dried (Na₂SO₄) and concentrated to provide the title compound.

MS (APCI+) m/z 415 (M+H)⁺.

### Example 46D

### N-{3-[(Z)-1-(1H-imidazol-4-yl)-2-methoxyethenyl]phenyl}ethanesulfonamide, Maleate

A solution of Example 46C (0.13 g, 0.32 mmol) in tetrahydrofuran (10 mL) was treated with 1M HCl (15 mL), heated to 50° C. for 16 hours, cooled to ambient temperature, neutralized with sodium bicarbonate solution and extracted with ethyl acetate (2×). The combined ethyl acetate extracts were washed with brine, dried (Na₂SO₄) and concentrated. Purification of the residue on silica gel with 10% methanol/dichloromethane provided the desired product, which was converted to the maleic acid salt.

mp 146-148° C.;

¹H NMR (DMSO-d₆) δ 1.20 (t, 3H), 3.10 (q, 2H), 3.88 (s, 3H), 6.05 (s, 2H), 6.81 (s, 1H), 7.06 (d, 1H), 7.11 (s, 1H), 7.15 (d, 1H), 7.34 (dd, 1H), 7.42 (s, 1H), 8.81 (s, 1H), 9.81 (s, 1H);

MS (APCI+) m/z 308 (M+H)⁺;

MS (APCI−) m/z 306 (M−H)⁻;

Anal. calcd for C₁₄H₁₇N₃O₃S.C₄H₄O₄: C, 51.06; H, 5.00; N, 9.92. Found: C, 51.03; H, 5.05; N, 9.79.

### EXAMPLE 47

### N-[5-(1H-imidazol-4-yl)-7,8-dihydro-1-naphthalenyl]methanesulfonamide, Maleate

Example 15B was processed as in Example 8B except that after addition of the 2M HCl, the mixture was heated to reflux for 6 hours. Purification of the residue on silica gel with 10% ethanol/ammonia saturated dichloromethane provided the desired product, which was converted to the maleic acid salt.

mp 161-165° C.;

¹H NMR (DMSO-d₆) δ 2.28-2.38 (m, 2H), 2.85 (t, 2H), 2.98 (s, 3H), 6.07 (s, 2H), 6.49 (t, 1H), 7.11 (dd, 1H), 7.19-7.29 (m, 2H), 7.61 (s, 1H), 8.78 (s, 1H), 9.21 (s, 1H); MS (DCI/NH₃) m/z 290 (M+H)⁺, 307 (M+NH₄)⁺;

Anal. calcd for C₁₄H₁₅N₃O₂S C₄H₄O₄: C, 53.33; H, 4.72; N, 10.36. Found: C, 53.28; H, 4.83; N, 10.20.

### EXAMPLE 55

### N-[3-(1-hydroxy-1-(1H-imidazol-4-yl)propyl)phenyl]ethanesulfonamide

### Example 55A

### 4-[hydroxy(3-nitrophenyl)methyl]-N,N-dimethyl-1H-imidazole-1-sulfonamide

3-Nitrobenzaldehyde was substituted for 6-methoxy-5-nitro-1-tetralone and processed as described in Example 1A to provide the desired product.

MS (DCI/NH₃) m/z 327 (M+H)⁺.

### Example 55B

### N,N-dimethyl-4-(3-nitrobenzoyl)-1H-imidazole-1-sulfonamide

A mixture of Example 55A (9.78 g, 30 mmol) and barium manganate (40 g, 150 mmol) in toluene (200 mL) was refluxed for 30 minutes. The solid was filtered off and washed with dioxane (500 mL). The filtrate and washings were combined and were concentrated under reduced pressure to provide 9.7 g (84%) of the title compound.

¹H NMR (300 MHz, DMSO-d₆) δ 2.92 (s, 6H), 7.87 (t, J=9 Hz, 1H), 8.50 (m, 3H), 8.59 (m, 1H), 9.08 (m, 1H);

MS (APCI+) m/z 325 (M+H)⁺; MS (APCI−) m/z 359 (M+Cl)⁻.

### Example 55C

### 4-(3-aminobenzoyl)-N,N-dimethyl-1H-imidazole-1-sulfonamide

To a mixture of Example 55B (3.24 g, 10 mmol) and NH₄Cl (540 mg, 10 mmol) in water (15 mL) and ethanol (35 mL) was added iron powder (3.92 g, 70 mmol) and the mixture was refluxed for 1 hour. The mixture was filtered, the solid was washed with THF, and the combined filtrate and washings were removed under vacuum to provide 3 g (˜100%) of the title compound.

### Example 55D

### 4-{3-[(ethylsulfonyl)amino]benzoyl}-N,N-dimethyl-1H-imidazole-1-sulfonamide

A solution of Example 55C in pyridine (30 mL) was treated with ethanesulfonyl chloride (0.11 mL, 11 mmol) at 0° C. The mixture was stirred at room temperature for the next 16 hours and then concentrated under vacuum. The residue was purified by column chromatography (silica gel, ethyl acetate)to provide 2.31 g (57%) of the title compound.

MS (APCI+) m/z 387 (M+H)⁺; MS (APCI−) m/z 385 (M−H)⁻, m/z 421 (M+Cl)⁻.

### Example 55E

### N-[3-(1H-imidazol-4-ylcarbonyl)phenyl]ethanesulfonamide

Example 55D (193 mg, 0.5 mmol) in dioxane (5 mL), methanol (5 mL), and water (5 mL) was treated with 1N HCl (5 mL) and the resulting mixture was refluxed for 35 minutes. The mixture was concentrated under vacuum and the residue was passed through Dowex® 50x8-400 ion exchange resin and eluted with 5% NH₄OH. The ammonia solution was concentrated under vacuum and the residue was purified on column (silica gel, 4:1 CH₂Cl₂-methanol) to provide 85 mg (60%) of the title compound.

MS (APCI+) m/z 280 (M+H)⁺; MS (APCI−) m/z 278 (M−H)⁻, m/z 314 (M+Cl)⁻.

### Example 55F

### N-[3-(1-hydroxy-1-(1H-imidazol-4-yl)propyl)phenyl]ethanesulfonamide

To a solution of Example 55E (84 mg, 0.3 mmol) in THF (10 mL) at 0° C. was added dropwise a 2M solution of ethyl magnesium bromide in ether (0.6 mL, 1.2 mmol) and the resulting mixture was allowed to warm to room temperature for 6 hours. The mixture was quenched with saturated NH₄Cl and concentrated under vacuum. The residue was passed through a Dowex® 50x8-400 ion exchange resin with 5% NH₄OH as eluent. The ammonia solution was concentrated under vacuum and purified again by chromatography (silica gel, 9:1 CH₂Cl₂:ethanol) to provide 20 mg of the desired product.

mp 120-124° C.;

¹H NMR (300 MHz, DMSO-d₆) 80.71 (t, J=7 Hz, 3H), 1.15 (t, J=7 Hz, 3H), 2.12 (m, 2H), 3.04 (q, J=7 Hz, 2H), 5.67 (bs, 1H), 7.09 (m, 3H), 7.22 (m, 2H), 7.38 (m, 1H), 8.20 (bs, 1H), 9.71 (bs, 1H);

MS (APCI+) m/z 310 (M+H)⁺; MS (APCI−) m/z 308 (M−H)⁻, m/z 344 (M+Cl)⁻.

### EXAMPLE 56

### N-[3-(cyclohexylidene-(1H-imidazol-4-ylmethyl)phenyl]ethanesulfonamide

### Example 56A

### 4-(cyclohexyl {3-[(ethylsulfonyl)amino]phenyl}hydroxymethyl)-N,N-dimethyl-1H-imidazole-1-sulfonamide

To a solution of Example 55D (154 mg, 0.4 mmol) in THF (10 mL) at 0° C. was added 1M solution in Et₂O of cyclohexylmagnesium chloride (1 mL, 1 mmol) and the mixture was left at room temperature for 6 hours. The mixture was quenched with saturated NH₄Cl and concentrated under vacuum. The residue was extracted with ethyl acetate, dried (MgSO₄) and concentrated under vacuum. Column chromatography (silica gel, 3:5 hexanes:ethyl acetate) provided 160 mg (68%) of alcohol.

MS (APCI+) m/z 471 (M+H)⁺; MS (APCI−) m/z 469 (M−H)⁻, m/z 505 (M+Cl)⁻.

### Example 56B

### N-{3-[cyclohexyl(hydroxy) 1H-imidazol-4-ylmethyl]phenyl}ethanesulfonamide

Example 56A was dissolved in dioxane (10 mL) and treated with 2% KOH (2 mL) at reflux for 48 hours. The mixture was concentrated under vacuum and the residue was chromatographed (silica gel, 9:1 CH₂Cl₂:ethanol and a few drops of concentrated NH₄OH) to provide 90 mg (62%) of the title compound.

MS (APCI+) m/z 364 (M+H)⁺; MS (APCI−) m/z 362 (M−H)⁻, m/z 398 (M+Cl)⁻.

### Example 56C

### N-[3-(cyclohexylidene-(1H-imidazol-4-ylmethyl)phenyl]ethanesulfonamide

Example 56B was first acetylated with acetic anhydride (2 mL) in pyridine (5 mL) at 0° C. for 6 hours. The mixture was concentrated under vacuum and then immediately treated with 1N HCl (10 mL) at reflux for 15 hours. The mixture was concentrated under vacuum, and the residue was treated with 5% NH₄OH and concentrated under vacuum. The residue was purified by column chromatography (silica gel, 9:1 CH₂Cl₂:methanol) to provide 20 mg (24%) of the desired product.

mp 75-78° C.;

¹H NMR (300 MHz, DMSO-d₆) δ 1.16 (t, J=7 Hz, 3H), 1.55 (m, 6H), 2.06 (m, 2H), 2.55 (m, 2H), 3.03 (q, J=7 Hz, 2H), 6.61 (s, 1H), 6.80 (m, 1H), 6.98 (m, 1H), 7.07 (m, 1H), 7.24 (t, J=9 Hz, 1H), 7.55 (m, 1H), 9.72 (s, 1H);

MS (APCI+) m/z 346 (M+H)⁺; MS (APCI−) m/z 344 (M−H)⁻, m/z 380 (M+Cl)⁻.

### EXAMPLE 61

### N-[5-(H-imidazol-5-yl)-5,6,7,8-tetrahydro-1-nqphthalepyl]-3,5-dimethyl-4-isoxazolesulfonamide

### Example 61A

### tert-butyl 4-(5-1{[(3,5-dimethyl-4-isoxazolyl)sulfonyl]amino}-1,2,3,4-tetrahydro-1-naphthalenyl)-1H-imidazole-1-carboxylate

To a manually agitated 23° C. solution of Example 12C (100 mg, 0.32 mmol) in methylene chloride (5 mL) was added pyridine (0.078 mL, 0.96 mmol) and 3,5-dimethylisoxazole-4-sulfonyl chloride (65.4 mg, 0.34 mmol), and the homogeneous reaction mixture allowed to stand for 10 minutes. The methylene chloride was removed under vacuum. The resultant thick oil was allowed to stand and additional 2 hours and was then chromatographed on flash silica gel (1:1 ethyl acetate/hexanes) to provide the title compound (150 mg, 0.318 mmol, >99% yield).

### Example 61B

### N-[5-(1H-imidazol-4-yl)-5,6,7,8-tetrahydro-1-naphthalenyl]-3,5-dimethyl-4-isoxazolesulfonamide

A 0° C. solution of Example 61A (150 mg, 0.318 mmol) in methylene chloride (10 mL) was treated with trifluoroacetic acid (3.2 mL) and stirred for 1.5 hours. The reaction mixture was warmed to room temperature for 2 hours and then cooled to −20° C. for 16 hours. The reaction mixture was warmed to ambient temperature and diluted with methylene chloride and water and neutralized with aqueous saturated NaHCO₃. The methylene chloride layer was separated and the aqueous phase extracted twice more with methylene chloride. The combined extracts were dried (MgSO₄), filtered, and concentrated under vacuum. The residue was chromatographed on flash silica gel (79:20:1 methylene chloride/methanol/ammonium hydroxide) to provide the title compound (87 mg, 0.23 mmol, 74% yield).

mp 85-2100C;

¹H NMR (300 MHz, CD₃OD) δ 1.67 (m, 2H), 1.98 (m, 2H), 2.17 (s, 3H), 2.29 (m, 3H), 6.62 (m, 2H), 4.12 (dd, J=6.9, 6.9 Hz, 1H), 6.52 (bs, 1H), 7.01 (m, 3H), 7.61 (d, J=1.2 Hz, 1H);

MS (APCI+) m/z 373 (M+H)⁺.

### EXAMPLE 63

### N-[5-(1H-imidazol-5-yl)-5,6,7,8-tetrahydro-1-naphthalenyl]-1-propanesulfonamide

To a solution of 1-propanesulfonyl chloride (20.5 mg, 0.14 mmol) in dichloromethane (250 mL) was added pyridine (78 mL, 0.96 mmol) followed 5-(1H-imidazol-4-yl)-5,6,7,8-tetrahydro-1-naphthalenamine (30 mg, 0.096 mmol) dissolved in CH₂Cl₂ (1 mL). The CH₂Cl₂ was removed under vacuum and the reaction gently shaken at ambient temperature overnight. To the reaction was added 1.0 mL of CH₂Cl₂ followed by 200 mg of polymer supported trisamine (Argonaut laboratories). The reaction was shaken at room temperature for 30 minutes, the filtrate collected and the volume brought to 5 mL with dichloromethane. The organic layer was extracted with 10% aqueous citric acid (3×4 mL), brine (2×4 mL), filtered (Varian CE1000M)® and the solvent removed under vacuum. The resulting oil was dissolved in 2 mL of acetonitrile and 0.5 g of Amberlyst resin was added. The reaction was shaken at room temperature for 72 hours and filtered. The resin was washed with acetonitrile (2×2 mL), methanol (2×2 mL), and suspended in 2 M methanolic ammonia (2 mL) for 2 hours. The resin was filtered, washed with 0.5 mL of methanol and then retreated with ammonia as described. The ammonia and methanol filtrates were combined and the solvent removed under vacuum. The crude material was purified using reverse phase preparative HPLC. (6.7 mg, 21.9% yield).

¹H NMR (500 MHz, DMSO-d₆) δ 0.99 (t, J=7.5 Hz, 3H), 1.67 (m, 1H), 1.74 (m, 3H), 1.88 (m, 1H), 2.02 (m, 1H), 2.74 (m, 1H), 2.79 (m, 1H), 3.06 (t, J=7.7 Hz, 2H), 4.00 and 4.12 (2 m, 2.4:1, 1H), 6.44 and 6.54 (2 bs, 1:2.4, 1H), 6.75 and 6.91 (2 bd, 1:2.4, J=7.7, 1H), 7.02 (m, 1H), 7.10 (m, 1H), 7.49 and 7.51 (2 bs, 1:2.4, 1H), 8.85 (bs, 1H), 11.70 and 11.84 (2 bs, 2.4: 1, 1H);

MS (APCI−) m/z 319 (M−H)⁻.

### EXAMPLE 64

### N-[5-(1H-imidazol-5-yl)-5,6,7,8-tetrahydro-1-naphthalenyl]-1-butanesulfonamide:

The desired product was prepared according to the method of Example 63above substituting 1-butanesulfonyl chloride for 1-propanesulfonyl chloride (7.5 mg, 23.5% yield).

¹H NMR (500 MHz, DMSO-d₆) δ 0.88 (t, J=7.4 Hz, 3H), 1.40 (m, 2H), 1.69 (m, 3H), 1.76 (m, 1H), 1.89 (m, 1H), 2.02 (m, 1H), 2.74 (m, 1H), 2.79 (m, 2H), 3.08 (t, J=7.7 Hz, 2H), 4.00 and 4.13 (2 m, 2:1, 1H), 6.43 and 6.53 (2 bs, 1:2, 1H), 6.76 and 6.92 (2 bd, 1:2, J=7.7, 1H), 7.03 (m, 1H), 7.10 (m, 1H), 7.49 and 7.51 (2 bs, 1:2, 1H), 8.85 (bs, 1H), 11.70 and 11.85 (2 bs, 2:1, 1H);

MS (APCI−) m/z 333 (M−H)⁻.

### EXAMPLE 65

### 3-Chloro-N-[5-(1H-imidazol-5-yl)-5,6,7,8-tetrahydro-1-naphthalenyl]-1-propanesulfonamide

The desired product was prepared according to the method of Example 63 above substituting 2-chloropropanesulfonyl chloride for 1-propanesulfonyl chloride (7.4 mg, 21.8% yield).

¹H NMR (500 MHz, DMSO-d₆) δ 1.68 (m, 1H), 1.77 (m, 1H), 1.89 (m, 1H), 2.00 (m, 1H), 2.18(q, J=6.8 Hz, 2H), 2.80 (m, 2H), 3.25 (m, 2H), 3.77 (t, J=5.0 Hz, 2H), 4.05 (m, 1H), 6.51 (m, 1H), 6.91 (m, 1H), 7.05 (t, J=7.0 Hz, 1H), 7.10 (d, J=7.0 Hz, 1H), 7.51 (d, J=1.9 Hz, 1H), 9.02 (s, 1H), 11.72 and 11.91 (2 bs, 1:2, 1H);

MS (APCI−) m/z 705 (2M−H)⁻.

### EXAMPLE 66

### N-[5-(1H-imidazol-5-yl)-5,6,7,8-tetrahydro-1-naphthalenyl]-1-methyl-1H-imidazole-4-sulfonamide

The desired product was prepared according to the method of Example 63 above substituting 1-methyl-1H-imidazole-4-sulphonyl chloride for 1-propanesulfonyl chloride (5.0 mg, 14.6% yield).

¹H NMR (500 MHz, DMSO-d₆) δ 1.56 (m, 1H), 1.64 (m, 1H), 1.80 (m, 1H), 1.97 (m, 1H), 2.63 (m, 1H), 2.67 (m, 1H), 3.65 (s, 3H), 3.97 (m, 1H), 6.34 and 6.43 (bs, 1:1, 1H), 6.46 (s, 1H), 6.9 (m, 2H), 7.49 (m, 1H), 7.57 (s, 1H), 7.77 (s, 1H), 9.15 (bs, 1H), 11.67 and 11.82(2bs, 1:1, 1H);

MS (APCI−) m/z 357 (M−H)⁻.

### EXAMPLE 67

### N-[5-(1H-imidazol-5-yl)-5,6,7,8-tetrahydro-1-naphthalenyl](phenyl)methanesulfonamide

The desired product was prepared according to the method of Example 63 above substituting phenylmethanesulfonyl chloride for 1-propanesulfonyl chloride (6.4 mg, 18.2% yield).

¹H NMR (500 MHz, DMSO-d₆) δ 1.64 (m, 1H), 1.73 (m, 1H), 1.88 (m, 1H), 2.02 (m, 1H), 2.63 (m, 1H), 2.67 (m, 1H), 4.00 and 4.13 (m, 2:1,1H), 4.43 (s, 2H), 6.45 and 6.54 (2 bs, 1:2, 1H), 6.83 (m, 1H), 7.02 (m, 1H), 7.10 (m, 1H), 7.35 (s, 5H), 7.49 and 7.52 (2 bs, 1:2, 1H), 8.85 (bs, 1H), 11.70 and 11.83 (2 bs, 2:1, 1H); MS (APCI−) m/z 367 (M−H)⁻.

### EXAMPLE 68

### N-[5-(1H-imidazol-5-yl)-5,6,7,8-tetrahydro-1-naphthalenyl]-4-methylbenzenesulfonamide

The desired product was prepared according to the method of Example 63 above substituting p-toluenesulfonyl chloride for 1-propanesulfonyl chloride (10.9 mg, 31.0% yield).

¹H NMR (500 MHz, DMSO-d₆) δ 1.50 (m, 2H), 1.78 (m, 1H), 1.93 (m, 1H), 2.36 (s, 3H), 2.41 (m, 1H), 2.46 (m, 1H), 4.00 (m, 1H), 6.33 and 6.42 (2 bs, 1:2, 1H), 6.77 (m, 1H), 6.86 (m, 1H), 6.92 (m, 1H), 7.33 (d, J=8.1 Hz, 2H), 7.48 (m, 1H), 7.54 (d, J=8.0 Hz, 2H), 9.31 (bs, 1H), 11.68 and 11.80 (2 bs, 2:1, 1H);

MS (APCI−) m/z 367 (M−H)⁻.

### EXAMPLE 69

### N-[5-(1H-imidazol-5-yl)-5,6,7,8-tetrahydro-1-naphthalenyl]-2-methylbenzenesulfonamide

The desired product was prepared according to the method of Example 63 above substituting o-toluenesulfonyl chloride for 1-propanesulfonyl chloride (10.8 mg, 30.7% yield).

¹H NMR (500 MHz, DMSO-d₆) δ 1.59 (m, 1H), 1.65 (m, 1H), 1.86 (m, 1H), 2.01 (m, 1H), 2.62 (s, 3H), 4.15 (m, 1H), 6.49(bs, 1H), 6.79 and 6.87 (m, 2:1, 1H), 6.99 (m, 2H), 7.5 (m, 5H), 7.77 (d, J=5.6 Hz, 1H), 9.47 (bs, 1H), 11.75 and 11.80 (2 bs, 2:1, 1H); MS (APCI−) m/z 367 (M−H)⁻.

### EXAMPLE 70

### N-[5-(1H-imidazol-5-yl)-5,6,7,8-tetrahydro-1-naphthalenyl]-2-phenyl-1-ethenesulfonamide

The desired product was prepared according to the method of Example 63 above substituting (E)-2-phenylethenesulfonyl chloride for 1-propanesulfonyl chloride (12.2 mg, 33.6% yield).

¹H NMR (500 MHz, DMSO-d₆) δ 1.63 (m, 1H), 1.70 (m, 1H), 1.84 (m, 1H), 1.95 (m, 1H), 2.80 (m, 2H), 4.00 (bs, 1H), 6.45 (bs, 1H), 6.89 (bs, 1H), 7.01 (t, J=7.5 Hz, 1H), 7.08 (m, 1H), 7.24 (d, J=15.3 Hz, 1H), 7.30 (d, J=15.4 Hz, 1H), 7.42 (m, 3H), 7.49 (bs, 1H), 7.68 (m, 2H), 9.15 (bs, 1H); 11.67 and 11.82 (2 bs, 2:1, 1H);

MS (APCI−) m/z 379 (M−H)⁻.

### EXAMPLE 71

### N-[5-(1H-imidazol-5-yl)-5,6,7,8-tetrahydro-1-naphthalenyl]-4-methoxybenzenesulfonamide

The desired product was prepared according to the method of Example 63 above substituting 4-methoxybenzenesulfonyl chloride for 1-propanesulfonyl chloride (3.0 mg, 8.2% yield).

¹H NMR (500 MHz, DMSO-d₆) δ 1.50 (m, 2H), 1.78 (m, 1H), 1.94 (m, 1H), 2.44 (m, 2H), 3.80 (s, 3H), 4.00 (m, 1H), 6.32 and 6.42 (2 bs, 1:2, 1H), 6.79 (m, 1H), 6.87 (m, 1H), 6.93 (m, 1H), 7.05 (d, J=8.8 Hz, 2H), 7.49 (m, 1H), 7.58 (d, J=8.8 Hz, 2H), 9.24 (bs, 1H), 11.67 and 11.80 (2 bs, 2:1, 1H);

MS (APCI−) m/z 383 (M−H)⁻.

### EXAMPLE 72

### 5-Chloro-N-[5-(1H-imidazol-5-yl)-5,6,7,8-tetrahydro-1-naphthalenyl]-2-thiophenesulfonamide

The desired product was prepared according to the method of Example 63 above substituting 5-chlorothiophene-2-sulfonyl chloride for 1-propanesulfonyl chloride (2.8 mg, 7.4% yield).

¹H NMR (500 MHz, DMSO-d₆) δ 1.54 (m, 1H), 1.60 (m, 1H), 1.82 (m, 1H), 1.93 (m, 1H), 2.50 (m, 2H), 4.00 (m, 1H), 6.43 (s, 1H), 6.89 (m, 2H), 7.02 (t, J=7.9 Hz, 1H), 7.20 (d, J=4.0 Hz, 1H), 7.30 (d, J=4.0 Hz, 1H), 7.51 (d, J=1.1 Hz, 1H), 9.86 (bs, 1H), 11.70 (bs, 1H);

MS (APCI−) m/z 393 (M−H)⁻.

### EXAMPLE 73

### N-[5-(1H-imidazol-5-yl)-5,6,7,8-tetrahydro-1-naphthalenyl]-8-quinolinesulfonamide

The desired product was prepared according to the method of Example 63 above substituting 8-quinolinesulfonyl chloride for 1-propanesulfonyl chloride (4.0 mg, 10.3% yield).

¹H NMR (500 MHz, DMSO-d₆) δ 1.49 (m, 1H), 1.58 (m, 1H), 1.78 (m, 1H), 1.91 (m, 1H), 2.58 (m, 1H), 2.65 (m, 1H), 3.89 and 4.02 (m, 2:1, 1H), 6.32 and 6.42 (m, 1:2, 1H), 6.56 (m, 1H), 6.63 (m, 1H), 6.78 (m, 2H), 7.47 (s, 1H), 7.72 (t, J=6.4 Hz, 1H), 7.76 (dd, J=3.2, 6.8 Hz, 1H), 8.25 (dd, J=1.2, 6.0 Hz, 1H), 8.31 (d, J=6.4 Hz, 1H), 8.58 (dd, J=1.6, 6.8 Hz, 1H), 9.2 (bs, 1H), 9.13 (dd, J=1.2, 3.2 Hz, 1H), 11.66 and 11.80 (2 bs, 2:1, 1H);

MS (APCI−) m/z 404 (M−H)⁻.

### EXAMPLE 74

### 5-Chloro-N-[5-(1H-imidazol-5-yl)-5,6,7,8-tetrahydro-1-naphthalenyl]-1,3-dimethyl-1H-pyrazole-4-sulfonamide

The desired product was prepared according to the method of Example 63 above substituting 5-chloro-1,3-dimethyl-4-pyrazolosulfonyl chloride for 1-propanesulfonyl chloride (9.6 mg, 24.7% yield).

¹H NMR (500 MHz, DMSO-d₆) δ 1.55 (m, 2H), 1.82 (m, 1H), 1.98 (m, 1H), 2.08 (s, 3H), 2.52 (m, 2H), 3.71 (s, 3H), 3.97 and 4.08 (m, 2:1, 1H), 6.28 and 6.39 (m, 1:2, 1H), 6.97 (m, 3H), 7.50 (s, 1H), 9.45 (m, 1H), 11.69 and 11.84 (bs, 1:1, 1H);

MS (APCI−) m/z 405 (M−H)⁻.

### EXAMPLE 75

### Methyl 2-({[5-(1H-imidazol-5-yl)-5,6,7,8-tetrahydro-1-naphthalenyl]amino}sulfonyl)-3-thiophenecarboxylate

The desired product was prepared according to the method of Example 63 above substituting 2-methoxycarbonyl-3-thiophenesulfonyl chloride for 1-propanesulfonyl chloride (3.6 mg, 9.0% yield).

¹H NMR (500 MHz, DMSO-d₆) δ 1.46 (m, 1H), 1.54 (m, 1H), 1.69 (m, 1H), 1.80 (m, 1H), 2.48 (m, 2H), 3.70 (s, 3H), 3.86 (m, 1H), 6.32 (bs, 1H), 6.66 (d, J=8.1 Hz, 1H), 6.72 (m, 1H), 6.81 (t, J=7.7 Hz, 1H), 7.19 (dd, J=5.1 Hz, J=0.8 Hz, 1H), 7.36 (s, 1H), 7.87 (d, J=5.1 Hz, 1H), 8.89 (bs, 1H), 11.58 (bs, 1H); MS (APCI−) m/z 417 (M−H)⁻.

### EXAMPLE 76

### N-[5-({[5-(1H-imidazol-5-yl)-5,6,7,8-tetrahydro-1-naphthalenyl]amino}sulfonyl)-4-methyl-1,3-thiazol-2-yl]acetamide

The desired product was prepared according to the method of Example 63 above substituting 2-acetamido-4-methyl-5-thiazolesulfonyl chloride for l-propanesulfonyl chloride (6.3 mg, 15.3% yield).

¹H NMR (500 MHz, DMSO-d₆) δ 1.54 (m, 1H), 1.58 (m, 1H), 1.81 (m, 1H), 1.90 (s, 3H), 1.93 (m, 1H), 2.13 (s, 3H), 2.15 (s, 3H), 2.56 (m, 2H), 4.00 (m, 1H), 6.38 (bs, 1H), 6.82 (bs, 1H), 6.87 (d, J=6.0 Hz, 1H), 6.96 (t, J=6.0 Hz, 1H), 7.49 (d, J=1.0 Hz, 1H), 10.3 (bs, 1H), 11.7 (bs, 1H);

MS (APCI−) m/z 431 (M−H)⁻.

### EXAMPLE 77

### 5-Chloro-N-[5-(1H-imidazol-5-yl)-5,6,7,8-tetrahydro-1-naphthalenyl]-3-methyl-2,3-dihydro-1-benzothiophene-2-sulfonamide

The desired product was prepared according to the method of Example 63 above substituting 5-chloro-3-methylbenzo[2,3-b]thiopene-2-sulphonyl chloride for 1-propanesulfonyl chloride (5.8 mg, 13.2% yield).

¹H NMR (500 MHz, DMSO-d₆) δ 1.36 (m, 1H), 1.42 (m, 1H), 1.73 (m, 1H), 1.85 (m, 1H), 2.29 (s, 3H), 2.41 (m, 1H), 2.55 (m, 1H), 3.97 (m, 1H), 6.41 (bs, 1H), 6.87 (m, 2H), 6.96 (m, 1H), 7.49 (d, J=0.8 Hz, 1H), 7.55 (dd, J=0.8, 6.8 Hz, 1H), 7.96 (s, 1H), 8.06 (d, J=6.8 Hz, 1H), 9.9 (bs 1H), 11.7 (bs, 1H);

MS (APCI−) m/z 379 (M−H)⁻.

### EXAMPLE 78

### 2,2,2-trifluoro-N-[3-(1H-imidazol-4-ylmethyl)phenyl]ethanesulfonamide, Maleate

Example 21C was processed as in Example 21D but substituting 2,2,2-trifluoroethanesulfonyl chloride for methanesulfonyl chloride to provide the title compound, which was converted to the maleic acid salt.

mp 161-162° C.;

¹H NMR (DMSO-d₆) δ 4.00 (s, 2H), 4.51 (q, 2H), 6.05 (s, 2H), 7.03 (d, 1H), 7.07-7.13 (m, 2H), 7.28-7.34 (m, 1H), 7.36 (d, 1H), 8.81 (d, 1H), 10.46 (bs, 1H), 14.10 (bs, 1H);

MS (DCI/NH₃) m/z 320 (M+H)⁺; Anal. Calcd for C₁₂H₁₂N₃O₂SF₃C₄H₄O₄: C, 44.14; H, 3.70; N, 9.65. Found: C, 44.18; H, 3.72; N, 9.59.

### EXAMPLE 79

### N-[4-(1H-imidazol-4-yl)-3,4-dihydro-2H-chromen-8-yl]ethanesulfonamide

Example 19C was processed as in Example 12D to provide the title compound.

¹H NMR (DMSO-d₆) δ 1.25 (t, 3H), 2.05-2.30 (m, 2H), 3.01 (q, 2H), 4.06 (t, 1H), 4.22 (m, 2H), 6.69 (s, 1H), 6.74 (t, 1H), 6.89 (d, 1H), 7.08 (d, 1H), 7.56 (s, 1H), 8.75 (s, 1H);

MS (APCI+) m/z 308 (M+H)⁺;

Anal. Calcd for C₁₄H₁₇N₃O₃S: C, 54.71; H, 5.57; N, 13.67. Found: C, 54.43; H, 5.63; N, 13.54.

### EXAMPLE 80

### N-[6-fluoro-4-(1H-imidazol-4-yl)-3,4-dihydro-2H-chromen-8-yl]ethanesulfonamide, Maleate

### Example 80A

### 6-fluoro-8-nitro-2,3-dihydro-4H-chromen-4-one

Concentrated sulfuric acid (5 mL) was cooled to −15° C., treated with 6-fluoro-2,3-dihydro-4H-chromen-4-one (1.0 g, 6.0 mmol), treated with a mixture of 70% nitric acid (1.8 mL) and concentrated sulfuric acid (2.8 mL), stirred at 0° C. for 2 hours and poured into water. The resulting solid was collected by filtration, washed with water and dried under vacuum. Purification of the residue on silica gel eluting with 1:1 ethyl acetate:hexanes provided the title compound.

MS (APCI−) 210 (M−H)⁻.

### Example 80B

### 4-(6-fluoro-4-hydroxy-8-nitro-3,4-dihydro-2H-chromen-4-yl)-N,N-dimethyl-1H-imidazole-1-sulfonamide

Example 80A was processed as in Example 1A to provide the title compound.

### Example 80C

### 4-(6-fluoro-8-nitro-2H-chromen-4-yl)-N,N-dimethyl-1H-imidazole-1-sulfonamide

Example 80B was processed as in Example 31B to provide the title compound. MS (APCI+) m/z 369 (M+H);

### Example 80D

### 4-(8-amino-6-fluoro-3,4-dihydro-2H-chromen-4-yl)-N,N-dimethyl-1H-imidazole-1-sulfonamide

Example 80C was processed as in Example 1C but substituting ethyl acetate for methanol as the solvent to provide the title compound.

### Example 80E

### N-[6-fluoro-4-(1H-imidazol-4-yl)-3,4-dihydro-2H-chromen-8-yl]ethanesulfonamide, Maleate

Example 80D was processed as in Example 3 ID but substituting ethanesulfonyl chloride for methanesulfonyl chloride to provide the title compound, which was converted to the maleic acid salt.

¹H NMR (DMSO-d₆) δ 1.25 (t, 3H), 2.19 (m, 2H), 3.12 (q, 2H), 4.22 (m, 2H), 4.35 (t, 1H), 6.06 (s, 2H), 6.62 (dd, 1H), 7.01 (dd, 1H), 7.27 (s, 1H), 8.69 (s, 1H), 9.12 (s, 1H); MS (APCI+) m/z 308 (M+H)⁺;

Anal. Calcd for C₁₄H₁₆N₃O₃SFC₄H₄O₄: C, 48.98; H, 4.57; N, 9.52. Found: C, 49.25; H, 4.73; N, 9.33.

### EXAMPLE 81

### N-{3-[(E)-1-(1H-imidazol-4-yl)-2-methoxyethenyl]phenyl}ethanesulfonamide

### Example 81A

### 4-[(E)-1-(3-aminophenyl)-2-methoxyethenyl]-N,N-dimethyl-1H-imidazole-1-sulfonamide

The more polar product from Example 46A was processed as described in Example 46B except that the product was purified on silica gel eluting with 9:1 hexanes:ethyl acetate to provide the title compound.

MS (APCI+) m/z 323 (M+H)⁺;

### Example 81B

### 4-((E)-1-{3-[(ethylsulfonyl)amino]phenyl}-2-methoxyethenyl)-N,N-dimethyl-1H-imidazole-1-sulfonamide

The product from Example 81A was processed as described in Example 46C except that the residue was kept at room temperature for 77 days during which time a portion of the title compound decomposed to the unprotected imidazole. Purification on silica gel eluting with ethyl acetate provided the title compound as the less polar product as well as a more polar product, which contained the unprotected imidazole.

MS (APCI+) m/z 415 (M+H)⁺;

### Example 81C

### N-{3-[(E)-1-(1H-imidazol-4-yl)-2-methoxyethenyl]phenyl}ethanesulfonamide

The more polar product from Example 81B was purified again on silica gel eluting with 10% ethanol/ammonia-saturated dichloromethane to provide the title compound.

¹H NMR (DMSO-d₆) δ 1.19 (t, 3H), 3.05 (q, 2H), 3.67 (s, 3H), 6.65 (d, 1H), 6.85 (bs, 1H), 7.07 (m, 2H), 7.22-7.30 (m, 2H), 7.58 (s, 1H), 9.68 (s, 1H), 11.91 (bs, 1H);

MS (APCI+) m/z 308 (M+H)⁺;

Anal. calcd for C₁₄H₁₇N₃O₃S0.5H₂O: C, 53.15; H, 5.73; N. 13.28. Found: C, 53.25; H, 5.49; N, 13.28.

### EXAMPLE 82

### N-[3-(1H-imidazol-4-ylmethyl)-2-methoxyphenyl]ethanesulfonamide

### Example 82A

### 2-methoxy-3-nitrobenzaldehyde

2-Hydroxy-3-nitrobenzaldehyde (5 g, 30 mmol) in dimethylformamide (10 mL) was treated with potassium carbonate (116.5 g, 120 mmol), and iodomethane (10 mL). After stirring for 16 hours with a mechanical stirrer, the mixture was treated with a second portion of iodomethane (10 mL) and heated for 1 hour at 50° C. A third portion of jodomethane (10 mL) was added to the mixture and heating continued at 500° C. for 1 hour. The mixture was allowed to cool ambient temperature, diluted with diethyl ether (500 mL), washed with water (2×, 500 mL), washed with brine, dried (MgSO₄), filtered, and concentrated to provide 4.8 g of the title compound.

### Example 82B

### 4-[hydroxy(2-methoxy-3-nitrophenyl)methyl-N,N-dimethyl-1H-imidazole-1-sulfonamide

The product from Example 82A (4.0 g, 22 mmol) was processed as described in Example 21A to provide the title compound which was not purified but carried onto the next step.

MS (DCI/NH₃) m/z 357 (M+H)⁺.

### Example 82C

### 4-(2-methoxy-3-nitrobenzyl)-N,N-dimethyl-1H-imidazole-1-sulfonamide

The product from Example 82B was processed as described in Example 28C. Purification of the residue on silica gel with 1:1 ethyl acetate:hexane and then 2:1 ethyl acetate:hexane provided the title compound.

MS (DCI/NH₃) m/z 341 (M+H)⁺.

### Example 82D

### 4-(3-amino-2-methoxybenzal)-N,N-dimethyl-1H-imidazole-1-sulfonamide

The product from Example 82C was processed as described in Example 1C. Purification of the residue on silica gel with 1: 1 ethyl acetate:hexane and then 2:1 ethyl acetate:hexane and then ethyl acetate provided the title compound.

¹H NMR (CDCl₃) δ 2.82 (s, 6H), 3.24 (bs, 2H), 3.74 (s, 3H), 3.94 (s, 2H), 6.62 (dd, 1H), 6.66 (dd, 1H), 6.85-6.92 (m, 2H), 7.84 (d, 1H).

### Example 82E

### 4-{3-[(ethylsulfonyl)amino]-2-methoxybenzyl}-N,N-dimethyl-1H-imidazole-1-sulfonamide

The product from Example 82D and ethanesulfonyl chloridewas processed as described in Example 46C to provide the title compound.

MS (DCI/NH₃) m/z 341 (M+H)⁺.

### Example 82F

### N-[3-(1H-imidazol-4-ylmethyl)-2-methoxyphenyl]ethanesulfonamide

The product from Example 82E was processed as described in Example 46D except that after cooling to ambient temperature the mixture was concentrated to dryness and directly purified on silica gel using 2% methanol/ammonia-saturated dichloromethane to provide the title compound.

mp 185-186° C.;

¹H NMR (DMSO-d₆) δ 1.26 (t, 3H), 3.15 (q, 2H), 3.73 (s, 3H), 3.85 (s, 2H), 6.73 (bs, 1H), 6.92-6.96 (m, 1H), 6.99 (t, 1H), 7.20 (dd, 1H), 7.52 (d, 1H), 9.01 (bs, 1H), 11.81 (bs, 1H);

MS (DCI/NH₃) m/z 296 (M+H)^{+,}

Anal. Calcd for C₁₃H₁₇N₃O₃S: C, 52.87; H, 5.80; N, 14.23. Found: C, 52.79; H, 5.91; N, 14.12.

### EXAMPLE 83

### N-[2-hydroxy-3-(1H-imidazol-4-ylmethyl)phenyl]ethanesulfonamide, Maleate

The product from Example 82D was processed as described in Example 2. Prior to chromatography, the residue in tetrahydrofuran (5 mL) was treated with 2M HCl (30 mL) and heated at reflux for 16 hours. The mixture was allowed to cool to ambient temperature and concentrated. The residue was purified on silica gel with 2% and then 5% and then 10% methanol/ammonia-saturated dichloromethane to provide the title compound, which was converted to the maleic acid salt.

mp 155-157° C.;

¹H NMR (DMSO-d₆) δ 1.23 (t, 3H), 3.05 (q, 2H), 3.95 (s, 2H), 6.07 (s, 2H), 6.79 (t, 1H), 6.92 (dd, 1H), 7.17 (dd, 1H), 7.23 (d, 1H), 8.69 (s, 1H), 8.73 (s, 1H), 12.70 (bs, 1H);

MS (DCI/NH₃) m/z 282 (M+H)⁺,

Anal. Calcd for C₁₂HN₅N₃O₃S.C₄H₄O₄: C, 48.36; H, 4.82; N, 10.57. Found: C, 48.55; H, 4.86; N, 10.46.

### EXAMPLE 84

### N-[5-(2-methyl-1H-imidazol-4-yl)-5,6,7,8-tetrahydro-1-naphthalenyl]ethanesulfonamide, Maleate

### Example 84A

### 2-methyl-4-(5-nitro-3,4-dihydro-1-naphthalenyl)-1H-imidazole

4-Iodo-2-methyl-1-triphenylmethylimidazole, prepared as descibed in (Cliff, Matthew D, Synthesis, 7, 1994, 681-682) and 5-nitrotetralone for 8-methoxy-5-nitro-3,4-dihydro-1 (2H)-naphthalenone, from Example 26A, were processed as described in Example 26B to provide the title compound, which was used without purification.

### Example 84B

### tert-butyl 2-methyl-4-(5-nitro-3,4-dihydro-1-naphthalenyl)-1H-imidazole-1-carboxylate

The product from Example 84A was processed as described in Example 26C to provide the title compound.

MS (DCI/NH₃) m/z 356 (M+H)⁺;

### Example 84C

### tert-butyl 4-(5-amino-3,4-dihydro-1-naphthalenyl)-2-methyl-1H-imidazole-1-carboxylate

The product from Example 84B in ethyl acetate was processed as described in Example 1C to provide the title compound.

MS (ESI+) m/z 272 (M+H)⁺;

### Example 84D

### N-[5-(2-methyl-1H-imidazol-4-yl)-5,6,7,8-tetrahydro-1-naphthalenyl]ethanesulfonamide Maleate

The product from Example 84C was processed as described in Example 12D to provide the title compound.

mp 73-77° C.;

¹H NMR (DMSO-d₆) δ 1.28 (t, 3H), 1.66-1.86 (m, 2H), 1.86-2.06 (m, 2H), 2.83 (t, 2H), 3.12 (q, 2H), 4.24 (t, 1H), 6.02 (s, 2H), 6.82 (d, 1H), 7.08 (s, 1H), 7.12 (t, 1H), 7.19 (dd, 1H), 8.99 (s, 1H), 13.60 (bs, 1H);

Anal. Calcd for C₁₆H₂₁N₃O₂S.C₄H₄O₄ 0.25H₂O: C, 54.60; H, 5.84; N, 9.55. Found: C, 54.38; H, 5.83; N, 9.31.

### EXAMPLE 85

### (+) N-{3-[1-(1H-imidazol-4-yl)ethyl]phenyl}methanesulfonamide Hydrochloride

### Example 85A

### 4-[1-(3-nitrophenyl)vinyl]-1H-imidazole

The product from Example 31B (1.6 g, 5.0 mmol) in tetrahydrofuran (5 mL) was treated with 1M HCl and heated at refluxed for 4 hours. The mixture was allowed to cool to ambient temperature, neutralized with solid sodium bicarbonate, and extracted three times with a mixture 9:1 dichloromethane:methanol. The extractions were combined, dried (MgSO₄), filtered, and concentrated to provide the title compound.

### Example 85B

### tert-butyl 4-[1-(3-nitrophenyl)vinyl]-1H-imidazole-1-carboxylate

The product from Example 85A was processed as described in Example 26C to provide the title compound.

### Example 85C

### tert-butyl 4-[1-(3-aminophenyl)ethyl]-1H-imidazole-1-carboxylate

The product from Example 85B in ethyl acetate was processed as described in Example 1C to provide the title compound.

MS (DCI/NH₃) m/z 288 (M+H)⁺.

### Example 85D

### tert-butyl 4-(1-{3-[(methylsulfonyl)amino]phenyl}ethyl)-1H-imidazole-1-carboxylate

The product from Example 85C and methanesulfonyl chloride were processed as described in Example 33A to provide the title compound.

MS (DCI/NH₃) m/z 366 (M+H)⁺.

### Example 85E

### (+) N-{3-[1-(1H-imidazol-4-yl)ethyl]phenyl}methanesulfonamide Hydrochloride

The enantiomers of Example 85D were separated by chiral chromatography on a Chiracel OJ column using 85:15 hexane:ethanol as the mobile phase. The fractions containing the faster moving enantiomer were concentrated and the residue processed as described in Example 33C to provide the title compound, which was converted to the hydrochloride salt.

mp 195-196° C.;

[α]²³_{D}+32.60 (c 1.0, methanol);

¹H NMR (DMSO-d₆) δ 1.57 (d, 3H), 2.99 (s, 3H), 4.24 (q, 1H), 7.00 (d, 1H), 7.05-7.12 (m, 2H), 7.31 (t, 1H), 7.54 (s, 1H), 9.04 (d, 1H), 9.79 (s, 1H), 14.42 (bs, 1H);

MS (ESI+) m/z 266 (M+H)⁺;

MS (ESI−) m/z 264 (M−H)⁻;

Anal. Calcd for C₁₂H₁₅N₃O₂S.HCl: C, 47.76; H, 5.34; N, 13.92. Found: C, 47.63; H, 5.30; N, 13.63.

### EXAMPLE 86

### (−) N-{3-[1-(1H-imidazol-4-yl)ethyl]phenyl}methanesulfonamide Hydrochloride

The slower moving enantiomer from Example 85E was processed as described in Example 33C to provide the title compound, which was converted to the hydrochloride salt.

mp 195-196° C.;

[α]²³_{D}−32.1° (c 1.0, methanol);

¹HNMR (DMSO-d₆) δ 1.57 (d, 3H), 2.99 (s, 3H), 4.24 (q, 1H), 7.00 (d, 1H), 7.05-7.12 (m, 2H), 7.31 (t, 1H), 7.54 (s, 1H), 9.04 (d, 1H), 9.79 (s, 1H), 14.42 (bs, 1H);

MS (ESI+) m/z 400 (M+H)⁺;

MS (ESI−) m/z 398 (M−H)⁻;

Anal. Calcd for C₁₂H₁₅N₃O₂S.HCl: C, 47.76; H, 5.34; N, 13.92. Found: C, 47.64; H, 5.27; N, 13.68.

### Example 87

### N-[1-(1H-imidazol-4-yl)-1,3-dihydro-2-benzofuran-4-yl]ethanesulfonamide Maleate

### Example 87A

### 4-[2-(hydroxymethyl)-3-nitrobenzoyl]-N,N-dimethyl-1H-imidazole-1-sulfonamide

4-Iodo-N,N-dimethyl-1H-imidazole-1-sulfonamide (3.0 g, 10 mmol), prepared as described in (R. M. Turner, J. Org. Chem. (1991), 56, 5739-5740) and 4-nitro-2-benzofuran-1(3H)-one, prepared as described in (Stanetty, Peter J.Prakt.Chem./Chem.-Ztg 335, 1993, 17-22) were processed as described in Example 1A to provide the title compound.

MS (ESI+) m/z 355 (M+H)⁺;

MS (ESI−) m/z 353 (M−H)⁻.

### Example 87B

### N,N-dimethyl-4-(4-nitro-1,3-dihydro-2-benzofuran-1-yl)-1H-imidazole-1-sulfonamide

The product from Example 87A (0.50 g, 1.4 mmol) was treated with trifluoroacetic acid (10 mL) and triethylsilane (2.5 mL) at ambient temperature. After 1 hour of stirring, the mixture was concentrated to an oil. The residue was purified on silica gel with 1:1 ethyl acetate:hexane to provide the title compound.

MS (ESI+) m/z 339 (M+H)⁺.

### Example 87C

### 4-(4-amino-1,3-dihydro-2-benzofuran-1-yl)-N,N-dimethyl-1H-imidazole-1-sulfonamide

The product from Example 87B in ethyl acetate was processed as described in Example 1C to provide the title compound.

MS (ESI+) m/z 309 (M+H)⁺.

### Example 87D

### N-[1-(1H-imidazol-4-yl)-1,3-dihydro-2-benzofuran-4-yl]ethanesulfonamide Maleate

The product from Example 87C and ethanesulfonyl chloride were processed as described in Example 31D. The residue was purified on silica gel with 5% and then 10% and then 20% methanol/ammonia-saturated dichloromethane to provide the title compound, which was converted to the maleic acid salt.

mp 95-98° C.;

¹H NMR (DMSO-d₆) δ 1.25 (t, 3H), 3.14 (q, 2H), 5.12 (d, 1H), 5.26 (dd, 1H), 6.09 (s, 2H), 6.31 (s, 1H), 6.98 (dd, 1H), 7.25-7.36 (m, 2H), 7.51 (bs, 1H), 8.67 (bs, 1H), 9.59 (s, 1H), 14.6 (bs, 1H);

MS (ESI+) m/z 294 (M+H)⁺;

MS (ESI−) m/z 292 (M−H)⁻;

Anal. Calcd for C₁₃H₁₅N₃O₃S.C₄H₄O₄ 0.5 C₄H₈O₂: C, 50.33; H, 5.11; N, 9.27. Found: C, 50.42; H, 4.79; N, 9.23.

### EXAMPLE 88

### 2,2,2-trifluoro-N-[4-(1H-imidazol-4-yl)-3,4-dihydro-2H-chromen-8-yl]ethanesulfonamide

### Example 88A

### tert-butyl 4-(8-{[(2,2,2-trifluoroethyl)sulfonyl]amino}-3,4-dihydro-2H-chromen-4-yl)-1H-imidazole-1-carboxylate

The product from Example 19C (0.60 g, 1.9 mmol) was treated with pyridine (0.46 mL, 5.7 mmol) and 2,2,2-trifluoroethanesulfonyl chloride (0.23 mL, 2.1 mmol). After stirring for 16 hours, the mixture was concentrated. The residue was purified on silica gel using 1:1 hexane:ethyl acetate to provide the desired compound.

### Example 88B

### 2,2,2-trifluoro-N-[4-(1H-imidazol-4-yl)-3,4-dihydro-2H-chromen-8-yl]ethanesulfonamide

The enantiomers of Example 88A were separated by chiral chromatography on a Chiralcel OJ chiral column using 95:5 hexane:ethanol as the mobile phase. The faster moving enantiomer was processed as described in Example 33C to provide the title compound, which was converted to the maleic acid salt.

mp 173-176° C.;

¹H NMR (DMSO-d₆) δ 2.20 (m, 2H), 4.15-4.48 (m, 5H), 6.06 (s, 2H), 6.85 (m, 2H), 7.15 (dd, 1H), 7.26 (s, 1H), 8.75 (s, 1H), 9.65 (s, 1H);

MS (APCI+) m/z 362 (M+H)⁺;

Anal. Calcd for C₁₄H₁₄F₃N₃O₃S C₄H₄O₄: C, 45.28; H, 3.80; N, 8.80. Found: C, 45.68; H, 3.68; N, 8.63.

### EXAMPLE 89

### N-[4-(1H-imidazol-4-yl)-3,4-dihydro-2H-thiochromen-8-yl]ethanesulfonamide Maleate

### Example 89A

### 4-(4-hydroxy-8-nitro-3,4-dihydro-2H-thiochromen-4-yl)-N,N-dimethyl-1H-imidazole-1-sulfonamide

4-Iodo-N,N-dimethyl-1H-imidazole-1-sulfonamide (3.0 g, 10 mmol), prepared as described in (R. M. Turner, J. Org. Chem. (1991), 56, 5739-5740) and 8-nitrothiochroman-4-one, prepared as described in (Schaefer, Ted Can.J.Chem. 65, 1987, 908-914) were processed as described in Example 1A to provide the title compound.

### Example 89B

### N,N-dimethyl-4-(8-nitro-2H-thiochromen-4-yl)-1H-imidazole-1-sulfonamide

The product from Example 89A was processed as described in Example 31B to provide the title compound.

MS (APCI+) m/z 367 (M+H)⁺.

### Example 89C

### 4-(8-amino-3,4-dihydro-2H-thiochromen-4-yl)-N,N-dimethyl-1H-imidazole-1-sulfonamide

The product from Example 89B in ethyl acetate was processed as described in Example 1C to provide the title compound.

MS (DCI/NH₃) m/z 339 (M+H)⁺.

### Example 89D

### N-[4-(1H-imidazol-4-yl)-3,4-dihydro-2H-thiochromen-8-yl]ethanesulfonamide Maleate

The product from Example 89C and ethanesulfonyl chloride were processed as described in Example 3 ID to provide the title compound, which was converted to the maleic acid salt.

mp 248-251° C.;

¹H NMR (DMSO-d₆) δ 1.30 (t, 3H), 2.01 (m, 1H), 2.44 (m, 1H), 2.90 (m, 2H), 3.11 (q, 2H), 4.16 (m, 1H), 6.40 (s, 1H), 6.95 (m, 2H), 7.11 (m, 1H), 7.80 (s, 1H), 9.0 (s, 1H), 11.81 (bs, 1H);

MS (APCI+) m/z 324 (M+H)

Anal. Calcd for C]₄H₁₇N₃O₂S 0.25H₂O: C, 51.28; H, 5.38; N, 12.81. Found: C, 50.92; H, 5.21; N, 12.65.

### EXAMPLE 90

### N-[6-fluoro-4-(1H-imidazol-4-yl)-3,4-dihydro-2H-chromen-8-yl]methanesulfonamide Maleate

The product from Example 80D and methanesulfonyl chloride were processed as described in Example 31D to provide the title compound, which was converted to the maleic acid salt.

mp 187-190° C.;

¹H NMR (DMSO-d₆) δ 2.2 (m, 2H), 3.04 (s, 3H), 4.22 (m, 2H), 4.36 (t, 1H), 6.07 (s, 2H), 6.63 (d, 1H), 7.01 (d, 1H), 7.29 (s, 1H), 8.70 (s, 1H), 9.09 (s, 1H); MS (APCI+) m/z 312 (M+H)⁺;

Anal. Calcd for C₁₃H₁₄FN₃O₃S C₄H₄O₄: C, 47.77; H, 4.25; N, 9.83. Found: C, 47.76; H, 4.40; N, 9.70.

### EXAMPLE 91

### 2,2,2-trifluoro-N-{3-[1-(1H-imidazol-4-yl)vinyl]phenyl}ethanesulfonamide Maleate

The product from Example 45A and 2,2,2-trifluoroethanesulfonyl chloride were processed as described in Example 31D to provide the title compound, which was converted to the maleic acid salt.

mp 149-153° C.;

¹H NMR (DMSO-d₆) δ 4.55 (q, 2H), 5.42 (s, 1H), 5.81 (s, 1H), 6.12 (s, 2H), 7.25 (m, 3H), 7.31 (s, 1H), 7.41 (dd, 1H), 8.56 (s, 1H), 10.5 (s, 1H);

MS (APCI+) m/z 332 (M+H)⁺;

Anal. Calcd for C₁₃H₁₂F₃N₃O₂S C₄H₄O₄: C, 45.64; H, 3.61; N, 9.39. Found: C, 45.43; H, 3.59; N, 9.33.

### EXAMPLE 92

### N-{3-[1-(1H-imidazol-4-yl)vinyl]phenyl}methanesulfonamide

The product from Example 45A and methanesulfonyl chloride were processed as described in Example 31D to provide the title compound, which was converted to the maleic acid salt.

mp 167-170° C.;

¹H NMR (DMSO-d₆) δ 3.02 (s, 3H), 5.44 (s, 1H), 5.81 (s, 1H), 6.12 (s, 2H), 7.18 (d, 1H), 7.24 (d, 1H), 7.26 (s, 1H), 7.33 (s, 1H), 7.39 (dd, 1H), 8.62 (s, 1H), 9.82 (s, 1H);

MS (APCI+) m/z 264 (M+H)⁺;

Anal. Calcd for C₁₂H₁₃N₃O₂S C₄H₄O₄: C, 50.65; H, 4.52; N, 11.07. Found: C, 50.53; H, 4.69; N, 10.88.

### EXAMPLE 93

### (+) N-[4-(1H-imidazol-4-yl)-3,4-dihydro-2H-chromen-8-yl]methanesulfonamide Maleate

### Example 93A

### tert-butyl 4-{8-[(methylsulfonylamino]-3,4-dihydro-2H-chromen-4-yl}-1H-imidazole-1-carboxylate

The product from Example 19C and methanesulfonyl chloride were processed as described in Example 88A to provide the title compound. MS (APCI+) m/z 394 (M+H)⁺;

### Example 93B

### (+) N-[4-(1H-imidazol-4-yl)-3,4-dihydro-2H-chromen-8-yl]methanesulfonamide Maleate

The enantiomers from Example 93A were separated by chiral chromatography on a Chiralcel OJ column eluting with 92:8 hexane: ethanol. The faster moving enantiomer was processed as described in Example 33C to provide the title compound, which was converted to the maleic acid salt.

mp 205-208° C.;

[α]²³_{D}+68.00 (c 1.0, methanol);

¹H NMR (DMSO-d₆) δ 2.17 (m, 2H), 2.95 (s, 3H), 4.07 (m, 1H), 4.24 (m, 2H), 6.69 (s, 1H), 6.75 (dd, 1H), 6.90 (d, 1H), 7.08 (d, 1H), 7.56 (s, 1H), 8.77 (s, 1H);

MS (APCI+) m/z 294 (M+H)⁺;

Anal. Calcd for C₁₃H₁₅N₃O₃S 0.5H₂O: C, 51.64; H, 5.33; N, 13.90. Found: C, 51.46; H, 5.05; N, 13.88.

### EXAMPLE 94

### N-{3-[1-(1H-imidazol-4-yl)-2-methyl-1-propenyl]phenyl}ethanesulfonamide

### Example 94A

### 4-[1-(3-aminophenyl)-2-methyl-1-propenyl]-N,N-dimethyl-1H-imidazole-1-sulfonamide

The product from Example 55C (0.40 g, 1.4 mmol) in tetrahydrofuran (5.4 mL) at 0° C. under a nitrogen atmosphere was treated with a solution of 2M isopropylmagnesium chloride in ether (3.4 mL, 6.8 mmol), warmed to ambient temperature, stirred for 1 hour, treated with aqueous ammonium chloride and extracted three times with ethyl acetate. The combined ethyl acetate extractions were washed with brine, dried (Na₂SO₄), concentrated, treated with trifluoroacetic acid (5 mL), stirred at ambient temperature for 16 hours, neutralized with sodium bicarbonate solution and extracted three times with ethyl acetate. The combined ethyl acetate extractions were washed with brine, dried (Na₂SO₄) and concentrated to provide the title compound which was not purified but carried on to the next step.

MS (APCI+) m/z 321 (M+H)⁺.

### Example 94B

### N-{3-[1-(1H-imidazol-4-yl)-2-methyl-1-propenyl]phenyl}ethanesulfonamide

The product from Example 94A (0.036 g, 0.17 mmol) in dichloromethane (2 mL) was treated with pyridine (0.055 mL, 0.68 mmol) and ethanesulfonyl chloride (0.034 mL, 0.35 mmol). After stirring for 3 hours, the reaction mixture was quenched with water and treated with a small amount of concentrated HCl. The mixture was extracted three times with ethyl acetate. The combined ethyl acetate extractions were washed with brine, dried (Na2SO4) and concentrated. The residue in methanol (2 mL) was treated with a solution of 50% sodium hydroxide (5 drops). After stirring for 2 hours, the mixture was treated with aqueous ammonium chloride solution and extracted three times with ethyl acetate. The combined ethyl acetate extractions were washed with brine, dried (Na₂SO₄) and concentrated. The residue was purified on silica gel eluting with 10% ethanol/ammonia-saturated dichloromethane to provide the title compound.

mp 152-155° C.;

¹H NMR (DMSO-d₆) δ 1.16 (t, 3H), 1.65 (m, 3H),1.82-2.15 (m, 3H), 3.05 (q, 2H), 6.52-6.77 (m, 1H), 6.81 (d, 1H), 6.96 (s, 1H), 7.08 (m, 1H), 7.25 (m, 1H), 7.52 (m, 1H), 9.70 (s, 1H);

MS (APCI+) m/z 306 (M+H⁺);

Anal. Calcd for C₁₅H₁₉N₃O₂S: C, 58.99; H, 6.27; N, 13.75. Found: C, 58.61; H, 6.24; N, 13.38.

### EXAMPLE 95

### (+) N-[4-(1H-imidazol-4-yl)-3,4-dihydro-2H-chromen-8-yl]ethanesulfonamide

### Example 95A

### tert-butyl 4-{8-[(ethylsulfonyl)amino]-3,4-dihydro-2H-chromen-4-yl}-1H-imidazole-1-carboxylate

The product from Example 19C and ethanesulfonyl chloride were processed as described in Example 88A to provide the title compound.

### Example 95B

### (+) N-[4-(1H-imidazol-4-yl)-3,4-dihydro-2H-chromen-8-yl]ethanesulfonamide

The enantiomers from Example 95A were separated by chiral chromatography on a Chiralcel OJ column eluting with 9% ethanol in hexane. The faster moving enantiomer was processed as described in Example 33C to provide the title compound.

mp 223-226° C.;

[α]²³_{D}+65.90 (c 1.0, methanol);

¹H NMR (DMSO-d₆) δ 1.25 (t, 3H), 2.18 (m, 2H), 3.02 (q, 2H), 4.11 (t, 1H), 4.22 (m, 2H), 6.67 (s, 1H), 6.74 (dd, 1H), 6.86 (d, 1H), 7.09 (d, 1H), 7.56 (s, 1H), 8.71 (s, 1H), 11.87 (s, 1H);

MS (APCI+) m/z 308 (M+H)⁺;

Anal. Calcd for C₁₄H₁₇N₃O₃S 0.5H₂O: C, 53.15; H, 5.73; N, 13.28. Found: C, 53.49; H, 5.41; N, 13.14.

### EXAMPLE 96

### N-[2,5-dichloro-3-(1H-imidazol-4-ylmethyl)phenyl]ethanesulfonamide

### Example 96A

### 2,5-dichloro-3-nitrobenzaldehyde

2,5-Dichloro-3-nitrobenzoic acid (1.0 g, 4.24 mmol) in diethyl ether (5 mL) and tetrahydrofuran (5 mL) at ambient temperature was treated dropwise with neat borane-dimethylsulfide complex (0.41 mL, 4.24 mmol). During addition the reaction mixture gently refluxed, and the reflux was continued with an oil bath for 1 hour. The reaction mixture was allowed to cool to ambient temperature and concentrated under reduced pressure. The residue in dichloromethane (5 mL×2) was added to a rapidly stirring suspension of pyridinium chlorochromate (1.01 g, 4.66 mmol) in dichloromethane (10 mL) at ambient temperature. Upon complete addition, the temperature was raised to reflux for 1 hour. The reaction mixture was allowed to cool to ambient temerature, filtered through a Celite plug, concentrated under reduced pressure. The residue was chromatographed on flash silica gel eluting with 10% ethyl acetate/dichloromethane to afford 690 mg (74%) of the title compound.

¹H NMR (300 MHz, CDCl₃) δ 8.02 (d, J=2.7 Hz, 1H), 8.11 (d, J=2.7 Hz, 1H), 10.48 (s, 1H).

### Example 96B

### 4-[(2,5-dichloro-3-nitrophenal)(hydroxy)methyl]-N,N-dimethyl-1H-imidazole-1-sulfonamide

The product from Example 96A and 4-iodo-N,N-dimethyl-1H-imidazole-1-sulfonamide (0.90 g, 3 mmol), prepared as described in (R. M. Turner, J. Org. Chem. (1991) 56, 5739-5740) were processed as described in Example 1A to provide 850 mg (79%) of the title product.

¹H NMR (300 MHz, DMSO-d₆) δ 2.77 (s, 6H), 5.98 (d, J=5.1 Hz, 1H), 6.45 (d, J=5.1 Hz, 1H), 7.58 (bs, 1H), 7.98 (d, J=2.4 Hz, 1H), 8.09 (d, J=0.9 Hz, 1H), 8.23 (d, J=2.4 Hz, 1H);

MS (APCI+) m/z 395 (M+H)⁺.

### Example 96C

### 4-(2,5-dichloro-3-nitrobenzyl)-N,N-dimethyl-1H-imidazole-1-sulfonamide

The product from Example 96B (473 mg, 1.20 mmol), triethylsilane (4 mL), and trifluoroacetic acid (3 mL) were brought to vigorous reflux for 3 hours. The reaction mixture was allowed to cool to ambient temperature and concentrated under reduced pressure. The remaining oil was triturated with hexanes and then chromatographed on flash silica gel with 5% methanol-dichloromethane to afford 300 mg (66%) of the title compound.

¹H NMR (300 MHz, DMSO-d₆) δ 2.78 (s, 6H), 4.10 (s, 2H), 7.48 (d, J=0.7 Hz, 1H), 7.83 (d, J=2.4 Hz, 1H), 8.12 (d, J=0.9 Hz, 1H), 8.17 (d, J=2.4 Hz, 1H);

MS (APCI+) m/z 379 (M+H)⁺.

### Example 96D

### 4-(3-amino-2,5-dichlorobenzyl)-N,N-dimethyl-1H-imidazole-1-sulfonamide

The product from Example 96C (300 mg, 0.79 mmol) in water (5 mL) and ethanol (10 mL) was treated with ammonium chloride (46 mg, 0.87 mmol) and iron (338 mg, 6.0 mmol). The mixture was refluxed for 30 minutes, allowed to cool to ambient temperature, filtered through Celite, concentrated under reduced pressure to near dryness, redissolved in dichloromethane, dried (Na₂SO₄), filtered, and reconcentrated under reduced pressure. The residue was chromatographed on flash silica gel with 5% methanol-dichloromethane to afford 200 mg (72%) of the title compound.

¹H NMR (300 MHz, DMSO-d₆) δ 2.78 (s, 6H), 3.86 (s, 2H), 5.65 (s, 2H), 6.47 (d, J=2.4 Hz, 1H), 6.73 (d, J=2.4 Hz, 1H), 7.34 (bs, 1H), 8.09 (d, J=0.7 Hz, 1H); MS (APCI+) m/z 349 (M+H)⁺.

### Example 96E

### 4-{2,5-dichloro-3-[(ethylsulfonyl)amino]benzyl}-N,N-dimethyl-1H-imidazole-1-sulfonamide

The product from Example 96D (200 mg, 0.57 mmol) and ethanesulfonyl chloride were processed as described in Example 88A to provide 150 mg (59%) of the title product.

### Example 96F

### N-[2,5-dichloro-3-(1H-imidazol-4-ylmethyl)phenyl]ethanesulfonamide

The product from Example 96E (130 mg, 0.30 mmol) in dioxane (3 mL) was treated with 2N HCl (1 mL) at reflux for 3 hours. After cooling to ambient temperature, the dioxane was removed under reduced pressure. The residual solution was loaded onto a Dowex ion exchange resin and the resin washed with water until the rinse was neutral. The eluant was then changed to 1:15% aqueous ammonium hydroxide:ethanol to provide 62 mg (63%) of the title product.

mp 182-184° C.;

¹H NMR (300 MHz, CD₃OD) δ 1.34 (t, J=7.5 Hz, 3H), 3.15 (q, J=7.5 Hz, 2H), 4.06 (s, 2H), 6.86 (bs, 1H), 7.07 (d, J=2.7 Hz, 1H), 7.51 (d, J=2.7 Hz, 1H), 7.64 (d, J=0.7 Hz, 1H);

MS (APCI+) m/z 334 (M+H)⁺;

FAB HRMS m/z for C₁₂H₁₄N₃O₂Cl₂S (M+H)⁺: calculated 334.0184, observed 334.0182.

### EXAMPLE 97

### N-[5-(1H-imidazol-4-ylmethyl)-2-methylphenyl]ethanesulfonamide

### Example 97A

### 4-[hydroxy(4-methyl-3-nitrophenpl)methyl]-N,N-dimethyl-1H-imidazole-1-sulfonamide

4-Methyl-3-nitrobenzaldehyde and 4-iodo-N,N-dimethyl-1H-imidazole-1-sulfonamide (0.90 g, 3 mmol), prepared as described in (R. M. Turner, J. Org. Chem. (1991) 56, 5739-5740) were processed as described in Example 1A to provide 2.0 g (97%) of the title compound.

¹H NMR (300 MHz, CDCl₃) δ 2.61 (s, 6H), 5.87 (s, 1H), 7.02 (bs, 1H), 7.37 (d, J=7.8 Hz, 1H), 7.62 (dd, J=0.9, 7.8 Hz, 1H), 7.93 (bs, 1H), 8.07 (d, J=0.9 Hz, 1H);

MS (APCI+) m/z 341 (M+H)⁺.

### Example 97B

### N,N-dimethyl-4-(4-methyl-3-nitrobenzyl)-1H-imidazole-1-sulfonamide

The product from Example 97A was processed as described in Example 96C to provide 770 mg (99%) of the title compound.

¹H NMR (300 MHz, CDCl₃) δ 2.61 (s, 6H), 4.12 (s, 2H), 7.02 (bs, 1H), 7.35 (d, J=7.8 Hz, 1H), 7.46 (dd, J=0.7, 7.8 Hz, 1H), 7.86 (bs, 1H), 8.54 (bs, 1H);

MS (APCI+) m/z 325 (M+H)⁺.

### Example 97C

### 4-(3-amino-4-methylbenzyl)-N.N-dimethyl-1H-imidazole-1-sulfonamide

The product from Example 97B (200 mg, 0.62 mmol) and zinc (401 mg, 6.2 mmol) in methanol (1.5 mL) were added dropwise to a solution of concentrated HCl (1.3 mL) and methanol (1.3 mL) at 0° C. The reaction mixture bubbled vigorously. After 15 minutes, the mixture was treated with saturated aqueous sodium bicarbonate solution and solid sodium chloride until saturated and extracted multiple times with ethyl acetate. The combined ethyl acetate extracts were dried (Na₂SO₄), filtered and concentrated under reduced pressure to afford 140 mg (77%) of the title compound.

### Example 97D

### 4-{3-[(ethylsulfonyl)amino]-4-methylbenzyl}-N,N-dimethyl-1H-imidazole-1-sulfonamide

The product from Example 97C and ethanesulfonyl chloride were processed as described in Example 88A to provide 164 mg (88%) of the title compound.

¹H NMR (300 MHz, CDCl₃) δ 1.36 (t, J=7.5 Hz, 3H), 2.85 (s, 6H), 3.13 (q, J=7.5 Hz, 1H), 3.91 (s, 2H), 6.92 (d, J=0.7 Hz, 1H), 7.02 (dd, J=0.9, 7.8 Hz, 1H), 7.15 (d, J=7.8 Hz, 1H), 7.33 (d, J=0.9 Hz, 1H), 7.86 (d, J=0.7 Hz, 1H);

MS (APCI+) m/z 387 (M+H)⁺.

### Example 97E

### N-[5-(1H-imidazol-4-ylmethyl)-2-methylphenyl]ethanesulfonamide

The product from Example 97D was processed as described in Example 96E to provide 164 mg (88%) of the title compound.

mp 140-152° C.

¹H NMR (300 MHz, CD₃OD) δ 1.33 (t, J=7.2 Hz, 3H), 2.32 (s, 3H), 3.07 (q, J=7.2 Hz, 2H), 3.88 (s, 2H), 6.77 (d, J=0.6 Hz, 1H), 7.02 (dd, J=0.9, 7.5 Hz, 1H), 7.14 (d, J=7.5 Hz, 1H), 7.18 (d, J=0.9 Hz, 1H), 7.58 (d, J=0.6 Hz, 1H);

MS (APCI+) m/z 280 (M+H)⁺;

FAB HRMS m/z for C₁₃H₁₁N₃O₂S (M+H)⁺: calculated 280.1120, observed 280.1124.

### EXAMPLE 98

### N-[5-(1H-imidazol-4-ylmethyl)-2-methylphenyl]methanesulfonamide

### Example 98A

### N,N-dimethyl-4-{4-methyl-3-[(methylsulfonyl)amino]benzyl}-1H-imidazole-1-sulfonamide

The product from Example 97C and methanesulfonyl chloride were processed as described in Example 88A to provide 214 mg (81%) of the title compound.

### Example 98B

### N-[5-(1H-imidazol-4-ylmethyl)-2-methylphenyl]methanesulfonamide

The product from Example 98A was processed as described in Example 96F to provide 110 mg (76%) of the title compound as a foamy oil.

¹H NMR (300 MHz, CD₃OD) δ 2.32 (s, 3H), 2.93 (s, 3H). 3.89 (s, 2H), 6.77 (bs, 1H), 7.03 (dd, J=0.9, 7.5 Hz, 1H), 7.17 (d, J=7.5 Hz, 1H), 7.20 (d, J=0.9 Hz, 1H), 7.58 (d, J=0.6 Hz, 1H); MS (APCI+) m/z 266 (M+H)⁺;

FAB HRMS m/z for C₁₂H₁₆N₃O₂S (M+H)⁺: calculated 266.0963, observed 266.0974.

### EXAMPLE 99

### N-[3-(1H-imidazol-4-ylmethyl)-2,5-dimethylphenyl]ethanesulfonamide

### Example 99A

### 2,5-dimethyl-3-nitrobenzaldehyde

2,5-Dimethylbenzaldehyde (500 mg, 3.73 mmol) was slowly added to a solution of sulfuric acid (4 mL) at −5° C. After stirring until homogeneous, the mixture was treated with sodium nitrate (762 mg, 8.96 mmol) which was added in small aliquots via a spatula. After 30 minutes, the reaction mixture was poured into crushed ice and water and sodium chloride was added until saturation was reached. The mixture was extracted with ethyl acetate. The organics were combined, dried (Na₂SO₄), filtered, and concentrated under reduced pressure to provide 200 mg (30%) of an intractable mixture of 2,5-dimethyl-3-nitrobenzaldehyde (desired/minor) and 3,6-dimethyl-2-nitrobenzaldehyde (undesired/major).

¹H NMR (300 MHz, CDCl₃) δ 2.33 (s, 3H, major), 2.48 (s, 3H, minor), 2.64 (s, 3H, major), 2.73 (s, 3H, minor), 7.32 (d, J=8.1 Hz, 1H, major), 7.41 (d, J=8.1 Hz, 1H, major), 7.78 (bs, 1H, minor), 7.87 (bs, 1H, minor), 10.22 (s, 1H, major), 10.36 (s, 1H, minor); MS (APCI+) m/z 180 (M+H)⁺.

### Example 99B

### 4-[(2,5-dimethyl-3-nitrophenyl)(hydroxy)methyl]-N,N-dimethyl-1H-imidazole-1-sulfonamide

The product from Example 99A and 4-iodo-N,N-dimethyl-1H-imidazole-1-sulfonamide (0.90 g, 3 mmol), prepared as described in (R. M. Turner, J. Org. Chem. (1991) 56, 5739-5740), were processed as described in Example 1A to provide 260 mg (26%) of the title compound.

¹H NMR (300 MHz, DMSO-d₆) δ 2.26 (s, 3H), 2.34 (s, 3H), 2.79 (s, 6H), 5.89 (d, J=4.5 Hz, 1H), 6.06 (d, J=4.5 Hz, 1H), 7.40 (bs, 1H), 7.55 (bs, 1H), 7.62 (bs, 1H), 8.07 (d, J=0.9 Hz, 1H); MS (APCI+) m/z 355 (M+H)⁺.

### Example 99C

### 4-(2,5-dimethyl-3-nitrobenzyl)-N,N-dimethyl-1H-imidazole-1-sulfonamide

The product from Example 99B was processed as described in Example 96C to provide 181 mg (73%) of the title compound.

¹H NMR (300 MHz, DMSO-d₆) δ 2.29 (s, 3H), 2.32 (s, 3H), 2.78 (s, 6H), 3.94 (s, 2H), 7.37 (bs, 2H), 7.54 (bs, 1H), 8.09 (d, J=0.9 Hz, 1H);

MS (APCI+) m/z 339 (M+H)⁺.

### Example 99D

### 4-(3-amino-2,5-dimethylbenzyl)-N,N-dimethyl-1H-imidazole-1-sulfonamide

The product from Example 99C was processed as described in Example 97C to provide 140 mg (88%) of the title compound.

¹H NMR (300 MHz, DMSO-d₆) δ 1.93 (s, 3H), 2.09 (s, 3H), 2.76 (s, 6H), 3.71 (s, 2H), 4.65 (bs, 2H), 6.23 (bs, 1H), 6.32 (bs, 1H), 7.04 (bs, 1H), 8.03 (bs, 1H);

MS (APCI+) m/z 309 (M+H)⁺.

### Example 99E

### 4-{3-[(ethylsulfonyl)amino]-2,5-dimethylbenzyl}-N,N-dimethyl-1H-imidazole-1-sulfonamide

The product from Example 99D and ethanesulfonyl chloride were processed as described in Example 88A to provide 153 mg (84%) of the title compound.

¹H NMR (300 MHz, CDCl₃) δ 1.40 (t, J=7.5 Hz, 3H), 2.20 (s, 3H), 2.31 (s, 3H), 2.82 (s, 6H), 3.15 (q, J=7.5 Hz, 2H), 3.92 (s, 2H), 6.02 (bs, 1H), 6.72 (bs, 1H), 6.93 (bs, 1H), 7.17 (bs, 1H), 7.88 (bs, 1H);

MS (APCI+) m/z 401 (M+H)⁺.

### Example 99F

### N-[3-(1H-imidazol-4-ylmethyl)-2,5-dimethylphenyl]ethanesulfonamide

The product from Example 99E was processed as described in Example 96F to provide 53 mg (48%) of the title compound.

mp 167-169° C.;

¹H NMR (300 MHz, CD₃OD) δ 1.36 (t, J=7.5 Hz, 3H), 2.24 (s, 3H), 2.27 (s, 3H), 3.08 (q, J=7.5 Hz, 2H), 3.91 (s, 2H), 6.57 (bs, 1H), 6.93 (bs, 1H), 7.04 (bs, 1H), 7.59 (bs, 1H);

MS (APCI+) m/z 294 (M+H)⁺;

FAB HRMS m/z for C₁₄H₂₀N₃O₂S (M+H)⁺: calculated 294.1276, observed 294.1263.

### EXAMPLE 100

### N-[3-(1H-imidazol-4-ylmethyl)-2,5-dimethylphenyl]methanesulfonamide

### Example 100A

### 4-{2,5-dimethyl-3-[(methylsulfonyl)amino]benzyl}-N,N-dimethyl-1H-imidazole-1-sulfonamide

The product from Example 99D and methanesulfonyl chloride were processed as described in Example 88A to provide the title compound.

### Example 100B

### N-[3-(1H-imidazol-4-ylmethyl)-2,5-dime hylphenyl]methanesulfonamide

The product from Example 100A was processed as described in Example 96F to provide 37 mg (20% overall for two steps) of the title compound.

mp 197-199° C.;

¹H NMR (300 MHz, CD₃OD) δ 2.24 (s, 3H), 2.27 (s, 3H), 2.92 (s, 3H), 3.91 (s, 2H), 6.57 (d, J=0.7 Hz, 1H), 6.93 (bs, 1H), 7.07 (bs, 1H), 7.58 (d, J=0.7 Hz, 1H);

MS (APCI+) m/z 280 (M+H)⁺.

### EXAMPLE 101

### N-[3-cyclohexyl-5-(1H-imidazol-4-yl)-5,6,7,8-tetrahydro-1-naphthalenyl]ethanesulfonamide

### Example 101A

### 4-(4-cyclohexylphenyl)-4-oxobutanoic Acid

3-(4-Cyclohexylbenzoyl)acrylic acid (5 g, 19.3 mmol) in methanol (200 mL) was treated with 10% Pd/C (3.6 g) under a hydrogen atmosphere (4 atmospheres) for 5 hours. The catalyst was filtered and the filtrate was concentrated under reduced pressure to provide (5 g, 100%) title compound.

¹H NMR (300 MHz, CDCl₃) δ 1.38 (m, 4H), 1.85 (m, 4H), 1.97 (quintet, J=7 Hz, 2H), 2.38,J=7 Hz, 2H), 2.46 (m,1H), 2.63 (t, J=7 Hz, 2H), 7.11 (m, 4H);

MS (DCI/NH₃) m/z 261 (M+H)⁺.

### Example 101B

### 4-(4-cyclohexylphenyl)butanoic Acid

The product from Example 101A in ethylene glycol (50 mL) was treated with hydrazine hydrate (4 mL) and solid potassium hydroxide (4 g) and refluxed for 3 hours. The mixture was poured into ice-water, treated with 12M HCl, and extracted with diethyl ether. The organic layer was washed with water, brine, dried (MgSO₄), filtered, and concentrated to provide (4 g, 84%) the title compound.

### Example 101C

### 7-cyclohexyl-3,4-dihydro-1 (2H)-naphthalenone

The product from Example 101B (4 g, 16 mmol) in xylenes (150 mL) was treated with polyphosphoric acid (6 g) and refluxed for 7 hours. The reaction mixture was allowed to cool to ambient temperature and poured into water. The xylene layer was separated, dried (MgSO₄), filtered and concentrated under reduced pressure. The residue was purified by column chromatography (silica gel, 3:1 hexane:ethyl acetate) to provide (3.8 g, 98%) the title compound.

¹H NMR (300 MHz, DMSO-d₆) δ 1.36 (m, 5H), 1.75 (m, 5H), 2.03 (m, 2H), 2.54 (q, J=7 Hz, 3H), 2.9 (t, J=7 Hz, 2H), 7.25 (d, J=9 Hz,1H), 7.40 (d-d, J=3 and 9 Hz,1H), 7.70 (d, J=3 Hz, 1H);

MS (DCI/NH₃) m/z 229 (M+H)⁺, 246 (M+NH₄)⁺.

### Example 101D

### 7-cyclohexyl-5-nitro-3,4-dihydro-1(2H)-naphthalenone

The product from Example 101C (3.8 g, 16.6 mmol) in concentrated H₂SO₄ (35 mL) at −5° C. was treated in portions with solid sodium nitrate (1.7 g, 20 mmol). After stirring at 0° C. for 2 hours, the mixture was poured into ice and extracted with ethyl acetate. The ethyl acetate layer was dried (MgSO₄), filtered and concentrated. The residue was purified by column chromatography (silica gel, 3:1 hexane:ethyl acetate) to provide the title compound (1.5 g) contaminated with starting material. It was used without further purification.

### Example 101E

### 4-(7-cyclohexyl-5-nitro-3,4-dihydro-1-naphthalenyl)-N,N-dimethyl-1H-imidazole-1-sulfonamide

The product from Example 101D and 4-iodo-N,N-dimethyl-1H-imidazole-1-sulfonamide (0.90 g, 3 mmol), prepared as described in (R. M. Turner, J. Org. Chem. (1991) 56, 5739-5740), were processed as described in Example 1A to provide an intermediate alcohol which was further processed as described in Example 31B to provide the title compound as a crude product (1.1 g).

MS (APCI+) m/z 431 (M+H)⁺;

MS (APCI−) m/z 465 (M+Cl)⁻.

### Example 101F

### 4-{7-cyclohexyl-5-[(ethylsulfonyl)amino]-1,2,3,4-tetrahydro-1-naphthalenyl}-N,N-dimethyl-1H-imidazole-1-sulfonamide

The product from Example 101E was hydrogenated over 10% Pd/C in ethanol:1,4-dioxane (4:1) (20 mL) at ambient temperature for 15 hours. The catalyst was filtered off and the filtrate was concentrated under reduced pressure and the residue redissolved in pyridine (10 mL). The resulting solution was treated at 0° C. with ethanesulfonyl chloride (0.5 mL, 5 mmol) dropwise. The mixture was allowed to warm to ambient temperature. After 8 hours, the mixture was concentrated under reduced pressure and the residue purified by column chromatography (silica gel, 1:1 hexane:ethyl acetate) to provide 670 mg (56%) of the title compound.

¹H NMR (300 MHz, DMSO-d₆) δ 1.28 (t+m, 9H), 1.70 (m, 8H), 2.00 (m, 2H), 2.34 (m, 1H), 3.10 (q, J=7 Hz, 2H), 4.05 (t, J=7 Hz, 1H), 6.76 (s, 1H), 6.96 (d, J=1.5 Hz, 1H), 7.04 (s, 1H), 8.10 (d, J=1.5 Hz, 1H), 8.85 (s, 1H);

MS (APCI+) m/z 495 (M+H)⁺,

MS (APCI−) m/z 493 (M−H)⁻, 529 (M+Cl)⁻.

### Example 101G

### N-[3-cyclohexyl-5-(1H-imidazol-4-yl)-5,6,7,8-tetrahydro-1-naphthalenyl]ethanesulfonamide

The product from Example 101F (670 mg, 1.36 mmol) and 1 N HCl (5 mL) in tetrahydrofuran (10 mL) were refluxed for 2 hours. The mixture was allowed to cool to ambient temperature and the volume concentrated under reduced pressure. Solid sodium bicarbonate was added to the mixture to provide a solid. The solid was filtered, dried under reduced pressure and purified on a silica gel column (12:1 dichloromethane:methanol) to provide the title compound (365 mg).

mp 207-209° C.;

¹H NMR (300 MHz, DMSO-d₆) δ 1.26 (m, 8H), 1.70 (m, 7H), 1.93 (m, 2H), 2.33 (m, 1H), 2.72 (m, 2H), 3.10 (q, J=7 Hz, 2H), 4.03 (m, 1H), 6.5 (s, 1H), 6.75 (s,1H), 6.95 (s, 1H), 7.53 (s, 1H), 8.80 (s, 1H);

MS (APCI+) m/z 388 (M+H)⁺;

MS (APCI−) m/z 386 (M−H)⁻, 422 (M+Cl)⁻.

### EXAMPLE 102

### N-[5-(1H-imidazol-4-yl)-2-methyl-5,6,7,8-tetrahydro-1-naphthalenyl]ethanesulfonamide

### Example 102A

### 4-(3-methylphenyl)-4-oxo-2-butenoic Acid

3-Methylacetophenone (2.8 mL, 20 mmol), glyoxylic acid hydrate (2.76 g, 30 mmol) and 2N potassium hydroxide solution (17 mL) in methanol (30 mL) were stirred at ambient temperature for 12 hours and concentrated under reduced pressure. The aqueous residue was adjusted to pH 3 with the addition of citric acid and then extracted with ethyl acetate. The ethyl acetate layer was dried (MgSO₄), filtered and concentrated under reduced pressure to provide the title compound which was used immediately in the next step.

### Example 102B

### methyl 4-(3-me thlphenyl)-4-oxo-2-butenoate

The product from Example 102A in DMF (35 mL) was treated with sodium bicarbonate (4.2 g, 50 mmol) and methyl iodide (3 mL). After stirring for 24 hours, the mixture was diluted with water and extracted with ethyl acetate. The ethyl acetate layer was washed with water, brine, dried (MgSO₄), filtered and concentrated under reduced pressure. The residue was purified by column chromatography (silica gel, 3:1 hexane:ethyl acetate) to provide the title compound (1.2 g).

¹H NMR (300 MHz, DMSO-d₆) δ 2.41 (s, 3H), 3.80 (s, 3H), 6.74 (d, J=15 Hz, 1H), 7.50 (m, 2H), 7.84 (m, 2H), 7.96 (d, J=15 Hz, 1H);

MS (APCI+) m/z 205 (M+H)⁺.

### Example 102C

### 4-(3-methalphenyl)butanoic Acid

The product from Example 102B (1.2 g, 6 mmol) in methanol (12 mL) was treated with concentrated HCl (2 drops) and 20% Pd(OH)₂/C (121 mg). The mixture was hydrogenated under 60 psi pressure for 4 hours. The catalyst was filtered off and the filtrate was concentrated under reduced pressure to provide almost pure (1.1 g, 95%) saturated ester. The ester was dissolved in methanol and treated with 1M sodium hydroxide solution (10 mL). After stirring at ambient temperature for 6 hours, the mixture was acidified with concentrated HCl and extracted with dithyl ether. The ether layer was washed with brine, dried (MgSO₄), filtered and concentrated to provide (1 g, 100%) the title compound.

¹H NMR (300 MHz, DMSO-d₆) δ 1.77 (quintet, J=7 Hz, 2H), 2.20 (t, J=7 Hz, 2H), 2.30 (s, 3H), 2.53 (m, 2H), 7.00 (m, 3H), 7.17 (m, 1H); MS (DCI/NH₃) m/z 196 (M+NH₄)+.

### Example 102D

### 6-methyl-3,4-dihydro-1 (2H)-naphthalenone

The product from Example 102C (976 mg, 5.47 mmol) in dichloromethane (100 mL) under a nitrogen atmosphere was treated with boron trifluoride diethyl etherate (1.86 mL, 15 mmol) and trifluoroacetic anhydride (2.12 mL, 15 mmol). After stirring at ambient temperature for 12 hours, the mixture was concentrated and the residue was purified using column chromatography (silica gel, 3:2 hexane:ether) to provide (860 mg, 98%) the title compound.

¹H NMR (300 MHz, CDCl₃) δ 2.13 (quintet, J=7 Hz, 2H), 2.38 (s, 3H), 2.63 (t, J=7 Hz, 2H), 2.92 (t, J=7 Hz, 2H), 7.07 (m, 1H), 7.12 (m, 1H), 7.94 (d, J=9 Hz, 1H); MS (DCI/NH₃) m/z 161 (M+H)⁺, 178 (M+NH₄)⁺.

### Example 102E

### 6-methyl-5-nitro-3,4-dihydro-1(2H)-naphthalenone

The product from Example 102D was processed as described in Example 101 D. The residue was purified by column chromatography (silica gel, 6.5:3.5 hexane:ethyl acetate) to provide (360 mg, 33%) the title compound.

¹H NMR (300 MHz, CDCl₃) δ 2.06(quintet, J=7 Hz, 2H), 2.35 (s, 3H), 2.65 (t, J=7 Hz, 2H), 2.82 (t, J=7 Hz, 2H), 7.5 (d, J=9 Hz, 1H), 8.00 (d, J=9 Hz,1H).

### Example 102F

### N,N-dimethyl-4-(6-methyl-5-nitro-3,4-dihydro-1-naphthalenyl)-1H-imidazole-1-sulfonamide

The product from Example 102E (360 mg, 1.7 mmol) and 4-iodo-N,N-dimethyl-1H-imidazole-1-sulfonamide (0.90 g, 3 mmol), prepared as described in (R. M. Turner, J. Org. Chem. (1991) 56, 5739-5740), were processed as described in Example 101E to provide (175 mg) the title compound.

### Example 102G

### N-[5-(1H-imidazol-4-yl)-2-methyl-5,6,7,8-tetrahydro-1-naphthalenyl]ethanesulfonamide

The product from Example 102F in methanol (5 mL) was treated with 10% Pd/C under a hydrogen atmosphere (60 psi) at ambient temperature for 33 hours. The catalyst was removed by filtration and the filtrate was concentrated under reduced pressure. The residue was dissolved in dichloromethane (4 ml) and pyridine (0.08 mL), cooled to 0° C., and treated with ethanesulfonyl chloride (0.5 mL, 5 mmol) dropwise. After stirring at ambient temperature for 18 hours, the mixture was concentrated under reduced pressure. The residue was treated with 1N HCl (3 mL) and 1,4-dioxane (5 mL) and refluxed for 2 hours. The volume was reduced under reduced pressure and the remaining aqueous solution was neutralized with solid sodium bicarbonate and extracted with ethyl acetate. The ethyl acetate layer was dried (MgSO₄), filtered, concentrated under reduced pressure, and the residue purified on silica gel column (12:1 dichloromethane:methanol) to provide (20 mg) the title compound.

mp 196-199° C.;

¹H NMR (300 MHz, DMSO-d₆) δ 1.47 (m, 3H), 1.80 (m, 2H), 2.01 (m, 2H), 2.23 (m, 1H), 2.38 (s, 3H), 2.45 (m, 2H), 3.25 (q, J=7 Hz, 2H), 4.12 (t, J=7.5 Hz, 2H), 6.85 (d, J=9 Hz, 1H), 7.00 (d, J=9 Hz, 1H), 7.59 (s, 1H); MS (APCI+) m/z 320 (M+H)⁺.

### EXAMPLE 103

### N-[5-bromo-3-(1H-imidazol-4-ylmethyl)-2-methylphenyl]ethanesulfonamide

### Example 103A

### 5-bromo-2-methyl-3-nitrobenzaldehyde

2-Methyl-3-nitro benzyl alcohol (3.58 g, 21.6 mmol), prepared as described in (Gallagher, J. Med. Chem. 28, (1985) 1533-1536) in chloroform (75 ml) was treated with manganese (IV) oxide (1.86 g, 216 mmol). After 18 hours at reflux, The mixture was allowed to cool to ambient temperature, filtered through a bed of celite, and concentrated under reduced pressure to provide 2-methyl-3-nitrobenzaldehyde (2.75 g, 77%). The crude aldehyde was dissolved in trifluoroacetic acid (25 mL) and treated with sulfuric acid (7 mL) and N-bromosuccinimide (4.4 g, 24.8 mmol) portionwise. After stirring at 40° C. for 48 hours, the mixture was poured into ice water and the resultant solid was filtered and dried under reduced pressure to provide (3.48 g, 87%) the title compound.

¹H NMR (300 MHz, DMSO-d₆) δ 2.60 (s, 3H), 8.25 (d, J=3 Hz, 1H), 8.42 (d, J=3 Hz, 1H), 10.25 (s, 1H).

### Example 103B

### 4-[(5-bromo-2-methyl-3-nitrophenyl)(hydroxy)methyl]-N,N-dimethyl-1H-imidazole-1-sulfonamide

The product from Example 103A and 4-iodo-N,N-dimethyl-1H-imidazole-1-sulfonamide (0.90 g, 3 mmol), prepared as described in (R. M. Turner, J. Org. Chem. (1991) 56, 5739-5740), were processed as described in Example 1A except that after treatment with ammonium chloride solution the product was collected by filtration and dried under reduced pressure to provide (5.36 g, 90%) the title compound.

### Example 103C

### 4-(5-bromo-2-methyl-3-nitrobenzyl)-N,N-dimethyl-1H-imidazole-1-sulfonamide

The product from Example 103B was processed as described in Example 96C except that the crude product was kept under high vacuum instead of being chromatographed on silica gel to provide (4.21 g) crude product.

MS (APCI+) m/z 404 (M+H)⁺;

MS (APCI−) 438 (M+Cl)⁻.

### Example 103D

### 4-(3-amino-5-bromo-2-methylbenzyl)-N,N-dimethyl-1H-imidazole-1-sulfonamide

The product from Example 103C (1.2 g, 3 mmol) was processed as described in Example 96D except that after the reaction mixture was filtered through celite, the filtrate was concentrated and directly chromatographed on silica gel to provide 735 mg (66.8%) of title compound.

¹H NMR (300 MHz, DMSO-d₆) δ 1.94 (s, 3H), 2.78 (s, 6H), 3.73 (s, 2H), 5.19 (s, 2H), 6.53 (d, J=3 Hz, 1H), 6.69 (d, J=3 Hz, 1H), 7.21 (d, J=1.5 Hz, 1H), 8.05 (d, J=1.5 Hz, 1H); MS (APCI+) m/z 374 (M+H)⁺;

MS (APCI−) 408 (M+Cl)⁻.

### Example 103E

### N-[5-bromo-3-(1H-imidazol-4-ylmethyl)-2-methylphenyl]ethanesulfonamide

The product from Example 103D and ethanesulfonyl chloride were processed as described in Example 102G to provide 435 mg (61.5%) of the title compound.

mp 202-204° C.;

¹H NMR (300 MHz, DMSO-d₆) δ 1.36 (t, J=9 Hz, 3H), 2.25 (s, 3H), 3.1 (q, J=9 Hz, 2H), 3.93 (s, 2H), 6.53 (d, J=0.9 Hz, 1H), 7.2 (d, J=3 Hz, 1H), 7.42 (d, J=3 Hz, 1H), 7.6 (d,J=0.9 Hz, 1H);

MS (APCI+) m/z 359 (M+H)⁺;

MS (APCI−) m/z 357 (M−H)⁺393 (M+Cl)⁻.

### EXAMPLE 104

### N-[2-chloro-5-(1H-imidazol-4-ylmethyl)phenyl]ethanesulfonamide

The title compound was prepared according to the method of Example 21, substituting 4-chloro-5-nitrobenzaldehyde for 3-nitrobenzaldehyde in Example 21A and ethanesulfonyl chloride in place of methanesulfonyl chloride in Example 21D.

mp 159-160° C.;

¹H NMR (300 MHz, DMSO-d₆) 61.25(t, J=9 Hz, 3H), 3.10 (q, 2H), 3.83 (s, 2H), 6.79 (s, 1H), 7.10 (dd, J=1.5 Hz, 9 Hz 1H), 7.11 (d, J=1.5 Hz, 1H), 7.40 (d, J=1.5 Hz, 1H), 7.53 (s, 1H), 9.35 (bs, 1H);

MS (DCI/NH₃) m/z 300 (M+H)⁺.

### EXAMPLE 105

### N-[4-chloro-3-(1H-imidazol-4-ylmethyl)phenallethanesulfonamide

The title compound was prepared according to the method of Example 21, substituting 2-chloro-5-nitrobenzaldehyde for 3-nitrobenzaldehyde in Example 21A and ethanesulfonyl chloride in place of methanesulfonyl chloride in Example 21D.

¹H NMR (300 MHz, DMSO-d₆) δ 1.23 (t, J=9 Hz, 3H), 3.45 (q, 2H), 3.95 (s, 2H), 6.49 (dd, J=1.5 Hz, 9 Hz, 1H), 7.59 (m, 1H), 6.83 (s, 1H), 7.12 (d, J=9 Hz, 1H), 7.58 (s, 1H); MS (DC₁—NH₃) m/z 300 (M+H)⁺.

### EXAMPLE 106

### N-[2-chloro-3-(1H-imidazol-4-ylmethyl)phenyl]ethanesulfonamide

### Example 106A

### 2-chloro-3-nitrobenzaldehyde

A solution of of 2-chloro-3-nitrobenzoic acid (2.17 g, 12.0 mmol) in tetrahydrofuran (7.5 mL) and diethyl ether (7.5 mL) under nitrogen was heated to reflux, treated dropwise with of borane-methyl sulfide complex (0.95 g, 12 mmol), refluxed for 1 hour, cooled to ambient temperature and concentrated under reduced pressure to an oily residue. The residue was dissolved in dichloromethane (5 mL) and added to a rapidly stirred suspension of pyridinium chlorochromate (3.5 g, 16.5 mmol) in dichloromethane (20 ml) at ambient temperature. This mixture was refluxed for 2 hours, cooled to ambient temperature, filtered through celite and concentrated. The residue was purified by chromatography on silica gel eluting with 9:1 dichloromethane:ethyl acetate to provide 1.56 g of the title compound.

### Example 106B

### N-[2-chloro-3-(1H-imidazol-4-ylmethyl)phenyl]ethanesulfonamide

The title compound was prepared according to the method of Example 21, substituting the product from Example 106A for 3-nitrobenzaldehyde in Example 21A and ethanesulfonyl chloride in place of methanesulfonyl chloride in Example 21D.

mp 182-184° C.;

¹H NMR (300 MHz, DMSO-d₆) δ 1.26(t, J=9 Hz, 3H), 3.13 (q, 2H), 3.94 (s, 2H), 6.73 (s, 1H), 7.13 (dd, J=1.5 Hz, 9 Hz 1H), 7.23 (t, J=9 Hz, 1H), 7.33 (dd, J=1.5 Hz, 9 Hz 1H), 7.52 (s, 1H) 9.45 (bs, 1H);

MS (DCI/NH₃) m/z 300 (M+H)⁺.

### EXAMPLE 107

### N-[3-(1H-imidazol-4-ylmethyl)-4-methylphenyl]ethanesulfonamide

### Example 107A

### 2-methyl-5-nitrobenzaldehyde

The title compound was prepared according to the method described in Example 106A substituting 2-methyl-5-nitrobenzoic acid for 2-chloro-3-nitrobenzoic acid.

### Example 107B

### N-[3-(1H-imidazol-4-ylmethyl)-4-methylphenyl]ethanesulfonamide

The title compound was prepared according to the method of Example 21, substituting the product from Example 107A for 3-nitrobenzaldehyde in Example 21A and ethanesulfonyl chloride in place of methanesulfonyl chloride in Example 21D.

mp 194-196° C.;

1H NMR (300 MHz, DMSO-d₆) δ 1.16(t, J=9 Hz, 3H), 2.11 (s, 3H), 2.99 (q, 2H), 3.78 (s, 2H), 6.73 (s, 1H), 6.98 (m, 2H), 7.08 (m, 2H), 7.52 (s, 1H), 9.53 (bs, 1H);

MS (DCI/NH₃) m/z 280 (M+H)⁺.

### EXAMPLE 108

### N-[2-chloro-3-(1H-imidazol-4-ylmethyl)phenvyl]methanesulfonamide

The title compound was prepared according to the method of Example 21, substituting the product from Example 106A for 3-nitrobenzaldehyde in Example 21A.

mp 194-196° C.;

¹H NMR (300 MHz, DMSO-d₆) δ 3.03(s, 3H), 3.95 (s, 2H), 6.76 (s, 1H), 7.14 (dd, J=3 Hz, 9 Hz 1H), 7.24(t, J=9 Hz, 1H), 7.33 (dd, J=1.5 Hz, 9 Hz 1H), 7.53 (m, 1H) 9.45 (bs, 1H); MS (DCI/NH₃) m/z 286 (M+H)⁺.

### EXAMPLE 109

### N-[2-fluoro-5-(1H-imidazol-4-ylmethyl)phenyl]ethanesulfonamide

The title compound was prepared according to the method of example Example 106 substituting 4-fluoro-3-nitrobenzoic acid for 2-chloro-3-nitrobenzoic acid in Example 106A.

mp 122-123° C.;

¹H NMR (300 MHz, DMSO-d₆) δ 1.23(t, J=9 Hz, 3H), 3.08 (q, 2H), 3.81 (s, 2H), 6.79 (s, 1H), 7.08 (m, 1H), 7.16 (m, 1H), 7.24 (dd, J=3 Hz, 9 Hz, 1H), 7.55 (s, 1H), 9.51 (bs, 1H); MS (DCI/NH₃) m/z 284 (M+H)⁺.

### EXAMPLE 110

### N-[3-bromo-5-(1H-imidazol-4-ylmethyl)phenyl]ethanesulfonamide

The title compound was prepared according to the method of Example 21, substituting 5-bromo-3-nitrobenzaldehyde for 3-nitrobenzaldehyde in Example 21A and ethanesulfonyl chloride in place of methanesulfonyl chloride in Example 21D.

mp 194-196° C.;

¹H NMR (300 MHz, DMSO-d₆) δ 1.18(t, J=9 Hz, 3H), 3.13 (q, 2H), 3.81 (s, 2H), 6.81 (s, 1H), 7.08 (m, 1H), 7.12 (t, J=1 Hz, 1H), 7.20 (t, J=1 Hz, 1H), 7.54 (s, 1H), 9.96 (bs, 1H), 11.86 (bs, 1H); MS (DCI/NH₃) m/z 346 (M+H)⁺.

### Example 111

### N′-[5-(1H-imidazol-4-yl)-5,6,7,8-tetrahydro-1-naphthalenvyl]-N,N-dimethylsulfamide

The product from Example 12C and dimethylsulfamoyl chloride were processed as described in Example 12D to provide the title compound.

mp 208-210° C.;

¹H NMR (300 MHz, DMSO-d₆) δ 1.85(m, 4H), 2.75 (s, 6H), 2.81 (m, 2H), 4.05 (t, J=9 Hz, 1H), 6.53 (s, 1H), 6.84 (d, J=9 Hz, 1H), 7.03(t, J=9 Hz, 1H), 7.15(d, J=9 Hz, 1H), 7.54 (s, 1H), 8.86 (bs, 1H);

MS (DCI/NH₃) m/z 321 (M+H)⁺.

### EXAMPLE 112

### N′-[5-(1H-imidazol-4-yl)-5,6,7,8-tetrahydro-1-naphthalenyl]-N,N-dipropylurea

### Example 112A

### tert-butyl 4-{5-[(phenoxycarbonyl)amino]-1,2,3,4-tetrahydro-1-naphthalenyl}-1H-imidazole-1-carboxylate

A mixture of the polymer supported diisopropylamine (2 eq) in dichloromethane (25 mL) was treated with phenyl chloroformate (1.5 mL, 11.97 mmol), mixed sufficiently, treated with the product from Example 12C (2.50 g, 8.0 mmol), shaken at ambient temperature overnight, treated with polymer bound tris(2-aminoethyl)amine (5 eq) and shaken for 2 hours. The resin was filtered and washed with dichloromethane (2×25 mL). The combined filtrates were concentrated and purified by chromatography on silica gel eluting with ethyl acetate:hexane (1:1) to provide 2.79 g (81%) of the title compound.

### Example 112B

### N′-[5-(1H-imidazol-4-yl)-5,6,7,8-tetrahydro-1-naphthalenyl]-N,N-dipropylurea

A solution of dipropylamine (12.8 mg, 0.13 mmol) in methyl sulfoxide (0.3 mL) was treated with the product from Example 112A in methyl sulfoxide (0.55 mL), shaken for 16 hours, concentrated to dryness under reduced pressure, treated with 30% trifluoroacetic acid in dichloromethane(1.5 mL), shaken for 16 hours and concentrated under reduced pressure. The residue was purified by reverse phase preparative HPLC to provide 0.054 g (100%) of the title compound.

¹H NMR (500 MHz, DMSO-d₆) δ 0.87 (t, J=7.3 Hz, 6H), 1.55 (m, 4H), 1.74 (m, 2H), 1.98 (m, 2H), 2.67 (m, 2H), 3.24 (t, J=7.7 Hz, 4H), 4.31 (t, J=6.4 Hz, 1H), 6.71 (d, J=7.7 Hz, 1H), 7.06 (t, J=7.9 Hz, 1H), 7.14 (d, J=7.7 Hz, 1H), 7.19 (s, 1H), 7.58 (s, 1H), 9.02 (d, J=1.4 Hz, 1H), 14.26 (bs, 1H).

MS (ESI+) m/z 341 (M+H)⁺.

### EXAMPLE 113

### N-cyclohexyl-N-ethyl-N′-[5-(1H-imidazol-4-yl)-5,6,7,8-tetrahydro-1-naphthalenyl]urea

The product from Example 112A and N-cyclohexyl-N-ethylamine were processed as described in Example 112B to provide the title compound (17.3 mg, 31% yield).

¹H NMR (500 MHz, DMSO-d₆) δ 1.04 (t, J=7.0 Hz, 3H), 1.21 (m, 2H), 1.37 (m, 2H), 1.54 (m, 4H), 1.66 (m, 4H), 1.89 (m, 2H), 2.58 (m, 2H), 3.2 (q, J=7.2 Hz, 2H), 3.85 (m, 2H)), 4.22 (t, J=6.4 Hz, 1H), 6.62 (d, J=7.6 Hz, 1H), 6.97 (t, J=7.85 Hz, 1H), 7.61 (d, J=7.2 Hz, 1H), 7.11 (d, J=0.9 Hz, 1H), 7.50 (s, 1H), 8.95 (d, J=1.7 Hz, 1H), 14.10 (bs, 0.5H), 14.31(bs, 0.5H). MS (ESI+) m/z 367 (M+H)⁺.

### EXAMPLE 114

### N-[5-(1H-imidazol-4-yl)-5,6,7,8-tetrahydro-1-naphthalenyl]-1-piperidinecarboxamide

The product from Example 112A and piperidine were processed as described in Example 112B to provide the title compound (20.7 mg, 41% yield).

¹H NMR (500 MHz, DMSO-d₆) δ 1.50 (m, 4H), 1.59 (m, 2H), 1.74(m, 2H), 1.98 (m, 2H), 2.66 (t, J=6.6 Hz, 2H), 3.41 (t, J=5.3 Hz, 4H), 4.31 (t, J=6.6 Hz, 1H), 6.7 (d, J=7.6 Hz, 1H), 7.06 (t, J=7.65 Hz, 1H), 7.11 (m, 1H), 7.23 (d, J=1.3 Hz, 1H), 7.91(s, 1H), 9.04 (d, J=1.7 Hz, 1H), 14.16 (bs, 0.5H), 14.39(bs, 0.5H).

MS (ESI+) m/z 325 (M+H)⁺.

### EXAMPLE 115

### N-[5-(1H-imidazol-4-yl)-5,6,7,8-tetrahydro-1-naphthalenyl]-3,5-dimethyl-1-piperidinecarboxamide

The product from Example 112A and 3,5-dimethylpiperidine were processed as described in Example 112B to provide the title compound (47.6 mg, 88% yield).

¹H NMR (500 MHz, DMSO-d₆) δ 0.74 (m, 0.5H), 0.86 (d, J=6.6 Hz, 4H), 0.92 (d, J=7.0 Hz, 2H), 1.41 (m, 0.5H), 1.55(m, 1H), 1.78 (m, 3H), 1.99 (m, 2H), 2.25 (t, J=12.1 Hz, 1H), 2.67 (m, 2H), 3.12 (m, 1H), 3.47 (m, 1.5H), 4.05 (m, 1.5H), 4.32 (t, J=6.2 Hz, 1H), 6.71 (d, J=7.3 Hz, 1H), 7.06 (t, J=7.70 Hz, 1H), 7.09(m, 1H), 7.21 (bs, 1H), 7.79(s, 0.3H), 7.92(s, 0.7H), 9.0 (s, 1H), 14.22 (bs, 1H).

MS (ESI+) m/z 353 (M+H)⁺.

### EXAMPLE 116

### N′-[5-(1H-imidazol-4-yl)-5,6,7,8-tetrahydro-1-naphthalenyl]-N,N-bis(2-methoxyethyl)urea

The product from Example 112A and bis(2-methoxyethyl)amine were processed as described in Example 112B to provide the title compound (56.7 mg, 100% yield).

¹H NMR (500 MHz, DMSO-d₆) δ 1.78 (m, 2H), 1.97 (m, 2H), 2.59 (m, 2H), 3.32 (s, 6H), 3.52 (m, 8H), 4.31 (t, J=6.2 Hz, 1H), 6.61 (d, J=7.3 Hz, 1H), 7.04 (t, J=7.9 Hz, 1H), 7.23 (s, 1H), 7.39 (d, J=8.1 Hz, 1H), 7.83 (s, 1H), 9.00 (s, 1H), 14.20 (bs, 1H).

MS (EST+) m/z 373 (M+H)⁺.

### EXAMPLE 117

### N-[5-(1H-imidazol-4-yl)-5,6,7,8-tetrahydro-1-naphthalenyl]-4-morpholinecarboxamide

The product from Example 112A and morpholine were processed as described in Example 112B to provide the title compound (47.9 mg, 94% yield).

¹H NMR (500 MHz, DMSO-d₆) δ 1.75 (m, 2H), 1.98 (m, 2H), 2.67 (t, J=6.4 Hz, 2H), 3.41 (t, J=4.8 Hz, 4H), 3.62 (t, J=4.8 Hz, 4H), 4.32 (t, J=6.5 Hz, 1H), 6.72 (d, J=7.7 Hz, 1H), 7.07 (t, J=7.9 Hz, 1H), 7.14 (d, J=7.4 Hz, 1H), 7.22 (d, J=0.7 Hz, 1H), 7.99 (s, 1H), 9.02 (d, J=1.4 Hz, 1H), 14.28 (bs, 1H). MS (ESI+) m/z 327 (M+H)⁺.

### EXAMPLE 118

### N-ethyl-N′-[5-(1H-imidazol-4-yl)-5,6,7,8-tetrahydro-1-naphthalenyl]-N-isopropylurea

The product from Example 112A and N-ethyl-N-isopropylamine were processed as described in Example 112B to provide the title compound (34.5 mg, 68% yield).

¹H NMR (500 MHz, DMSO-d₆) δ 1.14 (d, J=6.5 Hz, 6H), 1.08 (m, 3H), 1.75 (m, 2H), 1.98 (m, 2H), 2.68 (m, 2H), 3.27 (m, 2H), 4.34 (m, 2H), 6.70 (d, J=7.6 Hz, H), 7.06 (t, J=7.7 Hz, 1H), 7.17 (d, J=7.6 Hz, 1H), 7.20 (s, 1H), 7.52 (s, 1H), 9.01 (d,J=1.1 Hz, 1H), 14.21 (bs, 1H).

MS (ESI+) m/z 327 (M+H)⁺.

### EXAMPLE 119

### methyl 5-(1H-imidazol-4-yl)-5,6,7,8-tetrahydro-1-naphthalenylcarbamate

Polymer supported diisopropylamine (2 equivalents) was treated with dichloromethane (0.75 mL) and methyl chloroformate (25.3 mg, 0.27 mmol, 1 equivalent), mixed well, treated with a solution of the product from Example 12C in dichloromethane (1 mL), shaken for 16 hours, treated with polymer bound tris(2-aminoethyl)amine (5 equivalents) and shaken for 2 hours. The resin was removed by filtration and was washed with dichloromethane (2×, 1 mL). The combined filtrates were concentrated under reduced pressure to dryness, treated with 30% trifluoroacetic acid in dichloromethane (1.5 mL), shaken for 16 hours and concentrated under reduced pressure. The residue was purified using reverse phase preparative HPLC to provide the title compound (47.4 mg, 69% yield).

¹H NMR (500 MHz, DMSO-d₆) δ 1.75 (m, 2H), 1.97 (m, 2H), 2.69 (t, J=6.4 Hz, 2H), 3.65 (s, 3H), 4.31 (t, J=6.6 Hz, 1H), 6.71 (d, J=7.7 Hz, 1H), 7.10 (t, J=7.9 Hz, 1H), 7.27 (m, 2H), 8.79 (s, 1H), 8.97 (s, 1H), 14.20 (bs, 1H). MS (ESI+) m/z 272 (M+H)⁺.

### EXAMPLE 120

### ethyl 5-(1H-imidazol-4-yl)-5,6,7,8-tetrahydro-1-naphthalenylcarbamate

The product from Example 12C and ethyl chloroformate were processed as described in Example 119 to provide the title compound (54.3 mg, 76% yield).

¹H NMR (500 MHz, DMSO-d₆) δ 1.24 (t, J=7.0 Hz, 3H), 1.75 (m, 2H), 1.96 (m, 2H), 2.69 (t, J=6.4 Hz, 2H), 4.10 (q, J=7.1 Hz, 2H), 4.31 (t, J=6.6 Hz, 1H), 6.71 (d, J=7.6 Hz, 1H), 7.09 (t, J=7.9 Hz, 1H), 7.26 (d, J=1.1 Hz, 1H), 7.28 (s, 1H), 8.75 (s, 1H), 8.97 (s, 1H), 14.20 (bs, 1H).

MS (ESI+) m/z 286 (M+H)⁺.

### EXAMPLE 121

### 2,2,2-trichloroethyl 5-(1H-imidazol-4-yl)-5,6,7,8-tetrahydro-1-naphthalenylcarbamate

The product from Example 12C and 2,2,2-trichloroethyl chloroformate were processed as described in Example 119 to provide the title compound (81.0 mg, 90% yield).

¹H NMR (500 MHz, DMSO-d₆) δ 1.76 (m, 2H), 1.97 (m, 2H), 2.73 (t, J=6.6 Hz, 2H), 4.31 (t, J=6.6 Hz, 1H), 4.92 (s, 2H), 6.79 (d, J=7.7 Hz, 1H), 7.12 (t, J=7.9 Hz, 1H), 7.21 (m, 2H), 8.86 (s, 1H), 9.36 (s, 1H), 14.10 (bs, 1H).

MS (ESI+) m/z 388 (M+H)⁺.

### EXAMPLE 122

### 2,2,2-trichloro-1-dimethylethyl 5-(1H-imidazol-4-yl)-5,6,7,8-tetrahydro-1-naphthalenylcarbamate

The product from Example 12C and 2,2,2-trichloro-1,1-dimethylethyl chloroformate were processed as described in Example 119 to provide the title compound (81.1 mg, 86% yield).

¹H NMR (500 MHz, DMSO-d₆) δ 1.75 (m, 2H), 1.889 (s, 3H), 1.893 (s, 3H), 1.96 (m, 2H), 2.71 (t, J=6.45 Hz, 2H), 4.31 (t, J=6.55 Hz, 1H), 6.78 (d, J=7.7 Hz, 1H), 7.10 (t, J=7.7 Hz, 1H), 7.18 (m, 2H), 8.95 (m, 2H), 14.20 (bs, 1H).

MS (ESI+) m/z 416 (M+H)⁺.

### EXAMPLE 123

### (1S,2R,5S)-2-isopropyl-5-methylcyclohexyl 5-(1H-imidazol-4-yl)-5,6,7,8-tetrahydro-1-naphthalenylcarbamate

The product from Example 12C and (+) menthyl chloroformate were processed as described in Example 119 to provide the title compound (59.9 mg, 66% yield).

¹H NMR (500 MHz, DMSO-d₆) δ 0.78 (d, J=6.6 Hz, 3H), 0.91 (m, 7H), 1.04 (m, 2H), 1.37 (m, 1H), 1.47 (m, 1H), 1.70 (m, 4H), 1.96 (m, 4H), 2.67 (m, 2H), 4.31 (m, 1H), 4.54 (m, 1H), 6.71 (d, J=7.6 Hz, 1H), 7.09 (t, J=7.65 Hz, 1H), 7.26 (m, 2H), 8.72 (d, J=0.7 Hz, 1H), 8.99 (s, 1H), 14.20 (bs, 1H).

MS (ESI+) m/z 396 (M+H)⁺.

### EXAMPLE 124

### 4-methylphenyl 5-(1H-imidazol-4-yl)-5,6,7,8-tetrahydro-1-naphthalenylcarbamate

The product from Example 12C and p-tolyl chloroformate were processed as described in Example 119 to provide the title compound. (5.3 mg, 30% yield).

¹H NMR (500 MHz, DMSO-d₆) δ 1.73 (m, 2H), 1.91 (m, 2H), 2.24 (s, 3H), 2.72 (t, J=6.66 Hz, 2H), 4.31 (t, J=6.55 Hz, 1H), 6.70 (d, J=7.6 Hz, 1H), 7.01 (d, J=8.4 Hz, 2H), 7.06 (t, J=7.9 Hz, 1H), 7.14 (d, J=8.4 Hz, 2H), 7.19 (s, 1H), 7.26 (d, J=7.7 Hz, 1H), 8.87 (s, 1H), 9.28 (d, J=1.1 Hz, 1H), 14.20 (bs, 1H).

MS (ESI+) m/z 348 (M+H)⁺.

### EXAMPLE 125

### methyl 3-(1H-imidazol-4-ylmethyl)phenylcarbamate

Polymer supported diiospropylamine (2 equivalents) was treated with dichloromethane (0.75 mL) and methyl chloroformate (25.3 mg, 0.27 mmol, 1 equivalent), mixed well, treated with the product from Example 21C (75 mg, 0.27 mmol) in dichloromethane (1 mL), shaken for 16 hours, treated with polymer bound tris(2-aminoethyl)amine (5 equivalents) and shaken for 2 hours. The resin was removed by filtration and washed with dichloromethane (2×, 1 mL). The combined filtrates were concentrated under reduced pressure to dryness, treated with 1,4-dioxane (0.75 mL) and 4M hydrochloric acid in 1,4-dioxane (0.75 mL), shaken at 75° C. for 16 hours, cooled and concentrated to dryness. The crude material was purified using reverse phase preparative HPLC to provide the title compound (12.2 mg, 20% yield).

¹H NMR (500 MHz, DMSO-d₆) δ 3.65 (s, 3H), 3.99 (s, 2H), 6.88 (d, J=7.9 Hz, 1H), 7.24 (t, J=7.7 Hz, 1H), 7.30 (m, 1H), 7.36 (s, 1H), 7.42 (d, J=1.1 Hz, 1H), 8.93(d, J=1.5 Hz, 1H), 9.61 (s, 1H), 14.20 (bs, 1H).

MS (ESI+) m/z 232 (M+H)⁺.

### EXAMPLE 126

### 2,2,2-trichloroethyl 3-(1H-imidazol-4-ylmethyl)phenylcarbamate

The product from Example 21C and 2,2,2-trichloroethyl chloroformate were processed as described in Example 125 to provide the title compound. (69.0 mg, 84% yield).

¹H NMR (500 MHz, DMSO-d₆) δ 4.01 (s, 2H), 4.93 (s, 2H), 6.95 (d, J=7.7 Hz, 1H), 7.28 (t, J=7.9 Hz, 1H), 7.36 (m, 1H), 7.43 (m, 2H), 8.95 (d, J=1.5 Hz, 1H), 10.12 (s, 1H), 14.20 (bs, 1H).

MS (ESI+) m/z 348 (M+H)⁺.

### EXAMPLE 127

### 2-chloroethyl 3-(1H-imidazol-4-ylmethyl)phenylcarbamate

The product from Example 21C and 2-chloroethyl chloroformate were processed as described in Example 125 to provide the title compound (20.5 mg, 75% yield).

¹H NMR (500 MHz, DMSO-d₆) δ 3.94 (t, J=5.15 Hz, 2H), 4.08 (s, 2H), 4.42 (t, J=5.1 Hz, 2H), 6.98 (d, J=7.7 Hz, 1H), 7.33 (t, J=7.85 Hz, 1H), 7.41(d, J=8.4 Hz, 1H), 7.48 (s, 1H), 7.52 (s, 1H), 9.05 (m, 1H), 9.87 (s, 1H), 14.20 (bs, 1H).

MS (ESI+) m/z 280 (M+H)⁺.

### EXAMPLE 128

### N-[3-(1H-imidazol-4-ylmethyl)phenyl]propanamide

Propionic acid (23.8 mg, 1.5 equivalents) in dichloromethane (4 ml) was treated with 1-hydroxybenzotriazole hydrate (1.7 equivalents) in a 1:1 mixture of dichloromethane and N,N-dimethylformamide (1 mL), N-cyclohexylcarbodiimide, N′-methyl polystyrene resin (2.0 eq, Novabiochem), agitated for 20 minutes, treated with the product from Example 21C in dichloromethane (1 mL), shaken at ambient temperature over night, treated with polymer bound tris(2-aminoethyl)amine (5 equivalents) and shaken for 2 hours. The resin was removed by filtration and washed with dichloromethane (2×1 mL). The combined filtrates were concentrated under reduced pressure to dryness, treated with 1,4-dioxane (0.75 mL) and 4M hydrochloric acid in 1,4-dioxane (0.75 mL), shaken at 75° C. for 6 hours and concentrated to dryness. The crude material was purified using reverse phase preparative HPLC to provide the title compound (14.2 mg, 19% yield).

¹H NMR (500 MHz, DMSO-d₆) δ 1.06 (t, J=7.5 Hz, 3H), 2.29 (q, J=7.6 Hz, 2H), 4.00 (s, 2H), 6.92 (d, J=7.3 Hz, 1H), 7.25 (t, J=7.9 Hz, 1H), 7.41 (d, J=7.8 Hz, 1H), 7.44 (s, 1H), 7.53 (s, 1H), 8.96 (s, 1H), 9.80 (s, 1H), 14.12 (bs, 1H).

MS (ESI+) m/z 230 (M+H)⁺.

### EXAMPLE 129

### N-[3-(1H-imidazol-4-ylmethyl)phenyl]butanamide

The product from Example 21C and butyric acid were processed as described in Example 128 to provide the title compound (20.5 mg, 27% yield).

¹H NMR (500 MHz, DMSO-d₆) δ 0.90 (t, J=7.5 Hz, 3H), 1.59 (m, 2H), 2.26 (t, J=7.4 Hz, 2H), 3.99 (s, 2H), 6.92 (d, J=7.7 Hz, 1H), 7.25 (t, J=7.9 Hz, 1H), 7.42 (m, 2H), 7.54 (s, 1H), 8.95 (d, J=1.1 Hz, 1H), 9.83 (s, 1H), 14.12 (bs, 1H).

MS (ESI+) m/z 244 (M+H)⁺.

### EXAMPLE 130

### 2,2,2-trifluoro-N-[3-(1H-imidazol-4-ylmethyl)phenyl]acetamide

The product from Example 21C and trifluoroacetic acid were processed as described in Example 128 to provide the title compound (11.1 mg, 14% yield).

¹H NMR (500 MHz, DMSO-d₆) δ 4.06 (s, 2H), 7.14 (d, J=7.9 Hz, 1H), 7.38 (t, J=7.9 Hz, 1H), 7.46 (s, 1H), 7.54 (m, 2H), 8.97(d, J=1.2 Hz, 1H), 11.23 (s, 1H), 14.16 (bs, 1H).

MS (ESI+) m/z 270 (M+H)⁺.

### EXAMPLE 131

### N-[3-fluoro-5-(1H-imidazol-4-yl)-5,6,7,8-tetrahydro-1-naphthalenyl]ethanesulfonamide

### Example 131A

### 7-fluoro-3,4-dihydro-1 (2H)-naphthalenone Oxime

A solution of 7-fluoro-3,4-dihydro-1 (2H)-naphthalenone (prepared as described in Newman, Melvin S. J. Org. Chem., 45, 2, 1980, 348-349) (2.45 g, 14.9 mmol) was treated with hydroxylamine hydrochloride (3.13 g, 45 mmol) and sodium acetate (3.7 g, 45 mmol) in water (3 mL) and heated at reflux for 24 hours. The mixture was allowed to cool to ambient temperature, concentrated and triturated with water. The resulting solid was collected by filtration and dried to provide (2.4 g, 100%) the title compound.

MS (DCI/NH₃) m/z 180 (M+H)⁺.

### Example 131B

### 8-fluoro-1,3,45-tetrahydro-2H-1-benzazepin-2-one

A solution of polyphosphoric acid (0.5 g) in toluene (5 mL) was heated to 85° C. and treated with the product from Example 131A (0.18 g, 1 mmol). After 30 minutes at reflux, the mixture was allowed to cool to ambient temperature, diluted with water, and extracted with ethyl acetate. The ethyl acetate layer was dried (MgSO₄), filtered, and concentrated to provide 0.16 g (89%) of the title compound. MS (DCI/NH₃) m/z 180 (M+NH₄)+.

### Example 131C

### 4-{2-[(ethylsulfonyl)amino]-4-fluorophenyl}butanoic Acid

Sodium hydride (60% dispersion) (0.72 g, 18 mmol) was washed with hexane, suspended in tetrahydrofuran (10 mL), cooled to 0° C., treated dropwise with a solution of the product from Example 131B (2.16 g, 12 mmol) in tetrahydrofuran (40 mL). After stirring at 0° C. for 1.5 hours, the mixture was treated with ethanesulfonyl chloride (1.93 g, 15 mmol). After stirring at ambient temperature for 2.5 hours, the mixture was treated with water (5 mL) and 1 M sodium hydroxide solution (24 mL) and extracted with diethyl ether. The aqueous layer was acidified with 1M HCl (25 mL) and extracted with dichloromethane. The dichloromethane layer was dried (MgSO₄), filtered and concentrated to provide the title compound (2.9 g, 84%).

MS (DCI/NH₃) m/z 307 (M+NH₄)+.

### Example 131 D

### N-(3-fluoro-5-oxo-5,6,7,8-tetrahydro-1-naphthalenyl)ethanesulfonamide

The product from Example 131 C (2.47 g, 8.5 mmol) in dichloromethane (25 mL) and dimethylformamide (0.025 mL) was treated with oxalyl chloride (2.16 g, 17 mmol) and stirred at ambient temperature for 24 hours. This solution was added to a 0° C. suspension of aluminum chloride (4.53 g, 34 mmol) in dichloromethane (25 mL). The mixture was stirred at ambient temperature for 60 hours, treated with water (50 mL) and extracted with dichloromethane. The dichloromethane layer was dried (MgSO₄), filtered and concentrated. The residue was purified by chromatography on silica gel eluting with 3:7 ethyl acetate:hexane to provide the title compound. is 15 MS (DCI/NH₃) m/z 289 (M+NH₄)+.

### Example 131E

### tert-butyl ethylsulfonyl(3-fluoro-5-oxo-5,6,7,8-tetrahydro-1-naphthalenyl)carbamate

The product from Example 131D (0.38 g, 1.4 mmol) in dichloromethane (7 mL) was treated with triethylamine (0.22 mL, 1.6 mmol), 4-dimethylaminopyridine (0.012 g, 0.1 mmol), and di-tert-butyl dicarbonate (0.33 g, 1.5 mmol). After stirring for 1.5 hours, the mixture was concentrated and the residue was purified by filtration through a pad of silica gel eluting with dichloromethane to provide the title compound.

### Example 131F

### N-[3-fluoro-5-(1H-imidazol-4-yl)-7,8-dihydro-1-naphthalenyl]ethane Sulfonamide

4-Iodo-N,N-dimethyl-1H-imidazole-1-sulfonamide (0.90 g, 3 mmol), prepared as described in (R. M. Turner, J. Org. Chem. (1991), 56, 5739-5740), in dichloromethane (10 mL) at 0° C. under nitrogen was treated with ethyl magnesium bromide (3.0M in diethyl ether, 1.1 mL). After stirring for 75 minutes at ambient temperature, the mixture was cooled to −10° C. and treated with the product from Example 131 E in dichloromethane (5 mL), stored over night at 0° C., warmed to ambient temperature, treated with methanol and 1M HCl (1 mL), washed with water, dried (MgSO₄), filtered and concentrated. The residue was treated with methanol (10 mL) and 1M HCl (10 mL), heated to reflux for 5 hours, cooled, diluted with water and washed with dichloromethane. The aqueous layer was neutralized with Na₂CO₃ solution and extracted with ethyl acetate. The combined ethyl acetate layers were dried (MgSO₄), filtered and concentrated to provide 0.29 g of the title compound.

MS (ESI+) m/z 322 (M+H)⁺;

MS (ESI−) m/z 320 (M−H)⁻.

### Example 131G

### N-[3-fluoro-5-(1H-imidazol-4-yl)-5,6,7,8-tetrahydro-1-naphthalenyl]ethanesulfonamide

The product from Example 131 F in ethanol was processed as described in Example 1 C to provide the title compound.

¹H NMR (CD₃OD) δ 1.36 (t, 3H), 1.74-1.82 (m, 1H), 1.84-1.93 (m, 1H), 2.00-2.06 (m, 2H), 2.72-2.81 (m, 2H), 3.16 (q, 2H), 4.13 (t, 1H), 6.57 (dd, 1H), 6.63 (s, 1H), 7.04 (dd, 1H), 7.59 (s, 1H);

MS (APCI+) m/z 324 (M+H)⁺;

MS (APCI−) m/z 322 (M−H)⁻;

Anal. Calcd for C₁₅H₈FN₃O₂S 0.25H₂O 0.1 EtOH: C, 54.91; H, 5.79; N, 12.64. Found: C, 54.84; H, 5.81; N, 12.65.

### EXAMPLE 132

### N-[3-chloro-5-(1H-imidazol-4-yl)-5,6,7,8-tetrahydro-1-naphthalenyl]ethanesulfonamide

7-Chloro-3,4-dihydro-2H-naphthalen-1-one, prepared as described in (Owton, W. Martin, Synth.Commun., 21; 8/9; 1991; 981-987), was processed as described in Example 131 except that the reaction time in Example 131 G was 2.5 hours instead of 16 hours to provide the title compound.

¹H NMR (CD₃OD) δ 1.37 (t, 3H), 1.73-1.83 (m, 1H), 1.83-1.93 (m, 1H), 1.98-2.08 (m, 2H), 2.75-2.85 (m, 2H), 3.16 (q, 2H), 4.13 (t, 1H), 6.64 (s, 1H), 6.85 (d, 1H), 7.27 (d, 1H), 7.63 (s, 1H);

MS (APCI+) m/z 340 (M+H)⁺;

MS (APCI−) m/z 338 (M−H)⁻;

Anal. Calcd for C₁₅H₁₈ClN₃O₂S 0.3H₂O 0.2 EtOH: C, 52.18; H, 5.63; N, 11.85. Found: C, 52.11; H, 5.54; N, 11.79.

### In vitro Binding Assays

For purposes of discussing a, adrenoceptor subtypes, the IUPHAR convention of using lower case letters to define molecular clones and upper case letters to indicate pharmacologically defined adrenoceptors has been followed. Compounds of formula I were evaluated in radioligand binding assays specific for α_{1A} (rat submaxillary gland), α_{1b} (hamster receptor expressed in mouse fibroblasts) and α_{1d} (rat receptor expressed in mouse fibroblasts) using [³H]-prazosin as the radioligand as described in Knepper, et al. J. Pharm. Exp. Ther. (1995), 274, 97-103. The results are shown in Table 1.

TABLE 1
Radioligand Binding Ki (nM)

| Example | α_{1A} (Rat) | α_{1b} (Hamster) | α_{1d} (Rat) |
|---|---|---|---|
| 1 | 500 | 1700 | 482 |
| 2 | 36.1 | 2520 | 1260 |
| 3 | 991 | 50400 | 8290 |
| 4 | 24600 | 31600 | 31600 |
| 5 | 136 | 6140 | 1550 |
| 6 | 2080 | 100000 | 16700 |
| 7 | 661 | 100000 | 3780 |
| 8 | 176 | 4620 | 1590 |
| 9 | 91.0 | 2000 | 1910 |
| 10 | 1150 | 10400 | 1690 |
| 12 | 95.9 | 6980 | 1670 |
| 14 | 277 | 2020 | 1040 |
| 15 | 204 | 10000 | 701 |
| 16 | 520 | 10000 | 10000 |
| 17 | 167 | 10000 | 3300 |
| 18 | 1140 | 10000 | 2050 |
| 19 | 475 | 10000 | 1150 |
| 20 | 253 | 10000 | 10000 |
| 22 | 91.9 | 2030 | 761 |
| 23 | 391 | 10000 | 2670 |
| 24 | 712 | 10000 | 10000 |
| 25 | 2460 | 10000 | 10000 |
| 26 | 1680 | 10000 | 10000 |
| 27 | 531 | 10000 | 2670 |
| 33 | 46.8 | 1300 | 1080 |
| 34 | 1300 | 10000 | 842 |
| 35 | 2180 | 10000 | 10000 |
| 36 | 124 | 10000 | 10000 |
| 38 | 1360 | 10000 | 2540 |
| 39 | 22.6 | 2010 | 969 |
| 40 | 1340 | 10000 | 10000 |
| 42 | 359 | 10000 | 2280 |
| 43 | 445 | 10000 | 2860 |
| 44 | 310 | 10000 | 3050 |
| 45 | 565 | 1730 | 971 |
| 46 | 553 | 1140 | 358 |
| 47 | 830 | 10000 | 2140 |
| 56 | 229 | 16.1 | 2.46 |
| 61 | 1560 | 10000 | 10000 |
| 62 | 1910 | 10000 | 10000 |
| 63 | 1720 | 10000 | 10000 |
| 64 | 4150 | 10000 | 10000 |
| 65 | 1160 | 4400 | 5300 |
| 66 | 366 | 1130 | 458 |
| 67 | 901 | 2390 | 3740 |
| 68 | 5280 | | |
| 69 | 4470 | 10000 | 10000 |
| 70 | 430 | 387 | 353 |
| 71 | 1540 | 1050 | 2290 |
| 72 | 1510 | 10000 | 2280 |
| 73 | 1020 | 1190 | 2230 |
| 74 | 5060 | | |
| 75 | 4600 | | |
| 76 | 3210 | | |
| 77 | 650 | 320 | 111 |
| 79 | 638 | 10000 | 10000 |
| 80 | 378 | 10000 | 1590 |
| 84 | 792 | 10000 | 10000 |
| 87 | 936 | 10000 | 10000 |
| 88 | 573 | 10000 | 10000 |
| 89 | 1010 | 10000 | 10000 |
| 90 | 237 | 10000 | 10000 |
| 91 | 1520 | 10000 | 10000 |
| 92 | 689 | 10000 | 1980 |
| 93 | 198 | 10000 | 10000 |
| 94 | 1940 | 1180 | 173 |
| 95 | 183 | 10000 | 10000 |
| 101 | 269 | 886 | 449 |
| 111 | 579 | 10000 | 1590 |
| 112 | 1240 | 10000 | 10000 |
| 113 | 536 | 10000 | 2110 |
| 114 | 1050 | 10000 | 5620 |
| 115 | 1160 | 10000 | 10000 |
| 116 | 2980 | 10000 | 10000 |
| 117 | 1100 | 10000 | 10000 |
| 118 | 329 | 10000 | 920 |
| 119 | 91.0 | 10000 | 3040 |
| 120 | 164 | 10000 | 2910 |
| 121 | 229 | 1850 | 963 |
| 122 | 1030 | 10000 | 1270 |
| 123 | 1280 | 1360 | 692 |
| 124 | 451 | 1470 | 787 |
| 131 | 90 | 10000 | 2164 |
| 132 | 47 | 10000 | 367 |

### In vitro Functional Assays

Compounds of formula I also were evaluated for their ability to stimulate contraction of smooth muscle tissues containing α_{1A} (rat epididymal vas deferens), α_{1B} (rat spleen) and α_{1D} (rat aorta) receptors as described in (Knepper, et al. J. Pharm. Exp. Ther. (1995), 274, 97-103), except that the endothelium was removed from the rat aorta strips. Most of the compounds were tested for α_{1A} functional activity using rabbit urethra as follows. Female New Zealand white rabbits (2.0-3.5 Kg) were sedated with CO₂ and decapitated. The entire urethra was removed and immediately placed into Krebs Ringer bicarbonate solution with the following mM concentrations: 120 NaCl, 20 NaHCO₃, 11 dextrose, 4.7 KCl, 2.5 CaCl₂, 1.5 MgSO₄, 1.2 KH₂PO₄, 0.01 K₂EDTA, equilibrated with 5% CO₂: 95% O₂ (pH=7.4 at 37° C.). Subsequent experimental conditions were as described above for the other tissues. Agonist concentration response curves were cumulative except for the vas deferens assay in which the transient response made such measurements impractical.

The in vitro functional data are shown in Table 2.

TABLE 2
Agonist Tissue Response (pD2)

| Example | α_{1A} (rab ureth) | α_{1B} (rat spleen) | α_{1D} (rat aorta) |
|---|---|---|---|
| 1 | <3.00* | 3.02 | 5.26 |
| 2 | 7.77* | 6.64 | 5.72 |
| 3 | 5.71* | 4.89 | 4.55 |
| 4 | 3.51* | <3.00 | 4.02 |
| 5 | 6.29 | 5.03 | 5.48 |
| 7 | <3.00* | <3.00 | |
| 8 | 6.35 | 5.29 | 4.37 |
| 9 | 6.45 | <3.00 | <3.00 |
| 10 | 4.67 | 4.19 | 4.19 |
| 12 | 6.20 | <3.00 | <3.00 |
| 14 | 5.25 | <3.00 | <3.00 |
| 15 | 6.16 | 5.65 | <3.00 |
| 16 | 5.89 | 4.84 | <3.00 |
| 17 | 6.28 | 5.47 | <3.00 |
| 18 | 5.17 | 4.85 | 4.96 |
| 19 | 6.00 | 5.09 | 5.08 |
| 20 | 5.55 | <3.00 | <3.00 |
| 22 | 6.78 | 5.52 | 5.07 |
| 23 | 4.40 | <3.00 | <3.00 |
| 24 | 4.61 | <3.00 | <3.00 |
| 25 | 4.48 | 3.12 | <3.00 |
| 26 | 4.81 | 4.74 | <3.00 |
| 27 | 5.25 | | |
| 33 | 6.77 | 5.42 | <3.00 |
| 34 | 4.55 | <3.00 | <3.00 |
| 35 | 4.09 | <3.00 | <3.00 |
| 36 | 5.73 | <3.00 | <3.00 |
| 38 | 4.72 | | |
| 39 | 7.18 | <3.00 | <3.00 |
| 42 | <3.00 | | |
| 43 | 4.35 | <3.00 | <3.00 |
| 44 | 5.31 | <3.00 | <3.00 |
| 45 | 5.70 | <3.00 | <3.00 |
| 46 | 4.90 | | |
| 47 | 4.54 | <3.00 | <3.00 |
| 56 | 4.79 | 3.73 | <3.00 |
| 67 | 3.97 | | |
| 70 | 4.09 | | |
| 73 | 4.75 | | |
| 77 | 4.54 | | |
| 90 | 6.31 | | |
| 92 | 5.49 | | |
| 93 | 6.00 | 4.58 | |
| 95 | 5.86 | 4.55 | |
| 112 | 5.10 | | |
| 113 | 5.40 | | |
| 114 | 5.04 | | |
| 115 | 5.26 | | |
| 116 | 5.03 | | |
| 117 | 5.27 | | |
| 121 | 5.43 | | |
| 123 | 5.21 | | |

*test performed using rat epididymal vas deferens

In vivo Functional Assays-Assessment of Intrurethral Pressure (IUP) and Mean Arterial Pressure (MAP) in Anesthetized Dogs

Female Beagle dogs (Marshall Farms, North Rose, N.Y.) greater that 2 years of age and weighing between 12 and 15 kg were used in these studies. At least 2 weeks prior to any agonist dosing, dogs were instrumented for the chronic measurement of arterial blood pressure by implanting a telemetry transducer/transmitter (TA11PA-C40, Data Sciences International, St. Paul, Minn.) into a carotid artery.

On the test day, dogs fasted since the previous afternoon were pre-anesthetized with thiopental sodium 15 mg/kg i.v. (Pentothal™, Abbott) and intubated. Anesthesia was maintained by allowing the dog to spontaneously breathe a mixture of isoflurane (2.5 to 3 volume %) and oxygen delivered by a Narkomed Standard anesthesia system (North American Drager, Telford, Pa.). An Abbocath-T™ i.v. catheter (18-G, Abbott) was inserted into the cephalic vein for the administration of agonists. A telemetry receiver (RA1310, DataSciences) was placed under the head of each dog and was interfaced to a computerized data acquisition system (Modular Instruments Inc.(MI2), Malvern, Pa.) which allowed for the continuous calibrated recording of arterial blood pressure which was electronically filtered to determine its mean value (MAP).

Dogs with chronic telemetry implants anesthetized as described above were placed in dorsal recumbency and a balloon catheter was inserted into the urethral orifice and advanced approximately 15 cm until the tip was well inside the bladder. The balloon was then inflated with 1 ml of room air and the catheter slowly withdrawn until resistance (corresponding to the bladder neck) was evident. The balloon was then deflated and the catheter withdrawn additional 2 cm. The balloon was then reinflated and its catheter port connected to a Gould Statham P23Dd pressure transducer interfaced to a computerized data acquisition system (Modular Instruments, Inc., Malvern, Pa.) for the measurement of the intraurethral pressure. Increasing iv doses of test agonists were administered and the maximum effect of each dose on IUP was recorded. The effect of each dose was allowed to return to baseline before the next dose was given.

From the resulting dose response curve, an ED₅ value, for the dose causing a maximum increase in IUP of 5 mm Hg, could be estimated. An ED₂₀ value for the dose causing a maximum increase in MAP of 20 mm Hg could also be estimated. A selectivity ratio of MAP ED₂₀ vs. IUP ED, was calculated. The mean of the selectivity ratio of MAP ED₂₀ vs. IUP ED₅ displayed in Table 3.

TABLE 3
IUP ED₅ Values for Test Compounds

| | Mean | Mean | Mean |
|---|---|---|---|
| | IUP ED₅ | MAP ED₂₀ | Selectivity Ratio |
| Example | (nmol/kg) | (nmol/kg) | MAP ED₂₀/IUP ED₅ |
| 8 | 25.5 | 102 | 4.8 |
| 9 | 20.4 | 48.7 | 4.4 |
| 10 | >1000 | >1000 | |
| 12 | 10.7 | 69.3 | 7.6 |
| 14 | >1000 | >1000 | |
| 15 | 91.9 | 225 | 2.5 |
| 16 | 68.4 | 220 | 3.1 |
| 17 | 187.7 | 216 | 1.1 |
| 19 | 67.1 | 164 | 2.3 |
| 20 | 208.9 | 885 | 6.1 |
| 26 | 653 | 1850 | 2.6 |
| 33 | 10.5 | 34.0 | 3.3 |
| 34 | >1000 | >1000 | |
| 36 | 156 | 508 | 3.6 |
| 39 | 4.4 | 5.6 | 1.3 |
| 44 | 892 | 588 | 1.0 |
| 45 | 273 | 633 | 2.6 |
| 80 | 113.28 | 125.78 | 1.12 |
| 92 | 397.22 | 652.06 | 1.73 |
| 93 | 38 | 75.23 | 2.28 |
| 95 | 112.5 | 109.1 | 0.99 |
| 113 | | 733.7 | |
| 114 | 1679.25 | 1074.07 | 0.97 |
| 115 | 500 | 500 | 1.05 |
| 117 | 873 | 645 | 0.75 |
| 132 | 19 | 47.7 | 2.5 |

### Assessment of Urethral Pressure Profile in Anesthetized Dogs

Dogs instrumented and anesthetized as decribed above were placed in left lateral recumbency and a dual pressure sensor catheter (SPC-771, Millar Instruments, Houston, Tex.) was inserted into the urethra and advanced into the bladder. The proximal pressure sensor was interfaced to a M12 computerized data acquisition system for the measurement of lower urinary tract pressures. At a resting intravesical pressure of approximately 5 cm of H₂O, urethral pressure was measured from the sensor as the catheter was withdrawn using a modified syringe pump (Model 22, Harvard Apparatus, South Natick, Mass.) at its maximal rate of 0.83 mm/sec. Measurement from the proximal sensor allowed easy reinsertion as the distal 5 cm of the catheter remains in the urethra after the total profile has been obtained. Three resting urethral pressure profiles were obtained at 5 minute intervals before dosing, then a single profile was initiated 30 sec after each increasing iv dose during the time corresponding to the maximum arterial pressure effects of that dose. The increase in arterial pressure seen after each agonist dose was allowed to return to baseline before the next dose was given.

FIG. 1 displays the urethral pressure profile for Example 8 from this invention. The Y axis displays the urethral pressure. The X axis displays the distance along the length of the urethra from the proximal to the distal end. FIG. 1 illustrates that increasing concentrations of Example 8 result in corresponding increases in the urethral pressure.

[image]

The results from Tables 1 and 2 show that the compounds of the invention bind to, stimulate, and show specificity for the UIA adrenoceptor and therefore may have utility in the treatment of diseases prevented by or ameliorated with compounds which activate the α_{1A} adrenoceptor. Table 3 illustrates that the compounds of this invention are efficacious in constricting the urethra. Table 3 also illustrates that these compounds are selective for constricting the urethra over increasing the mean arterial pressure. FIG. 1 illustrates that the compounds of this invention are efficacious in constricting the urethra in a manner, which is considered to be clinically relevant for the treatment of urinary incontinence.

The data in Table 3 demonstrates that compounds of the invention contract the smooth muscle of the urethra and hence may be useful for treating conditions such as retrograde ejaculation that result from deficient smooth muscle tone of the urethra and bladder neck.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions, which comprise compounds of the present invention formulated together with one or more non-toxic pharmaceutically acceptable carriers. Further included within the scope of the present invention are pharmaceutical compositions, comprising one or more of the compounds of formula I-VIII prepared and formulated in combination with one or more non-toxic pharmaceutically acceptable compositions. The pharmaceutical compositions can be formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions, which can be used, include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions, which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions, which can be used, include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention may include ointments, pastes, creams, lotions, gels, powders, solutions, sprays, inhalants or patches. The active component is admixed under sterile conditions with a pharmaceutically acceptable carrier and any needed preservatives or buffers as may be required. It is known that some agents may require special handling in the preparation of transdermal patch formulations. For example, compounds that are volatile in nature may require admixture with special formulating agents or with special packaging materials to assure proper dosage delivery. In addition, compounds, which are very rapidly absorbed through the skin, may require formulation with absorption-retarding agents or barriers. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The term “pharmaceutically acceptable cation,” as used herein, refers to a positively-charged inorganic or organic ion that is generally considered suitable for human consumption. Examples of pharmaceutically acceptable cations are hydrogen, alkali metal (lithium, sodium and potassium), magnesium, calcium, ferrous, ferric, ammonium, alkylammonium, dialkylammonium, trialkylammonium, tetraalkylammonium, diethanolammonium, and choline. Cations may be interchanged by methods known in the art, such as ion exchange. Where compounds of the present invention are prepared in the carboxylic acid form, addition of a base (such as a hydroxide or a free amine) will yield the appropriate cationic form.

The term “pharmaceutically acceptable salt, ester, amide, and prodrug,” as used herein, refers to carboxylate salts, amino acid addition salts, zwitterions, esters, amides, and prodrugs of compounds of formula I-VIII which are within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the invention include phosphate, tris and acetate.

The term “pharmaceutically acceptable ester” or “ester,” as used herein, refers to esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of formula I-VIII may be prepared according to conventional methods.

The term “pharmaceutically acceptable amide” or “amide,” as used herein, refers to non-toxic amides of the present invention derived from ammonia, primary C,-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula I-VIII may be prepared according to conventional methods.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to the parent compound of the above formula, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987), hereby incorporated by reference.

The term “prodrug ester group,” as used herein refers, to any of several ester-forming groups that are hydrolyzed under physiological conditions. Examples of prodrug ester groups include pivoyloxymethyl, acetoxymethyl, phthalidyl, indanyl and methoxymethyl, as well as other such groups known in the art. Other examples of prodrug ester groups can be found in the book “Pro-drugs as Novel Delivery Systems,” by Higuchi and Stella, cited above.

The present invention contemplates pharmaceutically active metabolites formed by in vivo biotransformation of compounds of formula I-VIII. The term pharmaceutically active metabolite, as used herein, refers to a compound formed by the in vivo biotransformation of compounds of formula I-VIII. The present invention contemplates compounds of formula I-VIII and metabolites thereof. A thorough discussion of biotransformation is provided in Goodman and Gilman's, The Pharmacological Basis of Therapeutics, seventh edition, hereby incorporated by reference.

The compounds of the invention, including but not limited to those specified in the examples, are α_{1A} adrenergic agonists. As α_{1A} agonists, the compounds of the present invention are useful for the treatment and prevention of diseases such as urinary incontinence and ejaculatory dysfunction such as retrograde ejaculation.

The ability of the compounds of the invention to treat urinary incontinence can be demonstrated according to the methods described (Testa, R. Eur. J. Pharmacol. (1993), 249, 307-315) and (Cummings, J. M. Drugs of Today (1996), 32, 609-614).

Aqueous liquid compositions of the present invention are particularly useful for the treatment and prevention of urinary incontinence and ejaculatory dysfunction.

When used in the above or other treatments, a therapeutically effective amount of one of the compounds of the present invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable excipients. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal may range from about 0.003 to about 10 mg/kg/day. For purposes of oral administration, more preferable doses can be in the range of from about 0.01 to about 5 mg/kg/day. If desired, the effective daily dose can be divided into multiple doses for purposes of administration; consequently, single dose compositions may contain such amounts or submultiples thereof to make up the daily dose.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated. However, it is within the skill of the art to start doses of the compound at levels lower than required for to achieve the desired therapeutic effect and to gradually increase the dosage until the desired effect is achieved.

What is claimed is:

1. A compound of formula I: 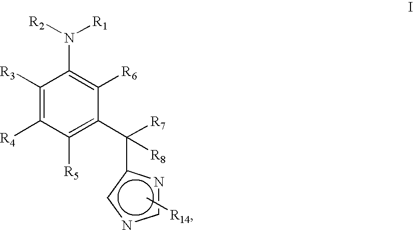
or a pharmaceutically acceptable salt thereof, wherein
R₁ is selected from the group consisting of —S(O)₂R₉ and —C(O)R₁₁;
R₉ is selected from the group consisting of alkenyl, alkyl, alkynyl, aryl, arylalkenyl, arylalkyl, cycloalkyl, cycloalkylalkyl, haloalkyl, heterocycle, and —NZ₁Z₂ wherein Z₁ and Z₂ are independently selected from the group consisting of hydrogen, alkyl, aryl, and arylalkyl;
R₁₀ is selected from the group consisting of alkenyl, alkoxy, alkyl, aryl, arylalkyl, aryloxy, cycloalkyl, cycloalkylalkyl, cycloalkyloxy, haloalkoxy, haloalkyl, and —NZ₃Z₄ wherein Z₃ and Z₄ are independently selected from the group consisting of hydrogen, alkoxyalkyl, alkyl, aryl, arylalkyl, and cycloalkyl, or Z₃ and Z₄ taken together with the nitrogen atom to which they are attached form a heterocycle selected from the group consisting of azetidin-1-yl, piperazin-1-yl, piperidin-1-yl, pyrrolidin-1-yl, and morpholin-4-yl wherein azetidin-1-yl, piperazin-1-yl, piperidin-1-yl, pyrrolidin-1-yl, and morpholin-4-yl are unsubstituted or substituted with 1 or 2 substituents independently selected from the group consisting of alkoxy, lower alkyl, and hydroxy;
R₂ is selected from the group consisting of hydrogen, lower alkyl, aryl, arylalkyl, cycloalkyl, cycloalkylalkyl, and haloalkyl;
R₃, R₄, R₅, and R₆ are independently selected from the group consisting of hydrogen, lower alkoxy, lower alkenyl, lower alkyl, lower haloalkyl, cycloalkyl, halo, and hydroxy; or
R₆ and R₇ together with the carbon atoms to which they are attached form a 5, 6, or 7 membered carbocyclic ring; or
R₆ and R₇ together with the carbon atoms to which they are attached form a 5 or 6 membered ring containing 1 heteroatom selected from the group consisting of O, NR₁₁, and S(O), wherein n is 0-2;
R₁₁, is selected from the group consisting of hydrogen, alkoxycarbonyl, alkyl, alkylcarbonyl, arylalkyl, formyl, —C(O)NZ₃Z₄, and —SO₂NZ₁Z₂;
R₈ is absent or hydrogen; or
R₇ and R₈ together form 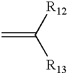
wherein R₁₂ and R₁₃ are independently selected from the group consisting of hydrogen, lower alkoxy, lower alkyl, aryl, arylalkyl, cycloalkyl, and cycloalkylalkyl;
provided that when R₇ and R, together form 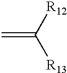
and R₁₂ and R₁₃ are independently selected from the group consisting of hydrogen, lower alkoxy, lower alkyl, aryl, arylalkyl, cycloalkyl, and cycloalkylalkyl then R₁ is S(O)₂R₉; or
R₁₂ and R₁₃ together with the carbon atom to which they are attached form a 3, 4, 5, 6, or 7 membered carbocyclic ring; or
R₁₂ and R₆ together with the carbon atoms to which they are attached form a 5, 6, or 7 membered carbocyclic ring;
provided that when R₁₂ and R₆ together with the carbon atoms to which they are attached form a 5, 6, or 7 membered carbocyclic ring then R₁₃ is hydrogen; or
R₁₂ and R₆ together with the carbon atoms to which they are attached form a 5 or 6 membered ring containing 1 heteroatom selected from the group consisting of O, NR₁₁, and S(O)ₙ;
provided that when R₁₂ and R₆ together with the carbon atoms to which they are attached form a 5 or 6 membered ring containing 1 heteroatom selected from the group consisting of O, NR₁₁, and S(O)ₙ then R₁₃ is hydrogen; and
R₁₄ is selected from the group consisting of hydrogen and lower alkyl.

2. A compound according to claim 1 wherein
R₁ is selected from the group consisting of —S(O)₂R, and —C(O)R₁₀;
R₉ is selected from the group consisting of alkyl, aryl, arylalkenyl, arylalkyl, cycloalkyl, haloalkyl, heterocycle, and —NZ₁Z₂ wherein Z₁ and Z₂ are independently selected from the group consisting of hydrogen and alkyl;
R₁₀ is selected from the group consisting of alkoxy, alkyl, aryloxy, cycloalkyl, cycloalkyloxy, haloalkoxy, haloalkyl, and —NZ₃Z₄ wherein Z₃ and Z₄ are independently selected from the group consisting of hydrogen, alkoxyalkyl, alkyl, and cycloalkyl, or Z₃ and Z₄ taken together with the nitrogen atom to which they are attached form a heterocycle selected from the group consisting of piperidin-1-yl and morpholin-4-yl wherein piperidin-1-yl, may be unsubstituted or substituted with 1 or 2 substituents selected from lower alkyl;
R₂ is selected from the group consisting of hydrogen and lower alkyl;
R₃ is selected from the group consisting of hydrogen, lower alkoxy, lower alkyl, lower haloalkyl, halo, and hydroxy;
R₄ is selected from the group consisting of hydrogen, lower alkoxy, lower alkyl, lower haloalkyl, cycloalkyl, halo, and hydroxy;
R₅ is selected from the group consisting of hydrogen, lower alkoxy, lower alkyl, lower haloalkyl, halo, and hydroxy;
R₆ is selected from the group consisting of hydrogen, lower alkoxy, lower alkenyl, lower alkyl, lower haloalkyl, halo, and hydroxy; or
R₆ and R₇ together with the carbon atoms to which they are attached form a 5, 6, or 7 membered carbocyclic ring; or
R₆ and R₇ together with the carbon atoms to which they are attached form a 5 or 6 membered ring containing 1 heteroatom selected from the group consisting of O, NR11, and S(O)ₙ wherein n is 0-2;
R₁₁ is selected from the group consisting of hydrogen, alkoxycarbonyl, alkyl, alkylcarbonyl, arylalkyl, formyl, —C(O)NZ₃Z₄, and —SO₂NZ₁Z₂;
R₈ is absent or hydrogen; or
R₇ and R₈ together form 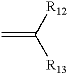
wherein R₁₂ and R₁₃ are independently selected from the group consisting of hydrogen, lower alkoxy, lower alkyl, aryl, arylalkyl, cycloalkyl, and cycloalkylalkyl; or
R₁₂ and R₁₃ together with the carbon atom to which they are attached form a 3, 4, 5, 6, or 7 membered carbocyclic ring; or
R₁₂ and R₆ together with the carbon atoms to which they are attached form a 5, 6, or 7 membered carbocyclic ring; or
R₁₂ and R₆ together with the carbon atoms to which they are attached form a 5 or 6 membered ring containing 1 heteroatom selected from the group consisting of O, NR₁₁, and S(O)ₙ; and
R₁₄ is selected from the group consisting of hydrogen and lower alkyl.

3. A compound according to claim 1 wherein
R₁ is selected from the group consisting of —S(O)₂R₉ and —C(O)R₁₀;
R₉ is selected from the group consisting of alkyl, aryl wherein aryl is selected from the group consisting of 2-methylphenyl, 4-methylphenyl, 4-methoxyphenyl, arylalkenyl wherein arylalkenyl is 2-phenylethenyl, arylalkyl wherein arylalkyl is benzyl, cycloalkyl wherein cycloalkyl is cyclopropyl, haloalkyl, heterocycle wherein heterocycle is selected from the group consisting of 3,5-dimethylisoxazol-4-yl, 1-methyl-1H-imidazol-4-yl, 5-chlorothien-2-yl, 5-chloro-1,3-dimethyl-1H-pyrazol-4-yl, quinolin-8-yl, 2-(methoxycarbonyl)thien-3-yl, 4-methyl-2-(acetylamino)thiazol-5-yl, and 5-chloro-3-methyl-1-benzothien-2-yl, and —NZ₁Z₂ wherein Z. and Z₂ are independently selected from the group consisting of hydrogen and alkyl;
R₁₀ is selected from the group consisting of alkoxy, alkyl, aryloxy wherein aryloxy is 4-methylphenoxy, cycloalkyloxy wherein cycloalkyloxy is ((1R,2S,5R)-2-isopropyl-5-methylcyclohexyl)oxy, haloalkoxy, haloalkyl, and —NZ₃Z₄ wherein Z₃ and Z₄ are independently selected from the group consisting of hydrogen, alkoxyalkyl, alkyl, and cycloalkyl wherein cycloalkyl is cyclohexyl, or Z₃ and Z₄ taken together with the nitrogen atom to which they are attached form a heterocycle selected from the group consisting of piperidin-1-yl and morpholin-4-yl wherein piperidin-1-yl may be unsubstituted or substituted with 1 or 2 substituents independently selected from lower alkyl;
R₂ is selected from the group consisting of hydrogen and lower alkyl;
R₃ is selected from the group consisting of hydrogen, lower alkoxy, lower alkyl, and hydroxy;
R₄ is selected from the group consisting of hydrogen, cycloalkyl wherein cycloalkyl is cyclohexyl, and halo;
R₅ is selected from the group consisting of hydrogen, lower alkoxy, lower alkyl, halo, and hydroxy;
R₆ is hydrogen; or
R₆ and R₇ together with the carbon atoms to which they are attached form a 5, 6, or 7 membered carbocyclic ring; or
R₆ and R₇ together with the carbon atoms to which they are attached form a 5 or 6 membered ring containing 1 heteroatom selected from the group consisting of O and S(O), wherein n is 0-2;
R₈ is absent or hydrogen; or
R₇ and R₈ together form 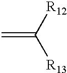
wherein R₁₂ and R₁₃ are independently selected from the group consisting of hydrogen, lower alkoxy, and lower alkyl; or R₁₂ and R₁₃ together with the carbon atom to which they are attached form a 6 membered carbocyclic ring; or
R₁₂ and R₆ together with the carbon atoms to which they are attached form a 6 membered carbocyclic ring; and
R₁₄ is selected from the group consisting of hydrogen and lower alkyl.

4. A compound according to claim 1 of formula II 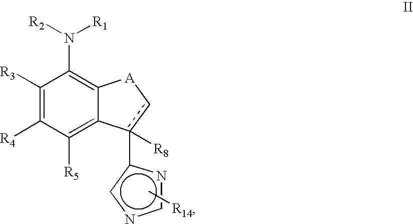
or a pharmaceutically acceptable salt thereof, wherein
A is selected from the group consisting of —CH₂—, —CH₂CH₂—, and —CH₂CH₂CH₂—; and [symbol]
represents a single bond or a double bond.

5. A compound according to claim 4 wherein
A is —CH₂—; [symbol]
is a single bond;
R₁ is C(O)R₁₀; and
R₈ is hydrogen.

6. A compound according to claim 4 wherein
A is —CH₂—; [symbol]
is a single bond;
R₁ is S(O)₂R₉; and
R₈ is hydrogen.

7. A compound according to claim 6 that is selected from the group consisting of
N-(1-(1H-imidazol-4-yl)-2,3-dihydro-1H-inden-4-yl)methanesulfonamide and
N-(1-(1H-imidazol-4-yl)-2,3-dihydro-1H-inden-4-yl)ethanesulfonamide.

8. A compound according to claim 4 wherein
A is —CH₂CH₂—; [symbol]
is a double bond;
R₁ is C(O)R₁₀; and
R₈ is absent.

9. A compound according to claim 4 wherein
A is —CH₂CH₂—; [symbol]
is a double bond;
R₁ is S(O)₂R₉; and
R₈ is absent.

10. A compound according to claim 9 that is N-(5-(1H-imidazol-4-yl)-7,8-dihydro-1-naphthalenyl)methanesulfonamide.

11. A compound according to claim 4 wherein
A is —CH₂CH₂—; [symbol]
is a single bond;
R₁ is C(O)R₁₀; and
R₈ is hydrogen.

12. A compound according to claim 11 selected from the group consisting of
N-[5,6,7,8-tetrahydro-5-(1H-imidazol-4-yl)-1-naphthalenyl]acetamide;
2,2,2-trifluoro-N-[5-(1H-imidazol-4-yl)-5,6,7,8-tetrahydro-1-naphthalenyl]acetamide;
5 N′-[5-(1H-imidazol-4-yl)-5,6,7,8-tetrahydro-1-naphthalenyl]-N,N-dipropylurea;
N-cyclohexyl-N-ethyl-N′-[5-(1H-imidazol-4-yl)-5,6,7,8-tetrahydro-1-naphthalenyl]urea;
N-[5-(1H-imidazol-4-yl)-5,6,7,8-tetrahydro-1-naphthalenyl]-1-piperidinecarboxamide;
N-[5-(1H-imidazol-4-yl)-5,6,7,8-tetrahydro-1-naphthalenyl]-3,5-dimethyl-1-piperidinecarboxamide;
N′-[5-(1H-imidazol-4-yl)-5,6,7,8-tetrahydro-1-naphthalenyl]-N,N-bis(2-methoxyethyl)urea;
N-[5-(1H-imidazol-4-yl)-5,6,7,8-tetrahydro-1-naphthalenyl]-4-morpholinecarboxamide;
N-ethyl-N′-[5-(1H-imidazol-4-yl)-5,6,7,8-tetrahydro-1-naphthalenyl]-N-isopropylurea;
methyl 5-(1H-imidazol-4-yl)-5,6,7,8-tetrahydro-1-naphthalenylcarbamate;
ethyl 5-(1H-imidazol-4-yl)-5,6,7,8-tetrahydro-1-naphthalenylcarbamate;
2,2,2-trichloroethyl 5-(1H-imidazol-4-yl)-5,6,7,8-tetrahydro-1-naphthalenylcarbamate;
2,2,2-trichloro-1,1-dimethylethyl 5-(1H-imidazol-4-yl)-5,6,7,8-tetrahydro-1-naphthalenylcarbamate;
(1S,2R,5 S)-2-isopropyl-5-methylcyclohexyl 5-(1H-imidazol-4-yl)-5,6,7, 8-tetrahydro-1-naphthalenylcarbamate; and
4-methylphenyl 5-(1H-imidazol-4-yl)-5,6,7,8-tetrahydro-1-naphthalenylcarbamate.

13. A compound according to claim 4 wherein
A is —CH₂CH₂—; [symbol]
is a single bond;
R₁ is S(O)₂R₉; and
R₈ is hydrogen.

14. A compound according to claim 13 selected from
N-[5-(1H-imidazol-4-yl)-2-methoxy-5,6,7,8-tetrahydro-1-naphthalenyl]methanesulfonamide;
N-[2-hydroxy-5-(1H-imidazol-4-yl)-5,6,7,8-tetrahydro-1-naphthalenyl]methanesulfonamide;
N-[2-hydroxy-5-(2-methyl-1H-imidazol-4-yl)-5,6,7,8-tetrahydro-1-naphthalenyl]methanesulfonamide;
N-[2-hydroxy-5-(1-methyl-1H-imidazol-5-yl)-5,6,7,8-tetrahydro-1-naphthalenyl]methanesulfonamide;
N-[2-hydroxy-5-(1-methyl-1H-imidazol-4-yl)-5,6,7,8-tetrahydro-1-naphthalenyl]methanesulfonamide;
N-[5-(1-ethyl-1H-imidazol-4-yl)-2-hydroxy-5,6,7, 8-tetrahydro-1-naphthalenyl]methanesulfonamide;
N-[2-hydroxy-5-(1-propyl-1H-imidazol-4-yl)-5,6,7,8-tetrahydro-1-naphthalenyl]methanesulfonamide;
N-[5-(1H-imidazol-4-yl)-5,6,7,8-tetrahydro-1-naphthalenyl]methanesulfonamide;
(R)-N-[5-(1H-imidazol-4-yl)-5,6,7,8-tetrahydro-1-naphthalenyl]methanesulfonamide;
(S)-N-[5-(1H-imidazol-4-yl)-5,6,7,8-tetrahydro-1-naphthalenyl]methanesulfonamide;
N-[5-(1H-imidazol-4-yl)-5,6,7,8-tetrahydro-1-naphthalenyl]ethanesulfonamide;
N-[5,6,7,8-tetrahydro-5-(1-methyl-1H-imidazol-4-yl)-1-naphthalenyl]methanesulfonamide;
N-[5,6,7,8-tetrahydro-5-(1H-imidazol-4-yl)-1-naphthalenyl]-N-methylmethanesulfonmamide;
N-[5,6,7,8-tetrahydro-5-(1H-imidazol-4-yl)-1-naphthalenyl]-2-methylethanesulfonamide;
N-[5,6,7,8-tetrahydro-5-(1H-imidazol-4-yl)-1-naphthalenyl]-2,2,2-trifluoroethanesulfonamide;
N-[5,6,7,8-tetrahydro-5-(1H-imidazol-4-yl)-4-methyl-1-naphthalenyl]methanesulfonamide;
N-[5,6,7,8-tetrahydro-4-hydroxy-5-(1H-imidazol-4-yl)-1-naphthalenyl]methanesulfonamide;
N-[5,6,7,8-tetrahydro-(1H-imidazol-4-yl)-4-methoxy-1-naphthalenyl]ethanesulfonamide;
N-[5,6,7,8-tetrahydro-(1H-imidazol-4-yl)-4-methoxy-1-naphthalenyl]methanesulfonamide;
N-[5,6,7,8-tetrahydro-(1H-imidazol-4-yl)-1-naphthalenyl]cyclopropanesulfonamide;
(+)-N-[5-(1H-imidazol-4-yl)-5,6,7,8-tetrahydro-1-naphthalenyl]ethanesulfonamide;
(−)-N-[5-(1H-imidazol-4-yl)-5,6,7,8-tetrahydro-1-naphthalenyl]ethanesulfonamide;
(−)-N-[5,6,7,8-tetrahydro-5-(1H-imidazol-4-yl)-1-naphthalenyl]-2,2,2-trifluoroethanesulfonamide;
(+)-N-[5,6,7,8-tetrahydro-5-(1H-imidazol-4-yl)-1-naphthalenyl]-2,2,2-trifluoroethanesulfonamide;
N-[4-chloro-5-(1H-imidazol-4-yl)-5,6,7,8-tetrahydro-1-naphthalenyl]ethanesulfonamide;
N-[4-chloro-5-(1H-imidazol-4-yl)-5,6,7,8-tetrahydro-1-naphthalenyl]methanesulfonamide;
N-[4-fluoro-5-(1H-imidazol-4-yl)-5,6,7,8-tetrahydro-1-naphthalenyl]methanesulfonamide;
N-[5-(1H-imidazol-4-yl)-5,6,7,8-tetrahydro-1-naphthalenyl]-3,5-dimethyl-4-isoxazolesulfonamide;
N-[5-(1H-imidazol-4-yl)-5,6,7,8-tetrahydro-1-naphthalenyl]-1-propanesulfonamide;
N-[5-(1H-imidazol-4-yl)-5,6,7,8-tetrahydro-1-naphthalenyl]-1-butanesulfonamide;
3-chloro-N-[5-(1H-imidazol-4-yl)-5,6,7,8-tetrahydro-1-naphthalenyl]-1-propanesulfonamide;
N-[5-(1H-imidazol-4-yl)-5,6,7,8-tetrahydro-1-naphthalenyl]-1-methyl-1H-imidazole-4-sulfonamide;
N-[5-(1H-imidazol-4-yl)-5,6,7,8-tetrahydro-1-naphthalenyl](phenyl)methanesulfonamide;
N-[5-(1H-imidazol-4-yl)-5,6,7,8-tetrahydro-1-naphthalenyl]-4-methylbenzenesulfonamide;
N-[5-(1H-imidazol-4-yl)-5,6,7,8-tetrahydro-1-naphthalenyl]-2-methylbenzenesulfonamide;
N-[5-(1H-imidazol-4-yl)-5,6,7,8-tetrahydro-1-naphthalenyl]-2-phenyl-1-ethenesulfonamide;
N-[5-(1H-imidazol-4-yl)-5,6,7,8-tetrahydro-1-naphthalenyl]-4-methoxybenzenesulfonamide;
5-chloro-N-[5-(1H-imidazol-4-yl)-5,6,7,8-tetrahydro-1-naphthalenyl]-2-thiophenesulfonamide;
N-[5-(1H-imidazol-4-yl)-5,6,7,8-tetrahydro-1-naphthalenyl]-8-quinolinesulfonamide;
5-chloro-N-[5-(1H-imidazol-4-yl)-5,6,7,8-tetrahydro-1-naphthalenyl]-1,3-dimethyl-1H-pyrazole-4-sulfonamide;
methyl 2-{[(5-(1H-imidazol-4-yl)-5,6,7,8-tetrahydro-1-naphthalenyl)amino] sulfonyl}-3-thiophenecarboxylate;
N-(5-{[(5-(1H-imidazol-4-yl)-5,6,7,8-tetrahydro-1-naphthalenyl)amino]sulfonyl}-4-methyl-1,3-thiazol-2-yl)acetamide;
5-chloro-N-[5-(1H-imidazol-4-yl)-5,6,7,8-tetrahydro-1-naphthalenyl]-3-methyl-2,3-dihydro-1-benzothiophene-2-sulfonamide;
N-[5-(2-methyl-1H-imidazol-4-yl)-5,6,7,8-tetrahydro-1-naphthalenyl] ethanesulfonamide;
N-[3-cyclohexyl-5-(1H-imidazol-4-yl)-5,6,7,8-tetrahydro-1-naphthalenyl]ethanesulfonamide;
N-[5-(1H-imidazol-4-yl)-2-methyl-5,6,7,8-tetrahydro-1-naphthalenyl]ethanesulfonamide;
N′-[5-(1H-imidazol-4-yl)-5,6,7,8-tetrahydro-1-naphthalenyl]-N,N-dimethylsulfamide;
N-[3-fluoro-5-(1H-imidazol-4-yl)-5,6,7,8-tetrahydro-1-naphthalenyl]ethanesulfonamide; and
N-[3-chloro-5-(1H-imidazol-4-yl)-5,6,7,8-tetrahydro-1-naphthalenyl]ethanesulfonamide.

15. A compound according to claim 4 wherein
A is —CH₂CH₂CH₂—; [symbol]
is a single bond;
R₁ is C(O)R₁₀; and
R₈ is hydrogen.

16. A compound according to claim 4 wherein
A is —CH₂CH₂CH₂—; [symbol]
is a single bond;
R₁ is S(O)₂R₉; and
R₈ is hydrogen.

17. A compound according to claim 16 selected from the group consisting of
N-[5-(1H-imidazol-4-yl)-6,7,8,9-tetrahydro-5H-benzo[a]cyclohepten-1-yl]methanesulfonamide and
N-[5-(1H-imidazol-4-yl)-6,7,8,9-tetrahydro-5H-benzo[a]cyclohepten-1-yl]ethanesulfonamide.

18. A compound according to claim 1 of formula III 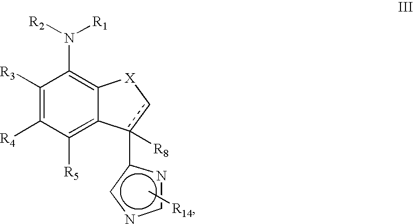
or a pharmaceutically acceptable salt thereof, wherein
X is selected from the group consisting of O, NR₁₁, and S(O)ₙ; and [symbol]
represents a single bond or a double bond.

19. A compound according to claim 1 of formula IV 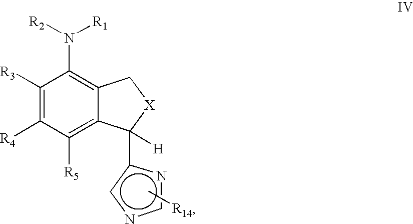
or a pharmaceutically acceptable salt thereof, wherein
X is selected from the group consisting of O, NR₁₁, and S(O)ₙ.

20. A compound according to claim 19 wherein
X is O; and
R₁ is C(O)R₁₀.

21. A compound according to claim 19 wherein
X is O; and
R₁ is S(O)₂R₉.

22. A compound according to claim 21 that is N-1-(1H-imidazol-4-yl)-1,3-dihydro-2-benzofuran-4-yl]ethane sulfonamide.

23. A compound according to claim 1 of formula V 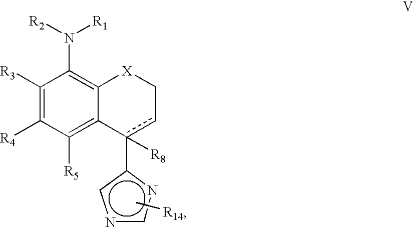
or a pharmaceutically acceptable salt thereof, wherein
X is selected from the group consisting of O, NR₁₁, and S(O)ₙ; and [symbol]
represents a single bond or a double bond.

24. A compound according to claim 23 wherein [symbol]
is a single bond;
R₁ is C(O)R₁₀; and
R₈ is hydrogen.

25. A compound according to claim 23 wherein [symbol]
is a single bond;
X is selected from the group consisting of O and S;
R₁ is S(O)₂R₉; and
R₈ is hydrogen.

26. A compound according to claim 25 that is selected from the group consisting of
N-[4-(H-imidazol-4-yl)-3,4-dihydro-2H-chromen-8-yl]methanesulfonamide;
N-[4-(1H-imidazol-4-yl)-3,4-dihydro-2H-chromen-8-yl]ethanesulfonamide;
N-[6-fluoro-4-(1H-imidazol-4-yl)-3,4-dihydro-2H-chromen-8-yl]ethanesulfonamide;
2,2,2-trifluoro-N-[4-(1H-imidazol-4-yl)-3,4-dihydro-2H-chromen-8-yl]ethanesulfonamide;
N-[4-(1H-imidazol-4-yl)-3,4-dihydro-2H-thiochromen-8-yl] ethanesulfonamide;
N-[6-fluoro-4-(1H-imidazol-4-yl)-3,4-dihydro-2H-chromen-8-yl]methanesulfonamide;
(+) N-[4-(1H-imidazol-4-yl)-3,4-dihydro-2H-chromen-8-yl]methanesulfonamide; and
(+) N-[4-(1H-imidazol-4-yl)-3,4-dihydro-2H-chromen-8-yl]ethanesulfonamide.

27. A compound according to claim 1 of formula VI 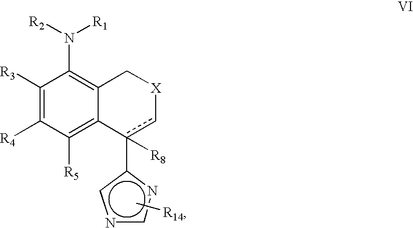
or a pharmaceutically acceptable salt thereof, wherein
X is selected from the group consisting of O, NR₁₁, and S(O)ₙ; and [symbol]
represents a single bond or a double bond.

28. A compound according to claim 1 of formula VII 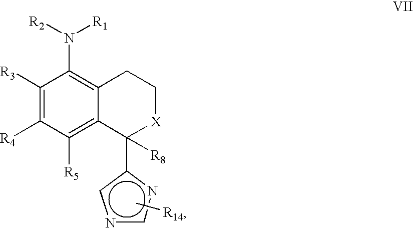
or a pharmaceutically acceptable salt thereof, wherein
X is selected from the group consisting of O, NR₁₁, and S(O)ₙ.

29. A compound according to claim 1 of formula VIII 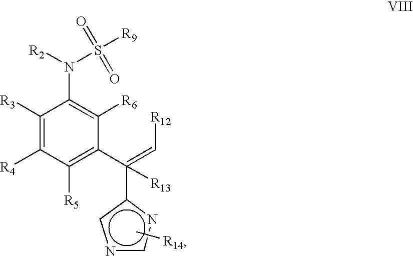
or a pharmaceutically acceptable salt thereof, wherein
R₆ is selected from the group consisting of hydrogen, lower alkoxy, lower alkenyl, lower alkyl, lower haloalkyl, halo, and hydroxy.

30. A compound according to claim 29 wherein
R₆ is hydrogen; and
R₁₂ and R₁₃ are independently selected from the group consisting of hydrogen, lower alkoxy, and lower alkyl.

31. A compound according to claim 30 selected from the group consisting of
N-[3-(1-(1H-imidazol-4-yl)vinyl)phenyl]ethanesulfonamide;
N-{3-[1-(1H-imidazol-4-yl)-2-methoxyethenyl]phenyl}ethanesulfonamide;
2,2,2-trifluoro-N-{3-[1-(1H-imidazol-4-yl)vinyl]phenyl}ethanesulfonamide;
N-{3-[1-(1H-imidazol-4-yl)vinyl]phenyl}methanesulfonamide; and
N-{3-[1-(1H-imidazol-4-yl)-2-methyl-1-propenyl]phenyl}ethanesulfonamide.

32. A compound according to claim 29 wherein
R₆ is hydrogen; and
R₁₂ and R₁₃ together with the carbon atom to which they are attached form a 3, 4, 5, 6, or 7 membered carbocyclic ring.

33. A compound according to claim 32 that is N-(3-(cyclohexylidene-(1H-imidazol-4-ylmethyl)phenyl)-1-ethanesulfonamide.

34. A pharmaceutical composition comprising a therapeutically effective amount of a compound of claim 1 in combination with a pharmaceutically acceptable carrier.

35. A method of activating a 1 adrenoceptors in a host mammal in need of such treatment comprising administering a therapeutically effective amount of a compound of claim 1.

36. A method of treating a disease in a host mammal in need of such treatment comprising administering a therapeutically effective amount of a compound of claim 1.

37. The method of claim 36 wherein the disease is urinary incontinence.

38. The method of claim 36 wherein the disease is retrograde ejaculation.
